SEQUENCE LISTING (7 sequences)

SEQ ID NO: 1 - DNA, 24 bp, Artificial Sequence
  Antisense primer HBV
tgcagaggtgaagcgaagtgcaca

SEQ ID NO: 2 - DNA, 24 bp, Artificial Sequence
  Sense primer HBV
gacgtcctttgtttacgtcccgtc

SEQ ID NO: 3 - DNA, 25 bp, Artificial Sequence
  Hybprobe FL HBV
acggggcgcacctctctttacgcgg

SEQ ID NO: 4 - DNA, 26 bp, Artificial Sequence
  Hybprobe PH HBV
ctccccgtctgtgccttctcatctgc

SEQ ID NO: 5 - DNA, 21 bp, Artificial Sequence
  HBV forward primer
ctgtaccaaaccttcggacgg

SEQ ID NO: 6 - DNA, 19 bp, Artificial Sequence
  HBV reverse primer
aggagaaacgggctgaggc

SEQ ID NO: 7 - DNA, 26 bp, Artificial Sequence
  FAM-labelled probe for HBV
ccatcatcctgggctttcggaaaatt

(19) United States
(12) Patent Application Publication
(10) Pub. No.: US 2022/0306647 A1
(43) Pub. Date: Sep. 29, 2022

(54) NOVEL INDOLE-2-CARBOXAMIDES ACTIVE AGAINST THE HEPATITUS B VIRUS (HBV)

(71) Applicant: AICURIS GMBH & CO. KG, Wuppertal (DE)

(72) Inventors: Susanne BONSMANN, Köln (DE); Alastair DONALD, Wuppertal (DE); Andreas URBAN, Sprockhövel (DE); Burkhard KLENKE, Wuppertal (DE); Jasper SPRINGER, Diepenveen (NL)

(73) Assignee: AICURIS GMBH & CO. KG, Wuppertal (DE)

(21) Appl. No.: 17/607,605

(22) PCT Filed: Apr. 29, 2020
(86) PCT No.: PCT/EP2020/061948

§ 371 Date: Oct. 29, 2021

(30) Foreign Application Priority Data
Apr. 30, 2019 (EP) 19172005.1
May 2, 2019 (EP) 19172398.0

(51) Int. Cl. C07D 498/04; C07D 487/04; C07D 471/04; C07D 471/14

(52) U.S. Cl. CPC C07D 498/04; C07D 487/04; C07D 471/04; C07D 471/14

(57) ABSTRACT

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes and intermediates for making the compounds.

### TECHNICAL FIELD

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes for making the compounds.

### BACKGROUND OF THE INVENTION

Chronic HBV infection is a significant global health problem, affecting over 5% of the world population (over 350 million people worldwide and 1.25 million individuals in the US). Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact.

The low cure rates of HBV are attributed at least in part to the fact that complete suppression of virus production is difficult to achieve with a single antiviral agent, and to the presence and persistence of covalently closed circular DNA (cccDNA) in the nucleus of infected hepatocytes. However, persistent suppression of HBV DNA slows liver disease progression and helps to prevent hepatocellular carcinoma (HCC).

Current therapy goals for HBV-infected patients are directed to reducing serum HBV DNA to low or undetectable levels, and to ultimately reducing or preventing the development of cirrhosis and HCC.

The HBV is an enveloped, partially double-stranded DNA (dsDNA) virus of the hepadnavirus family (Hepadnaviridae). HBV capsid protein (HBV-CP) plays essential roles in HBV replication. The predominant biological function of HBV-CP is to act as a structural protein to encapsidate pre-genomic RNA and form immature capsid particles, which spontaneously self-assemble from many copies of capsid protein dimers in the cytoplasm.

HBV-CP also regulates viral DNA synthesis through differential phosphorylation states of its C-terminal phosphorylation sites. Also, HBV-CP might facilitate the nuclear translocation of viral relaxed circular genome by means of the nuclear localization signals located in the arginine-rich domain of the C-terminal region of HBV-CP.

In the nucleus, as a component of the viral cccDNA mini-chromosome, HBV-CP could play a structural and regulatory role in the functionality of cccDNA mini-chromosomes. HBV-CP also interacts with viral large envelope protein in the endoplasmic reticulum (ER), and triggers the release of intact viral particles from hepatocytes.

HBV-CP related anti-HBV compounds have been reported. For example, phenylpropenamide derivatives, including compounds named AT-61 and AT-130 (Feld J. et al. Antiviral Res. 2007, 76, 168), and a class of thiazolidin-4-ones from Valeant (WO2006/033995), have been shown to inhibit pre-genomic RNA (pgRNA) packaging.

F. Hoffmann-La Roche AG have disclosed a series of 3-substituted tetrahydro-pyrazolo[1,5-a]pyrazines for the therapy of HBV (WO2016/113273, WO2017/198744, WO2018/011162, WO2018/011160, WO2018/011163).

Shanghai Hengrui Pharma have disclosed a series of heteroaryl piperazines for HBV therapy (WO2019/020070). Shanghai Longwood Biopharmaceuticals have disclosed a series of bicyclic heterocycles active against HBV (WO2018/202155).

Zhimeng Biopharma have disclosed pyrazole-oxazolidinone compounds as being active against HBV (WO2017/173999).

Heteroaryldihydropyrimidines (HAPs) were discovered in a tissue culture-based screening (Weber et al., Antiviral Res. 2002, 54, 69). These HAP analogs act as synthetic allosteric activators and are able to induce aberrant capsid formation that leads to degradation of HBV-CP (WO 99/54326, WO 00/58302, WO 01/45712, WO 01/6840). Further HAP analogs have also been described (J. Med. Chem. 2016, 59 (16), 7651-7666).

A subclass of HAPs from F. Hoffman-La Roche also shows activity against HBV (WO2014/184328, WO2015/132276, and WO2016/146598). A similar subclass from Sunshine Lake Pharma also shows activity against HBV (WO2015/144093). Further HAPs have also been shown to possess activity against HBV (WO2013/102655, Bioorg. Med. Chem. 2017, 25(3) pp. 1042-1056, and a similar subclass from Enanta Therapeutics shows similar activity (WO2017/011552). A further subclass from Medshine Discovery shows similar activity (WO2017/076286). A further subclass (Janssen Pharma) shows similar activity (WO2013/102655).

A subclass of pyridazones and triazinones (F. Hoffman-La Roche) also show activity against HBV (WO2016/023877), as do a subclass of tetrahydropyridopyridines (WO2016/177655). A subclass of tricyclic 4-pyridone-3-carboxylic acid derivatives from Roche also show similar anti-HBV activity (WO2017/013046).

A subclass of sulfamoyl-arylamides from Novira Therapeutics (now part of Johnson & Johnson Inc.) also shows activity against HBV (WO2013/006394, WO2013/096744, WO2014/165128, WO2014/184365, WO2015/109130, WO2016/089990, WO2016/109663, WO2016/109684, WO2016/109689, WO2017/059059). A similar subclass of thioether-arylamides (also from Novira Therapeutics) shows activity against HBV (WO2016/089990). Additionally, a subclass of aryl-azepanes (also from Novira Therapeutics) shows activity against HBV (WO2015/073774). A similar subclass of arylamides from Enanta Therapeutics show activity against HBV (WO2017/015451).

Sulfamoyl derivatives from Janssen Pharma have also been shown to possess activity against HBV (WO2014/033167, WO2014/033170, WO2017/001655, J. Med. Chem, 2018, 61(14) 6247-6260).

A subclass of glyoxamide substituted pyrrolamide derivatives also from Janssen Pharma have also been shown to possess activity against HBV (WO2015/011281). A similar class of glyoxamide substituted pyrrolamides (Gilead Sciences) has also been described (WO2018/039531).

A subclass of sulfamoyl- and oxalyl-heterobiaryls from Enanta Therapeutics also show activity against HBV (WO2016/161268, WO2016/183266, WO2017/015451, WO2017/136403 & US20170253609).

A subclass of aniline-pyrimidines from Assembly Biosciences also show activity against HBV (WO2015/057945, WO2015/172128). A subclass of fused tri-cycles from Assembly Biosciences (dibenzo-thiazepinones, dibenzo-diazepinones, dibenzo-oxazepinones) show activity against HBV (WO2015/138895, WO2017/048950). A further series from Assembly Biosciences (WO2016/168619) also show anti-HBV activity.

A series of cyclic sulfamides has been described as modulators of HBV-CP function by Assembly Biosciences (WO2018/160878).

Arbutus Biopharma have disclosed a series of benzamides for the therapy of HBV (WO2018/052967, WO2018/172852). Also disclosed are compositions and uses of similar compounds in combination with a CYP3A inhibitor (WO2019/046287).

A series of thiophene-2-carboxamides from the University of Missouri have been described as HBV inhibitors (US2019/0092742).

It was also shown that the small molecule bis-ANS acts as a molecular ‘wedge’ and interferes with normal capsid-protein geometry and capsid formation (Zlotnick A et al. J. Virol. 2002, 4848).

Problems that HBV direct acting antivirals may encounter are toxicity, mutagenicity, lack of selectivity, poor efficacy, poor bioavailability, low solubility and difficulty of synthesis. There is a thus a need for additional inhibitors for the treatment, amelioration or prevention of HBV that may overcome at least one of these disadvantages or that have additional advantages such as increased potency or an increased safety window.

Administration of such therapeutic agents to an HBV infected patient, either as monotherapy or in combination with other HBV treatments or ancillary treatments, will lead to significantly reduced virus burden, improved prognosis, diminished progression of the disease and/or enhanced seroconversion rates.

### SUMMARY OF THE INVENTION

Provided herein are compounds useful for the treatment or prevention of HBV infection in a subject in need thereof, and intermediates useful in their preparation. The subject matter of the invention is a compound of Formula I

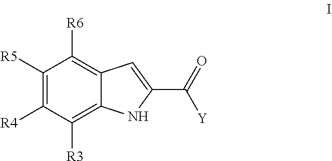

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

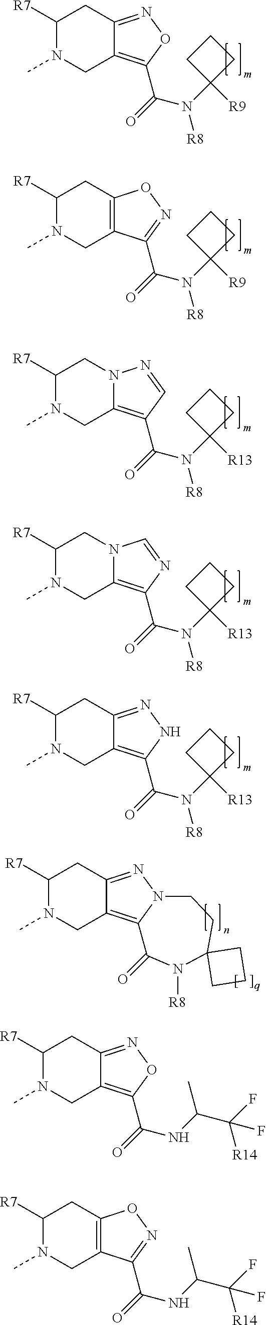

R7 is selected from the group comprising H, D, and C1-C6-alkyl
R8 is selected from the group comprising H, methyl, CD₃ ethyl, 2,2-difluoroethyl, 2,2,2-trifluoroethyl, 2-hydroxyethyl, CH₂CH₂—O—CH₂—C6-aryl, CH₂CH₂—O-C1-C3-alkyl, CH₂CH₂—N-(C1-C3-alkyl)₂, CH₂CH₂OCF₃, CH₂—C(O)—O-C1-C3-alkyl, 2-(4-methylpiperazin-1-yl)ethyl, 2-(morpholin-4-yl)ethyl and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, 1,3-dioxanyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂-0-C1-C3-alkyl optionally substituted with 1, 2 or 3 groups each independently selected from C1-C4-alkyl, OH, OCHF₂, OCF₃, carboxy, amino and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, CH₂-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
R14 is H or F
m is 0 or 1
n is 0, 1 or 2
q is 0 or 1,
wherein the dashed line is a covalent bond between C(O) and Y.

In one embodiment of the invention subject matter of the invention is a compound of Formula I in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

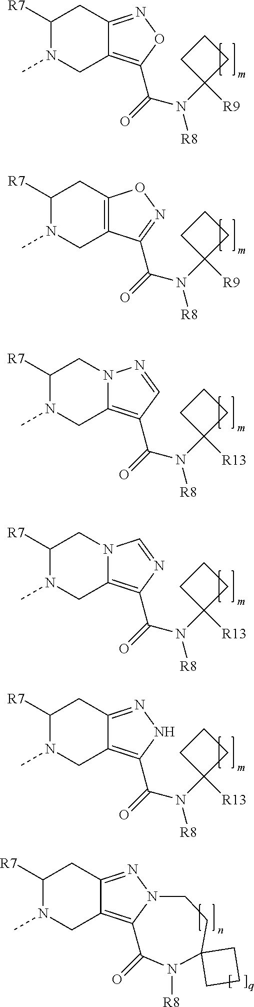

R7 is selected from the group comprising H, D, and C1-C6-alkyl
R8 is selected from the group comprising H, methyl, CD₃, ethyl, 2,2-difluoroethyl, 2,2,2-trifluoroethyl, 2-hydroxyethyl, and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂—O—C1-C3-alkyl optionally substituted with 1, 2 or 3 groups each independently selected from C1-C4-alkyl, OH, OCHF₂, OCF₃, carboxy and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
m is 0 or 1
n is 0, 1 or 2
q is 0 or 1,
wherein the dashed line is a covalent bond between C(O) and Y.

In one embodiment of the invention subject matter of the invention is a compound of Formula I in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

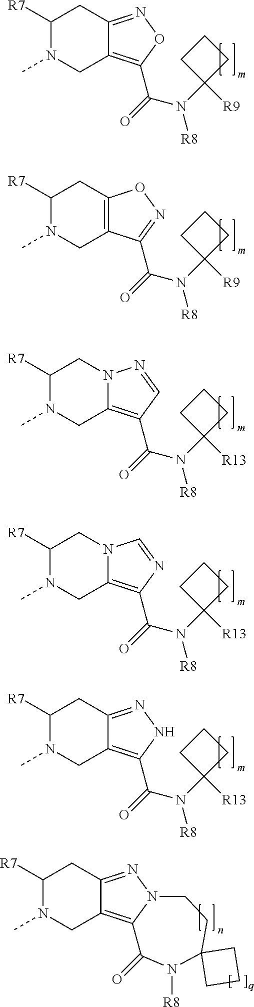

R7 is selected from the group comprising H, D, and C1-C6-alkyl
R8 is selected from the group comprising H, methyl, CD₃, ethyl, 2,2-difluoroethyl, 2,2,2-trifluoroethyl, 2-hydroxyethyl, and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂—O—C1-C3-alkyl optionally substituted with 1, 2 or 3 groups each independently selected from C1-C4-alkyl, OH, OCHF₂, OCF₃, carboxy and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
m is 0 or 1
n is 0, 1 or 2
q is 0 or 1,
   wherein the dashed line is a covalent bond between C(O) and Y.

In one embodiment of the invention subject matter of the invention is a compound of Formula I in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

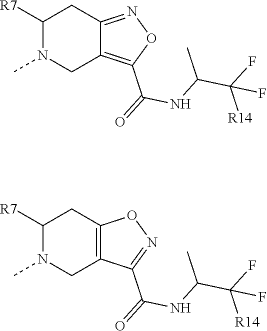

R7 is selected from the group comprising H, D, and C1-C6-alkyl
R14 is H or F
   wherein the dashed line is a covalent bond between C(O) and Y.

In one embodiment of the invention subject matter of the invention are stereoisomers of a compound of Formula I in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

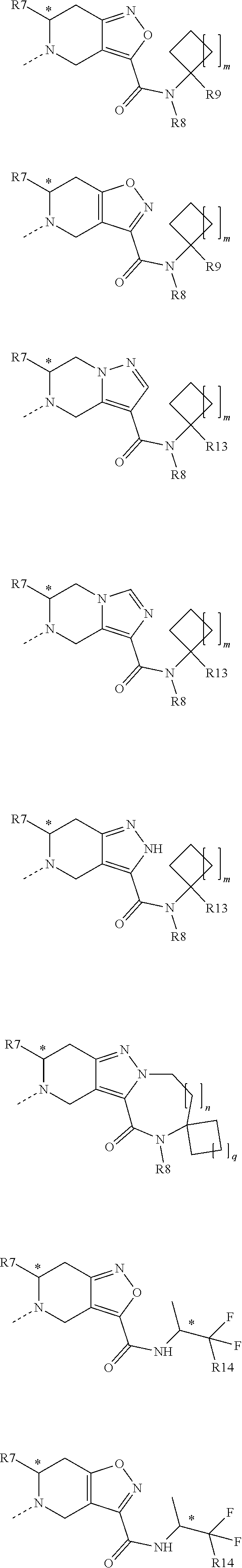

R7 is C1-C6-alkyl
R8 is selected from the group comprising H, methyl, CD₃ ethyl, 2,2-difluoroethyl, 2,2,2-trifluoroethyl, 2-hydroxyethyl, CH₂CH₂—O—CH₂—C6-aryl, CH₂CH₂—O—C1-C3-alkyl, CH₂CH₂—N-(C1-C3-alkyl)₂, CH₂CH₂OCF₃, CH₂—C(O)—O—C1-C3-alkyl, 2-(4-methylpiperazin-1-yl)ethyl, 2-(morpholin-4-yl)ethyl and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, 1,3-dioxanyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂—O—C1-C3-alkyl optionally substituted with 1, 2 or 3 groups each independently selected from C1-C4-alkyl, OH, OCHF₂, OCF₃, carboxy, amino and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, CH₂-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
R14 is H or F
m is 0 or 1
n is 0, 1 or 2
q is 0 or 1,
   wherein the dashed line is a covalent bond between C(O) and Y.

In one embodiment of the invention subject matter of the invention are stereoisomers of a compound of Formula I in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

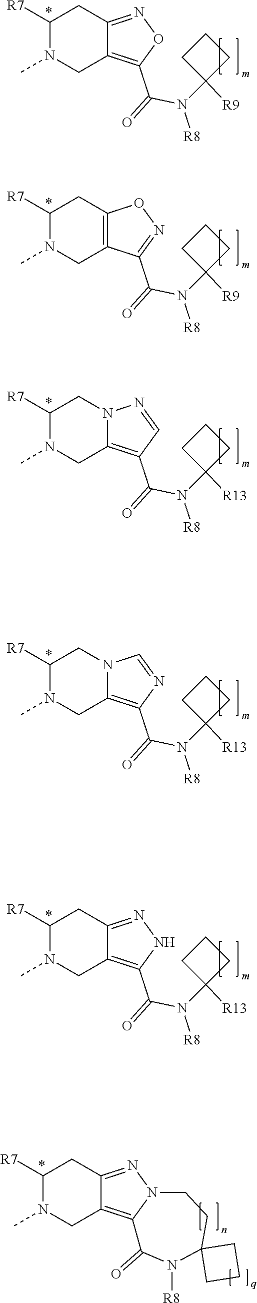

R7 is C1-C6-alkyl
R8 is selected from the group comprising H, methyl, CD₃, ethyl, 2,2-difluoroethyl, 2,2,2-trifluoroethyl, 2-hydroxyethyl, and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂—O—C1-C3-alkyl optionally substituted with 1, 2 or 3 groups each independently selected from C1-C4-alkyl, OH, OCHF₂, OCF₃, carboxy and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
m is 0 or 1
n is 0, 1 or 2
q is 0 or 1,
   wherein the dashed line is a covalent bond between C(O) and Y.

In one embodiment of the invention subject matter of the invention are stereoisomers of a compound of Formula I in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

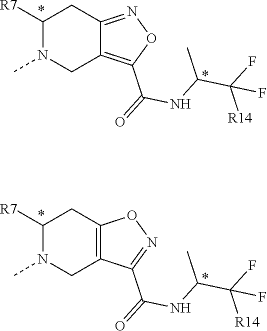

R7 is C1-C6-alkyl
R14 is H or F
   wherein the dashed line is a covalent bond between C(O) and Y.

One embodiment of the invention is a compound of Formula I or a pharmaceutically acceptable salt thereof according to the invention, for use in the prevention or treatment of an HBV infection in subject.

One embodiment of the invention is a pharmaceutical composition comprising a compound of Formula I or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula I or a pharmaceutically acceptable salt thereof according to the present invention.

A further embodiment of the invention is a compound of Formula I or a pharmaceutically acceptable salt thereof according to the invention, for use in the prevention or treatment of an HBV infection in subject in need thereof.

A further embodiment of the invention is a compound of Formula IIa or a pharmaceutically acceptable salt thereof according to the invention, for use in the prevention or treatment of an HBV infection in subject in need thereof.

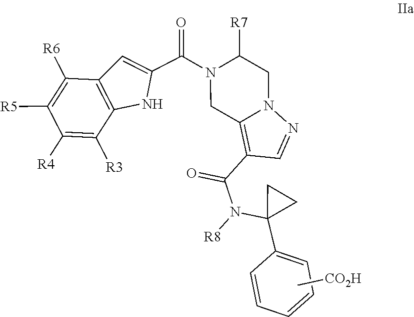

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

In one embodiment of the invention subject matter of the invention is a compound of Formula IIa in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

One embodiment of the invention is a compound of Formula IIa or a pharmaceutically acceptable salt thereof according to the invention, for use in the prevention or treatment of an HBV infection in subject.

One embodiment of the invention is a pharmaceutical composition comprising a compound of Formula IIa or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIa or a pharmaceutically acceptable salt thereof according to the present invention.

A further embodiment of the invention is a compound of Formula IIa or a pharmaceutically acceptable salt thereof according to the invention, for use in the prevention or treatment of an HBV infection in subject in need thereof.

A further embodiment of the invention is a compound of Formula IIb or a pharmaceutically acceptable salt thereof according to the invention, for use in the prevention or treatment of an HBV infection in subject in need thereof.

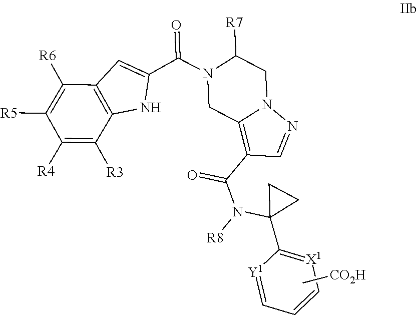

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X¹ and Y¹ are for each position independently selected from CH and N

In one embodiment of the invention subject matter of the invention is a compound of Formula IIb in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X¹ and Y¹ are for each position independently selected from CH and N

One embodiment of the invention is a compound of Formula IIb or a pharmaceutically acceptable salt thereof according to the invention, for use in the prevention or treatment of an HBV infection in subject.

One embodiment of the invention is a pharmaceutical composition comprising a compound of Formula IIb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIb or a pharmaceutically acceptable salt thereof according to the present invention.

A further embodiment of the invention is a compound of Formula IIb or a pharmaceutically acceptable salt thereof according to the invention, for use in the prevention or treatment of an HBV infection in subject in need thereof.

A further embodiment of the invention is a compound of Formula IIc or a pharmaceutically acceptable salt thereof according to the invention, for use in the prevention or treatment of an HBV infection in subject in need thereof.

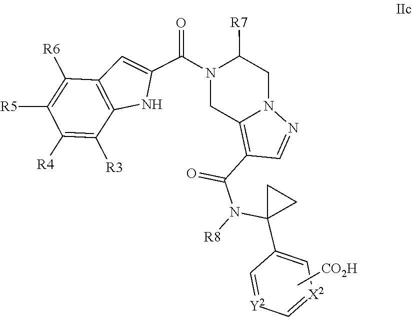

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X² and Y² are for each position independently selected from CH and N

### Position 1

In one embodiment of the invention subject matter of the invention is a compound of Formula IIIc in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X² and Y² are for each position independently selected from CH and N

One embodiment of the invention is a compound of Formula IIc or a pharmaceutically acceptable salt thereof according to the invention, for use in the prevention or treatment of an HBV infection in subject.

One embodiment of the invention is a pharmaceutical composition comprising a compound of Formula IIc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIc or a pharmaceutically acceptable salt thereof according to the present invention.

A further embodiment of the invention is a compound of Formula IIc or a pharmaceutically acceptable salt thereof according to the invention, for use in the prevention or treatment of an HBV infection in subject in need thereof.

A further embodiment of the invention is a compound of Formula IIIa or a pharmaceutically acceptable salt thereof according to the invention, for use in the prevention or treatment of an HBV infection in subject in need thereof.

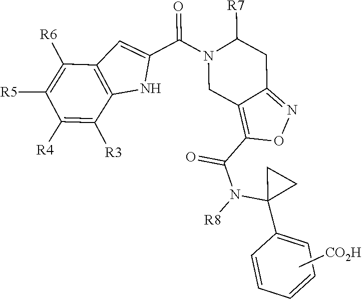

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

In one embodiment of the invention subject matter of the invention is a compound of Formula IIIa in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

One embodiment of the invention is a compound of Formula IIIa or a pharmaceutically acceptable salt thereof according to the invention, for use in the prevention or treatment of an HBV infection in subject.

One embodiment of the invention is a pharmaceutical composition comprising a compound of Formula IIIa or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIIa or a pharmaceutically acceptable salt thereof according to the present invention.

A further embodiment of the invention is a compound of Formula IIIa or a pharmaceutically acceptable salt thereof according to the invention, for use in the prevention or treatment of an HBV infection in subject in need thereof.

A further embodiment of the invention is a compound of Formula IIIb or a pharmaceutically acceptable salt thereof according to the invention, for use in the prevention or treatment of an HBV infection in subject in need thereof.

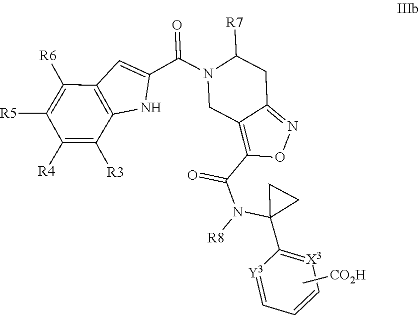

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X³ and Y³ are for each position independently selected from CH and N

In one embodiment of the invention subject matter of the invention is a compound of Formula IIIb in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X³ and Y³ are for each position independently selected from CH and N

One embodiment of the invention is a compound of Formula IIIb or a pharmaceutically acceptable salt thereof according to the invention, for use in the prevention or treatment of an HBV infection in subject.

One embodiment of the invention is a pharmaceutical composition comprising a compound of Formula IIIb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIIb or a pharmaceutically acceptable salt thereof according to the present invention.

A further embodiment of the invention is a compound of Formula IIIb or a pharmaceutically acceptable salt thereof according to the invention, for use in the prevention or treatment of an HBV infection in subject in need thereof.

A further embodiment of the invention is a compound of Formula IIIc or a pharmaceutically acceptable salt thereof according to the invention, for use in the prevention or treatment of an HBV infection in subject in need thereof.

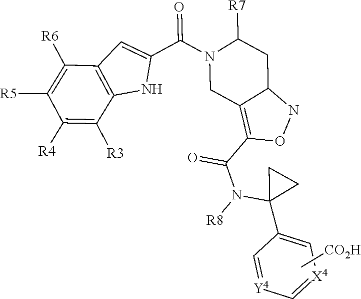

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁴ and Y⁴ are for each position independently selected from CH and N

In one embodiment of the invention subject matter of the invention is a compound of Formula IIIc in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁴ and Y⁴ are for each position independently selected from CH and N

One embodiment of the invention is a compound of Formula IIIc or a pharmaceutically acceptable salt thereof according to the invention, for use in the prevention or treatment of an HBV infection in subject.

One embodiment of the invention is a pharmaceutical composition comprising a compound of Formula IIIc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIIc or a pharmaceutically acceptable salt thereof according to the present invention.

A further embodiment of the invention is a compound of Formula IIIc or a pharmaceutically acceptable salt thereof according to the invention, for use in the prevention or treatment of an HBV infection in subject in need thereof.

A further embodiment of the invention is a compound of Formula IVa or a pharmaceutically acceptable salt thereof according to the invention, for use in the prevention or treatment of an HBV infection in subject in need thereof.

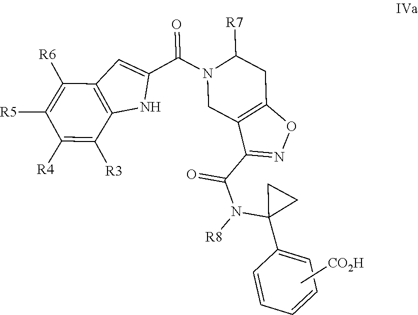

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

In one embodiment of the invention subject matter of the invention is a compound of Formula IVa in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

One embodiment of the invention is a compound of Formula IVa or a pharmaceutically acceptable salt thereof according to the invention, for use in the prevention or treatment of an HBV infection in subject.

One embodiment of the invention is a pharmaceutical composition comprising a compound of Formula IVa or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IVa or a pharmaceutically acceptable salt thereof according to the present invention.

A further embodiment of the invention is a compound of Formula IVa or a pharmaceutically acceptable salt thereof according to the invention, for use in the prevention or treatment of an HBV infection in subject in need thereof.

A further embodiment of the invention is a compound of Formula IVb or a pharmaceutically acceptable salt thereof according to the invention, for use in the prevention or treatment of an HBV infection in subject in need thereof.

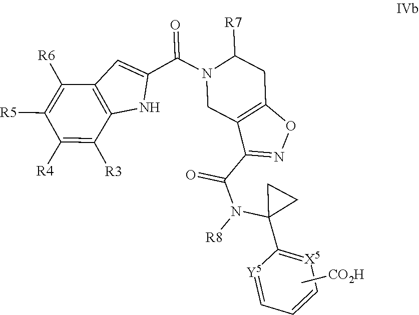

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁵ and Y⁵ are for each position independently selected from CH and N

In one embodiment of the invention subject matter of the invention is a compound of Formula IVb in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁵ and Y⁵ are for each position independently selected from CH and N

One embodiment of the invention is a compound of Formula IVb or a pharmaceutically acceptable salt thereof according to the invention, for use in the prevention or treatment of an HBV infection in subject.

One embodiment of the invention is a pharmaceutical composition comprising a compound of Formula IVb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IVb or a pharmaceutically acceptable salt thereof according to the present invention.

A further embodiment of the invention is a compound of Formula IVb or a pharmaceutically acceptable salt thereof according to the invention, for use in the prevention or treatment of an HBV infection in subject in need thereof.

A further embodiment of the invention is a compound of Formula IVc or a pharmaceutically acceptable salt thereof according to the invention, for use in the prevention or treatment of an HBV infection in subject in need thereof.

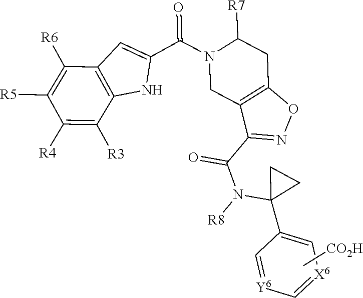

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁶ and Y⁶ are for each position independently selected from CH and N

In one embodiment of the invention subject matter of the invention is a compound of Formula IVc in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁶ and Y⁶ are for each position independently selected from CH and N

One embodiment of the invention is a compound of Formula IVc or a pharmaceutically acceptable salt thereof according to the invention, for use in the prevention or treatment of an HBV infection in subject.

One embodiment of the invention is a pharmaceutical composition comprising a compound of Formula IVc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IVc or a pharmaceutically acceptable salt thereof according to the present invention.

A further embodiment of the invention is a compound of Formula IVc or a pharmaceutically acceptable salt thereof according to the invention, for use in the prevention or treatment of an HBV infection in subject in need thereof.

A further embodiment of the invention is a compound of Formula Va or a pharmaceutically acceptable salt thereof according to the invention, for use in the prevention or treatment of an HBV infection in subject in need thereof.

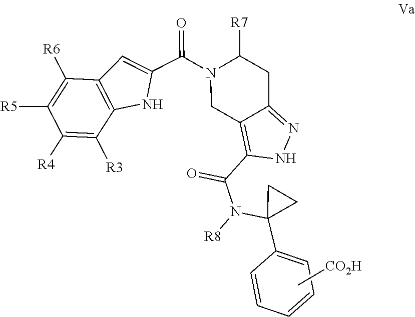

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

In one embodiment of the invention subject matter of the invention is a compound of Formula Va in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

One embodiment of the invention is a compound of Formula Va or a pharmaceutically acceptable salt thereof according to the invention, for use in the prevention or treatment of an HBV infection in subject.

One embodiment of the invention is a pharmaceutical composition comprising a compound of Formula Va or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Va or a pharmaceutically acceptable salt thereof according to the present invention.

A further embodiment of the invention is a compound of Formula Va or a pharmaceutically acceptable salt thereof according to the invention, for use in the prevention or treatment of an HBV infection in subject in need thereof.

A further embodiment of the invention is a compound of Formula Vb or a pharmaceutically acceptable salt thereof according to the invention, for use in the prevention or treatment of an HBV infection in subject in need thereof.

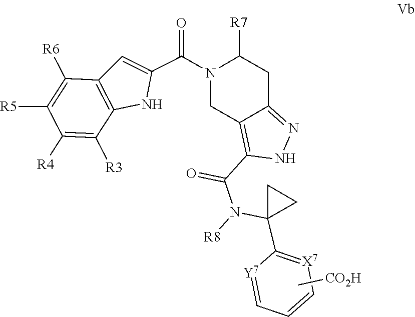

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁷ and Y⁷ are for each position independently selected from CH and N

In one embodiment of the invention subject matter of the invention is a compound of Formula Vb in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁷ and Y⁷ are for each position independently selected from CH and N

One embodiment of the invention is a compound of Formula Vb or a pharmaceutically acceptable salt thereof according to the invention, for use in the prevention or treatment of an HBV infection in subject.

One embodiment of the invention is a pharmaceutical composition comprising a compound of Formula Vb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Vb or a pharmaceutically acceptable salt thereof according to the present invention.

A further embodiment of the invention is a compound of Formula Vb or a pharmaceutically acceptable salt thereof according to the invention, for use in the prevention or treatment of an HBV infection in subject in need thereof.

A further embodiment of the invention is a compound of Formula Vc or a pharmaceutically acceptable salt thereof according to the invention, for use in the prevention or treatment of an HBV infection in subject in need thereof.

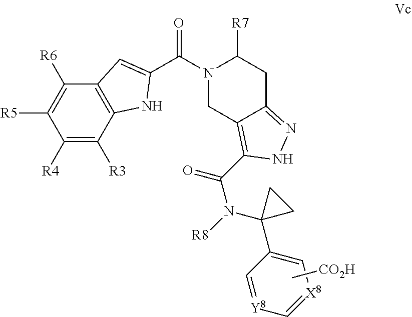

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁸ and Y⁸ are for each position independently selected from CH and N

In one embodiment of the invention subject matter of the invention is a compound of Formula Vc in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁸ and Y⁸ are for each position independently selected from CH and N

One embodiment of the invention is a compound of Formula Vc or a pharmaceutically acceptable salt thereof according to the invention, for use in the prevention or treatment of an HBV infection in subject.

One embodiment of the invention is a pharmaceutical composition comprising a compound of Formula Vc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Vc or a pharmaceutically acceptable salt thereof according to the present invention.

A further embodiment of the invention is a compound of Formula Vc or a pharmaceutically acceptable salt thereof according to the invention, for use in the prevention or treatment of an HBV infection in subject in need thereof.

A further embodiment of the invention is a compound of Formula VIa or a pharmaceutically acceptable salt thereof according to the invention, for use in the prevention or treatment of an HBV infection in subject in need thereof.

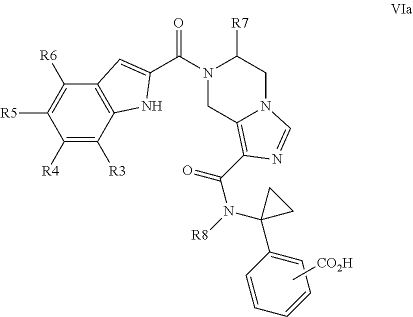

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

In one embodiment of the invention subject matter of the invention is a compound of Formula VIa in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl

One embodiment of the invention is a compound of Formula VIa or a pharmaceutically acceptable salt thereof according to the invention, for use in the prevention or treatment of an HBV infection in subject.

One embodiment of the invention is a pharmaceutical composition comprising a compound of Formula VIa or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VIa or a pharmaceutically acceptable salt thereof according to the present invention.

A further embodiment of the invention is a compound of Formula VIa or a pharmaceutically acceptable salt thereof according to the invention, for use in the prevention or treatment of an HBV infection in subject in need thereof.

A further embodiment of the invention is a compound of Formula VIb or a pharmaceutically acceptable salt thereof according to the invention, for use in the prevention or treatment of an HBV infection in subject in need thereof.

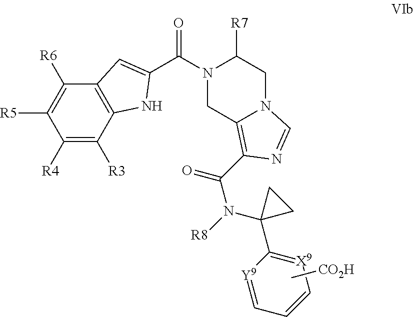

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁹ and Y⁹ are for each position independently selected from CH and N

In one embodiment of the invention subject matter of the invention is a compound of Formula VIb in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁹ and Y⁹ are for each position independently selected from CH and N

One embodiment of the invention is a compound of Formula VIb or a pharmaceutically acceptable salt thereof according to the invention, for use in the prevention or treatment of an HBV infection in subject.

One embodiment of the invention is a pharmaceutical composition comprising a compound of Formula VIb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VIb or a pharmaceutically acceptable salt thereof according to the present invention.

A further embodiment of the invention is a compound of Formula VIb or a pharmaceutically acceptable salt thereof according to the invention, for use in the prevention or treatment of an HBV infection in subject in need thereof.

A further embodiment of the invention is a compound of Formula VIc or a pharmaceutically acceptable salt thereof according to the invention, for use in the prevention or treatment of an HBV infection in subject in need thereof.

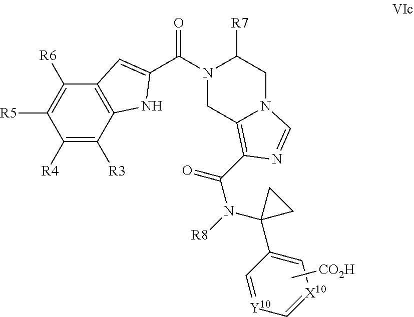

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X¹⁰ and Y¹⁰ are for each position independently selected from CH and N

In one embodiment of the invention subject matter of the invention is a compound of Formula VIc in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X¹⁰ and Y¹⁰ are for each position independently selected from CH and N

One embodiment of the invention is a compound of Formula VIc or a pharmaceutically acceptable salt thereof according to the invention, for use in the prevention or treatment of an HBV infection in subject.

One embodiment of the invention is a pharmaceutical composition comprising a compound of Formula VIc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VIc or a pharmaceutically acceptable salt thereof according to the present invention.

A further embodiment of the invention is a compound of Formula VIc or a pharmaceutically acceptable salt thereof according to the invention, for use in the prevention or treatment of an HBV infection in subject in need thereof.

A further embodiment of the invention is a compound of Formula VII or a pharmaceutically acceptable salt thereof according to the invention, for use in the prevention or treatment of an HBV infection in subject in need thereof.

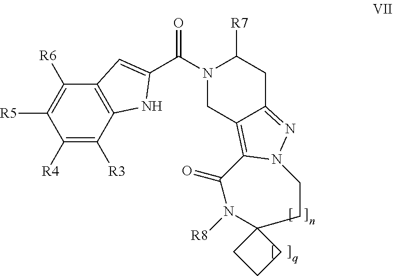

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
q is 0, or 1
n is 0, 1 or 2

In one embodiment of the invention subject matter of the invention is a compound of Formula VII in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
m is 0, 1, or 2
n is 0, 1 or 2

One embodiment of the invention is a compound of Formula VII or a pharmaceutically acceptable salt thereof according to the invention, for use in the prevention or treatment of an HBV infection in subject.

One embodiment of the invention is a pharmaceutical composition comprising a compound of Formula VII or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VII or a pharmaceutically acceptable salt thereof according to the present invention.

A further embodiment of the invention is a compound of Formula VII or a pharmaceutically acceptable salt thereof according to the invention, for use in the prevention or treatment of an HBV infection in subject in need thereof.

A further embodiment of the invention is a compound of Formula IX or a pharmaceutically acceptable salt thereof according to the invention, for use in the prevention or treatment of an HBV infection in subject in need thereof

in which

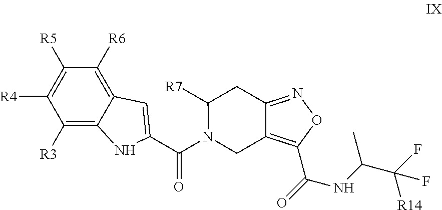

R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D, and C1-C6-alkyl R14 is H or F.

In one embodiment of the invention subject matter of the invention is a compound of Formula IX in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D, and C1-C6-alkyl R14 is H or F.

One embodiment of the invention is a compound of Formula IX or a pharmaceutically acceptable salt thereof according to the invention, for use in the prevention or treatment of an HBV infection in subject.

One embodiment of the invention is a pharmaceutical composition comprising a compound of Formula IX or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IX or a pharmaceutically acceptable salt thereof according to the present invention.

A further embodiment of the invention is a compound of Formula IX or a pharmaceutically acceptable salt thereof according to the invention, for use in the prevention or treatment of an HBV infection in subject in need thereof.

A further embodiment of the invention is a compound of Formula X or a pharmaceutically acceptable salt thereof according to the invention, for use in the prevention or treatment of an HBV infection in subject in need thereof

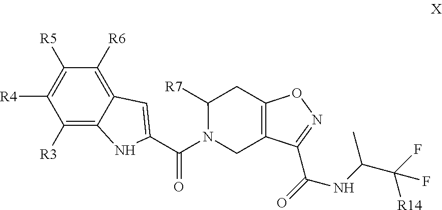

in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D, and C1-C6-alkyl
R14 is H or F.

In one embodiment of the invention subject matter of the invention is a compound of Formula X in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D, and C1-C6-alkyl
R14 is H or F.

One embodiment of the invention is a compound of Formula X or a pharmaceutically acceptable salt thereof according to the invention, for use in the prevention or treatment of an HBV infection in subject.

One embodiment of the invention is a pharmaceutical composition comprising a compound of Formula X or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula X or a pharmaceutically acceptable salt thereof according to the present invention.

A further embodiment of the invention is a compound of Formula X or a pharmaceutically acceptable salt thereof according to the invention, for use in the prevention or treatment of an HBV infection in subject in need thereof.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof. All before mentioned doses refer to daily doses per patient.

In general it is contemplated that an antiviral effective daily amount would be from about 0.01 to about 50 mg/kg, or about 0.01 to about 30 mg/kg body weight. It may be appropriate to administer the required dose as two, three, four or more sub-doses at appropriate intervals throughout the day. Said sub-doses may be formulated as unit dosage forms, for example containing about 1 to about 500 mg, or about 1 to about 300 mg or about 1 to about 100 mg, or about 2 to about 50 mg of active ingredient per unit dosage form.

The compounds of the invention may, depending on their structure, exist as salts, solvates or hydrates. The invention therefore also encompasses the salts, solvates or hydrates and respective mixtures thereof.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers.

Subject-matter of the present invention is a compound of Formula I, IIa, IIb, IIc, IIIa, IIIb, IIIc, IVa, IVb, IVc, Va, Vb, Vc, VIa, VIb, VIc, VII, IX, X or a pharmaceutically acceptable salt thereof or a solvate or a hydrate of said compound or a pharmaceutically acceptable salt of said solvate or hydrate or a prodrug of said compound or a pharmaceutically acceptable salt of said prodrug or a solvate or a hydrate of said prodrug or a pharmaceutically acceptable salt of said solvate or a hydrate of said prodrug.

Subject-matter of the present invention is a compound of Formula I, IIa, IIb, IIc, IIIa, IIIb, IIIc, IVa, IVb, IVc, Va, Vb, Vc, VIa, VIb, VIc, VII, IX, X or a pharmaceutically acceptable salt thereof or a solvate or a hydrate of said compound or a pharmaceutically acceptable salt of said solvate or hydrate or a prodrug of said compound or a pharmaceutically acceptable salt of said prodrug or a solvate or a hydrate of said prodrug or a pharmaceutically acceptable salt of said solvate or a hydrate of said prodrug for use in the prevention or treatment of an HBV infection in subject.

Subject-matter of the present invention is also a pharmaceutical composition comprising a compound of Formula I, IIa, IIb, IIc, IIIa, IIIb, IIIc, IVa, IVb, IVc, Va, Vb, Vc, VIa, VIb, VIc, VII, IX, X or a pharmaceutically acceptable salt thereof or a solvate or a hydrate of said compound or a pharmaceutically acceptable salt of said solvate or hydrate or a prodrug of said compound or a pharmaceutically acceptable salt of said prodrug or a solvate or a hydrate of said prodrug or a pharmaceutically acceptable salt of said solvate or a hydrate of said prodrug, together with a pharmaceutically acceptable carrier.

Subject-matter of the present invention is also a method of treating an HBV infection in an individual in need thereof, comprising administering to the individual a therapeutically effective amount of a compound of Formula I, IIa, IIb, IIc, IIIa, IIIb, IIIc, IVa, IVb, IVc, Va, Vb, Vc, VIa, VIb, VIc, VII, IX, X or a pharmaceutically acceptable salt thereof or a solvate or a hydrate of said compound or a pharmaceutically acceptable salt of said solvate or hydrate or a prodrug of said compound or a pharmaceutically acceptable salt of said prodrug or a solvate or a hydrate of said prodrug or a pharmaceutically acceptable salt of said solvate or a hydrate of said prodrug.

Subject matter of the present invention is also a method of preparing the compounds of the present invention. Subject matter of the invention is, thus, a method for the preparation of a compound of Formula I according to the present invention by reacting a compound of Formula VIII

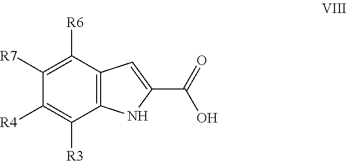

in which R3, R4, R5 and R6 are for each position independently selected from the group comprising H, F, Cl, Br, I, CF₃, CF₂H, C1-C4-alkyl, CF₂CH₃, cyclopropyl, cyano, and nitro, with a compound selected from

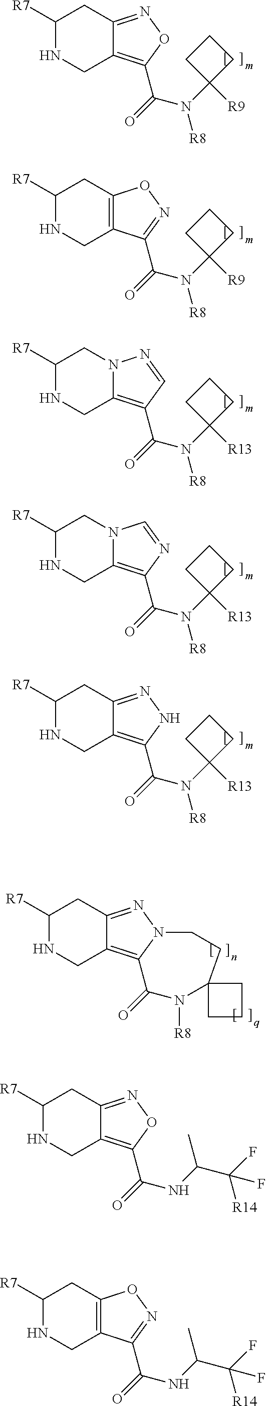

in which
R7 is selected from the group comprising H, D, and C1-C6-alkyl
R8 is selected from the group comprising H, methyl, CD₃ ethyl, 2,2-difluoroethyl, 2,2,2-trifluoroethyl, 2-hydroxyethyl, CH₂CH₂—O—CH₂—C6-aryl, CH₂CH₂—O—C1-C3-alkyl, CH₂CH₂—N-(C1-C3-alkyl)₂, CH₂CH₂OCF₃, CH₂—C(O)—O—C1-C3-alkyl, 2-(4-methylpiperazin-1-yl)ethyl, 2-(morpholin-4-yl)ethyl and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, 1,3-dioxanyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂—O—C1-C3-alkyl optionally substituted with 1, 2 or 3 groups each independently selected from C1-C4-alkyl, OH, OCHF₂, OCF₃, carboxy, amino and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, CH₂-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
R14 is H or F
m is 0 or 1
n is 0, 1 or 2
q is 0 or 1.

In one embodiment subject matter of the invention is a method for the preparation of a compound of Formula I according to the present invention by reacting a compound of Formula VIII

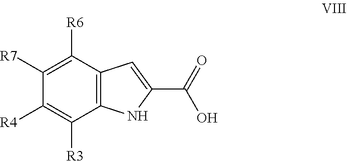

in which R3, R4, R5 and R6 are for each position independently selected from the group comprising H, F, Cl, Br, I, CF₃, CF₂H, C1-C4-alkyl, CF₂CH₃, cyclopropyl, cyano, and nitro, with a compound selected from

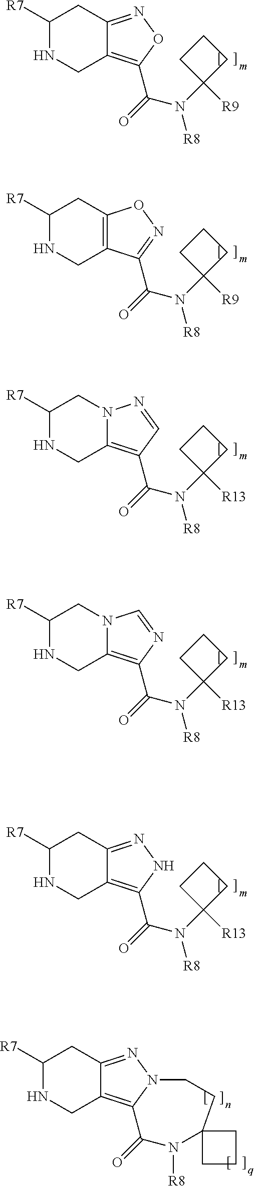

in which
R7 is selected from the group comprising H, D, and C1-C6-alkyl
R8 is selected from the group comprising H, methyl, CD₃ ethyl, 2,2-difluoroethyl, 2,2,2-trifluoroethyl, 2-hydroxyethyl, and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂—O—C1-C3-alkyl optionally substituted with 1, 2 or 3 groups each independently selected from C1-C4-alkyl, OH, OCHF₂, OCF₃, carboxy and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
m is 0 or 1
n is 0, 1 or 2
q is 0 or 1.

### Definitions

Listed below are definitions of various terms used to describe this invention. These definitions apply to the terms as they are used throughout this specification and claims unless otherwise limited in specific instances either individually or as part of a larger group.

Unless defined otherwise all technical and scientific terms used herein generally have the same meaning as commonly understood by one of ordinary skill in the art to which this invention belongs. Generally the nomenclature used herein and the laboratory procedures in cell culture, molecular genetics, organic chemistry and peptide chemistry are those well-known and commonly employed in the art.

As used herein the articles “a” and “an” refer to one or to more than one (i.e. to at least one) of the grammatical object of the article. By way of example, “an element” means one element or more than one element. Furthermore, use of the term “including” as well as other forms such as “include”, “includes” and “included”, is not limiting.

As used herein the term “capsid assembly modulator” refers to a compound that disrupts or accelerates or inhibits or hinders or delays or reduces or modifies normal capsid assembly (e.g. during maturation) or normal capsid disassembly (e.g. during infectivity) or perturbs capsid stability, thereby inducing aberrant capsid morphology or aberrant capsid function. In one embodiment, a capsid assembly modulator accelerates capsid assembly or disassembly thereby inducing aberrant capsid morphology. In another embodiment a capsid assembly modulator interacts (e.g. binds at an active site, binds at an allosteric site or modifies and/or hinders folding and the like), with the major capsid assembly protein (HBV-CP), thereby disrupting capsid assembly or disassembly. In yet another embodiment a capsid assembly modulator causes a perturbation in the structure or function of HBV-CP (e.g. the ability of HBV-CP to assemble, disassemble, bind to a substrate, fold into a suitable conformation or the like which attenuates viral infectivity and/or is lethal to the virus).

As used herein the term “treatment” or “treating” is defined as the application or administration of a therapeutic agent i.e., a compound of the invention (alone or in combination with another pharmaceutical agent) to a patient, or application or administration of a therapeutic agent to an isolated tissue or cell line from a patient (e.g. for diagnosis or ex vivo applications) who has an HBV infection, a symptom of HBV infection, or the potential to develop an HBV infection with the purpose to cure, heal, alleviate, relieve, alter, remedy, ameliorate, improve or affect the HBV infection, the symptoms of HBV infection or the potential to develop an HBV infection. Such treatments may be specifically tailored or modified based on knowledge obtained from the field of pharmacogenomics.

As used herein the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein the term “patient”, “individual” or “subject” refers to a human or a non-human mammal. Non-human mammals include for example livestock and pets such as ovine, bovine, porcine, feline, and murine mammals. Preferably the patient, subject, or individual is human.

As used herein the terms “effective amount”, “pharmaceutically effective amount”, and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein the term “pharmaceutically acceptable” refers to a material such as a carrier or diluent which does not abrogate the biological activity or properties of the compound and is relatively non-toxic i.e. the material may be administered to an individual without causing undesirable biological effects or interacting in a deleterious manner with any of the components of the composition in which it is contained.

As used herein the term “pharmaceutically acceptable salt” refers to derivatives of the disclosed compounds wherein the parent compound is modified by converting an existing acid or base moiety to its salt form. Examples of pharmaceutically acceptable salts include but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent or in a mixture of the two; generally nonaqueous media like ether, ethyl acetate, ethanol, isopropanol, or acetonitrile are preferred. Lists of suitable salts are found in Remington's Pharmaceutical Sciences 17^{th} ed. Mack Publishing Company, Easton, Pa., 1985 p. 1418 and Journal of Pharmaceutical Science, 66, 2 (1977), each of which is incorporated herein by reference in its entirety. Pharmaceutically acceptable salts of the compounds according to the invention include acid addition salts, for example, but not limited to, salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid. Pharmaceutically acceptable salts of the compounds according to the invention also include salts of customary bases, for example, but not limited to, alkali metal salts (for example sodium and potassium salts), alkaline earth metal salts (for example calcium and magnesium salts) and ammonium salts derived from ammonia or organic amines having 1 to 16 carbon atoms, such as, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

As used herein, the term “solvate” refers to compounds which form a complex in the solid or liquid state by coordination with solvent molecules. Suitable solvents include, but are not limited to, methanol, ethanol, acetic acid and water. Hydrates are a special form of solvates in which the coordination takes place with water.

As used herein the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including but not limited to intravenous, oral, aerosol, rectal, parenteral, ophthalmic, pulmonary and topical administration.

As used herein the term “pharmaceutically acceptable carrier” means a pharmaceutically acceptable material, composition or carrier such as a liquid or solid filler, stabilizer, dispersing agent, suspending agent, diluent, excipient, thickening agent, solvent or encapsulating material involved in carrying or transporting a compound useful within the invention within or to the patient such that it may perform its intended function. Typically such constructs are carried or transported from one organ, or portion of the body, to another organ or portion of the body. Each carrier must be “acceptable” in the sense of being compatible with the other ingredients of the formulation including the compound use within the invention and not injurious to the patient. Some examples of materials that may serve as pharmaceutically acceptable carriers include: sugars, such as lactose, glucose and sucrose; starches such as corn starch and potato starch; cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols such as propylene glycol; polyols such as glycerin, sorbitol, mannitol and polyethylene glycol; esters such as ethyl oleate and ethyl laurate; agar; buffering agents, such as magnesium hydroxide and aluminium hydroxide; surface active agents; alginic acid; pyrogen-free water; isotonic saline; Ringer's solution; ethyl alcohol; phosphate buffer solutions and other non-toxic compatible substances employed in pharmaceutical formulations.

As used herein “pharmaceutically acceptable carrier” also includes any and all coatings, antibacterial and antifungal agents and absorption delaying agents and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described for example in Remington's Pharmaceutical Sciences (Genaro, Ed., Mack Publishing Company, Easton, Pa., 1985) which is incorporated herein by reference.

As used herein, the term “substituted” means that an atom or group of atoms has replaced hydrogen as the substituent attached to another group.

As used herein, the term “comprising” also encompasses the option “consisting of”.

As used herein, the term “alkyl” by itself or as part of another substituent means, unless otherwise stated, a straight or branched chain hydrocarbon having the number of carbon atoms designated (i.e. C1-C6-alkyl means one to six carbon atoms) and includes straight and branched chains. Examples include methyl, ethyl, propyl, isopropyl, butyl, isobutyl, tert-butyl, pentyl, neopentyl, and hexyl. In addition, the term “alkyl” by itself or as part of another substituent can also mean a C1-C3 straight chain hydrocarbon substituted with a C3-C5-carbocylic ring. Examples include (cyclopropyl)methyl, (cyclobutyl)methyl and (cyclopentyl)methyl. For the avoidance of doubt, where two alkyl moieties are present in a group, the alkyl moieties may be the same or different.

As used herein the term “alkenyl” denotes a monovalent group derived from a hydrocarbon moiety containing at least two carbon atoms and at least one carbon-carbon double bond of either E or Z stereochemistry. The double bond may or may not be the point of attachment to another group. Alkenyl groups (e.g. C2-C8-alkenyl) include, but are not limited to for example ethenyl, propenyl, prop-1-en-2-yl, butenyl, methyl-2-buten-1-yl, heptenyl and octenyl. For the avoidance of doubt, where two alkenyl moieties are present in a group, the alkyl moieties may be the same or different.

As used herein, a C2-C6-alkynyl group or moiety is a linear or branched alkynyl group or moiety containing from 2 to 6 carbon atoms, for example a C2-C4 alkynyl group or moiety containing from 2 to 4 carbon atoms. Exemplary alkynyl groups include —C≡CH or —CH₂—C≡C, as well as 1- and 2-butynyl, 2-pentynyl, 3-pentynyl, 4-pentynyl, 2-hexynyl, 3-hexynyl, 4-hexynyl and 5-hexynyl. For the avoidance of doubt, where two alkynyl moieties are present in a group, they may be the same or different.

As used herein, the term “halo” or “halogen” alone or as part of another substituent means unless otherwise stated a fluorine, chlorine, bromine, or iodine atom, preferably fluorine, chlorine, or bromine, more preferably fluorine or chlorine. For the avoidance of doubt, where two halo moieties are present in a group, they may be the same or different.

As used herein, a C1-C6-alkoxy group or C2-C6-alkenyloxy group is typically a said C1-C6-alkyl (e.g. a C1-C4 alkyl) group or a said C2-C6-alkenyl (e.g. a C2-C4 alkenyl) group respectively which is attached to an oxygen atom.

As used herein the term “aryl” employed alone or in combination with other terms, means unless otherwise stated a carbocyclic aromatic system containing one or more rings (typically one, two or three rings) wherein such rings may be attached together in a pendant manner such as a biphenyl, or may be fused, such as naphthalene. Examples of aryl groups include phenyl, anthracyl, and naphthyl. Preferred examples are phenyl (e.g. C6-aryl) and biphenyl (e.g. C12-aryl). In some embodiments aryl groups have from six to sixteen carbon atoms. In some embodiments aryl groups have from six to twelve carbon atoms (e.g. C6-C12-aryl). In some embodiments, aryl groups have six carbon atoms (e.g. C6-aryl).

As used herein the terms “heteroaryl” and “heteroaromatic” refer to a heterocycle having aromatic character containing one or more rings (typically one, two or three rings). Heteroaryl substituents may be defined by the number of carbon atoms e.g. C1-C9-heteroaryl indicates the number of carbon atoms contained in the heteroaryl group without including the number of heteroatoms. For example a C1-C9-heteroaryl will include an additional one to four heteroatoms. A polycyclic heteroaryl may include one or more rings that are partially saturated. Non-limiting examples of heteroaryls include:

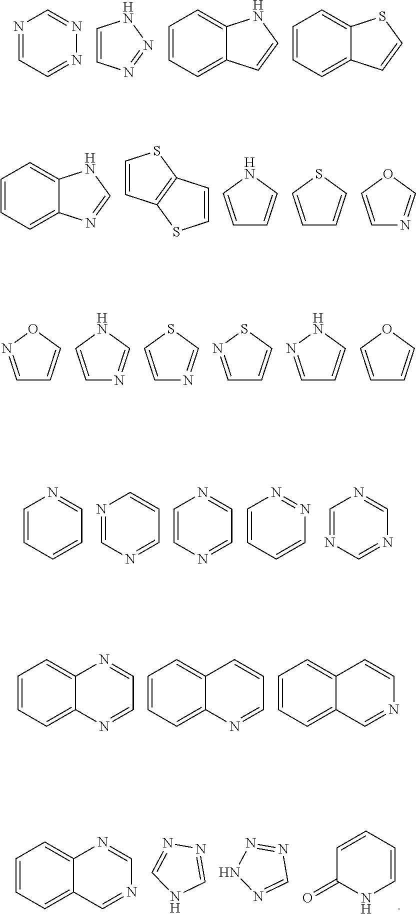

Additional non-limiting examples of heteroaryl groups include pyridyl, pyrazinyl, pyrimidinyl (including e.g. 2- and 4-pyrimidinyl), pyridazinyl, thienyl, furyl, pyrrolyl (including e.g., 2-pyrrolyl), imidazolyl, thiazolyl, oxazolyl, pyrazolyl (including e.g. 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyland 1,3,4-oxadiazolyl. Non-limiting examples of polycyclic heterocycles and heteroaryls include indolyl (including 3-, 4-, 5-, 6- and 7-indolyl), indolinyl, quinolyl, tetrahydroquinolyl, isoquinolyl (including, e.g. 1- and 5-isoquinolyl), 1,2,3,4-tetrahydroisoquinolyl, cinnolinyl, quinoxalinyl (including, e.g 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g. 3-, 4-, 5-, 6-, and 7-benzofuryl), 2,3-dihydrobenzofuryl, 1,2-benzisoxazolyl, benzothienyl (including e.g. 3-, 4-, 5-, 6-, and 7-benzothienyl), benzoxazolyl, benzothiazolyl (including e.g. 2-benzothiazolyl and 5-benzothiazolyl), purinyl, benzimidazolyl (including e.g., 2-benzimidazolyl), benzotriazolyl, thioxanthinyl, carbazolyl, carbolinyl, acridinyl, pyrrolizidinyl and quinolizidinyl.

As used herein the term “haloalkyl” is typically a said alkyl, alkenyl, alkoxy or alkenoxy group respectively wherein any one or more of the carbon atoms is substituted with one or more said halo atoms as defined above. Haloalkyl embraces monohaloalkyl, dihaloalkyl, and polyhaloalkyl radicals. The term “haloalkyl” includes but is not limited to fluoromethyl, 1-fluoroethyl, difluoromethyl, 2,2-difluoroethyl, 2,2,2-trifluoroethyl, trifluoromethyl, chloromethyl, dichloromethyl, trichloromethyl, pentafluoroethyl, difluoromethoxy, and trifluoromethoxy.

As used herein, a C1-C6-hydroxyalkyl group is a said C1-C6 alkyl group substituted by one or more hydroxy groups. Typically, it is substituted by one, two or three hydroxyl groups. Preferably, it is substituted by a single hydroxy group.

As used herein, a C1-C6-aminoalkyl group is a said C1-C6 alkyl group substituted by one or more amino groups. Typically, it is substituted by one, two or three amino groups. Preferably, it is substituted by a single amino group.

As used herein, a C1-C4-carboxyalkyl group is a said C1-C4 alkyl group substituted by carboxyl group.

As used herein, a C1-C4-carboxamidoalkyl group is a said C1-C4 alkyl group substituted by a substituted or unsubstituted carboxamide group.

As used herein, a C1-C4-acylsulfonamido-alkyl group is a said C1-C4 alkyl group substituted by an acylsulfonamide group of general formula C(═O)NHSO₂CH₃ or C(═O)NHSO₂-c-Pr.

As used herein, the term “carboxy” and by itself or as part of another substituent means, unless otherwise stated, a group of formula C(═O)OH.

As used herein, the term “cyano” by itself or as part of another substituent means, unless otherwise stated, a group of formula C≡N.

As used herein, the term “nitro” by itself or as part of another substituent means, unless otherwise stated, a group of formula NO₂.

As used herein, the term “carboxyl ester” by itself or as part of another substituent means, unless otherwise stated, a group of formula C(═O)OX, wherein X is selected from the group consisting of C1-C6-alkyl, C3-C7-cycloalkyl, and aryl.

As used herein, a carboxyphenyl group is a phenyl group substituted with a said carboxy group.

As used herein, a carboxypyridyl group is a pyridyl group substituted with a said carboxy group.

As used herein, a carboxypyrimidinyl group is a pyrimidinyl group substituted with a said carboxy group.

As used herein, a carboxypyrazinyl group is a pyrazinyl group substituted with a said carboxy group.

As used herein, a carboxypyridazinyl group is a pyridazinyl group substituted with a said carboxy group.

As used herein, a carboxytriazinyl group is a triazinyl group substituted with a said carboxy group.

As used herein, a carboxyoxazolyl group is an oxazolyl group substituted with a said carboxy group.

As used herein, a carboxyisoxazolyl group is an isoxazolyl group substituted with a said carboxy group.

As used herein, a carboxyimidazolyl group is an imidazolyl group substituted with a said carboxy group.

As used herein, a carboxypyrazolyl group is a pyrazolyl group substituted with a said carboxy group.

As used herein, the terms “pyridyl”, “pyrimidinyl”, “pyrazinyl”, “pyridazinyl”, “triazinyl”, “oxazolyl”, “isoxazolyl”, “imidazolyl”, and “pyrazolyl” when employed alone or in combination with one or more other terms encompasses, unless otherwise stated, positional isomers thereof.

As used herein an unsubstituted said pyridyl includes 2-pyridyl, 3-pyridyl and 4-pyridyl.

Examples of substituted pyridyl includes said 2-pyridyl, wherein further substitutions can be at the 3-, 4-, 5- or 6-positions. Further examples of substituted pyridyl also includes said 3-pyridyl, wherein further substitutions can be at the 2-, 4-, 5- or 6-positions, and said 4-pyridyl, wherein further substitutions can be at the 2-, 3-, 5- or 6-positions.

As used herein an unsubstituted said pyrimidinyl includes 2-pyrimidinyl, 4-pyrimidinyl and 5-pyrimidinyl. Examples of substituted pyrimidinyl includes said 2-pyrimidinyl, wherein further substitutions are on the 4-, 5- or 6-positions. Examples of substituted pyrimidinyl also includes said 4-pyrimidinyl, wherein further substitutions are on the 2-, 5- or 6-positions. Examples of substituted pyrimidinyl also includes said 5-pyrimidinyl, wherein further substitutions are on the 2-, 4- or 6-positions.

As used herein an unsubstituted said pyrazinyl is 2-pyrazinyl. Examples of substituted pyrazinyl include said 2-pyrimidinyl, wherein further substitutions are on the 3-, 5- or 6-positions.

As used herein an unsubstituted said pyridazinyl is 3-pyridazinyl. Examples of substituted pyrazinyl include said 3-pyrimidinyl, wherein further substitutions are on the 4-, 5- or 6-positions.

As used herein an unsubstituted said triazinyl is 2-triazinyl. A substituted triazinyl is a said 2-triazinyl with further substitutions on the 4- or 6-positions.

As used herein an unsubstituted said oxazolyl includes 2-oxazolyl and 4-oxazolyl. A substituted oxazolyl is either a said 2-oxazolyl with further substitutions on the 4- or 5-positions, or a said 4-oxazolyl with further substitutions on the 2-, or 5-positions.

As used herein an unsubstituted said isoxazolyl includes 3-isoxazolyl and 4-isoxazolyl. A substituted isoxazolyl is either a said 3-oxazolyl with further substitutions on the 4- or 5-positions, or a said 4-oxazolyl with further substitutions on the 3-, or 5-positions.

As used herein an unsubstituted said imidazolyl includes 2-imidazolyl and 4-imidazolyl. A substituted imidazolyl is either a said 2-imidazolyl with further substitutions on the N1-, N3-, 4- or 5-positions with the proviso that only one of N1- and N3- may be substituted, or a said 4-imidazolyl with further substitutions on the N1-, 2-, N3- or 5-positions, with the proviso that only one of N1- and N3- may be substituted.

As used herein an unsubstituted said pyrazolyl includes 3-pyrazolyl and 4-pyrazolyl. A substituted pyrazolyl is either a said 3-pyrazolyl with further substitutions on the N1-, N2-, 4- or 5-positions with the proviso that only one of N1- and N2- may be substituted, or a said 4-pyrazolyl with further substitutions on the N1-, N2-, 3- or 5-positions with the proviso that only one of N1- and N2- may be substituted.

As used herein the term “cycloalkyl” refers to a monocyclic or polycyclic nonaromatic group wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having 3 to 10 ring atoms (C3-C10-cycloalkyl), groups having 3 to 8 ring atoms (C3-C8-cycloalkyl), groups having 3 to 7 ring atoms (C3-C7-cycloalkyl) and groups having 3 to 6 ring atoms (C3-C6-cycloalkyl). Illustrative examples of cycloalkyl groups include, but are not limited to the following moieties:

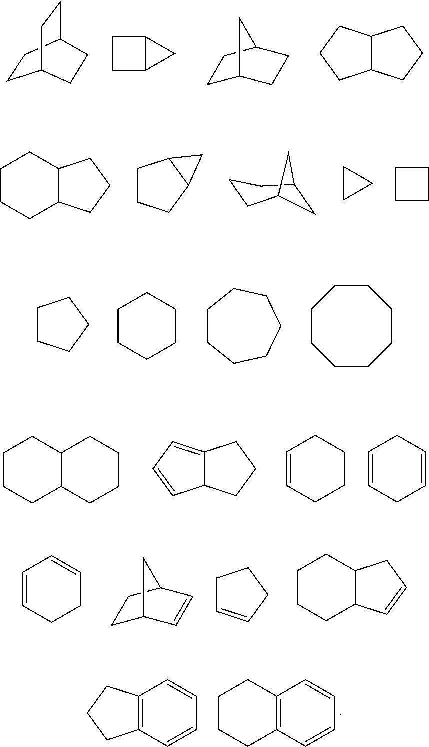

Monocyclic cycloalkyls include but are not limited to cyclopropyl, cyclobutyl, cyclopentyl, cyclohexyl, cycloheptyl, and cyclooctyl. Dicyclic cycloalkyls include but are not limited to tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups both of which refer to a nonaromatic carbocycle as defined herein which contains at least one carbon-carbon double bond or one carbon-carbon triple bond.

As used herein the term “halo-cycloalkyl” is typically a said cycloalkyl wherein any one or more of the carbon atoms is substituted with one or more said halo atoms as defined above. Halo-cycloalkyl embraces monohaloalkyl, dihaloalkyl, and polyhaloalkyl radicals. Halo-cycloalkyl embraces 3,3-difluoro-cyclobutyl, 3-fluorocyclobutyl, 2-fluorocyclobutyl, 2,2-difluorocyclobutyl, and 2,2-difluorocyclopropyl.

As used herein the terms “heterocycloalkyl” and “heterocyclyl” refer to a heteroalicyclic group containing one or more rings (typically one, two or three rings), that contains one to four ring heteroatoms each selected from oxygen, sulfur and nitrogen. In one embodiment each heterocyclyl group has from 3 to 10 atoms in its ring system with the proviso that the ring of said group does not contain two adjacent oxygen or sulfur atoms. In one embodiment each heterocyclyl group has a fused bicyclic ring system with 3 to 10 atoms in the ring system, again with the proviso that the ring of said group does not contain two adjacent oxygen or sulfur atoms. In one embodiment each heterocyclyl group has a bridged bicyclic ring system with 3 to 10 atoms in the ring system, again with the proviso that the ring of said group does not contain two adjacent oxygen or sulfur atoms. In one embodiment each heterocyclyl group has a spiro-bicyclic ring system with 3 to 10 atoms in the ring system, again with the proviso that the ring of said group does not contain two adjacent oxygen or sulfur atoms. Heterocyclyl substituents may be alternatively defined by the number of carbon atoms e.g. C2-C8-heterocyclyl indicates the number of carbon atoms contained in the heterocyclic group without including the number of heteroatoms. For example a C2-C8-heterocyclyl will include an additional one to four heteroatoms. In another embodiment the heterocycloalkyl group is fused with an aromatic ring.. In another embodiment the heterocycloalkyl group is fused with a heteroaryl ring. In one embodiment the nitrogen and sulfur heteroatoms may be optionally oxidized and the nitrogen atom may be optionally quaternized. The heterocyclic system may be attached, unless otherwise stated, at any heteroatom or carbon atom that affords a stable structure. An example of a 3-membered heterocyclyl group includes and is not limited to aziridine. Examples of 4-membered heterocycloalkyl groups include, and are not limited to azetidine and a beta-lactam. Examples of 5-membered heterocyclyl groups include, and are not limited to pyrrolidine, oxazolidine and thiazolidinedione. Examples of 6-membered heterocycloalkyl groups include, and are not limited to, piperidine, morpholine, piperazine, N-acetylpiperazine and N-acetylmorpholine. Other non-limiting examples of heterocyclyl groups are

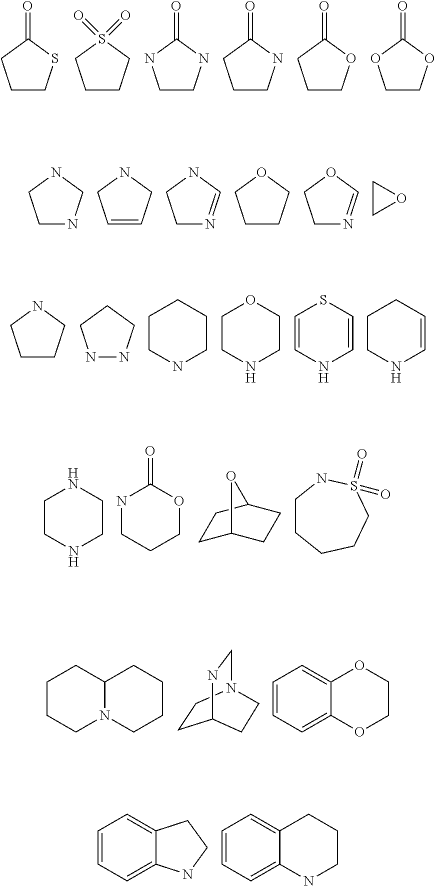

Examples of heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, 1,3-dioxolane, homopiperazine, homopiperidine, 1,3-dioxepane, 4,7-dihydro-1,3-dioxepin, and hexamethyleneoxide. The terms “C3-C7-heterocycloalkyl” includes but is not limited to tetrahydrofuran-2-yl, tetrahydrofuran-3-yl, 3-oxabicyclo[3.1.0]hexan-6-yl, 3-azabicyclo[3.1.0]hexan-6-yl, tetrahydropyran-4-yl, tetrahydropyran-3-yl, tetrahydropyran-2-yl, 1,3-dioxane-2-yl, 1,4-dioxane-2-yl, and azetidin-3-yl.

As used herein, the term “aromatic” refers to a carbocycle or heterocycle with one or more polyunsaturated rings and having aromatic character i.e. having (4n+2) delocalized 71(pi) electrons where n is an integer.

As used herein, the term “acyl”, employed alone or in combination with other terms, means, unless otherwise stated, to mean to an alkyl, cycloalkyl, heterocycloalkyl, aryl or heteroaryl group linked via a carbonyl group.

As used herein, the terms “carbamoyl” and “substituted carbamoyl”, employed alone or in combination with other terms, means, unless otherwise stated, to mean a carbonyl group linked to an amino group optionally mono or di-substituted by hydrogen, alkyl, cycloalkyl, heterocycloalkyl, aryl or heteroaryl. In some embodiments, the nitrogen substituents will be connected to form a heterocyclyl ring as defined above.

The term “prodrug” refers to a precursor of a drug that is a compound which upon administration to a patient, must undergo chemical conversion by metabolic processes before becoming an active pharmacological agent. Illustrative prodrugs of compounds in accordance with Formula I are esters and amides, preferably alkyl esters of fatty acid esters. Prodrug formulations here comprise all substances which are formed by simple transformation including hydrolysis, oxidation or reduction either enzymatically, metabolically or in any other way. A suitable prodrug contains e.g. a substance of general formula I bound via an enzymatically cleavable linker (e.g. carbamate, phosphate, N-glycoside or a disulfide group) to a dissolution-improving substance (e.g. tetraethylene glycol, saccharides, formic acids or glucuronic acid, etc.).Such a prodrug of a compound according to the invention can be applied to a patient, and this prodrug can be transformed into a substance of general formula I so as to obtain the desired pharmacological effect.

### EXAMPLES

The invention is now described with reference to the following Examples. These Examples are provided for the purpose of illustration only, and the invention is not limited to these Examples, but rather encompasses all variations that are evident as a result of the teachings provided herein.

The required substituted indole-2-carboxylic acids may be prepared in a number of ways; the main routes employed being outlined in Schemes 1-4. To the chemist skilled in the art it will be apparent that there are other methodologies that will also achieve the preparation of these intermediates.

Substituted indole-2-carboxylic acids can be prepared via the Hemetsberger-Knittel reaction (Organic Letters, 2011, 13(8) pp. 2012-2014, Journal of the American Chemical Society, 2007, pp. 7500-7501, and Monatshefte fir Chemie, 103(1), pp. 194-204) (Scheme 1).

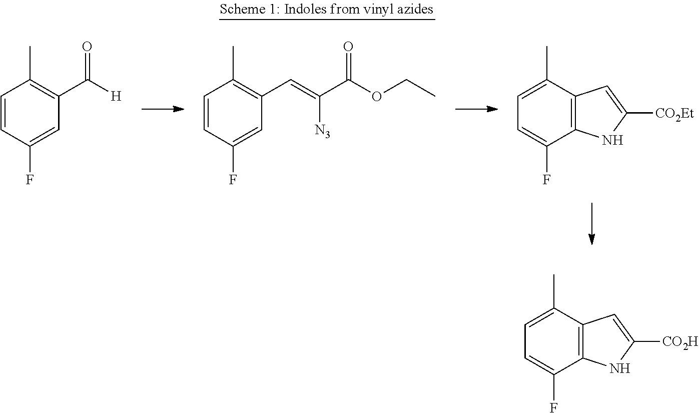

Substituted indoles may also be prepared using the Fischer method (Berichte der Deutschen Chemischen Gesellschaft. 17 (1): 559-568) (Scheme 2).

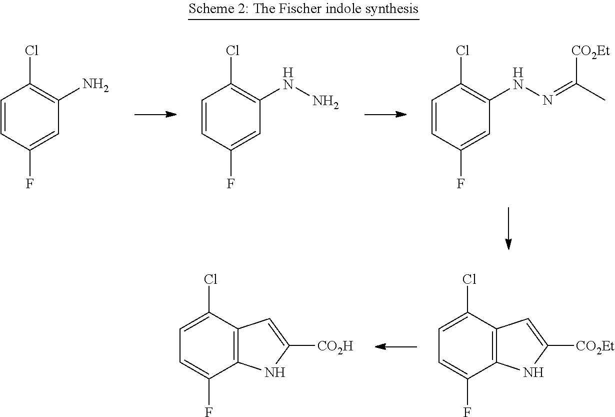 [SMILES: Nc1cc(F)ccc1Cl ; NNc1cc(F)ccc1Cl ; CCOC(=O)/C(C)=N\Nc1cc(F)ccc1Cl ; O=C(O)c1cc2c(Cl)ccc(F)c2[nH]1 ; CCOC(=O)c1cc2c(Cl)ccc(F)c2[nH]1]

A further method for the preparation of substituted indoles is the palladium catalysed alkyne annulation reaction (Journal of the American Chemical Society, 1991, pp. 6690-6692) (Scheme 3).

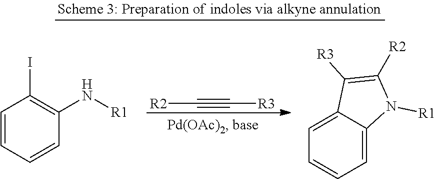

Additionally, indoles may be prepared from other suitably functionalized (halogenated) indoles (for example via palladium catalysed cross coupling or nucleophilic substitution reactions) as illustrated in Scheme 4.

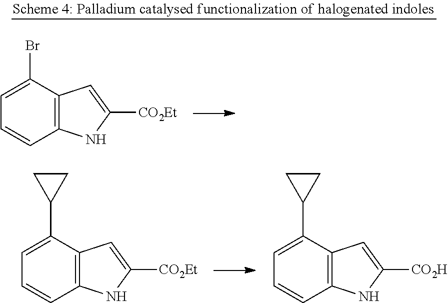

Chemists skilled in the art will appreciate that other methods are available for the synthesis of suitably functionalized indole-2-carboxylic acids and activated esters thereof.

In a preferred embodiment compounds of Formula 1 can be prepared as shown in Scheme 5.

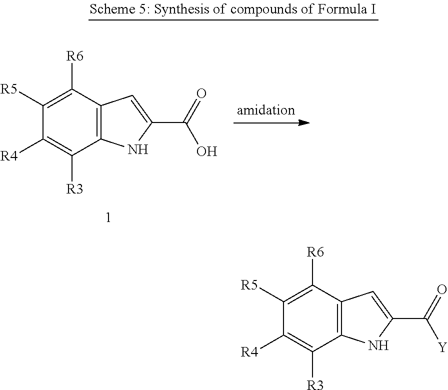

Compound 1 described in Scheme 5 is amidated in step 1 with methods known in literature (A. El-Faham, F. Albericio, Chem. Rev. 2011, 111, 6557-6602), e.g. with HATU resulting in compounds of Formula I.

In a further embodiment, compounds of Formula IIa can be prepared as shown in Scheme 6 below.

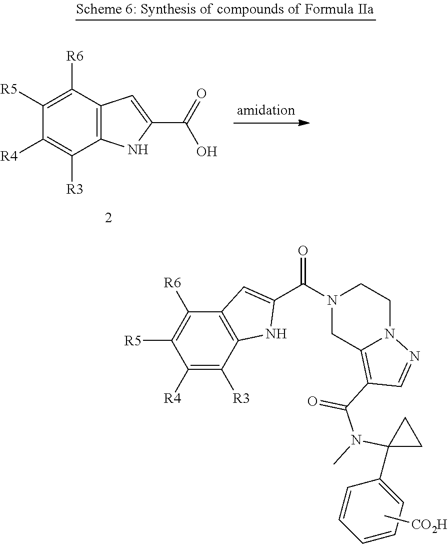

Compound 2 described in Scheme 6 is amidated in step 1 with methods known in literature (A. El-Faham, F. Albericio, Chem. Rev. 2011, 111, 6557-6602), e.g. with HATU resulting in compounds of Formula IIa.

In a further embodiment, compounds of Formula IIa can be prepared as shown in Scheme 7 below.

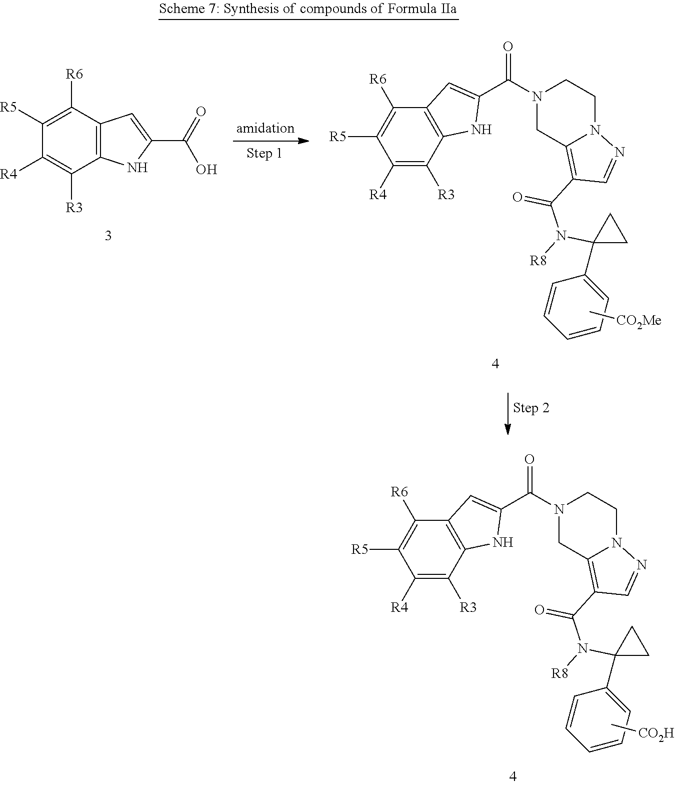

Compound 3 described in Scheme 7 is amidated in step 1 with methods known in literature (A. El-Faham, F. Albericio, Chem. Rev. 2011, 111, 6557-6602), e.g. with HATU resulting in compounds of general structure 4. The ester (drawn as but not limited to methyl) is then hydrolysed in step 2 with, for example, aqueous sodium hydroxide to give a compound of Formula IIa.

In a further embodiment, compounds of Formula IIb can be prepared as shown in Scheme 8 below.

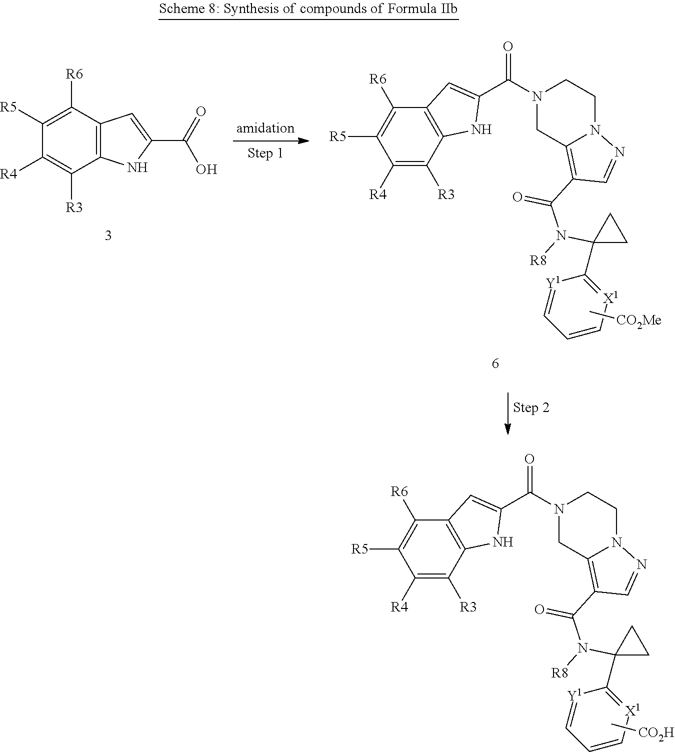

Compound 5 described in Scheme 7 is amidated in step 1 with methods known in literature (A. El-Faham, F. Albericio, Chem. Rev. 2011, 111, 6557-6602), e.g. with HATU resulting in compounds of general structure 6. The ester (drawn as but not limited to methyl) is then hydrolysed in step 2 with, for example, aqueous sodium hydroxide to give a compound of Formula IIb.

In a further embodiment, compounds of Formula IIc can be prepared as shown in Scheme 9 below.

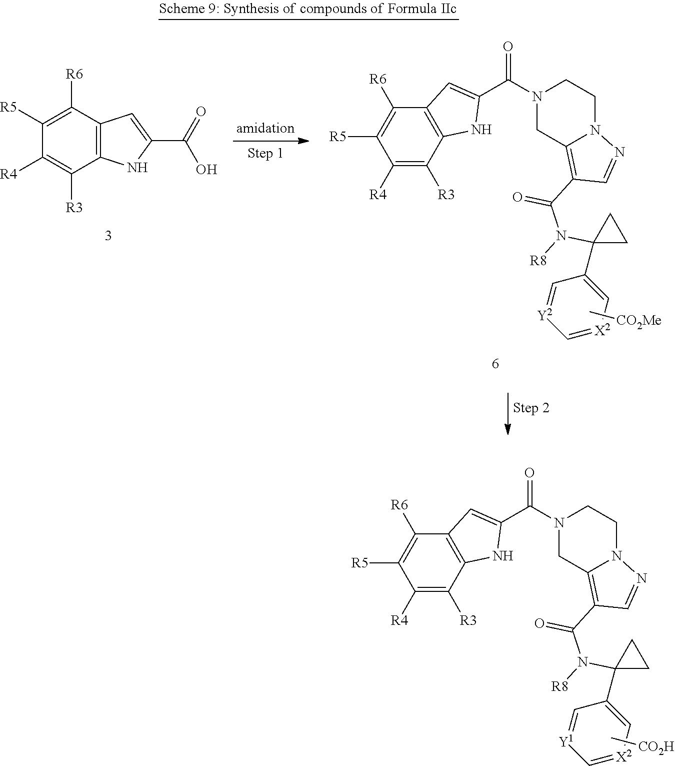

Compound 7 described in Scheme 9 is amidated in step 1 with methods known in literature (A. El-Faham, F. Albericio, Chem. Rev. 2011, 111, 6557-6602), e.g. with HATU resulting in compounds of general structure 8. The ester (drawn as but not limited to methyl) is then hydrolysed in step 2 with, for example, aqueous sodium hydroxide to give a compound of Formula IIc.

Chemists skilled in the art will appreciate that similar methods to those shown in Schemes 6-9 are suitable for the synthesis of compounds of Formula IIIa, IIIb, IIIc, IVa, IVb, IVc, Va,Vb,Vc, VIa, VIb, and VIc.

In a further embodiment, compounds of Formula VII can be prepared as shown in Scheme 10 below.

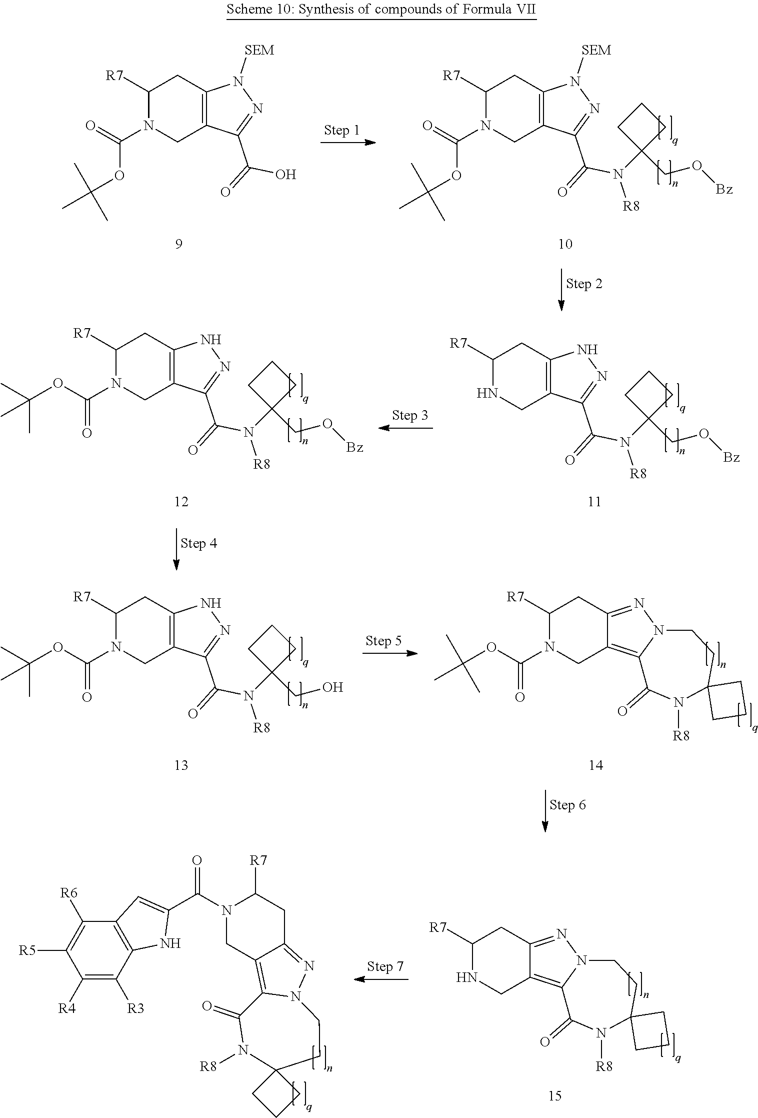

Compound 9 described in Scheme 10 is amidated in step 1 with methods known in literature (A. El-Faham, F. Albericio, Chem. Rev. 2011, 111, 6557-6602), e.g. with HATU resulting in compounds of general structure 10. Two of the three protecting groups (drawn as but not limited to Boc and SEM) are then removed in step 2 with, for example, HCl give a compound of general structure 11. The amine group is then re-protected in step 3 with a protecting group orthogonal to the alcohol protecting group (drawn as but not limited to benzoyl) as for example, a Boc group to give a compound of general structure 12. Removal of the alcohol protecting group, drawn as, but not limited to benzoyl with, for example, aqueous sodium hydroxide gives a compound of general structure 13. In step 5, Mitsunobu reaction of the alcohol with the pyrazole NH (WO2005/120516) gives a compound of general structure 14, which can then be deprotected (drawn as but not limited to Boc), with, for example HCl, to give a compound of general structure 15. The amine group of 15 can then be acylated with methods known in literature (A. El-Faham, F. Albericio, Chem. Rev. 2011, 111, 6557-6602), e.g. with HATU resulting in compounds of Formula VII.

The following examples illustrate the preparation and properties of some specific compounds of the invention.

The following abbreviations are used:
A—DNA nucleobase adenine
ACN—acetonitrile
Ar—argon
BODIPY-FL—4,4-difluoro-5,7-dimethyl-4-bora-3a,4a-diaza-s-indacene-3-propionic acid
(fluorescent dye)
Boc—tert-butoxycarbonyl
BnOH—benzyl alcohol
n-BuLi—n-butyl lithium
t-BuLi—t-butyl lithium
Bz—benzoyl
C—DNA nucleobase cytosine
Cbz—benzyloxycarbonyl
CC₅₀—half-maximal cytotoxic concentration
CO₂—carbon dioxide
CuCN—copper (I) cyanide
DABCO—1,4-diazabicyclo[2.2.2]octane
DCE—dichloroethane
DCM—dichloromethane
Dess-Martin periodinane—1,1,1-triacetoxy-1,1-dihydro-1,2-benziodoxol-3(1H)-one
DIAD—di-isopropylazodicarboxylate
DIPEA—diisopropylethylamine
DIPE—di-isopropyl ether
DMAP—4-dimethylaminopyridine
DMF—N,N-dimethylformamide
DMP—Dess-Martin periodinane
DMSO—dimethyl sulfoxide
DNA—deoxyribonucleic acid
DPPA—diphenylphosphoryl azide
DTT—dithiothreitol
EC₅₀—half-maximal effective concentration
EDCI—N-(3-dimethylaminopropyl)-N′-ethylcarbodiimide hydrochloride
Et₂O—diethyl ether
EtOAc—ethyl acetate
EtOH—ethanol
FL-—five prime end labled with fluorescein
NEt₃—triethylamine
ELS—Evaporative Light Scattering
g—gram(s)
G—DNA nucleobase guanine
HBV—hepatitis B virus
HATU—2-(1H-7-azabenzotriazol-1-yl)-1,1,3,3-tetramethyl uronium hexafluorophosphate
HCl—hydrochloric acid
HEPES—4-(2-hydroxyethyl)-1-piperazineethanesulfonic acid
HOAt —1-hydroxy-7-azabenzotriazole
HOBt—1-hydroxybenzotriazole
HPLC—high performance liquid chromatography
IC₅₀—half-maximal inhibitory concentration
LC640—3 prime end modification with fluorescent dye LightCycler® Red 640
LC/MS—liquid chromatography/mass spectrometry
LiAlH₄—lithium aluminium hydride
LiOH—lithium hydroxide
Me—methyl
MeOH—methanol
MeCN—acetonitrile
MgSO₄—magnesium sulfate
mg—milligram(s)
min—minutes
mol—moles
mmol—millimole(s)
mL—millilitre(s)
MTBE—methyl tert-butyl ether
N₂—nitrogen
Na₂CO₃—sodium carbonate
NaHCO₃—sodium hydrogen carbonate
Na₂SO₄—sodium sulfate
NdeI—restriction enzyme recognizes CA\hat{ }TATG sites
NEt₃—triethylamine
NaH—sodium hydride
NaOH—sodium hydroxide
NH₃—ammonia
NH₄Cl—ammonium chloride
NMR—nuclear magnetic resonance
PAGE—polyacrylamide gel electrophoresis
PCR—polymerase chain reaction
qPCR—quantitative PCR
Pd/C—palladium on carbon
PH—3 prime end phosphate modification
pTSA—4-toluene-sulfonic acid
Rt—retention time
r.t.—room temperature
sat.—saturated aqueous solution
SDS—sodium dodecyl sulfate
SI—selectivity index (=CC₅₀/EC₅₀)
STAB—sodium triacetoxyborohydride
T—DNA nucleobase thymine
TBAF—tetrabutylammonium fluoride
TEA—triethylamine
TFA—trifluoroacetic acid
THF—tetrahydrofuran
TLC—thin layer chromatography
TPPO—triphenylphosphine oxide
Tris—tris(hydroxymethyl)-aminomethane
XhoI—restriction enzyme recognizes C^{A}TCGAG sites

### Compound Identification—NMR

For a number of compounds, NMR spectra were recorded either using a Bruker DPX400 spectrometer equipped with a 5 mm reverse triple-resonance probe head operating at 400 MHz for the proton and 100 MHz for carbon, or using a Bruker DRX500 spectrometer equipped with a 5 mm reverse triple-resonance probe head operating at 500 MHz for the proton and 125 MHz for carbon. Deuterated solvents were chloroform-d (deuterated chloroform, CDCl₃) or d6-DMSO (deuterated DMSO, d6-dimethylsulfoxide). Chemical shifts are reported in parts per million (ppm) relative to tetramethylsilane (TMS) which was used as internal standard.

### Compound Identification—HPLC/MS

For a number of compounds, LC-MS spectra were recorded using the following analytical methods.

### Method A

Column—Reverse phase Waters Xselect CSH C18 (50×2.1 mm, 3.5 micron)
Flow—0.8 mL/min, 25 degrees Celsius
Eluent A—95% acetonitrile+5% 10 mM ammonium carbonate in water (pH 9)
Eluent B—10 mM ammonium carbonate in water (pH 9)
Linear gradient t=0 min 5% A, t=3.5 min 98% A. t=6 min 98% A

### Method A2

Column—Reverse phase Waters Xselect CSH C18 (50×2.1 mm, 3.5 micron)
Flow—0.8 mL/min, 25 degrees Celsius
Eluent A—95% acetonitrile+5% 10 mM ammonium carbonate in water (pH 9)
Eluent B—10 mM ammonium carbonate in water (pH 9)
Linear gradient t=0 min 5% A, t=4.5 min 98% A. t=6 min 98% A

### Method B

Column—Reverse phase Waters Xselect CSH C18 (50×2.1 mm, 3.5 micron)
Flow—0.8 mL/min, 35 degrees Celsius
Eluent A—0.1% formic acid in acetonitrile
Eluent B—0.1% formic acid in water
Linear gradient t=0 min 5% A, t=3.5 min 98% A. t=6 min 98% A

### Method B2

Column—Reverse phase Waters Xselect CSH C18 (50×2.1 mm, 3.5 micron)
Flow—0.8 mL/min, 40 degrees Celsius
Eluent A—0.1% formic acid in acetonitrile
Eluent B—0.1% formic acid in water
Linear gradient t=0 min 5% A, t=4.5 min 98% A. t=6 min 98% A

### Method C

Column—Reverse phase Waters Xselect CSH C18 (50×2.1 mm, 3.5 micron)
Flow—1 mL/min, 35 degrees Celsius
Eluent A—0.1% formic acid in acetonitrile
Eluent B—0.1% formic acid in water
Linear gradient t=0 min 5% A, t=1.6 min 98% A. t=3 min 98% A

### Method D

Column—Phenomenex Gemini NX C18 (50×2.0 mm, 3.0 micron)
Flow—0.8 mL/min, 35 degrees Celsius
Eluent A—95% acetonitrile+5% 10 mM ammonium bicarbonate in water
Eluent B—10 mM ammonium bicarbonate in water pH=9.0
Linear gradient t=0 min 5% A, t=3.5 min 98% A. t=6 min 98% A

### Method E

Column—Phenomenex Gemini NX C18 (50×2.0 mm, 3.0 micron)
Flow—0.8 mL/min, 25 degrees Celsius
Eluent A—95% acetonitrile+5% 10 mM ammonium bicarbonate in water
Eluent B—10 mM ammonium bicarbonate in water (pH 9)
Linear gradient t=0 min 5% A, t=3.5 min 30% A. t=7 min 98% A, t=10 min 98% A

### Method F

Column—Waters XSelect HSS C18 (150×4.6 mm, 3.5 micron)
Flow—1.0 mL/min, 25 degrees Celsius
Eluent A—0.1% TFA in acetonitrile
Eluent B—0.1% TFA in water
Linear gradient t=0 min 2% A, t=1 min 2% A, t=15 min 60% A, t=20 min 60% A

### Method G

Column—Zorbax SB-C18 1.8 μm 4.6×15 mm Rapid Resolution cartridge (PN 821975-932)
Flow—3 mL/min
Eluent A—0.1% formic acid in acetonitrile
Eluent B—0.1% formic acid in water
Linear gradient t=0 min 0% A, t=1.8 min 100% A

### Method H

Column—Waters Xselect CSH C18 (50×2.1 mm, 2.5 micron)
Flow—0.6 mL/min
Eluent A—0.1% formic acid in acetonitrile
Eluent B—0.1% formic acid in water
Linear gradient t=0 min 5% A, t=2.0 min 98% A, t=2.7 min 98% A

### Method J

Column—Reverse phase Waters Xselect CSH C18 (50×2.1 mm, 2.5 micron)
Flow—0.6 mL/min
Eluent A—100% acetonitrile
Eluent B—10 mM ammonium bicarbonate in water (pH 7.9)
Linear gradient t=0 min 5% A, t=2.0 min 98% A, t=2.7 min 98% A

### Synthesis of Indole-2-Carboxylic Acids

### Preparation of 4-chloro-7-fluoro-1H-indole-2-carboxylic acid

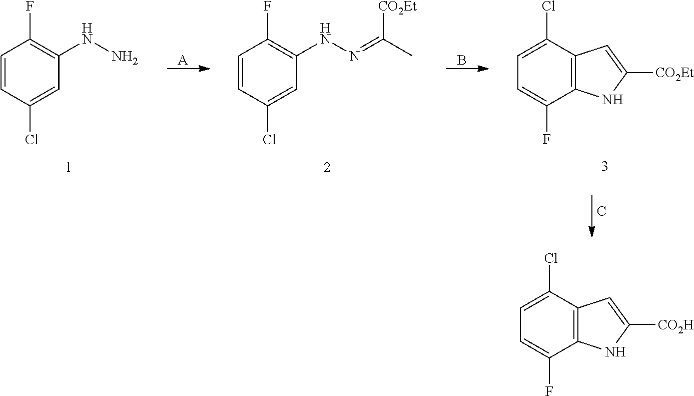

Step A: A mixture of compound 1-HCl (17.0 g, 86.2 mmol), sodium acetate (7.10 g, 86.6 mmol), and ethyl pyruvate (10.0 g, 86.1 mmol) in ethanol (100 mL) was refluxed for 1 h, cooled to r.t., and diluted with water (100 mL). The precipitated solid was collected by filtration and dried to obtain 20.0 g (77.3 mmol, 90%) of compound 2 as a mixture of cis- and trans-isomers.

Step B: A mixture of compound 2 (20.0 g, 77.3 mmol), obtained in the previous step, and BF₃-Et₂O (50.0 g, 352 mmol) in acetic acid (125 mL) was refluxed for 18 h and evaporated under reduced pressure. The residue was mixed with water (100 mL) and extracted with MTBE (2×50 mL). The combined organic extracts were dried over Na₂SO₄ and evaporated under reduced pressure. The residue was purified by silica gel column chromatography to give 3.00 g (12.4 mmol, 16%) of compound 3.

Step C: A mixture of compound 3 (3.00 g, 12.4 mmol) and NaOH (0.500 g, 12.5 mmol) in ethanol (30 mL) was refluxed for 30 min and evaporated under reduced pressure. The residue was mixed with water (30 mL) and the insoluble material was filtered off. The filtrate was acidified with concentrated hydrochloric acid (5 mL). The precipitated solid was collected by filtration, washed with water (3 mL), and dried to obtain 2.41 g (11.3 mmol, 91%) of 4-chloro-7-fluoro-1H-indole-2-carboxylic acid.

Rt (Method G) 1.24 mins, m/z 212 [M−H]⁻

### Preparation of 7-fluoro-4-methyl-1H-indole-2-carboxylic acid

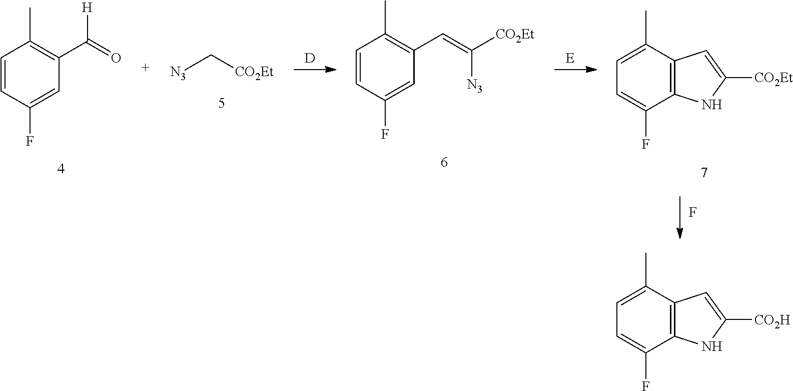

Step D: To a solution of sodium methoxide (21.6 g, 400 mmol) in methanol (300 mL) at at−10° C. was added dropwise a solution of compound 4 (26.4 g, 183 mmol) and compound 5 (59.0 g, 457 mmol) in methanol (100 mL). The reaction mass was stirred for 3 h maintaining temperature below 5° C. and then quenched with ice water. The resulting mixture was stirred for 10 min, filtered, and washed with water to afford 35.0 g (156 mmol, 72%) of compound 6 as a white solid.

Step E: A solution of compound 6, obtained in the previous step, (35.0 g, 156 mmol) in xylene (250 mL) was refluxed for 1 h under an argon atmosphere and then evaporated under reduced pressure. The residue was recrystallized form hexane-ethyl acetate mixture (60:40) to give 21.0 g (103 mmol, 60%) of compound 7.

Step F: To a solution of compound 7 (21.0 g, 101 mmol) in ethanol (200 mL) was added 2 N aqueous sodium hydroxide solution (47 mL). The mixture was stirred for 2 h at 60° C. The solvent was evaporated and the residue was acidified with aqueous hydrochloric acid to pH 5-6. The resulting precipitate was filtered, washed with water, and dried to obtain 18.0 g (93.2 mmol, 92%) of 7-fluoro-4-methyl-1H-indole-2-carboxylic acid.

Rt (Method G) 1.12 mins, m/z 192 [M−H]⁻

### Preparation of 6,7-difluoro-1H-indole-2-carboxylic acid

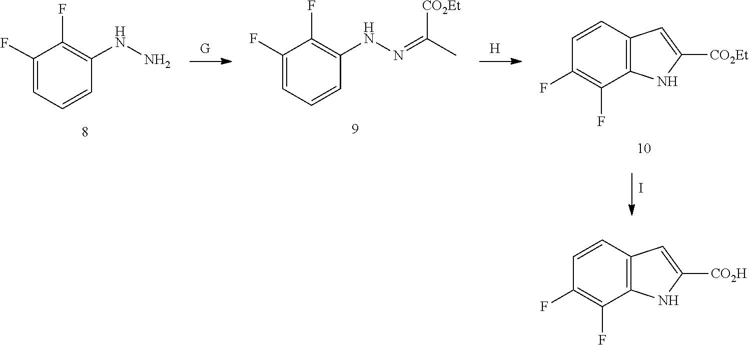

Step G: A mixture of compound 8 (5.00 g, 34.7 mmol), acetic acid (1 mL), and ethyl pyruvate (5.00 g, 43.1 mmol) in ethanol (20 mL) was refluxed for 1 h, cooled to r.t., and diluted with water (20 mL). The precipitated solid was collected by filtration and dried to obtain 5.50 g (22.7 mmol, 66%) of compound 9 as a mixture of cis- and trans-isomers.

Step H: A mixture of compound 9 (5.50 g, 22.7 mmol), obtained in the previous step, and BF₃-Et₂O (10.0 g, 70.5 mmol) in acetic acid (25 mL) was refluxed for 18 h and evaporated under reduced pressure. The residue was mixed with water (30 mL) and extracted with MTBE (2×30 mL). The combined organic extracts were dried over Na₂SO₄ and evaporated under reduced pressure. The residue was purified by silica gel column chromatography to give 0.460 g (2.04 mmol, 9%) of compound 10.

Step I: A mixture of compound 10 (0.450 g, 2.00 mmol) and NaOH (0.100 g, 2.50 mmol) in ethanol (10 mL) was refluxed for 30 min and evaporated under reduced pressure. The residue was mixed with water (10 mL) and the insoluble material was filtered off. The filtrate was acidified with concentrated hydrochloric acid (1 mL). The precipitated solid was collected by filtration, washed with water (3 mL), and dried to obtain 0.38 g (1.93 mmol, 95%) of 6,7-difluoro-1H-indole-2-carboxylic acid.

Rt (Method G) 1.10 mins, m/z 196 [M−H]⁻

### Preparation of 4-cyano-1H-indole-2-carboxylic acid

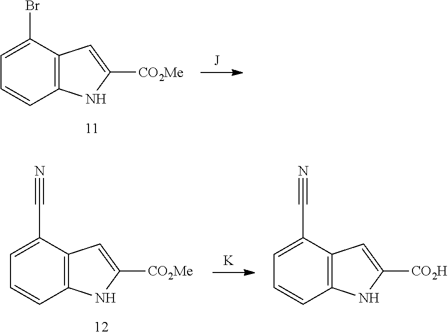

Step J: To a stirred solution of compound 11 (5.00 g, 19.7 mmol) in DMF (50 mL) was added CuCN (3.00 g, 33.5 mmol). The mixture was stirred for 4 h at 150° C. The mixture was then cooled to r.t., and water (100 mL) added. The resulting mixture was extracted with ethyl acetate (4×100 mL). The combined organic extracts were washed with water (50 mL) and brine (50 mL), dried over Na₂SO₄, and evaporated under reduced pressure to give 2.50 g (12.5 mmol, 63%) of compound 12, pure enough for the next step.

Step K: To a solution of compound 12 (2.50 g, 12.5 mmol) in ethanol (30 mL) was added LiOH.H₂O (0.600 g, 13.0 mmol). The mixture was refluxed for 10 h. The solvent was evaporated under reduced pressure and the residue diluted with water (50 mL). The aqueous layer was acidified to pH 6 with 10% aq. hydrochloric acid and the precipitated solid was collected by filtration. The residue was washed with water and dried under vacuum to afford 1.20 g (6.45 mmol, 52%) of 4-cyano-1H-indole-2-carboxylic acid as a white solid.

Rt (Method G) 1.00 mins, m/z 197 [M+H]⁺

### Preparation of 4-cyano-7-fluoro-1H-indole-2-carboxylic acid

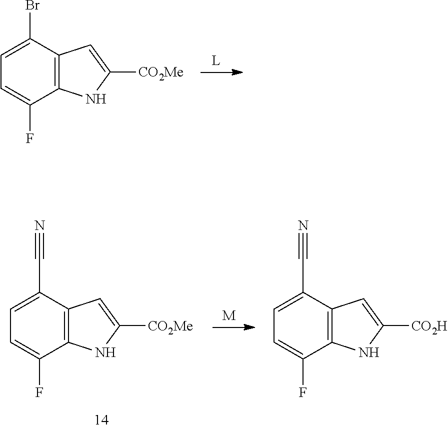

Step L: To a stirred solution of compound 13 (5.00 g, 18.4 mmol) in DMF (50 mL) was added CuCN (2.80 g, 31.2 mmol). The mixture was stirred for 4 h at 150° C. The mixture was then cooled to r.t., and water (100 mL) added. The resulting mixture was extracted with ethyl acetate (4×100 mL). The combined organic extracts were washed with water (50 mL) and brine (50 mL), dried over Na₂SO₄, and evaporated under reduced pressure to give 1.50 g (6.87 mmol, 37%) of compound 14, pure enough for the next step.

Step M: To a solution of compound 14 (1.50 g, 6.87 mmol) in ethanol (20 mL) was added LiOH.H₂O (0.400 g, 9.53 mmol). The mixture was refluxed for 10 h. The solvent was evaporated under reduced pressure and the residue diluted with water (40 mL). The aqueous layer was acidified to pH 6.0 with 10% aq. hydrochloric acid and the precipitate was collected by filtration. The residue was washed with water and dried under vacuum to afford 0.400 g (1.95 mmol, 28%) of 4-cyano-7-fluoro-1H-indole-2-carboxylic acid as a white solid.

Rt (Method G) 1.02 mins, m/z 203 [M−H]⁻

### Preparation of 4-cyano-5-fluoro-1H-indole-2-carboxylic acid

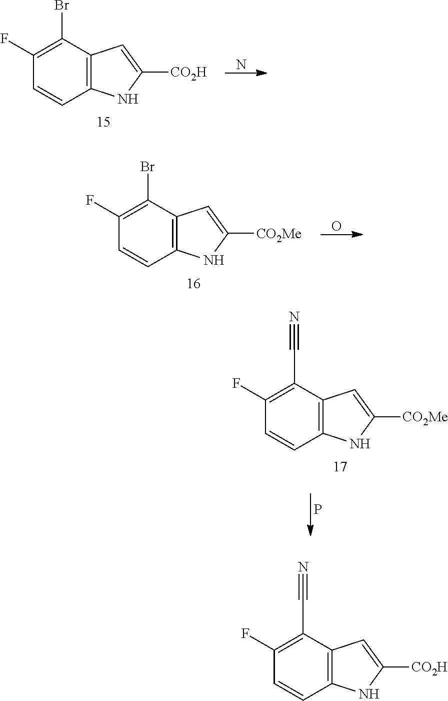

Step N: To a solution of compound 15 (5.00 g, 19.4 mmol) in DMF (50 mL) was added NaHCO₃(1.59 g, 18.9 mmol) and iodomethane (3 mL). The resulting mixture was stirred overnight at r.t., then diluted with water (50 mL) and extracted with diethyl ether (3×50 mL). The combined organic extracts were dried over Na₂SO₄, and evaporated under reduced pressure to obtain 4.90 g (18.0 mmol, 90%) of compound 16 as white solid.

Step O: To a stirred solution of compound 16 (4.80 g, 17.6 mmol) in DMF (50 mL) was added CuCN (2.70 g, 30.1 mmol). The mixture was stirred for 4 h at 150° C. The mixture was then cooled to r.t., water (100 mL) added. The resulting mixture was extracted with ethyl acetate (4×100 mL). The combined organic extracts were washed with water (50 mL) and brine (50 mL), dried over Na₂SO₄, and evaporated under reduced pressure to give 1.40 g (6.42 mmol, 36%) of compound 17, pure enough for the next step.

Step P: To a solution of compound 17 (1.40 g, 6.42 mmol) in ethanol (20 mL) was added LiOH.H₂O (0.350 g, 8.34 mmol). The mixture was refluxed for 10 h. The solvent was evaporated under reduced pressure and the residue diluted with water (30 mL). The aqueous layer was acidified to pH 6.0 with 10% aq. hydrochloric acid and the precipitate collected by filtration. The residue was washed with water and dried under vacuum to afford 0.500 g (2.45 mmol, 38%) of 4-cyano-5-fluoro-1H-indole-2-carboxylic acid as a white solid.

Rt (Method G) 1.10 mins, m/z 203 [M−H]⁻

### Preparation of 4,5,6-trifluoro-1H-indole-2-carboxylic acid

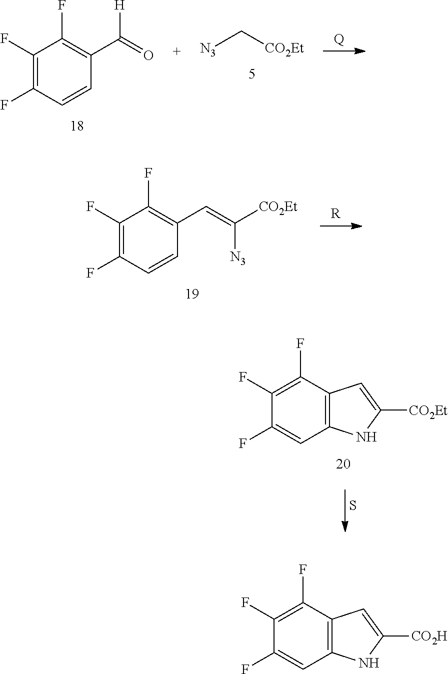

Step Q: To a solution of sodium methoxide (23.0 g, 426 mmol) in methanol (200 mL) at −10° C. was added dropwise a solution of compound 18 (15.0 g, 93.7 mmol) and compound 5 (26.0 g, 201 mmol) in methanol (100 mL). The reaction mixture was stirred for 3 h, maintaining the temperature below 5° C. and then quenched with ice water. The resulting mixture was stirred for 10 min, and the precipitate collected by filtration. The solid was washed with water and dried to afford 12.0 g (46.7 mmol, 72%) of compound 19 as a white solid.

Step R: A solution of compound 19, obtained in the previous step, (12.0 g, 46.7 mmol) in xylene (250 mL) was refluxed for 1 h under an argon atmosphere and then evaporated under reduced pressure. The residue was recrystallized form hexane-ethyl acetate mixture (60:40) to give 7.00 g (30.5 mmol, 65%) of compound 20.

Step S: To a solution of compound 20 (7.00 g, 30.5 mmol) in ethanol (50 mL) was added 2 N aqueous sodium hydroxide solution (18 mL). The mixture was stirred for 2 h at 60° C. The solvent was evaporated and the residue was acidified to pH 5-6 with aqueous hydrochloric acid. The resulting precipitate was collected by filtration, washed with water, and dried to obtain 5.00 g (23.2 mmol, 76%) 4,5,6-trifluoro-1H-indole-2-carboxylic acid.

1H NMR (400 MHz, d6-dmso) 7.17 (1H, s), 7.22 (1H, dd), 12.3 (1H, br s), 13.3 (1H, br s)

### Preparation of 4,6,7-trifluoro-1H-indole-2-carboxylic acid

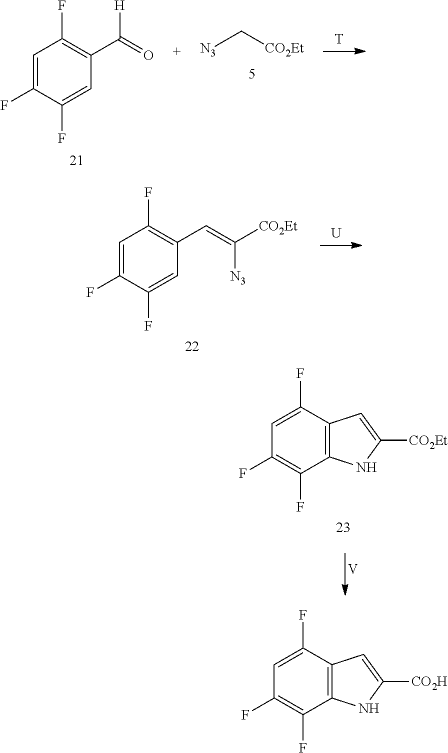

Step T: To a solution of sodium methoxide (23.0 g, 426 mmol) in methanol (200 mL) at −10° C. was added dropwise a solution of compound 21 (15.0 g, 90.3 mmol) and compound 5 (26.0 g, 201 mmol) in methanol (100 mL). The reaction mixture was stirred for 3 h maintaining the temperature below 5° C. and then quenched with ice water. The resulting mixture was stirred for 10 min. The precipitate was collected by filtration, washed with water and dried to afford 10.0 g (38.0 mmol, 42%) of compound 22 as a white solid.

Step U: A solution of compound 22, obtained in the previous step, (10.0 g, 38.0 mmol) in xylene (200 mL) was refluxed for 1 h under an argon atmosphere and then concentrated under reduced pressure. The residue was recrystallized form hexane-ethyl acetate mixture (60:40) to give 6.00 g (26.2 mmol, 69%) of compound 23.

Step V: To a solution of compound 23 (7.00 g, 30.5 mmol) in ethanol (40 mL) was added 2 N aqueous sodium hydroxide solution (16 mL). The mixture was stirred for 2 h at 60° C. The solvent was evaporated and the residue was acidified to pH 5-6 with aqueous hydrochloric acid. The resulting precipitate was collected by filtration, washed with water, and dried to obtain 4.10 g (19.1 mmol, 62%) of 4,6,7-trifluoro-1H-indole-2-carboxylic acid.

Rt (Method G) 1.16 mins, m/z 214 [M−H]⁻

### Preparation of 4-cyano-6-fluoro-1H-indole-2-carboxylic acid

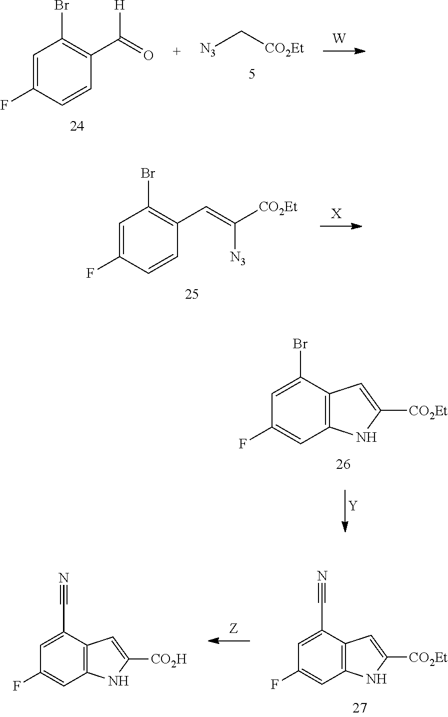

Step W: To a solution of sodium methoxide (65.0 g, 1203 mmol) in methanol (500 mL) at−10° C. was added dropwise a solution of compound 24 (60.0 g, 296 mmol) and compound 5 (85.0 g, 658 mmol) in methanol (200 mL). The reaction mixture was stirred for 3 h maintaining the temperature below 5° C. and then quenched with ice water. The resulting mixture was stirred for 10 min. The precipitate was collected by filtration, washed with water and dried to afford 45.0 g (143 mmol, 48%) of compound 25.

Step X: A solution of compound 25, obtained in the previous step, (35.0 g, 111 mmol) in xylene (250 mL) was refluxed for 1 h under an argon atmosphere and then evaporated under reduced pressure. The residue was recrystallized form hexane-ethyl acetate mixture (60:40) to give 11.0 g (38.4 mmol, 35%) of compound 26.

Step Y: To a stirred solution of compound 26 (11.0 g, 38.4 mmol) in DMF (20 mL) was added CuCN (6.60 g, 73.7 mmol). The mixture was stirred for 4 h at 150° C. The mixture was then cooled to r.t., and water (70 mL) added. The mixture was extracted with ethyl acetate (4×50 mL). The combined organic extracts were washed with water (50 mL) and brine (50 mL), dried over Na₂SO₄, and evaporated under reduced pressure to give 2.40 g (10.3 mmol, 27%) of compound 27, pure enough for the next step.

Step Z: To a solution of compound 27 (2.40 g, 6.42 mmol) in ethanol (30 mL) was added LiOH.H₂O (0.600 g, 14.3 mmol). The mixture was refluxed for 10 h. The mixture was concentrated under reduced pressure and the residue diluted with water (50 mL). The aqueous layer was acidified to pH 6 with 10% aq. hydrochloric acid and the precipitate was collected by filtration. The solid was washed with water and dried under vacuum to afford 1.20 g (5.88 mmol, 57%) of 4-cyano-6-fluoro-1H-indole-2-carboxylic acid as a white solid.

Rt (Method G) 1.06 mins, m/z 203 [M−H]⁻

### Preparation of 4-ethyl-1H-indole-2-carboxylic acid

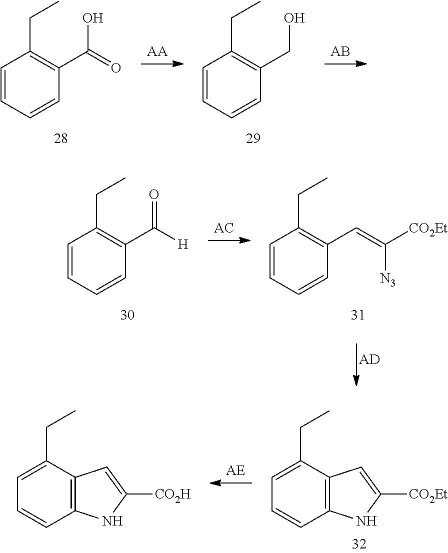

Step AA: A solution of compound 28 (70.0 g, 466 mmol) in dry THF (500 mL) was treated with 10 M solution of BH₃ in THF (53 mL, 53.0 mmol of BH₃) at 0° C. The reaction mass was stirred at r.t. for 24 h before methanol (150 mL) was slowly added thereto. The resulting mixture was stirred for 45 min, and evaporated under reduced pressure to yield 55.0 g (404 mmol, 87%) of compound 29, pure enough for the next step.

Step AB: To a cooled (0° C.) solution of compound 29 (55.0 g, 404 mmol) in CH₂Cl₂ (400 mL) was added Dess-Martin periodinane (177 g, 417 mmol) portionwise. After stirring for 1 h at r.t., the reaction mixture was quenched with saturated aqueous Na₂S₂O₃ (300 mL) and saturated aqueous NaHCO₃(500 mL). The mixture was extracted with CH₂Cl₂ (3×300 mL). The combined organic extracts were washed with water and brine, dried over Na₂SO₄ and concentrated to yield 51.0 g of crude compound 30 as a yellow solid.

Step AC: To a solution of sodium methoxide (107 g, 1981 mmol) in methanol (600 mL) at −10° C. was added dropwise a solution of compound 30, obtained in the previous step, (51.0 g) and compound 5 (126 g, 976 mmol) in methanol (300 mL). The reaction mixture was stirred for 4 h maintaining temperature below 5° C., then quenched with ice water. The resulting mixture was stirred for 10 min, and the precipitate collected by filtration. The solid was washed with water and dried to afford 35.0 g (151 mmol, 37% over 2 steps) of compound 31.

Step AD: A solution of compound 31, obtained in the previous step, (35.0 g, 151 mmol) in xylene (500 mL) was refluxed for 1 h under an argon atmosphere and then concentrated under reduced pressure. The residue was recrystallized form hexane-ethyl acetate mixture (60:40) to give 21.0 g (103 mmol, 68%) of compound 32.

Step AE: To a solution of compound 32 (21.0 g, 103 mmol) in ethanol (200 mL) was added 2 N aqueous sodium hydroxide solution (47 mL). The mixture was stirred for 2 h at 60° C. The mixture was concentrated under reduced pressure, and the residue acidified to pH 5-6 with aqueous hydrochloric acid. The precipitate was collected by filtration, washed with water, and dried to obtain 19 g (100 mmol, 97%) of 4-ethyl-1H-indole-2-carboxylic acid.

Rt (Method G) 1.20 mins, m/z 188 [M−H]⁻

1H NMR (400 MHz, d6-dmso) δ 1.25 (t, 3H), 2.88 (q, 2H), 6.86 (1H, d), 7.08-7.20 (2H, m), 7.26 (1H, d), 11.7 (1H, br s), 12.9 (1H, br s)

### Preparation of 4-cyclopropyl-1H-indole-2-carboxylic acid

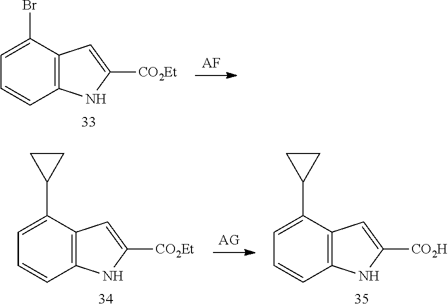

Step AF: To a degassed suspension of compound 33 (2.00 g, 7.80 mmol), cyclopropylboronic acid (0.754 g, 8.78 mmol), K₃PO₄ (5.02 g, 23.6 mmol), tricyclohexyl phosphine (0.189 g, 0.675 mmol), and water (2.0 mL) in toluene (60.0 mL) was added palladium (II) acetate (0.076 g, 0.340 mmol). The reaction mixture was stirred at 100° C. for 4 h. The reaction progress was monitored by diluting an aliquot of the reaction mixture with water and extracting with ethyl acetate. The organic layer was spotted over an analytical silica gel TLC plate and visualized using 254 nm UV light. The reaction progressed to completion with the formation of a polar spot. The R_{f} values of the starting material and product were 0.3 and 0.2, respectively. The reaction mixture was allowed to cool to r.t. and filtered through a pad of celite. The filtrate was concentrated under reduced pressure and the crude product was purified by flash column using 230-400 mesh silica gel and eluted with 10% ethyl acetate in petroleum ether to afford 1.10 g (5.11 mmol, 63%) of compound 34 as a brown liquid. TLC system: 5% ethyl acetate in petroleum ether.

Step AG: A mixture of compound 34 (1.10 g, 5.11 mmol) in ethanol (40 mL) and 2 N aqueous sodium hydroxide (15 mL) was stirred for 2 h at 60° C. The mixture was concentrated under reduced pressure, and the residue acidified to pH 5-6 with aqueous hydrochloric acid. The precipitate was collected by filtration, washed with water, and dried to yield 1.01 g (5.02 mmol, 92%) of 4-cyclopropyl-1H-indole-2-carboxylic acid.

Rt (Method G) 1.17 mins, m/z 200 [M−H]⁻

### Preparation of 4-chloro-5-fluoro-1H-indole-2-carboxylic acid

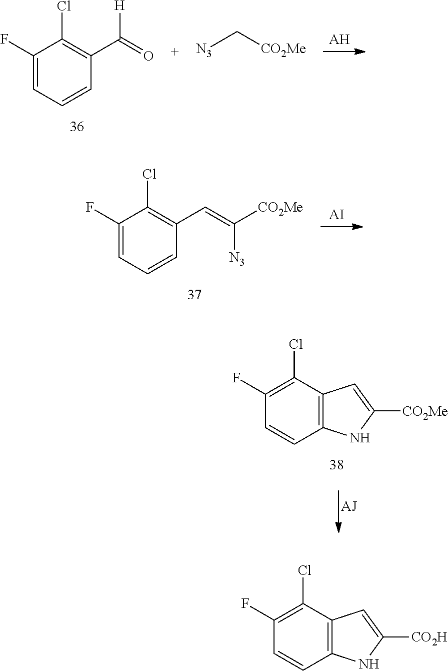

Step AH: To a solution of sodium methoxide (39.9 g, 738 mmol) in methanol (300 mL) at −10° C. was added dropwise a solution of compound 36 (28.8 g, 182 mmol) and methyl azidoacetate (52.1 g, 404 mmol) in methanol (150 mL). The reaction mixture was stirred for 3 h maintaining temperature below 5° C., then quenched with ice water. The resulting mixture was stirred for 10 min. The precipitate was collected by filtration, washed with water and dried to afford 20.0 g (78.2 mmol, 43%) of compound 37.

Step AI: A solution of compound 37 (19.4 g, 76.0 mmol) in xylene (250 mL) was refluxed for 1 h under an argon atmosphere and then concentrated under reduced pressure. The residue was recrystallized from hexane-ethyl acetate (50:50) to give 9.00 g (39.5 mmol, 52%) of compound 38.

Step AJ: To a solution of compound 38 (8.98 g, 39.4 mmol) in ethanol (100 mL) was added 2 N aqueous sodium hydroxide solution (18 mL). The mixture was stirred for 2 h at 60° C. The mixture was concentrated under reduced pressure, and the residue acidified to pH 5-6 with aqueous hydrochloric acid. The resulting precipitate was collected by filtration, washed with water, and dried to obtain 7.75 g (36.3 mmol, 92%) of 4-chloro-5-fluoro-1H-indole-2-carboxylic acid.

Rt (Method G) 1.15 mins, m/z 212 [M−H]⁻

1H NMR (400 MHz, d6-dmso) 7.08 (1H, s), 7.28 (1H, dd) 7.42 (1H, dd), 12.2 (1H, br s), 13.2 (1H, br s)

### Preparation of 5-fluoro-4-(1-hydroxyethyl)-1H-indole-2-carboxylic acid

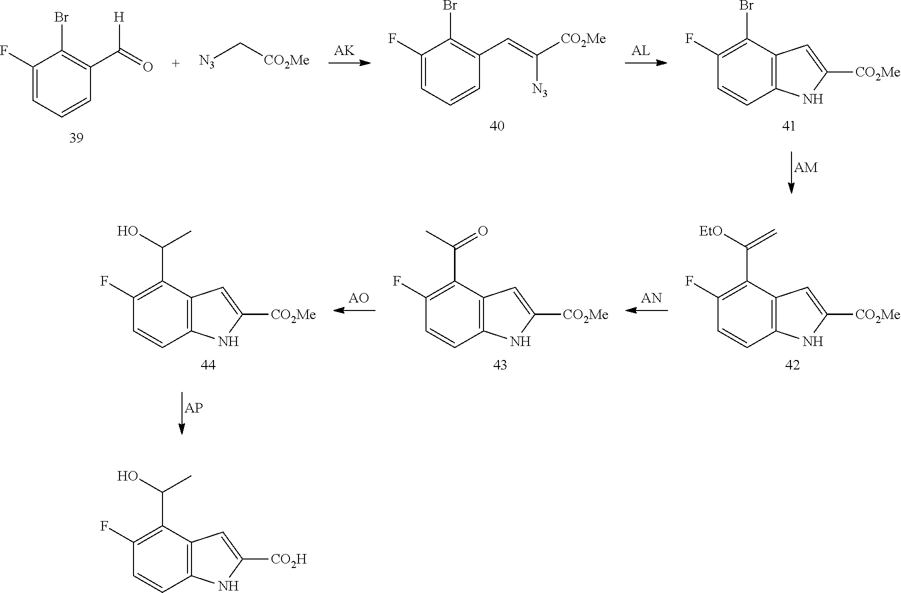

Step AK: To a solution of sodium methoxide (50.0 g, 926 mmol) in methanol (300 mL) at-10° C. was added dropwise a solution of compound 39 (45.0 g, 222 mmol) and methyl azidoacetate (59.0 g, 457 mmol) in methanol (100 mL). The reaction mixture was stirred for 3 h maintaining the temperature below 5° C., then quenched with ice water. The resulting mixture was stirred for 10 min. The precipitate was collected by filtration, washed with water and dried to afford 35.0 g (133 mmol, 60%) of compound 40 as a white solid.

Step AL: A solution of compound 40, obtained in the previous step, (35.0 g, 133 mmol) in xylene (250 mL) was refluxed for 1 h under an argon atmosphere and then evaporated under reduced pressure. The residue was recrystallized from hexane-ethyl acetate (60:40) to give 21.0 g (77.2 mmol, 58%) of compound 41.

Step AM: To a degassed solution of compound 41 (4.00 g, 14.7 mmol) and tributyl(1-ethoxyvinyl)stannane (5.50 g, 15.2 mmol) in toluene (50 mL) under nitrogen was added bis(triphenylphosphine) palladium(II) dichloride (1.16 g, 1.65 mmol). The reaction mixture was stirred at 60° C. for 20 h. The reaction mixture was cooled to room temperature and filtered. The filtrate was concentrated under under reduced pressure and the residue purified by silica gel chromatography to afford 2.50 g (9.50 mmol, 65%) of compound 42 as a pale yellow solid.

Step AN: To a solution of compound 42 (2.40 g, 9.12 mmol) in 1,4-dioxane (30 mL) was added 2M hydrochloric acid (15 mL). The resulting mixture was stirred at room temperature for 30 min. The mixture was concentrated under vacuum and the residue partitioned between ethyl acetate and water. The organic extract was washed with water and brine, dried over sodium sulfate, filtered, and evaporated. The residue was triturated with 5% ether in isohexane and dried to afford 1.80 g (7.65 mmol, 84%) of compound 43 as a white solid.

Step AO: A suspension of compound 43 (1.70 g, 7.23 mmol) and NaBH₄ (2.50 g, 66.1 mmol) in ethanol (13 mL) was refluxed for 2 h, then cooled to room temperature, and filtered. The filtrate was concentrated under reduced pressure and the residue dissolved in ethyl acetate. The solution was washed with 1N hydrochloric acid and brine, dried over Na₂SO₄, and evaporated under reduced pressure to give 1.60 g (6.74 mmol, 93%) of compound 44 as a colourless oil.

Step AP: To a solution of compound 44 (1.50 g, 6.32 mmol) in methanol (40 mL) was added 2N aqueous NaOH (10 mL). The mixture was stirred for 2 h at 60° C. The mixture was concentrated under reduced pressure and the residue acidified to pH 5-6 with 10% hydrochloric acid. The precipitate was collected by filtration, washed with water (3×15 mL), and dried to obtain 1.30 g (5.82 mmol, 92%) of 5-fluoro-4-(1-hydroxyethyl)-1H-indole-2-carboxylic acid.

Rt (Method G) 1.00 mins, m/z 222 [M−H]⁻

### Preparation of 4-ethyl-5-fluoro-1H-indole-2-carboxylic acid

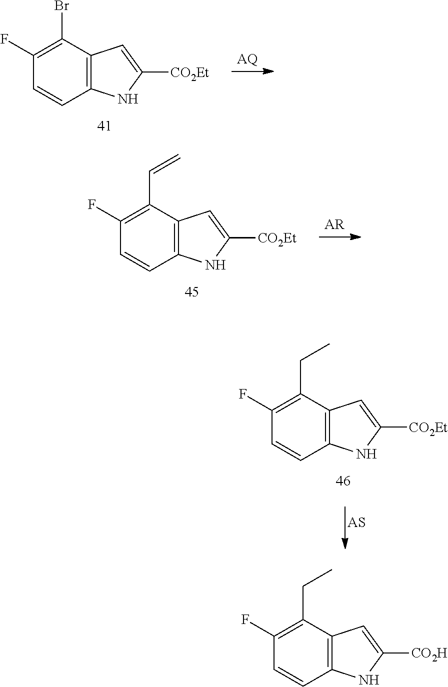

Step AQ: To a heated (90° C.) solution of compound 41 (4.00 g, 14.7 mmol) in anhydrous DMF under nitrogen (10 mL) were added tri-n-butyl(vinyl)tin (3.60 g, 11.4 mmol) and Pd(PPh₃)₂Cl₂ (0.301 g, 0.757 mmol). The resulting mixture was stirred at 90° C. for 1 h. The mixture was then cooled to room temperature and purified by silica gel column chromatography (60-80% ethyl acetate in hexane) to give 2.20 g (10.0 mmol, 68%) of compound 45 as yellow solid.

Step AR: A mixture of compound 45 (1.50 g, 6.84 mmol) and Pd/C (0.300 g, 10% wt.) in methanol (20 mL) was stirred under an atmosphere of hydrogen at room temperature for 16 h. The mixture was filtered, then concentrated under reduced pressure to give 1.45 g (6.55 mmol, 96%) of compound 46.

Step AS: To a solution of compound 46 (1.40 g, 6.33 mmol) in methanol (40 mL) was added 2N aqueous NaOH (10 mL). The mixture was stirred for 2 h at 60° C. The mixture was concentrated under vacuum, then the residue was acidified to pH 5-6 with 10% hydrochloric acid. The precipitate was collected by filtration, washed with water (3×15 mL), and dried to obtain 1.20 g (5.79 mmol, 91%) of target compound 4-ethyl-5-fluoro-1H-indole-2-carboxylic acid.

Rt (Method G) 1.33 mins, m/z 206 [M−H]⁻

### Preparation of 4-ethyl-6-fluoro-1H-indole-2-carboxylic acid

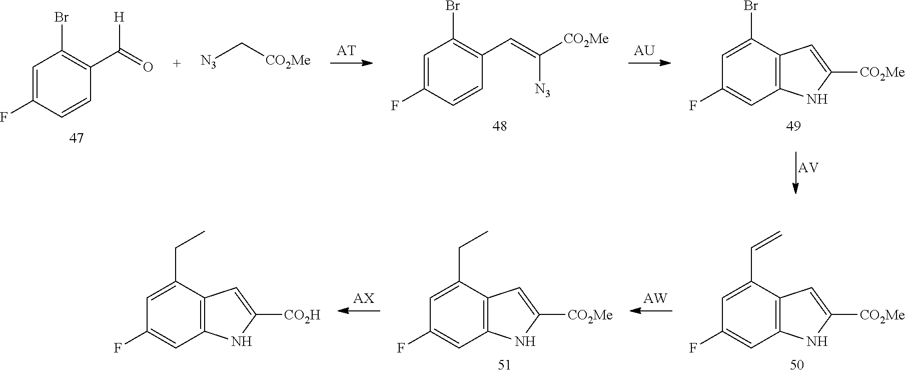

Step AT: To a solution of sodium methoxide (50.0 g, 926 mmol) in methanol (300 mL) at-10° C. was added dropwise a solution of compound 47 (45.0 g, 202 mmol) and methyl azidoacetate (59.0 g, 457 mmol) in methanol (100 mL). The reaction mixture was stirred for 3 h maintaining temperature below 5° C., then quenched with ice water. The resulting mixture was stirred for 10 min. The precipitate was collected by filtration, washed with water and dried to afford 38.5 g (128 mmol, 63%) of compound 48 as a white solid.

Step AU: A solution of compound 48, obtained in the previous step, (38.5 g, 128 mmol) in xylene (250 mL) was refluxed for 1 h under an argon atmosphere and then concentrated under reduced pressure. The residue was recrystallized hexane-ethyl acetate (60:40) to give 18.0 g (67.3 mmol, 53%) of compound 49.

Step AV: To a heated (90° C.) solution of compound 49 (4.00 g, 14.7 mmol) in anhydrous DMF under nitrogen (10 mL) were added tri-n-butyl(vinyl)tin (3.60 g, 11.4 mmol) and Pd(PPh₃)₂Cl₂ (0.301 g, 0.757 mmol). The resulting mixture was stirred at 90° C. for 1 h. The mixture was then cooled to room temperature and purified by silica gel column chromatography (60-80% ethyl acetate in hexane) to give 2.00 g (9.12 mmol, 62%) of compound 50 as yellow solid.

Step AW: A mixture of compound 50 (1.50 g, 6.84 mmol) and Pd/C (0.300 g, 10% wt.) in methanol (20 mL) was stirred under an atmosphere of hydrogen at room temperature for 16 h. The mixture was filtered and concentrated to give 1.40 g (6.33 mmol, 93%) of compound 51.

Step AX: To a solution of compound 51 (1.10 g, 4.97 mmol) in methanol (40 mL) was added 2N aqueous NaOH (10 mL). The mixture was stirred for 2 h at 60° C. The mixture was concentrated under reduced pressure, then acidified to pH 5-6 with 10% hydrochloric acid. The precipitate was collected by filtration, washed with water (3×15 mL), and dried to obtain 0.900 g (4.34 mmol, 87%) of target compound 4-ethyl-6-fluoro-1H-indole-2-carboxylic acid.

Rt (Method G) 1.29 mins, m/z 206 [M−H]⁻

### Preparation of 6-fluoro-4-(1-hydroxyethyl)-1H-indole-2-carboxylic acid

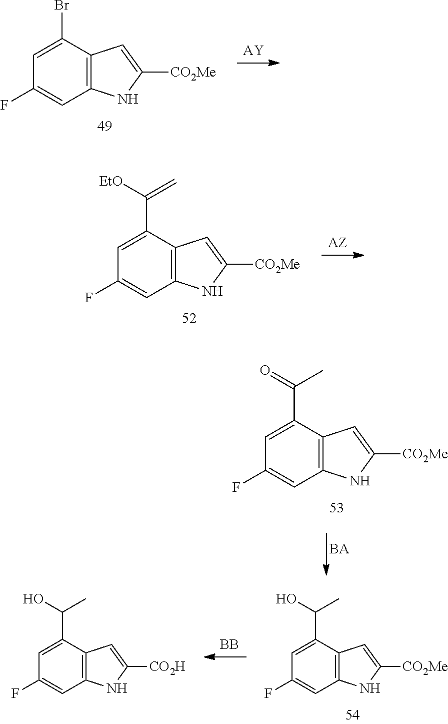

Step AY: To a degassed solution of compound 49 (4.00 g, 14.7 mmol) and tributyl(1-ethoxyvinyl)stannane (5.50 g, 15.2 mmol) in toluene (50 mL) under nitrogen were added bis(triphenylphosphine) palladium(II) dichloride (1.16 g, 1.65 mmol). The reaction mixture was stirred at 60° C. for 20 h. The reaction mixture was cooled to room temperature and filtered. The filtrate was concentrated under reduced pressure and the residue purified by silica gel chromatography to give 2.10 g (7.98 mmol, 54%) of compound 52 as a pale yellow solid.

Step AZ: To a solution of compound 52 (2.10 g, 7.98 mmol) in 1,4-dioxane (30 mL) was added 2M hydrochloric acid (15 mL). The resulting mixture was stirred at room temperature for 30 min. The mixture was concentrated under reduced pressure, and residue partitioned between ethyl acetate and water. The organic extract was washed with water and brine, dried over sodium sulfate, filtered, and concentrated. The residue was triturated with 5% ether in isohexane and dried to afford 1.70 g (7.23 mmol, 91%) of compound 53 as a white solid.

Step BA: A suspension of compound 53 (1.70 g, 7.23 mmol) and NaBH₄ (2.50 g, 66.1 mmol) in ethanol (13 mL) was refluxed for 2 h, cooled to room temperature, and filtered. The filtrate was concentrated under reduced pressure and the residue was dissolved in ethyl acetate. The solution was washed with 1N hydrochloric acid and brine, dried over Na₂SO₄, and concentrated under reduced pressure to give 1.60 g (6.74 mmol, 93%) of compound 54 as a colourless oil.

Step BB: To a solution of compound 54 (1.40 g, 5.90 mmol) in methanol (40 mL) was added 2N aqueous NaOH (10 mL). The mixture was stirred for 2 h at 60° C. The mixture was concentrated and the residue acidified to pH 5-6 with 10% hydrochloric acid. The precipitate was collected by filtration, washed with water (3×15 mL), and dried to obtain 1.10 g (4.93 mmol, 48%) of target compound 6-fluoro-4-(1-hydroxyethyl)-1H-indole-2-carboxylic acid.

Rt (Method G) 1.00 mins, m/z 222 [M−H]⁻

### Preparation of 4-ethyl-7-fluoro-1H-indole-2-carboxylic acid

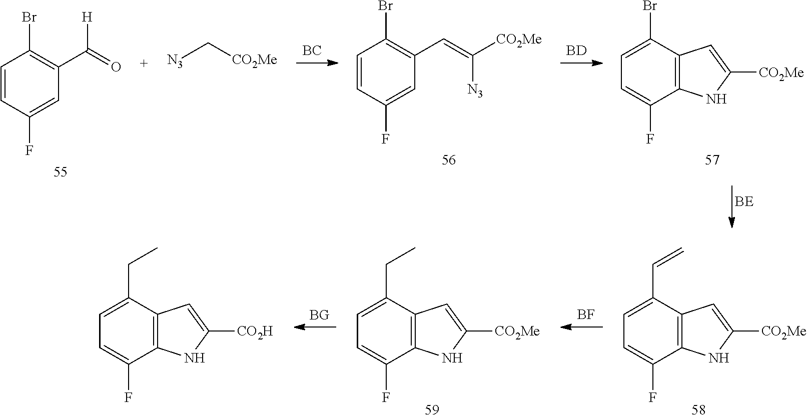

Step BC: To a solution of sodium methoxide (50.0 g, 926 mmol) in methanol (300 mL) −10° C. was added dropwise a solution of compound 55 (45.0 g, 222 mmol) and methyl azidoacetate (59.0 g, 457 mmol) in methanol (100 mL). The reaction mixture was stirred for 3 h maintaining temperature below 5° C., then quenched with ice water. The resulting mixture was stirred for 10 min. The precipitate was collected by filtration, washed with water and dried to afford 33.0 g (110 mmol, 50%) of compound 56 as a white solid.

Step BD: A solution of compound 56, obtained in the previous step, (33.0 g, 110 mmol) in xylene (250 mL) was refluxed for 1 h under an argon atmosphere and then concentrated under reduced pressure. The residue was recrystallized from hexane-ethyl acetate (60:40) to give 21.5 g (79.0 mmol, 72%) of compound 57.

Step BE: To a heated (90° C.) solution of compound 57 (4.00 g, 14.7 mmol) in anhydrous DMF under nitrogen (10 mL) were added tri-n-butyl(vinyl)tin (3.60 g, 11.4 mmol) and Pd(PPh₃)₂Cl₂ (0.301 g, 0.757 mmol). The resulting mixture was stirred at 90° C. for 1 h. The mixture was cooled to room temperature and purified by silica gel column chromatography (60-80% EtOAc in hexane). The combined product fractions of the product were concentrated, washed with water (3×100 mL), dried over Na₂SO₄, and concentrated to give 1.80 g (8.21 mmol, 56%) of compound 58 as yellow solid.

Step BF: A mixture of compound 58 (1.50 g, 6.84 mmol) and Pd/C (0.300 g, 10% wt.) in methanol (20 mL) was stirred under atmosphere of hydrogen at room temperature for 16 h. The mixture was filtered and concentrated to give 1.25 g (5.65 mmol, 83%) of compound 59.

Step BG: To a solution of compound 59 (1.40 g, 6.33 mmol) in methanol (40 mL) was added 2N aqueous NaOH (10 mL). The mixture was stirred for 2 h at 60° C. The mixture was concentrated under reduced pressure, and the residue acidified to pH 5-6 with 10% hydrochloric acid. The precipitate was collected by filtration, washed with water (3×15 mL), and dried to obtain 1.25 g (6.03 mmol, 95%) of target compound 4-ethyl-7-fluoro-1H-indole-2-carboxylic acid.

Rt (Method G) 1.27 mins, m/z 206 [M−H]⁻

### Preparation of 7-fluoro-4-(1-hydroxyethyl)-1H-indole-2-carboxylic acid

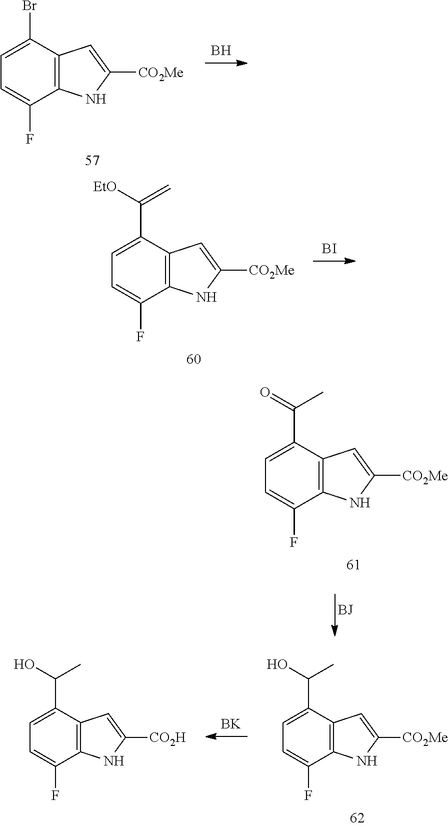

Step BH: To a degassed solution of compound 57 (4.00 g, 14.7 mmol) and tributyl(1-ethoxyvinyl)stannane (5.50 g, 15.2 mmol) in toluene (50 mL) under nitrogen was added bis(triphenylphosphine) palladium(II) dichloride (1.16 g, 1.65 mmol). The reaction mixture was stirred at 60° C. for 20 h. The mixture was cooled to room temperature and filtered. The filtrate was concentrated under reduced pressure and the residue purified by silica gel chromatography to afford 2.70 g (10.3 mmol, 70%) of compound 60 as a pale yellow solid.

Step BI: To a solution of compound 60 (2.40 g, 9.12 mmol) in 1,4-dioxane (30 mL) was added 2M hydrochloric acid (15 mL). The mixture was stirred at room temperature for 30 min. The majority of the solvent was evaporated and the residue was partitioned between ethyl acetate and water. The combined organic extracts were washed with water and brine, dried over sodium sulfate, filtered, and evaporated. The residue was triturated with 5% ether in isohexane and dried to afford 1.90 g (8.08 mmol, 86%) of compound 61 as a white solid.

Step BJ: A suspension of compound 61 (1.70 g, 7.23 mmol) and NaBH₄ (2.50 g, 66.1 mmol) in ethanol (13 mL) was refluxed for 2 h, cooled to room temperature, and filtered. The filtrate was evaporated under reduced pressure and the residue was dissolved in ethyl acetate. The solution was washed with 1N hydrochloric acid and brine, dried over Na₂SO₄, and evaporated under reduced pressure to give 1.50 g (6.32 mmol, 87%) of compound 62 as a colourless oil.

Step BK: To a solution of compound 62 (1.50 g, 6.32 mmol) in methanol (40 mL) was added 2N aqueous NaOH (10 mL). The mixture was stirred for 2 h at 60° C. The mixture was concentrated under reduced pressure and the residue acidified to pH 5-6 with 10% hydrochloric acid. The precipitate was collected by filtration, washed with water (3×15 mL), and dried to obtain 1.35 g (6.05 mmol, 96%) of target compound 7-fluoro-4-(1-hydroxyethyl)-1H-indole-2-carboxylic acid.

Rt (Method G) 0.90 mins, m/z 222 [M−H]⁻

### Preparation of 4-(hydroxymethyl)-1H-indole-2-carboxylic acid

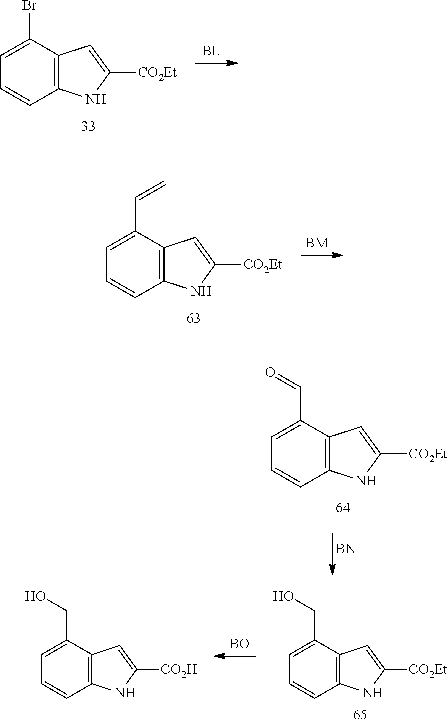

Step BL: To a solution of compound 33 (10.0 g, 39.4 mmol) in a mixture of dioxane (200 mL) and water (50 mL) were added potassium vinyltrifluoroborate (11.0 g, 82.1 mmol), triethylamine (30 mL, 248 mmol) and Pd(dppf)Cl₂ (1.0 g, 1.37 mmol). The mixture was stirred at 80° C. for 48 h. The mixture was concentrated under vacuum, and the residue was dissolved in ethyl acetate. The solution was washed with water and concentrated under reduced pressure.

The obtained material was purified by silica gel column chromatography to give 2.50 g (12.4 mmol, 38%) of compound 63.

Step BM: To a mixture of compound 63 (2.50 g, 12.4 mmol), acetone (200 mL), and water (40 mL) were added OsO₄ (0.100 g, 0.393 mmol) and NaIO₄ (13.4 g, 62.6 mmol). The reaction was stirred for 10 h at room temperature. The acetone was distilled off and the remaining aqueous solution extracted with dichloromethane. The organic layer was washed with saturated NaHCO₃ solution (2×50 mL) and brine (2×50 mL), dried over Na₂SO₄, and concentrated under reduced pressure to obtain 1.50 g (7.40 mmol, 60%) of compound 64.

Step BN: To a cooled (0° C.) solution of compound 64 (1.50 g, 7.38 mmol) in THF/methanol mixture (100 mL) was added NaBH₄ (0.491 g, 13.0 mmol). The reaction mixture was stirred for 12 h at room temperature. Then the mixture was cooled to 0° C., treated with 2N hydrochloric acid (40 mL), and concentrated. The residue was extracted with ethyl acetate. The organic extract was washed with water, dried over Na₂SO₄, and concentrated under reduced pressure to obtain 1.00 g (4.87 mmol, 65%) of compound 65, pure enough for the next step.

Step BO: To a solution of compound 65, obtained in the previous step, (1.00 g, 4.87 mmol) in THF (50 mL), was added 1N aqueous LiOH (9 mL). The resulting mixture was stirred for 48 h at room temperature, then concentrated and diluted with 1N aqueous NaHSO₄ (9 mL). The mixture was extracted with ethyl acetate. The organic extract was dried over Na₂SO₄, and concentrated under reduced pressure. The residue was recrystallized from MTBE to obtain 0.250 g (1.30 mmol, 27%) of target compound 4-(hydroxymethyl)-1H-indole-2-carboxylic acid.

Rt (Method G) 0.98 mins, m/z 190 [M−H]⁻

### Preparation of 4-(2-hydroxypropan-2-yl)-1H-indole-2-carboxylic acid

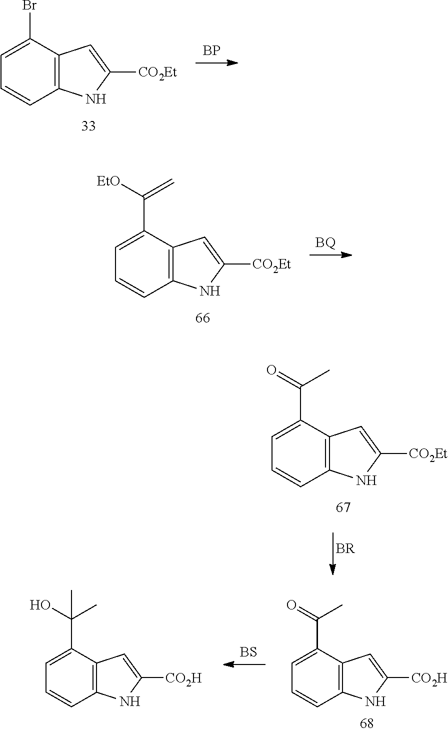

Steps BP and BQ: To a degassed solution of compound 33 (1.00 g, 3.94 mmol) and tributyl-(1-ethoxyvinyl)stannane (1.58 g, 4.37 mmol) in DMF (25 mL) under argon was added bis(triphenylphosphine)palladium(II) dichloride (0.100 g, 0.142 mmol). The reaction mixture was stirred at room temperature until TLC revealed completion of the reaction (approx. 7 days). The mixture was concentrated under reduced pressure and the residue partitioned between ethyl acetate and water. The organic layer was filtered through a plug of silica gel, dried over MgSO₄, and concentrated under reduced pressure. The resulting black oil was dissolved in methanol (100 mL), treated with 5N hydrochloric acid (100 mL), and stirred at room temperature overnight. The mixture was concentrated and the residue dissolved in ethyl acetate. The solution was washed with water, dried over Na₂SO₄, and concentrated under reduced pressure. The crude product was purified by silica gel column chromatography to give 0.500 g (2.30 mmol, 58%) of compound 67.

Step BR: To a solution of compound 67 (1.00 g, 4.60 mmol) in THF (50 mL), was added 1N aqueous LiOH (7 mL). The resulting mixture was stirred for 48 h at room temperature, then concentrated under reduced pressure and diluted with 1N aqueous NaHSO4 (7 mL). The mixture was extracted with ethyl acetate. The organic extract was dried over MgSO₄, and concentrated under reduced pressure. The residue was recrystallized from MTBE to obtain 0.900 g (4.43 mmol, 96%) of compound 68.

Step BS: To a cooled (0° C.) solution of compound 68 (0.900 g, 4.43 mmol) in THF (50 mL) under argon was added a 1N solution of MeMgCl (16 mL) in hexane. The resulting mixture was stirred for 48 h at room temperature. The mixture was carefully quenched with 1N NaHSO₄ and extracted with ethyl acetate. The organic extract was dried over Na₂SO₄, and concentrated under reduced pressure. The residue was recrystallized from MTBE to obtain 0.250 g (1.14 mmol, 26%) of target compound 4-(2-hydroxypropan-2-yl)-1H-indole-2-carboxylic acid.

Rt (Method G) 0.99 mins, m/z 202 [M−H]⁻

### Preparation of 4-(1-hydroxyethyl)-1H-indole-2-carboxylic acid

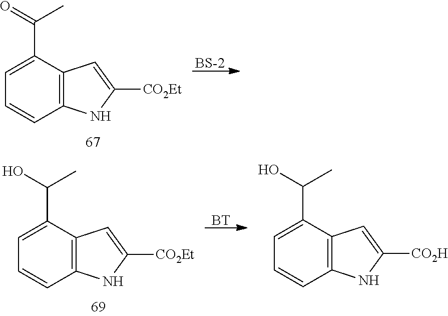 [SMILES: CCOC(=O)c1cc2c(C(C)=O)cccc2[nH]1 ; "BS-2" ; CCOC(=O)c1cc2c(C(C)O)cccc2[nH]1 ; "BT" ; CC(O)c1cccc2[nH]c(C(=O)O)cc12]

Step BS-2: To a cooled (0° C.) solution of compound 67 (1.00 g, 4.60 mmol) in THF/methanol mixture (50 mL) was added NaBH₄ (0.385 g, 10.2 mmol). The reaction mixture was stirred for 12 h at room temperature. The mixture was cooled to 0° C., treated with 2N hydrochloric acid (20 mL), and concentrated. The residue was extracted with ethyl acetate. The organic extract was washed with water, dried over Na₂SO₄, and evaporated under reduced pressure to obtain 0.800 g (3.65 mmol, 79%) of compound 69, pure enough for the next step.

Step BT: To a solution of compound 69, obtained in the previous step, (0.800 g, 3.65 mmol) in THF (50 mL), was added 1N aqueous LiOH (6 mL). The resulting mixture was stirred for 48 h at room temperature, then concentrated and diluted with 1N aqueous NaHSO₄ (6 mL). The mixture was extracted with ethyl acetate. The organic extract was dried over MgSO₄, and concentrated under reduced pressure. The residue was recrystallized from MTBE to obtain 0.300 g (1.46 mmol, 40%) of target compound 4-(1-hydroxyethyl)-1H-indole-2-carboxylic acid.

Rt (Method G) 0.82 mins, m/z 204 [M−H]⁻

### Preparation of 4-(propan-2-yl)-1H-indole-2-carboxylic acid

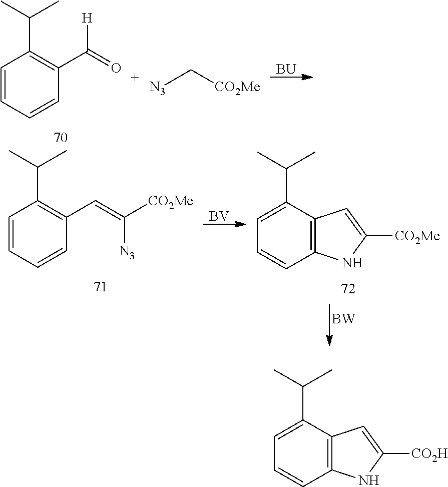

Step BU: To a solution of sodium methoxide (10.0 g, 185 mmol) in methanol (150 mL) at-10° C. was added dropwise a solution of compound 70 (15.0 g, 101 mmol) and methyl azidoacetate (12.0 g, 104 mmol) in methanol (100 mL). The reaction mixture was stirred for 3 h maintaining the temperature below 5° C., then quenched with ice water. The resulting mixture was stirred for 10 min. The precipitate was then collected by filtration, washed with water and dried to afford 7.00 g (23.3 mmol, 23%) of compound 71 as a white solid.

Step BV: A solution of compound 71, obtained in the previous step, (7.00 g, 23.3 mmol) in xylene (200 mL) was refluxed for 1 h under an argon atmosphere and then concentrated under reduced pressure. The residue was recrystallized from hexane-ethyl acetate (60:40) to give 3.50 g (16.1 mmol, 69%) of compound 72.

Step BW: To a solution of compound 72 (3.50 g, 16.1 mmol) in methanol (100 mL) was added 2N aqueous NaOH (40 mL). The mixture was stirred for 2 h at 60° C. The mixture was concentrated under reduced pressure, and then residue acidified to pH 5-6 with 10% hydrochloric acid. The precipitate was collected by filtration, washed with water (3×50 mL), and dried to obtain 2.70 g (13.3 mmol, 83%) of target compound 4-(propan-2-yl)-1H-indole-2-carboxylic acid.

Rt (Method G) 1.32 mins, m/z 202 [M−H]⁻

### Preparation of 4-ethenyl-1H-indole-2-carboxylic acid

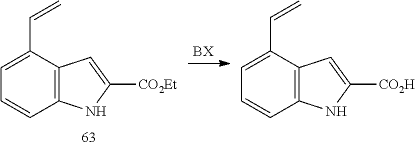 [SMILES: C=Cc1cccc2[nH]c(C(=O)OCC)cc12 ; C=Cc1cccc2[nH]c(C(=O)O)cc12]

Step BX: To a solution of compound 63 (0.900 g, 4.47 mmol) in THF (50 mL), was added 1N aqueous LiOH (8 mL). The resulting mixture was stirred for 48 h at room temperature, then concentrated under reduced pressure and diluted with 1N aqueous NaHSO4 (8 mL). The mixture was extracted with ethyl acetate. The organic extract was dried over MgSO₄ and concentrated under reduced pressure. The residue was recrystallized from MTBE to obtain 0.500 g (2.67 mmol, 59%) of target compound 4-ethenyl-1H-indole-2-carboxylic acid.

Rt (Method G) 1.14 mins, m/z 186 [M−H]⁻

### Preparation of 4-ethynyl-1H-indole-2-carboxylic acid

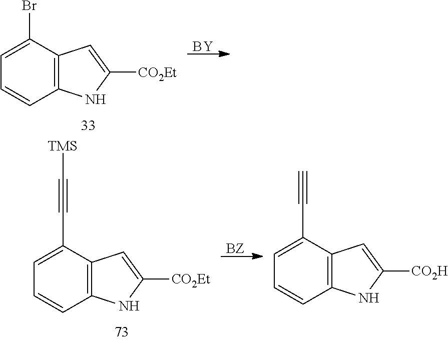

Step BY: To a solution of compound 33 (1.00 g, 3.94 mmol) in THF (50 mL) under argon were added TMS-acetylene (0.68 mL, 4.80 mmol), CuI (0.076 g, 0.399 mmol), triethylamine (2.80 mL, 20.0 mmol), and Pd(dppf)Cl₂ (0.100 g, 0.137 mmol). The mixture was stirred at 60° C. until TLC revealed completion of the reaction (approx. 5 days). The mixture was concentrated under reduced pressure, and the residue dissolved in ethyl acetate. The solution was washed with water, dried over Na₂SO₄, and concentrated under reduced pressure. The residue was purified by silica gel column chromatography to give 0.600 g (2.14 mmol, 56%) of compound 73.

Step BZ: To a solution of compound 73 (0.840 g, 3.10 mmol) in THF (50 mL), was added 1N aqueous LiOH (7 mL). The resulting mixture was stirred for 48 h at room temperature, then concentrated under reduced pressure and diluted with 1N aqueous NaHSO₄ (7 mL). The mixture was extracted with ethyl acetate. The organic extract was dried over MgSO₄ and concentrated under reduced pressure. The residue was recrystallized from MTBE to obtain 0.400 g (2.17 mmol, 70%) of target compound 4-ethynyl-1H-indole-2-carboxylic acid.

Rt (Method G) 1.12 mins, m/z 184 [M−H]⁻

### Preparation of 4-(1,1-difluoroethyl)-1H-indole-2-carboxylic acid

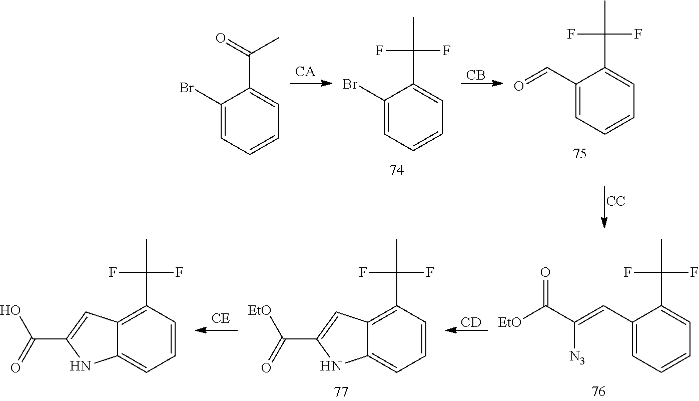

Step CA: To a mixture of 2-bromoacetophenone (63.0 g, 317 mmol), water (0.5 mL), and dichloromethane (100 mL) was added Morph-DAST (121 mL, 992 mmol). The resulting mixture was stirred for 28 days at room temperature. The reaction mixture was then poured into saturated aqueous NaHCO₃ (1000 mL) and extracted with ethyl acetate (2×500 mL). The organic layer was dried over Na₂SO₄ and concentrated under reduced pressure. The residue was purified by silica gel column chromatography to give 16.8 g (76.0 mmol, 12%) of compound 74.

Step CB: To a cooled (−85° C.) solution of compound 74 (16.8 g, 76.0 mmol) in THF (300 mL) under Ar was added 2.5M solution of n-BuLi in hexanes (36.5 mL, 91.5 mmol) over 30 min. The resulting mixture was stirred for 1 h at −85° C. DMF (8.80 mL, 114 mmol) was then added (maintaining temperature below −80° C.) and the reaction stirred for a further 45 min. The reaction was quenched with saturated aqueous NH₄Cl (100 mL) and diluted with water (600 mL). The obtained mixture was extracted with ethyl acetate (2×500 mL). The combined organic extracts were dried over Na₂SO₄, and concentrated under reduced pressure to obtain 12.5 g (73.6 mmol, 97%) of compound 75 (sufficiently pure for the next step).

Step CC: To a cooled (−30° C.) mixture of compound 75 (12.5 g, 73.5 mmol), ethanol (500 mL), and ethyl azidoacetate (28.5 g, 221 mmol) was added a freshly prepared solution of sodium methoxide (prepared by mixing Na (5.00 g, 217 mmol) and methanol (100 mL)) portionwise under Ar (maintaining the temperature below −25° C.). The reaction mixture was warmed to 15° C. and stirred for 12 h. The obtained mixture was poured into saturated aqueous NH₄Cl (2500 mL) and stirred for 20 min. The precipitate was collected by filtration, washed with water, and dried to obtain 10.0 g (35.6 mmol, 51%) of compound 76.

Step CD: A solution of compound 76 (10.0 g, 35.6 mmol) in xylene (500 mL) was refluxed until gas evolution ceased (approx. 2 h) and then concentrated under reduced pressure. The orange oil obtained was triturated with hexane/ethyl acetate (5:1), collected by filtration, and dried to obtain 1.53 g (6.04 mmol, 17%) of compound 77.

Step CE: To a solution of compound 77 (1.53 g, 6.04 mmol) in THF/water 9:1 mixture (100 mL) was added LiOH.H₂O (0.590 g, 14.1 mmol). The resulting mixture was stirred overnight at r.t. The volatiles were evaporated and the residue mixed with water (50 mL) and 1N hydrochloric acid (10 mL). The mixture was extracted with ethyl acetate (2×100 mL). The combined organic extracts were dried over Na₂SO₄, and concentrated under reduced pressure. The crude product was purified by silica gel column chromatography to give 0.340 g (1.33 mmol, 24%) of 4-(1,1-difluoroethyl)-1H-indole-2-carboxylic acid.

Rt (Method G) 1.16 mins, m/z 224 [M−H]⁻

### Preparation of 4-(trimethylsilyl)-1H-indole-2-carboxylic acid

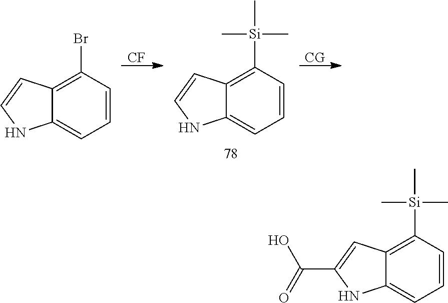

Step CF: To a cooled (−78° C.) solution of 4-bromo-1H-indole (5.00 g, 25.5 mmol) in THF (100 mL) under Ar was added a 2.5M solution of n-BuLi in hexanes (23 mL, 57.5 mmol). The resulting mixture was stirred for 30 min. TMSCl (16 mL, 126 mmol) was added and the reaction mixture warmed to room temperature. After 1 h the mixture was diluted with MTBE (250 mL), washed with water (2×200 mL) and brine (200 mL), then dried over Na₂SO₄, and concentrated under reduced pressure. The residue was refluxed in methanol (100 mL) for 1 h. The solvent was then distilled off to obtain 3.60 g (19.0 mmol, 74%) of compound 78.

Step CG: To a cooled (−78° C.) solution of compound 78 (1.50 g, 7.92 mmol) in THF (50 mL) under Ar was added a 2.5M solution of n-BuLi in hexanes (3.8 mL, 9.5 mmol). The resulting mixture was stirred for 20 min. CO₂ (2 L) was then bubbled through the mixture for 10 min, and the reaction mixture warmed to room temperature. The volatiles were evaporated and the residue dissolved in THF (50 mL). The solution was cooled to −78° C., and a 1.7M solution of t-BuLi (5.6 mL, 9.50 mmol) was added. The mixture was warmed to −30° C., then again cooled to −78° C. CO₂ (2 L) was bubbled through the solution for 10 min. The obtained solution was allowed to slowly warm to r.t. then concentrated under reduced pressure. The residue was dissolved in water (50 mL), washed with MTBE (2×50 mL), then acidified to pH 4, and extracted with ethyl acetate (2×50 mL). The organic extract was washed with water (2×50 mL), and brine (50 mL), dried over Na₂SO₄, and evaporated under reduced pressure. The crude product was washed with hexane and dried to obtain 1.24 g (5.31 mmol, 67%) of target compound 4-(trimethylsilyl)-1H-indole-2-carboxylic acid.

Rt (Method G) 1.47 mins, m/z 232 [M−H]⁻

### Preparation of 6-chloro-5-fluoro-1H-indole-2-carboxylic acid

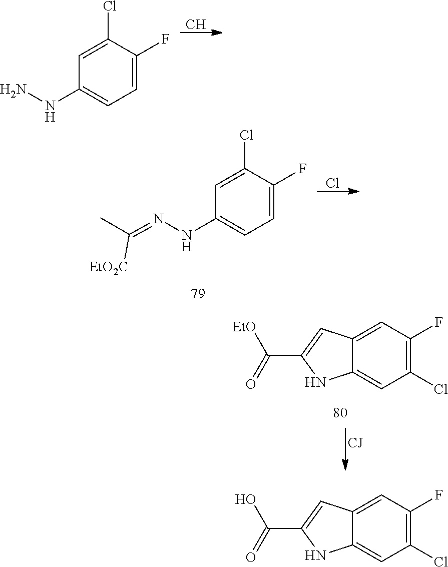

Step CH: To a solution of (3-chloro-4-fluorophenyl)hydrazine (80.0 g, 498 mmol) in ethanol (200 mL) was added ethyl pyruvate (58.0 g, 499 mmol). The mixture was refluxed for 1 h, then concentrated under reduced pressure, and diluted with water (300 mL). The solid was collected by filtration then dried to obtain 122 g (472 mmol, 95%) of compound 79.

Step CI: A suspension of compound 79 (122 g, 472 mmol) and pTSA (81.5 g, 473 mmol) in toluene (500 mL) was refluxed for 48 h, then cooled to room temperature. The precipitate was collected by filtration and purified by fractional crystallization from toluene to obtain 4.00 g (16.6 mmol, 4%) of compound 80.

Step CJ: To a refluxing solution of compound 80 (4.00 g, 16.6 mmol) in ethanol (30 mL) was added NaOH (0.660 g, 16.5 mmol). The mixture was refluxed for 1 h, then concentrated under reduced pressure. The residue was triturated with warm water (80° C., 50 mL) and the solution acidified (pH 2) with concentrated hydrochloric acid. The precipitate was collected by filtration, washed with water (2×10 mL), and dried to obtain 3.18 g (14.9 mmol, 90%) of target compound 6-chloro-5-fluoro-1H-indole-2-carboxylic acid.

Rt (Method G) 1.23 mins, m/z 212 [M−H]⁻

### Preparation of 4-(difluoromethyl)-6-fluoro-1H-indole-2-carboxylic acid

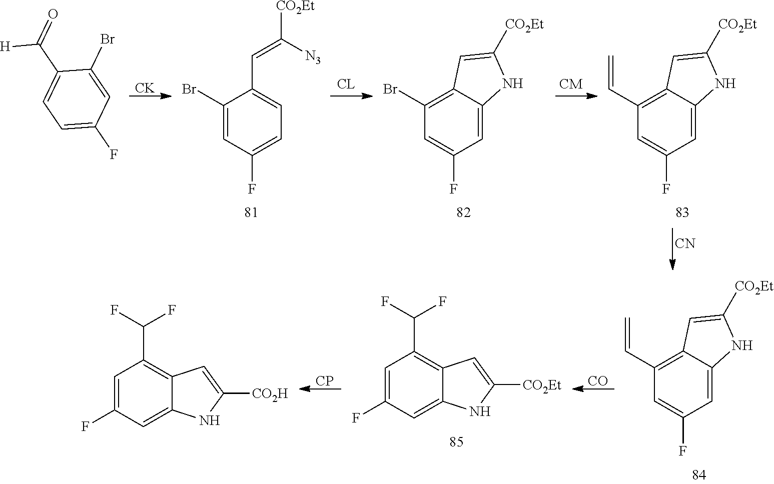

Step CK: To a solution of sodium methoxide (50.0 g, 926 mmol) in methanol (300 mL) at −10° C. was added dropwise a solution of 2-bromo-4-fluorobenzaldehyde (222 mmol) and methyl azidoacetate (59.0 g, 457 mmol) in methanol (100 mL). The reaction mixture was stirred for 3 h, maintaining the temperature below 5° C., then quenched with ice water. The resulting mixture was stirred for 10 min and the solid collected by filtration. The solid was washed with water to afford compound 81 as a white solid (62% yield).

Step CL: A solution of compound 81 (133 mmol) in xylene (250 mL) was refluxed for 1 h under an argon atmosphere and then concentrated under reduced pressure. The residue was recrystallized form hexane-ethyl acetate mixture (60:40) to give compound 82 (58% yield).

Step CM: To a heated (90° C.) solution of compound 82 (14.7 mmol) in anhydrous DMF (10 mL) tri-n-butyl(vinyl)tin (3.60 g, 11.4 mmol) and Pd(PPh3)₂C12 (0.301 g, 0.757 mmol) were added under nitrogen and the resulting mixture was stirred at 90° C. for 1 h. The mixture was cooled to room temperature and purified by silica gel column chromatography (60-80% ethyl acetate in hexane). The combined product fractions were concentrated, washed with water (3×100 mL), dried over Na₂SO₄, and concentrated under reduced pressure to afford compound 83 as a yellow solid (60% yield).

Step CN: To a mixture of compound 83 (12.4 mmol), acetone (200 mL), and water (40 mL) OsO₄ (0.100 g, 0.393 mmol) and NaIO4 (13.4 g, 62.6 mmol) were added and the reaction was stirred for 10 h at room temperature. Acetone was distilled off and the aqueous solution was extracted with dichloromethane. The combined organic layer was washed with saturated NaHCO₃ solution (2×50 mL) and brine (2×50 mL), dried over Na₂SO₄, and concentrated under reduced pressure to afford compound 84 (33% yield).

Step CO: To a solution of compound 84 (11.0 mmol) in dichloromethane (50 mL) was added Morph-DAST (4.10 mL, 33.6 mmol). The resulting mixture was stirred until NMR of an aliquot revealed completion of the reaction (2-5 days). The reaction mixture was added dropwise to a cold saturated NaHCO₃ solution (1000 mL). The mixture obtained was extracted with ethyl acetate. The organic layer was dried over MgSO₄ and concentrated. The residue was purified by column chromatography to give compound 85 as yellow solid (48% yield).

Step CP: To a solution of compound 85 (4.50 mmol) in THF (50 mL), was added 1N aqueous LiOH (8 mL). The resulting mixture was stirred for 48 h at room temperature then concentrated under reduced pressure and diluted with 1N aqueous NaHSO₄ (8 mL). The obtained mixture was extracted with ethyl acetate. The organic extract was dried over MgSO₄ and concentrated under reduced pressure. The residue was recrystallized from MTBE to obtain 4-(difluoromethyl)-6-fluoro-1H-indole-2-carboxylic acid (87%).

Rt (Method G) 1.22 mins, m/z 228 [M−H]⁻

### Preparation of 4-(difluoromethyl)-7-fluoro-1H-indole-2-carboxylic acid

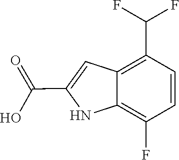 [SMILES: O=C(O)c1cc2c(C(F)F)ccc(F)c2[nH]1]

Prepared as described for 4-(difluoromethyl)-6-fluoro-1H-indole-2-carboxylic acid, starting from 2-bromo-5-fluorobenzaldehyde (2.5% overall yield).

Rt (Method G) 1.13 mins, m/z 228 [M−H]⁻

### Preparation of 4-(difluoromethyl)-1H-indole-2-carboxylic acid

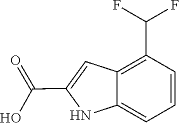 [SMILES: O=C(O)c1cc2c(C(F)F)cccc2[nH]1]

Prepared as described for 4-(difluoromethyl)-6-fluoro-1H-indole-2-carboxylic acid, starting from 4-bromo-1H-indole-2-carboxylic acid (11% overall yield).

Rt (Method G) 1.17 mins, m/z 210 [M−H]⁻

### Preparation of 4-(1,1-difluoroethyl)-6-fluoro-1H-indole-2-carboxylic acid

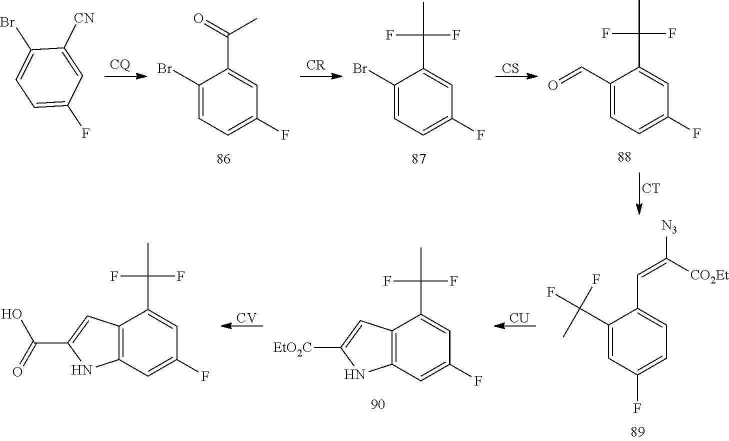

Step CQ: To a solution of 2-bromo-5-fluorobenzonitrile (10.0 g, 48.5 mmol) in anhydrous tetrahydrofuran (100 mL) under nitrogen was added methylmagnesium bromide (3.2M in ether, 19 mL, 60.0 mmol). The resulting mixture was heated to reflux for 4 h. The reaction mixture was then cooled, poured into 2N hydrochloric acid (100 mL), and diluted with methanol (100 mL). The organic solvents were removed and the crude product precipitated out. The reaction mixture was extracted with ethyl acetate, dried over MgSO₄, and concentrated. The residue was purified by column chromatography (heptane/dichloromethane) to give 4.88 g (21.9 mmol, 45%) of compound 86 as a pink oil.

Step CR: To a solution of compound 86 (110 mmol) in dichloromethane (50 mL) at room temperature was added Morph-DAST (41 mL, 336 mmol) and a few drops of water. The resulting mixture was stirred for 48 days at room temperature; every 7 days an additional portion of Morph-DAST (41 mL, 336 mmol) was added. After the reaction was complete, the mixture was carefully added dropwise to cold saturated aqueous NaHCO₃. The product was extracted with ethyl acetate and the organic extract dried over MgSO₄ and concentrated. The residue was purified by column chromatography to give 87 as a colorless liquid (37% yield).

Step CS: To a cooled (−80° C.) solution of compound 87 (21.0 mmol) in THF (150 mL) was added slowly a 2.5M solution of n-BuLi in hexanes (10.0 mL, 25.0 mmol of n-BuLi). The mixture was stirred for 1 h, then DMF (2.62 mL, 33.8 mmol) was added and the mixture stirred for a further 1 h. The reaction was quenched with saturated aqueous NH₄Cl (250 mL) and extracted with Et₂O (3×150 mL). The organic layer was dried over Na₂SO₄ and concentrated under reduced pressure. The residue was purified by silica gel chromatography (ethyl acetate/hexane 1:9) to give compound 88 (52% yield).

Step CT: To a solution of sodium methoxide (50.0 g, 926 mmol) in methanol (300 mL) at-10° C. was added dropwise a solution of compound 88 (222 mmol) and methyl azidoacetate (59.0 g, 457 mmol) in methanol (100 mL). The reaction mixture was stirred for 3 h, maintaining the temperature below 5° C., then quenched with ice water. The resulting mixture was stirred for 10 min. The solid obtained was collected by filtration, and washed with water to afford compound 89 as a white solid (66% yield).

Step CU: A solution of compound 89 (120 mmol) in xylene (250 mL) was refluxed for 1 h under an argon atmosphere and then concentrated under reduced pressure. The residue was recrystallized from hexane-ethyl acetate to give compound 90 (70% yield).

Step CV: To a solution of compound 90 (4.40 mmol) in THF (50 mL) was added 1N aqueous LiOH (8 mL). The resulting mixture was stirred for 48 h at room temperature, then concentrated under reduced pressure and diluted with 1N aqueous NaHSO₄ (8 mL). The residue obtained was extracted with ethyl acetate. The organic extract was dried over MgSO₄ and concentrated under reduced pressure. The residue was recrystallized from MTBE to obtain target compound 4-(1,1-difluoroethyl)-6-fluoro-1H-indole-2-carboxylic acid (95% yield).

Rt (Method G) 1.26 mins, m/z 242 [M−H]⁻

### Preparation of 4-(1,1-difluoroethyl)-7-fluoro-1H-indole-2-carboxylic acid

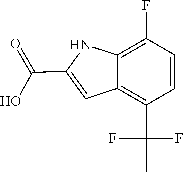 [SMILES: CC(F)(F)c1ccc(F)c2[nH]c(C(=O)O)cc12]

Prepared as described for 4-(1,1-difluoroethyl)-6-fluoro-1H-indole-2-carboxylic acid, starting from 2-bromo-4-fluoroacetophenone (3.6% overall yield).

Rt (Method G) 1.23 mins, m/z 242 [M−H]⁻

### Preparation of 5-[(tert-butoxy)carbonyl]-6-methyl-1-{[2-(trimethylsilyl)ethoxy]methyl}-1H,4H,5H,6H,7H-pyrazolo[4,3-c]pyridine-3-carboxylic acid

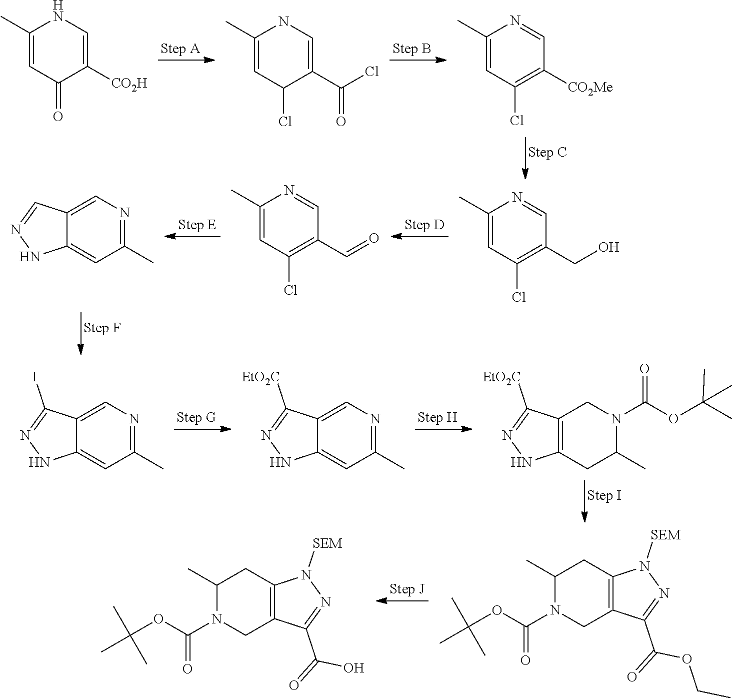

Step A: 6-Methyl-4-oxo-1,4-dihydropyridine-3-carboxylic acid (50.0 g, 326.51 mmol) was suspended in phosphoryl trichloride (500.0 g, 3.26 mol) and stirred at 95° C. for 16 h. After cooling, the excess phosphorus oxychloride was distilled off in vacuo, and obtained residue was evaporated with toluene (2×250 mL) to give 5-(carboxy)-4-chloro-2-methylpyridin-1-ium chloride (73.3 g, 95.0% purity, 307.46 mmol, 94.2% yield).

Step B: 5-(Carboxy)-4-chloro-2-methylpyridin-1-ium chloride (73.3 g, 323.64 mmol) was dissolved in THF (500 mL) and MeOH (500 mL) was added dropwise at 100° C. The mixture was stirred at r.t. for 2 h. The mixture was concentrated to give a residue which was dissolved in CH₂Cl₂ (700 mL) and washed with a saturated solution of NaHCO₃. The combined organic extracts were concentrated in vacuo to give an orange oil which was purified by column chromatography (MTBE-hexane 2:1) (Rf=0.8) to yield methyl 4-chloro-6-methylpyridine-3-carboxylate (57.7 g, 98.0% purity, 304.65 mmol, 94.1% yield) as a yellow oil that crystallized on standing to give a yellow solid.

Step C: To a cooled (−25° C.) suspension of lithium aluminium hydride (6 g) in THF (500 mL) was added dropwise a solution of methyl 4-chloro-6-methylnicotinate (33.0 g, 177.79 mmol) in tetrahydrofuran (100 mL). The mixture was stirred at 0° C. for 1.5 hours. Water (6 mL in 50 mL of THF), 15% aqueous solution of sodium hydroxide (6 mL) and water (18 mL) were dropped successively to the reaction mixture. The mixture was stirred at r.t. for 30 minutes, filtered and the filter cake washed with THF (2×200 mL).The filtrate was concentrated to give the title compound (4-chloro-6-methylpyridin-3-yl)methanol (26.3 g, 95.0% purity, 158.54 mmol, 89.2% yield) as a yellow solid that was used without further purification.

Step D: To a solution of (4-chloro-6-methylpyridin-3-yl)methanol (26.3 g, 166.88 mmol) in DCM (777 mL) was added 1,1,1-tris(acetoxy)-1,1-dihydro-1,2-benziodoxol-3(1H)-one (81.4 g, 191.92 mmol) in few portions, maintaining the temperature below 5° C. with an water/ice cooling bath. After the reaction was complete (monitored by 1H NMR) the mixture was poured into a stirred aqueous solution of sodium hydrogen carbonate (16.12 g, 191.91 mmol) and Na₂S₂O₃ and stirred until organic phase became transparent (about 2 h). The layers were separated and aqueous layer was extracted with DCM (3×300 mL), and the combined organic extracts were washed with brine, dried over Na₂SO₄ and concentrated under reduced pressure to give 4-chloro-6-methylpyridine-3-carbaldehyde (21.0 g, 90.0% purity, 121.48 mmol, 72.8% yield) that was used in the next step without further purification.

Step E: To a suspension of 4-chloro-6-methylpyridine-3-carbaldehyde (17.0 g, 109.27 mmol) (1 equiv.) in ethylene glycol dimethyl ether (300 mL) and 1,4-dioxane (300 ml) was added hydrazine hydrate (191.45 g, 3.82 mol) (98 percent) (35.00 equiv.). The mixture was refluxed for 96 h (1H NMR analysis). The layers were separated and the organic layer was concentrated under reduced pressure. Water (200 mL) was added to the residue, and the mixture was stirred at room temperature for 1 hour. Product was collected by filtration, washed with water (100 mL), then dried to give 6-methyl-1H-pyrazolo[4,3-c]pyridine (3.42 g, 98.0% purity, 25.17 mmol, 23% yield) as a yellow solid.

Step F: A suspension of 6-methyl-1H-pyrazolo[4,3-c]pyridine (1.91 g, 14.34 mmol) (1.00 equiv), iodine (7.28 g, 28.69 mmol) (2.00 equiv), and potassium hydroxide (2.9 g, 51.63 mmol) (3.60 equiv) in DMF (40 mL) was stirred at r.t. for 12 h. The reaction was quenched by addition of saturated aqueous Na₂S₂O₃, extracted with ethyl acetate (3×200 mL), dried over anhydrous sodium sulfate and concentrated under reduced pressure to give 3-iodo-6-methyl-1H-pyrazolo[4,3-c]pyridine (3.1 g, 98.0% purity, 11.73 mmol, 81.8% yield) as a yellow solid.

Step G: 3-Iodo-6-methyl-1H-pyrazolo[4,3-c]pyridine (5.05 g, 19.49 mmol), triethylamine (2.37 g, 23.39 mmol, 3.26 mL) and Pd(dppf)Cl₂ (3 mol %) were dissolved in ethanol (96%, 200 ml). The reaction mixture was heated at 120° C. in high pressure vessel at 40 atm CO pressure for 18 h. The mixture was then concentrated and water (100 ml) was added to the obtained residue. The mixture was stirred at room temperature for 1 hour and product collected by filtration. The solid was washed with water (100 mL), then dried to give ethyl 6-methyl-1H-pyrazolo[4,3-c]pyridine-3-carboxylate (2.7 g, 95.0% purity, 12.5 mmol, 64.1% yield) as an orange solid.

Step H: To a suspension of ethyl 6-methyl-1H-pyrazolo[4,3-c]pyridine-3-carboxylate (620.23 mg, 3.02 mmol) and di-tert-butyl dicarbonate (692.6 mg, 3.17 mmol) in methanol (133 mL) (plus 5 drops of Et₃N) was added 20% Pd(OH)₂ on carbon. The mixture was hydrogenated in an autoclave at 40 bar and then allowed to stir at r.t for 18 h. The reaction mixture was filtered through a thin pad of silica and the pad was washed with CH₃OH (30 mL). The filtrate was concentrated under reduced pressure to give 5-tert-butyl 3-ethyl 6-methyl-1H,4H,5H,6H,7H-pyrazolo[4,3-c]pyridine-3,5-dicarboxylate (888.89 mg, 98.0% purity, 2.82 mmol, 93.2% yield) as an oil.

Step I: To a cooled (0° C.) solution of 5-tert-butyl 3-ethyl 6-methyl-1H,4H,5H,6H,7H-pyrazolo[4,3-c]pyridine-3,5-dicarboxylate (1.1 g, 3.56 mmol) (1 eq.) in THF (75 ml) was added sodium hydride (60%, 1.33 eq) portionwise. The mixture was stirred at room temperature for 0.5 h. [2-(Chloromethoxy)ethyl]trimethylsilane (788.36 mg, 4.73 mmol) was added dropwise and the mixture stirred at room temperature for an additional 16 h. The mixture was quenched with water and extracted with EtOAc (3×30 mL). The combined organic extracts were dried over anhydrous Na₂SO₄, filtered and concentrated under reduced pressure to give 5-tert-butyl 3-ethyl 6-methyl-1-[2-(trimethylsilyl)ethoxy]methyl-1H,4H,5H,6H,7H-pyrazolo[4,3-c]pyridine-3,5-dicarboxylate (1.56 g, 64.0% purity, 2.26 mmol, 63.7% yield) as yellow oil that was used in the next step without further purification.

Step J: 5-Tert-butyl 3-ethyl 6-methyl-1-[2-(trimethylsilyl)ethoxy]methyl-1H,4H,5H,6H,7H-pyrazolo[4,3-c]pyridine-3,5-dicarboxylate (808.0 mg, 1.84 mmol) and lithium hydroxide monohydrate (231.25 mg, 5.51 mmol) were stirred in a mixture of THF:H₂O:CH₃OH (v/v 3:1:1, 50 mL) at 25° C. for 18 h. The reaction mixture was then concentrated under reduced pressure and acidified to pH 4 with a saturated aqueous solution of citric acid. The mixture was extracted with EtOAc (3×30 mL). The combined organic extracts were dried over anhydrous Na₂SO₄, filtered and concentrated under reduced pressure. The crude product was purified by HPLC to give 5-[(tert-butoxy)carbonyl]-6-methyl-1-[2-(trimethylsilyl)ethoxy]methyl-1H,4H,5H,6H,7H-pyrazolo[4,3-c]pyridine-3-carboxylic acid (505.0 mg, 99.0% purity, 1.21 mmol, 66.1% yield) as white solid.

Rt (Method G) 1.57 mins, m/z 412 [M+H]⁺

1H NMR (400 MHz, DMSO) S−0.07 (s, 9H), 0.80 (t, J=7.9 Hz, 2H), 1.02 (d, J=6.9 Hz, 3H), 1.41 (s, 9H), 2.69 (d, J=16.4 Hz, 1H), 2.83 (dd, J=16.3, 6.1 Hz, 1H), 3.48 (m, 2H), 3.98 (d, J=17.5 Hz, 1H), 4.71 (br.s, 1H), 4.88 (d, J=17.1 Hz, 1H), 5.39 (AB-system, 2H), 12.77 (br.s, 1H).

### Preparation of 5-[(tert-butoxy)carbonyl]-1-{[2-(trimethylsilyl)ethoxy]methyl}-1H,4H,5H,6H,7H-pyrazolo[4,3-c]pyridine-3-carboxylic acid

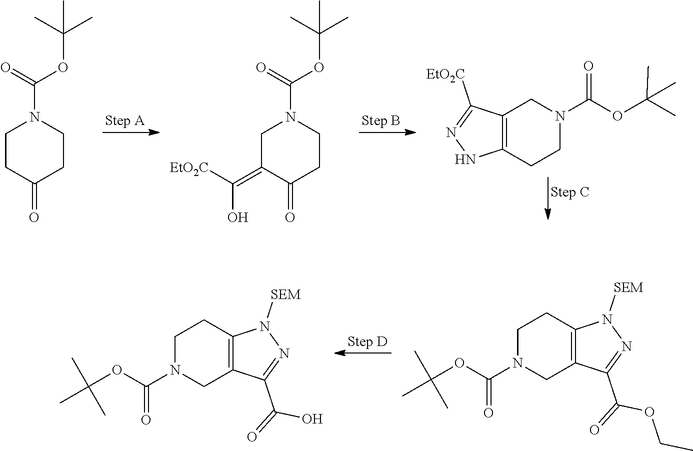 [SMILES: CC(C)(C)OC(=O)N1CCC(=O)CC1 ; CCOC(=O)/C(O)=C1\CN(C(=O)OC(C)(C)C)CCC1=O ; CCOC(=O)c1n[nH]c2c1CN(C(=O)OC(C)(C)C)CC2 ; CC(C)(C)OC(=O)N1CCc2c(c(C(=O)O)nn2COCC[Si](C)(C)C)C1 ; CCOC(=O)c1nn(COCC[Si](C)(C)C)c2c1CN(C(=O)OC(C)(C)C)CC2]

Step A: Lithium bis(trimethylsilyl)amide (8.4 g, 50.21 mmol, 50.21 mL) was dissolved in dry Et₂O (50 mL) and cooled to −78° C. (dry-ice/acetone). To the cooled mixture was added a solution of tert-butyl 4-oxopiperidine-1-carboxylate (10.0 g, 50.21 mmol) in dry Et₂O/THF (3:1) (60 mL).Once addition was complete, the mixture was stirred for 30 min. A solution of diethyl oxalate (7.34 g, 50.21 mmol, 6.82 mL) in dry Et₂O (20 mL) was added over 10 min. The mixture was stirred for 15 min at −78° C. after which the cooling was removed. The reaction mixture was stirred overnight at 20° C. The mixture was poured into 1M KHSO₄ (200 mL) and the layers were separated. The aqueous phase was extracted with EtOAc (2×100 mL). The combined organic layers were separated, washed with water, dried (Na₂SO₄), filtered and concentrated to give tert-butyl 3-(2-ethoxy-2-oxoacetyl)-4-oxopiperidine-1-carboxylate (14.1 g, 47.11 mmol, 93.8% yield) as orange oil, which was used in the next step without further purification.

Step B: To a stirred solution of tert-butyl 3-(2-ethoxy-2-oxoacetyl)-4-oxopiperidine-1-carboxylate (14.11 g, 47.14 mmol) in abs. EtOH (150 mL) was added acetic acid (4.53 g, 75.43 mmol, 4.32 mL) followed by portionwise addition of hydrazine hydrate (2.36 g, 47.14 mmol, 3.93 mL) The mixture was stirred for 5 h, then concentrated, and the residue obtained diluted with sat. NaHCO₃. The product was extracted with EtOAc (2×100 mL). The combined organic extracts were dried over Na₂SO₄, filtered and concentrated to afford 5-tert-butyl 3-ethyl 1H,4H,5H,6H,7H-pyrazolo[4,3-c]pyridine-3,5-dicarboxylate (11.2 g, 37.92 mmol, 80.4% yield) as yellow foam, crystallized with standing.

Step C: To a cooled (0° C.) suspension of sodium hydride (1.82 g, 0.045 mol, 60% dispersion in mineral oil) in dry THF (250 mL) under argon was added dropwise a solution of 5-tert-butyl 3-ethyl 1H,4H,5H,6H,7H-pyrazolo[4,3-c]pyridine-3,5-dicarboxylate (11.2 g, 37.92 mmol) in dry THF (50 mL). The mixture was stirred for 30 min at 0° C., then [2-(chloromethoxy)ethyl]trimethylsilane (7.59 g, 45.51 mmol) was added dropwise. The resulting mixture was stirred for 30 min at 0° C., and then warmed to room temperature. The mixture was poured in water (250 mL), and the product was extracted with EtOAc (2×200 mL). The combined organic extracts were washed with brine, dried over Na₂SO₄ and concentrated to afford crude 5-tert-butyl 3-ethyl 1-[2-(trimethylsilyl)ethoxy]methyl-1H,4H,5H,6H,7H-pyrazolo[4,3-c]pyridine-3,5-dicarboxylate (15.3 g, 35.95 mmol, 94.8% yield) as yellow oil which was used in the next step without further purification.

Step D: To a solution of 5-tert-butyl 3-ethyl 1-[2-(trimethylsilyl)ethoxy]methyl-1H,4H,5H,6H,7H-pyrazolo[4,3-c]pyridine-3,5-dicarboxylate (15.3 g, 35.95 mmol) in THF (100 mL)/water (50 mL) was added lithium hydroxide monohydrate (5.28 g, 125.82 mmol). The reaction mixture was stirred at 50° C. for 3 h, and then concentrated. The residue was carefully acidified with sat. aq. solution of KHSO₄ to pH 4-5 and product was extracted with EtOAc (2×200 mL). The combined organic extracts were dried with Na₂SO₄, filtered and evaporated. The solid residue was triturated with hexane. Product was collected by filtration and dried to afford 5-[(tert-butoxy)carbonyl]-1-[2-(trimethylsilyl)ethoxy]methyl-1H,4H,5H,6H,7H-pyrazolo[4,3-c]pyridine-3-carboxylic acid (7.5 g, 18.87 mmol, 52.5% yield) as yellow solid.

Rt (Method G) 1.52 mins, m/z 398 [M+H]⁺

1H NMR (400 MHz, CDCl₃) δ −0.05 (s, 9H), 0.87 (t, J=8.2 Hz, 2H), 1.47 (s, 9H), 2.78 (m, 2H), 3.55 (m, 2H), 3.71 (m, 2H), 4.62 (br.s, 2H), 5.43 (s, 2H), COOH is not observed.

### Preparation of 6,6-difluoro-4-azaspiro[2.4]heptane

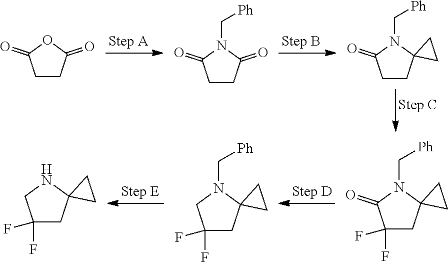 [SMILES: O=C1CCC(=O)O1 ; O=C1CCC(=O)N1Cc1ccccc1 ; O=C1CCC2(CC2)N1Cc1ccccc1 ; FC1(F)CNC2(CC2)C1 ; FC1(F)CN(Cc2ccccc2)C2(CC2)C1 ; O=C1N(Cc2ccccc2)C2(CC2)CC1(F)F]

Step A: To a solution of succinic anhydride (100 g, 1000 mmol) in toluene (3000 mL) was added benzylamine (107 g, 1000 mmol). The solution was stirred at room temperature for 24 h, and then heated at reflux with a Dean-Stark apparatus for 16 hours. The mixture was then concentrated under reduced pressure to give 1-benzylpyrrolidine-2,5-dione (170 g, 900 mmol, 90% yield).

Step B: To a cooled (0° C.) mixture of 1-benzylpyrrolidine-2,5-dione (114 g, 600 mmol) and Ti(Oi-Pr)₄ (170.5 g, 600 mmol) in dry THF (2000 mL) under argon atmosphere was added dropwise a 3.4M solution of ethyl magnesium bromide in THF (1200 mmol). The mixture was warmed to room temperature and stirred for 4 h. BF₃.Et₂O (170 g, 1200 mmol) was then added dropwise and the solution stirred for 6 h. The mixture was cooled (0° C.) and 3N hydrochloric acid (500 mL) was added. The mixture was extracted twice with Et₂O, and the combined organic extracts washed with brine, dried and concentrated under reduced pressure to give 4-benzyl-4-azaspiro[2.4]heptan-5-one (30.2 g, 150 mmol, 25% yield).

Step C: To a cooled (−78° C.) solution of 4-benzyl-4-azaspiro[2.4]heptan-5-one (34.2 g, 170 mmol) in dry THF (1000 mL) under argon was added LiHMDS in THF (1.1M solution, 240 mmol). The mixture was stirred for 1 h, and then a solution of N-fluorobenzenesulfonimide (75.7 g, 240 mmol) in THF (200 mL) was added dropwise. The mixture was warmed to room temperature and stirred for 6 h. The mixture was then re-cooled (−78° C.) and LiHMDS added (1.1M solution in THF, 240 mmol).

The solution was stirred for 1 h, and then N-fluorobenzenesulfonimide (75.7 g, 240 mmol) in THF (200 mL) was added dropwise. The mixture was warmed to room temperature and stirred for 6 h. The mixture was poured into a saturated solution of NH₄Cl (300 mL) and extracted twice with Et₂O. The combined organic extracts were washed with brine and concentrated under reduced pressure. Product was purified by column chromatography to provide 4-benzyl-6,6-difluoro-4-azaspiro[2.4]heptan-5-one (18 g, 75.9 mmol, 45% yield).

Step D: To a warmed (40° C.) solution of BH₃.Me₂S (3.42 g, 45 mmol) in THF (200 mL) was added dropwise 4-benzyl-6,6-difluoro-4-azaspiro[2.4]heptan-5-one (11.9 g, 50 mmol). The mixture was stirred for 24 h at 40° C., and then cooled to room temperature. Water (50 mL) was added dropwise, and the mixture extracted with Et₂O (2×200 mL). The combined organic extracts were washed brine, diluted with 10% solution of HCl in dioxane (50 mL) and evaporated under reduced pressure to give 4-benzyl-6,6-difluoro-4-azaspiro[2.4]heptane (3 g, 13.4 mmol, 27% yield).

Step E: 4-benzyl-6,6-difluoro-4-azaspiro[2.4]heptane (2.68 g, 12 mmol) and palladium hydroxide (0.5 g) in methanol (500 mL) were stirred at room temperature under an atmosphere of H₂ for 24 h. The mixture was filtered and then filtrate concentrated under reduced pressure to obtain 6,6-difluoro-4-azaspiro[2.4]heptane (0.8 g, 6.01 mmol, 50% yield).

### Synthesis of 1-[(difluoromethoxy)methyl]-N-methylcyclopropan-1-amine

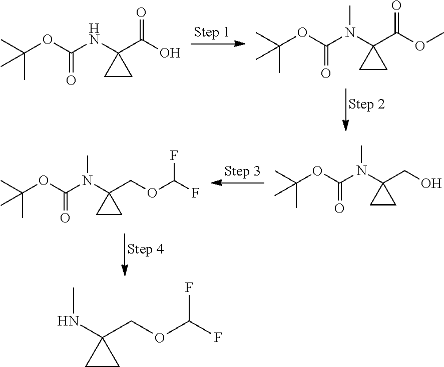

Step 1: Sodium hydride (0.596 g, 14.91 mmol) was added to a cooled (0° C.) solution of 1-((tertbutoxycarbonyl)amino)cyclopropane-1-carboxylic acid (1 g, 4.97 mmol) in dry N,N-dimethylformamide (15 mL). When gas evolution had ceased, iodomethane (0.932 mL, 14.91 mmol) was added. The cooling bath was removed and the mixture was stirred for 2 h. The mixture was then cooled to 0° C. and quenched by addition of water. The mixture was partitioned between water and ethyl acetate, the organic layer was washed with brine, concentrated and purified by flash chromatography (24 g silica gel), flowrate 30 ml/min, 15 to 50% ethyl acetate in heptane over 15 min to give the desired product as a colorless oil (1.056 g, 93% yield).

Step 2: To a solution of methyl 1-((tertbutoxycarbonyl)(methyl)amino)cyclopropane-1-carboxylate (1.05 g, 4.58 mmol) in dry THF (5 mL) under N₂ was added lithium borohydride (1.259 mL, 4M in THF, 5.04 mmol). The mixture was stirred at r.t. for 4 days. Sodium sulfate and water were added, the mixture was filtered over a pad of sodium sulfate which was rinsed with dichloromethane. The filtrate was concentrated, to give tert-butyl (1-(hydroxymethyl)cyclopropyl)(methyl)carbamate as a white solid (0.904 g, 95% yield).

Step 3: To a solution of tert-butyl (1-(hydroxymethyl)cyclopropyl)(methyl)carbamate (0.100 g, 0.497 mmol) and (bromodifluoromethyl)trimethylsilane (0.155 mL, 0.994 mmol) in dichloromethane (0.5 mL) was added one drop of a solution of potassium acetate (0.195 g, 1.987 mmol) in water (0.5 mL). The mixture was stirred for 40 h. The mixture was diluted with dichloromethane and water, the organic layer was separated and concentrated. Purifcation by flash chromatography (20% ethyl acetate in heptane) gave tert-butyl N-{1[(difluoromethoxy)methyl]cyclopropyl}-N-methylcarbamate as colorless oil (0.058 g, 46% yield)

Step 4: To tert-butyl (1-((difluoromethoxy)methyl)cyclopropyl)(methyl)carbamate (0.058 g, 0.231 mmol) was added HCl in dioxane (4M solution, 2 mL, 8.00 mmol). The mixture was stirred for 30 min at rt, then concentrated to yield the desired product which was used without further purification

LC-MS: m/z 152.2 (M+H)+

### Preparation of 6,6-difluoro-4-azaspiro[2.4]heptane

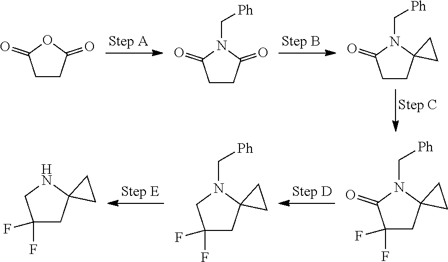 [SMILES: O=C1CCC(=O)O1 ; O=C1CCC(=O)N1Cc1ccccc1 ; O=C1CCC2(CC2)N1Cc1ccccc1 ; FC1(F)CNC2(CC2)C1 ; FC1(F)CN(Cc2ccccc2)C2(CC2)C1 ; O=C1N(Cc2ccccc2)C2(CC2)CC1(F)F]

Step A: To a solution of succinic anhydride (100 g, 1000 mmol) in toluene (3000 mL) was added benzylamine (107 g, 1000 mmol). The solution was stirred at room temperature for 24 h, then heated at reflux with a Dean-Stark apparatus for 16 hours. The mixture was then concentrated under reduced pressure to give 1-benzylpyrrolidine-2,5-dione (170 g, 900 mmol, 90% yield).

Step B: To a cooled (0° C.) mixture of 1-benzylpyrrolidine-2,5-dione (114 g, 600 mmol) and Ti(Oi-Pr)₄ (170.5 g, 600 mmol) in dry THF (2000 mL) under argon atmosphere was added dropwise a 3.4M solution of ethylmagnesium bromide in THF (1200 mmol). The mixture was warmed to room temperature and stirred for 4 h. BF₃.Et₂O (170 g, 1200 mmol) was then added dropwise and the solution stirred for 6 h. The mixture was cooled (0° C.) and 3N hydrochloric acid (500 mL) was added. The mixture was extracted twice with Et₂O, and the combined organic extracts washed with brine, dried and concentrated under reduced pressure to give 4-benzyl-4-azaspiro[2.4]heptan-5-one (30.2 g, 150 mmol, 25% yield).

Step C: To a cooled (−78° C.) solution of 4-benzyl-4-azaspiro[2.4]heptan-5-one (34.2 g, 170 mmol) in dry THF (1000 mL) under argon was added LiHMDS in THF (1.1M solution, 240 mmol). The mixture was stirred for 1 h, then a solution of N-fluorobenzenesulfonimide (75.7 g, 240 mmol) in THF (200 mL) was added dropwise. The mixture was warmed to room temperature and stirred for 6 h. The mixture was then re-cooled (−78° C.) and LiHMDS added (1.1M solution in THF, 240 mmol).

The solution was stirred for 1 h, then N-fluorobenzenesulfonimide (75.7 g, 240 mmol) in THF (200 mL) was added dropwise. The mixture was warmed to room temperature and stirred for 6 h. The mixture was poured into a saturated solution of NH₄Cl (300 mL) and extracted twice with Et₂O. The combined organic extracts were washed with brine and concentrated under reduced pressure. Product was purified by column chromatography to provide 4-benzyl-6,6-difluoro-4-azaspiro[2.4]heptan-5-one (18 g, 75.9 mmol, 45% yield).

Step D: To a warmed (40° C.) solution of BH3.Me2S (3.42 g, 45 mmol) in THF (200 mL) was added dropwise 4-benzyl-6,6-difluoro-4-azaspiro[2.4]heptan-5-one (11.9 g, 50 mmol). The mixture was stirred for 24 h at 40° C., then cooled to room temperature. Water (50 mL) was added dropwise, and the mixture extracted with Et₂O (2×200 mL). The combined organic extracts were washed brine, diluted with 10% solution of HCl in dioxane (50 mL) and evaporated under reduced pressure to give 4-benzyl-6,6-difluoro-4-azaspiro[2.4]heptane (3 g, 13.4 mmol, 27% yield).

Step E: 4-benzyl-6,6-difluoro-4-azaspiro[2.4]heptane (2.68 g, 12 mmol) and palladium hydroxide (0.5 g) in methanol (500 mL) were stirred at room temperature under an atmosphere of H₂ for 24 h. The mixture was filtered and then filtrate concentrated under reduced pressure to obtain 6,6-difluoro-4-azaspiro[2.4]heptane (0.8 g, 6.01 mmol, 50% yield).

### Preparation of 7,7-difluoro-4-azaspiro[2.4]heptane

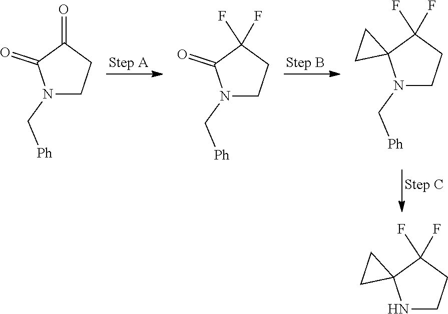 [SMILES: O=C1CCN(Cc2ccccc2)C1=O ; O=C1N(Cc2ccccc2)CCC1(F)F ; FC1(F)CCN(Cc2ccccc2)C12CC2 ; FC1(F)CCNC12CC2]

Step A: To a cooled (0° C.) solution of 1-benzylpyrrolidine-2,3-dione (8 g, 42.3 mmol) in DCM (100 mL) was added dropwise over 30 minutes DAST (20.4 g, 127 mmol). The mixture was stirred at room temperature overnight, then quenched by dropwise addition of saturated NaHCO₃. The organic layer was separated, and the aqueous fraction extracted twice with DCM (2×50 mL). The combined organic layers were dried over Na₂SO₄ and concentrated under reduced pressure to afford 1-benzyl-3,3-difluoropyrrolidin-2-one (26.0 mmol, 61% yield), which used in the next step without further purification.

Step B: To a solution of crude 1-benzyl-3,3-difluoropyrrolidin-2-one (5.5 g, 26 mmol) and Ti(Oi-Pr)₄ (23.4 mL, 78 mmol) in THF (300 mL) was added dropwise under argon atmosphere 3.4 M solution of EtMgBr in 2-MeTHF (45.8 mL, 156 mmol). After stirring for 12 h, water (10 mL) was added to obtain a white precipitate. The precipitate was washed with MTBE (3×50 mL). The combined organic fractions were dried over Na₂SO₄, concentrated and purified by flash chromatography (hexanes-EtOAc 9:1) to obtain 4-benzyl-7,7-difluoro-4-azaspiro[2.4]heptane (1.3 g, 5.82 mmol, 22% yield) as a pale yellow oil.

Step C: 4-benzyl-7,7-difluoro-4-azaspiro[2.4]heptane (0.55 g, 2.46 mmol) was dissolved in solution of CHCl₃ (1 mL) and MeOH (20 mL) and Pd/C (0.2 g, 10%) was added. This mixture was stirred under and an H₂ atmosphere for 5 h, then filtered. The filtrate was concentrated to give 7,7-difluoro-4-azaspiro[2.4]heptane (0.164 g, 1.23 mmol, 50% yield)

### Synthesis of N-methyl-1-(5-methyl-1,3,4-oxadiazol-2-yl)cyclopropan-1-amine

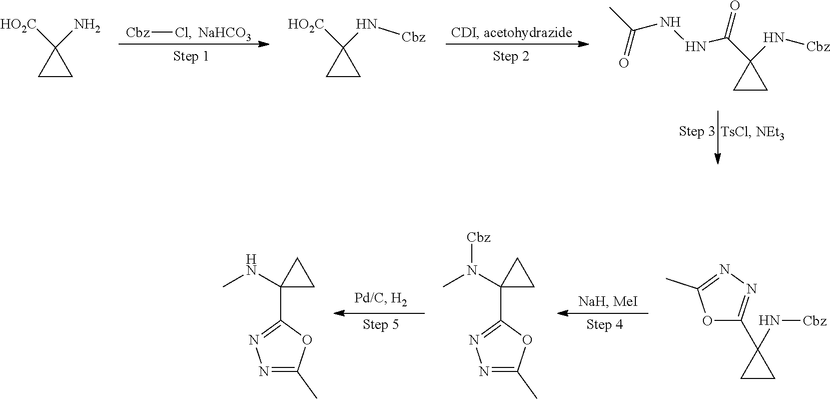

Step 1: 1-Aminocyclopropane-1-carboxylic acid (6.0 g, 59.34 mmol) and sodium hydrogen carbonate (19.94 g, 237.38 mmol) were dissolved in distilled water (50 mL) and the resulting mixture was diluted with THF (50 mL). The mixture was cooled to 0° C. with an icewater bath and a solution of benzyl chloroformate (11.14 g, 65.28 mmol, 9.28 mL) in THF (l0 mL) was added dropwise. The resulting mixture was stirred overnight then washed with EtOAc. The aqueous layer was separated, acidified to pH=1 with conc. HCl, and extracted with EtOAc (2×20 mL). The combine organic extracts were dried (Na₂SO₄) and concentrated under reduced pressure to give 1-[(benzyloxy)carbonyl]aminocyclopropane-1-carboxylic acid (6.0 g, 25.51 mmol, 43% yield) which was used for the next step without purification.

Step 2: To a solution of 1-[(benzyloxy)carbonyl]aminocyclopropane-1-carboxylic acid (6.0 g, 25.5 mmol) in DCM (100 mL) at r.t. was added 1-(1H-imidazole-1-carbonyl)-1H-imidazole (6.2 g, 38.3 mmol) in one portion. Upon completion of gas evolution (˜20 min) acetohydrazide (3.78 g, 51.01 mmol) was added and the reaction mixture stirred overnight. The precipitate formed was collected by filtration, washed with DCM and dried to give benzyl N-[1-(N′-acetylhydrazinecarbonyl)cyclopropyl]carbamate (4.0 g).

The filtrate was concentrated under reduced pressure. The residue was partitioned between EtOAc (100 mL) and aqueous sodium hydrogensulfate solution (100 mL). The organic phase was washed with water, brine, dried over sodium sulfate and concentrated under reduced pressure to afford second portion of product (2.5 g). Portions were combined to obtain benzyl N-[1-(N′-acetylhydrazinecarbonyl)cyclopropyl]carbamate (6.5 g, 22.31 mmol, 87.5% yield) as a white solid.

Step 3: Benzyl N-[1-(N′-acetylhydrazinecarbonyl)cyclopropyl]carbamate (6.5 g, 22.3 mmol) was suspended in DCM (100 mL). Triethylamine (4.97 g, 49.09 mmol, 6.84 mL) was added in one portion and the resulting mixture was cooled to 0° C. with an ice/water bath. A solution of 4-methylbenzene-1-sulfonyl chloride (4.47 g, 23.4 mmol) in DCM (50 mL) was added. The resulting mixture was then warmed, then heated at reflux. The resulting mixture was washed with water (2×10 mL), sat. aq. sodium bicarbonate, brine, dried over Na₂SO₄ and concentrated under reduced pressure. The residue was purified by column chromatography (1st run: Interchim, 220 g SiO2, MTBE/methanol with methanol from 0-10%, flow rate=100 mL/min, Rv=6 CV; 2nd run: Interchim, 80 g SiO2, chloroform/acetonitrile with acetonitrile from 0-50%, flow rate=60 m/min, Rv=10 CV) to obtain benzyl N-[1-(5-methyl-1,3,4-oxadiazol-2-yl)cyclopropyl]carbamate (2.69 g, 9.82 mmol, 44% yield) as yellow solid.

Step 4: Sodium hydride (126.49 mg, 5.27 mmol) was suspended in dry THF (30 mL). A solution of benzyl N-[1-(5-methyl-1,3,4-oxadiazol-2-yl)cyclopropyl]carbamate (1.2 g, 4.39 mmol) in dry THF (10 mL) was added dropwise at 15° C. (water bath). The resulting mixture was stirred until gas release was complete then cooled to 0° C. Iodomethane (748 mg, 5.27 mmol, 330 μl) was added dropwise, and the resulting mixture was warmed to r.t. and stirred overnight. The mixture was then extracted with EtOAc (2×20 mL), and the combined organic extracts were dried over sodium sulfate then concentrated under reduced pressure to obtain crude benzyl N-methyl-N-[1-(5-methyl-1,3,4-oxadiazol-2-yl)cyclopropyl]carbamate (1.33 g, 4.62 mmol, 105.2% yield) which was used for the next step without purification.

Step 5: To a solution of benzyl N-methyl-N-[1-(5-methyl-1,3,4-oxadiazol-2-yl)cyclopropyl]carbamate (1.33 g, 4.62 mmol) in dry methanol (20 mL) was added 10% Pd/C (100 mg). The resulting mixture was stirred under at atmosphere of H₂. When reaction was complete (according to 1H NMR of the reaction mixture) the mixture was filtered and the filtrate concentrated. The residue was purified by HPLC to obtain N-methyl-1-(5-methyl-1,3,4-oxadiazol-2-yl)cyclopropan-1-amine (140 mg, 913 μmol, 19.7% yield).

### Synthesis of N-methyl-1-(1,3-oxazol-2-yl)cyclopropan-1-amine

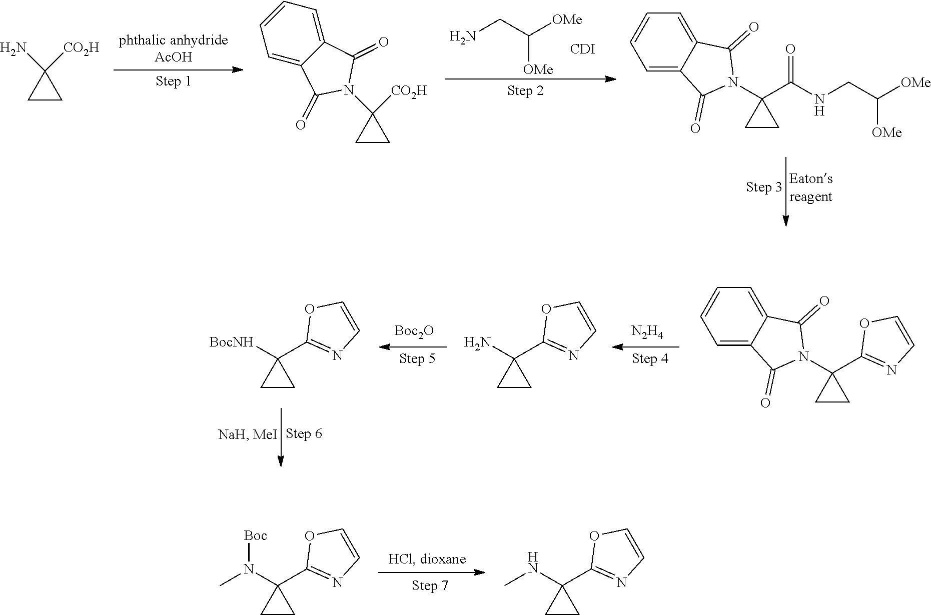

Step 1: 1-Aminocyclopropane-1-carboxylic acid (4.85 g, 48.0 mmol) was suspended in glacial acetic acid (50 mL). Phthalic anhydride (7.11 g, 48.0 mmol) was added and the resulting mixture was stirred at 110° C. overnight. stirring at 110° C. overnight. The mixture was cooled to r.t. and triturated with water (200 mL). The precipitate was collected by filtration, washed with water and dried to obtain 1-(1,3-dioxo-2,3-dihydro-1H-isoindol-2-yl)cyclopropane-1-carboxylic acid (8.8 g, 38.1 mmol, 79.3% yield) as white solid.

Step 2: To a solution of 1-(1,3-dioxo-2,3-dihydro-1H-isoindol-2-yl)cyclopropane-1-carboxylic acid (8.8 g, 38.1 mmol) in DCM (100 mL) and THF (10 mL) atr.t. was added 1-(1H-imidazole-1-carbonyl)-1H-imidazole (6.79 g, 41.9 mmol). After complete reaction (monitored by NMR), 2,2-dimethoxyethan-1-amine (4.4 g, 41.9 mmol, 4.56 mL) was added at r.t and the mixture stirred overnight. The mixture then was concentrated under reduced pressure and the residue was triturated with distilled water (15 mL). The resulting precipitate was collected by filtration, washed with water (2×15 mL) and dissolved in DCM. The organic layer was collected, dried (Na₂SO₄) and concentrated under reduced pressure to obtain N-(2,2-dimethoxyethyl)-1-(1,3-dioxo-2,3-dihydro-1H-isoindol-2-yl)cyclopropane-1-carboxamide (6.0 g, 18.9 mmol, 49.5% yield).

Step 3: N-(2,2-dimethoxyethyl)-1-(1,3-dioxo-2,3-dihydro-1H-isoindol-2-yl)cyclopropane-1-carboxamide (10.5 g, 33.0 mmol) was added to methanesulfonic acid (˜100 g) followed by addition of phosphorus pentoxide (7.7 g) and the mixture was stirred at 140° C. overnight. The resulting dark solution was cooled to r.t., poured into ice, and the pH of the resulting mixture was adjusted to 8 with saturated NaHCO₃ solution. The product was extracted with ethyl acetate (2×200 mL). The combined organic extracts were washed with brine, dried over sodium sulfate and evaporated.

The residue obtained was triturated with Et₂O and product collected by filtration. The resulting white solid was dried to obtain 2-[1-(1,3-oxazol-2-yl)cyclopropyl]-2,3-dihydro-1H-isoindole-1,3-dione (2.3 g, 9.05 mmol, 27.4% yield).

Step 4: To a solution of 2-[1-(1,3-oxazol-2-yl)cyclopropyl]-2,3-dihydro-1H-isoindole-1,3-dione (2.3 g, 9.05 mmol) in ethanol (50 mL) was added hydrazine hydrate (2.26 g, 45.23 mmol, 2.26 mL). The resulting mixture was stirred at 50° C. overnight. The resulting mixture was cooled to r.t. and concentrated in vacuo. The residue obtained was triturated with DCM. The resulting precipitate was filtered off and the filtrate concentrated under reduced pressure to obtain crude 1-(1,3-oxazol-2-yl)cyclopropan-1-amine (1.24 g, 10.0 mmol) as colorless oil, which was used in the next step without further purification.

Step 5: Di-tert-butyl dicarbonate (2.18 g, 10.0 mmol, 2.3 mL) was added dropwise to a solution of 1-(1,3-oxazol-2-yl)cyclopropan-1-amine (1.24 g, 10.0 mmol) in dry DCM (10 mL). The resulting mixture was stirred until completion (1H NMR), and concentrated under reduced pressure. The residue was purified by flash column chromatography (80 g SiO2, petroleum ether/MTBE with MTBE from 0-40%, flow rate=60 mL/min, Rv=8 CV) to obtain tert-butyl N-[1-(1,3-oxazol-2-yl)cyclopropyl]carbamate (400 mg, 1.78 mmol, 17.8% yield) as yellow oil.

Step 6: Sodium hydride (51.36 mg, 2.14 mmol) was suspended in 10 mL of dry THF. A solution of tert-butyl N-[1-(1,3-oxazol-2-yl)cyclopropyl]carbamate (400 mg, 1.78 mmol) in dry THF (2 mL) was added dropwise (water bath cooling). The resulting mixture was stirred until gas evolution ceased and was then cooled (0° C.). Iodomethane (304 mg, 2.14 mmol, 130 μL) was added dropwise and the resulting mixture was warmed to r.t. and stirred overnight. The reaction mixture was poured into saturated aq. ammonium chloride solution. The resulting mixture was extracted with EtOAc (2×10 mL) and the combined organic extracts were dried over sodium sulfate then concentrated unde reduced pressure. The residue was purified by HPLC (column: Waters SunFire C18, 5 mkm, 19 mm×100 mm; mobile phase: water-acetonitrile, 30 mL/min) to obtain tert-butyl N-methyl-N-[1-(1,3-oxazol-2-yl)cyclopropyl]carbamate (29 mg, 122 μmol, 6.8% yield).

Step 7: Tert-butyl N-methyl-N-[1-(1,3-oxazol-2-yl)cyclopropyl]carbamate (29.0 mg, 121.7 μmol) was dissolved in 4M HCl/dioxane (2 mL) at r.t. and the resulting mixture was stirred overnight. The resulting mixture was concentrated under reduced pressure to obtain N-methyl-1-(1,3-oxazol-2-yl)cyclopropan-1-amine hydrochloride (14 mg, 80.17 μmol, 83.3% yield).

### Synthesis of N-methyl-1-(1,3-oxazol-5-yl)cyclopropan-1-amine

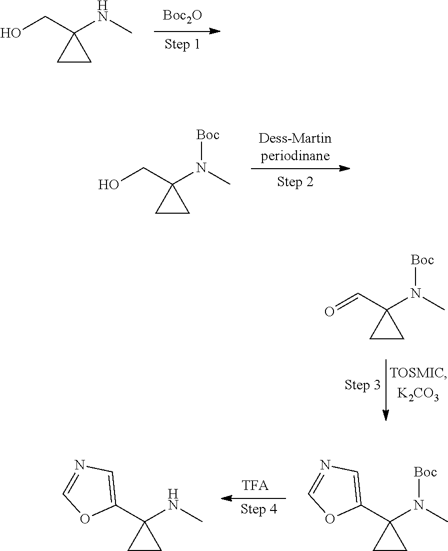

Step 1: Di-tert-butyl dicarbonate (1.75 g, 8.0 mmol) was added portionwise to a mixture of (1-(methylamino)cyclopropyl)methanol hydrochloride (1.0 g, 7.27 mmol) and triethylamine (957 mg, 9.46 mmol) in DCM (20 mL) and left to stir overnight at r.t. After reaction was complete (monitored by 1H NMR) the mixture was washed with water (10 mL), dried over Na₂SO₄ and concentrated in vacuum to give tert-butyl N-[1-(hydroxymethyl)cyclopropyl]-N-methylcarbamate (1.2 g, 5.97 mmol, 82% yield).

Step 2: To a cooled (0° C.) solution of tert-butyl N-[1-(hydroxymethyl)cyclopropyl]-N-methylcarbamate (500.01 mg, 2.48 mmol) in DCM (50 mL) was added 1,1,1-tris(acetoxy)-1,1-dihydro-1,2-benziodoxol-3(1H)-one (1.16 g, 2.73 mmol). When reaction was complete (monitored by 1H NMR) the mixture was poured into an aqueous solution of NaHCO₃ and Na₂S₂O₃, then stirred until organic phase became transparent (˜1 h). The layers were separated and the aqueous layer extracted with DCM (3×50 mL). The combined organic extracts were washed with brine, dried over Na₂SO₄ and concentrated under reduced pressure to give crude tert-butyl N-(1-formylcyclopropyl)-N-methylcarbamate (620 mg, 3.11 mmol) which was used for the next step without further purification.

Step 3: Tert-butyl N-(1-formylcyclopropyl)-N-methylcarbamate (477 mg, 2.39 mmol) was mixed with 1-isocyanomethanesulfonyl-4-methylbenzene (514 mg, 2.63 mmol) in dry methanol (50 mL) followed by addition of potassium carbonate (695 mg, 5.03 mmol). The resulting mixture was at reflux for 2 hours. Distilled water (20 mL) was then added to the hot reaction mixture and the resulting solution extracted with EtOAc (2×15 mL). The combined organic extracts were dried (sodium sulfate) and concentrated under reduced pressure. The residue was purified by column chromatography (40 g SiO2, chloroform/acetonitrile with acetonitrile from 0 to 20%, flow rate=40 mL/min) to obtain tert-butyl N-methyl-N-[1-(1,3-oxazol-5-yl)cyclopropyl]carbamate (400.0 mg, 1.68 mmol, 70.1% yield).

Step 4: Tert-butyl N-methyl-N-[1-(1,3-oxazol-5-yl)cyclopropyl]carbamate (370 mg, 1.55 mmol) was dissolved in TFA (5 mL) and the resulting mixture was left to stir at r.t. overnight. When the reaction was complete (monitored by LCMS of the reaction mixture) the excess of TFA was evaporated to obtain N-methyl-1-(1,3-oxazol-5-yl)cyclopropan-1-amine trifluoroacetate (360 mg, 2.1 mmol, 100% yield).

### Synthesis of N-methyl-1-(1,3-oxazol-4-yl)cyclopropan-1-amine

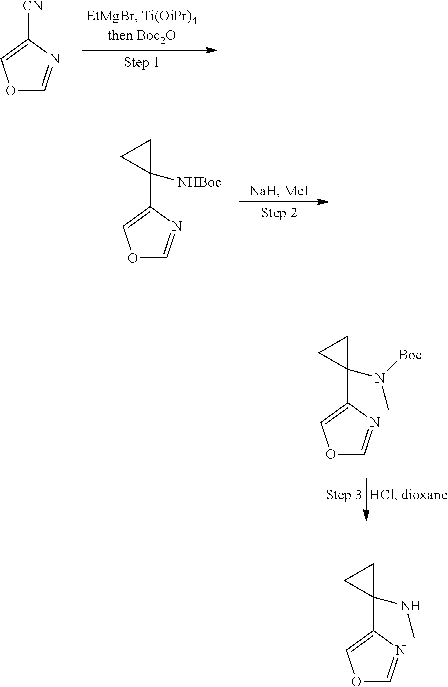

Step 1: To a cooled (−70° C.) solution of 1,3-oxazole-4-carbonitrile (4.0 g, 42.52 mmol) and titanium tetraisopropoxide (13.29 g, 46.77 mmol) in Et₂O (220 mL) was added ethylmagnesium bromide (11.9 g, 89.29 mmol). The resulting yellow solution was stirred for 10 min. The solution was warmed to r.t. and stirred for 1 h. Boron trifluoride-diethyl etherate (12.07 g, 85.04 mmol, 10.73 mL) was added and the mixture stirred for a further 1 h. 1N HCl (100 mL) and ether (200 mL) were added. NaOH (10% aq, 200 mL) was added to the resulting two clear phases, followed by addition of di-tert-butyl dicarbonate (46.4 g, 212.59 mmol, 48.84 mL). The resulting biphasic mixture was stirred vigorously overnight. The layers were separated and the aqueous phase was extracted with 300 mL of diethyl ether. The combined organic extracts were dried over Na₂SO₄, filtered and concentrated under reduced pressure to give viscous yellow oil, which mainly consisted of desired product and Boc₂O (shown by 1H NMR). This oil was dissolved in 100 mL of dioxane and the resulting solution was added dropwise to a solution of 2-aminoacetic acid (15.96 g, 212.59 mmol) and sodium carbonate (22.53 g, 212.59 mmol) in 200 mL of water at r.t. The resulting mixture was left stirring overnight before all volatiles were removed under vacuum. The residue was partitioned between 300 mL of water and 150 mL of MTBE. The organic phase was washed with 50 mL of water, brine, dried over Na₂SO₄ and concentrated under reduced pressure to give tert-butyl N-[1-(1,3-oxazol-4-yl)cyclopropyl]carbamate (7.2 g, 32.11 mmol, 75.5% yield) as light yellow crystalline solid.

Step 2: To a solution of tert-butyl N-[1-(1,3-oxazol-4-yl)cyclopropyl]carbamate (2.0 g, 8.92 mmol) in 50 mL of DMF was added sodium hydride (60%, 321.02 mg, 13.38 mmol) portionwise, maintaining the temperature below 25° C. (water cooling bath). After gas evolution was complete, iodomethane (3.16 g, 22.29 mmol, 1.39 mL) was added dropwise and the resulting mixture was left to stir overnight at r.t. The reaction mixture was poured into 500 mL of water and extracted with 150 mL of ethyl acetate. The organic phase was washed with water (2×100 mL), brine, dried over Na₂SO₄ and concentrated in vacuo to give tert-butyl N-methyl-N-[1-(1,3-oxazol-4-yl)cyclopropyl]carbamate (2.15 g, 90.0% purity, 8.12 mmol, 91.1% yield) as yellow crystalline solid.

Step 3: Tert-butyl N-methyl-N-[1-(1,3-oxazol-4-yl)cyclopropyl]carbamate (2.15 g, 9.02 mmol) was dissolved in 50 mL of 4M HCl/dioxane at r.t. and the resulting mixture was stirred overnight. The resulting mixture was diluted with 50 mL of diethyl ether and product collected by filtration. The solid was washed with 20 mL of ether, and dried in vacuo to obtain N-methyl-1-(1,3-oxazol-4-yl)cyclopropan-1-amine hydrochloride (1.32 g, 7.56 mmol, 83.8% yield) as light yellow powder.

### Synthesis of N-methyl-1-(1,2-oxazol-5-yl)cyclopropan-1-amine

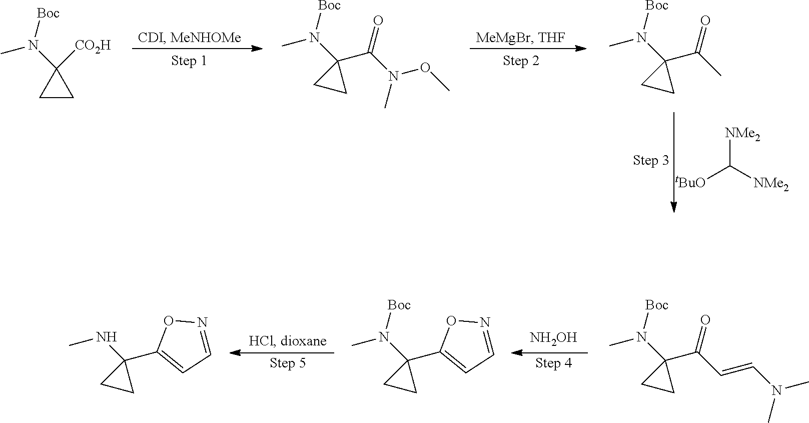

Step 1: To a solution of 1-[(tert-butoxy)carbonyl](methyl)aminocyclopropane-1-carboxylic acid (6.0 g, 27.88 mmol) in dry DCM (300 mL) atr.t. was added 1-(1H-imidazole-1-carbonyl)-1H-imidazole (6.78 g, 41.82 mmol). When gas evolution was complete (˜20 min), methoxy(methyl)amine hydrochloride (6.8 g, 69.7 mmol) was added and the resulting mixture was stirred overnight. The reaction mixture was diluted with petroleum ether (300 mL) and washed with water (3×300 mL). The organic phase was separated, washed with brine, dried over sodium sulfate and concentrated under reduced pressure to obtain tert-butyl N-1-[methoxy(methyl)carbamoyl]cyclopropyl-N-methylcarbamate (3.95 g, 96.0% purity, 14.7 mmol, 52.7% yield) as a colorless oil.

Step 2: To a solution of tert-butyl N-1-[methoxy(methyl)carbamoyl]cyclopropyl-N-methylcarbamate (3.77 g, 14.6 mmol) in 100 mL of THF at r.t. under argon atmosphere was added methylmagnesium bromide (5.22 g, 43.8 mmol, 13.7 mL). The mixture was stirred at r.t. overnight, quenched by addition of saturated aqueous NH₄Cl solution (50 mL) and concentrated under reduced pressure. The residue was partitioned between 200 mL of water and 200 mL of MTBE. The organic layer was washed with 100 mL of water, brine, dried over Na₂SO₄ and concentrated under reduced pressure to give tert-butyl N-(1-acetylcyclopropyl)-N-methylcarbamate (2.71 g, 96.0% purity, 12.2 mmol, 83.6% yield) as light yellow liquid.

Step 3: Tert-butyl N-(1-acetylcyclopropyl)-N-methylcarbamate (2.71 g, 12.71 mmol) was dissolved in tert-butoxy bis(dimethylamino)methane (50 mL) and heated at 75° C. overnight. The reaction mixture was concentrated under reduced pressure to obtain 6.65 g of an orange oil. 2 g of this oil were purified by flash chromatography (40 g SiO₂, petroleum ether/MTBE with MTBE from 15-100% and MTBE/methanol with methanol from 0-15%, flow rate=40 m/min, Rv=21.5 CV) to obtain tert-butyl N-1-[(2E)-3-(dimethylamino)prop-2-enoyl]cyclopropyl-N-methylcarbamate (580 mg, 2.16 mmol) as a colorless liquid.

Step 4: A mixture of tert-butyl N-1-[(2E)-3-(dimethylamino)prop-2-enoyl]cyclopropyl-N-methylcarbamate (580.0 mg, 2.16 mmol) and hydroxylamine hydrochloride (165 mg, 2.38 mmol) in dry methanol (20 mL) was heated at 50° C. under an argon atmosphere for 20 h. The reaction mixture was then concentrated under reduced pressure. The residue was partitioned between ethyl acetate (20 mL) and water (50 mL). The organic layer was washed with water, brine, dried over Na₂SO₄ and concentrated under reduced pressure to give tert-butyl N-methyl-N-[1-(1,2-oxazol-5-yl)cyclopropyl]carbamate (455 mg, 1.91 mmol, 88.3% yield) as light yellow oil.

Step 5: Tert-butyl N-methyl-N-[1-(1,2-oxazol-5-yl)cyclopropyl]carbamate (455 mg, 1.91 mmol) was dissolved in 10 mL of 4M HCl/dioxane at r.t. and the resulting mixture was stirred overnight. The resulting mixture was concentrated under reduced pressure and the residue was triturated with ethyl acetate (10 mL). The pale brown solid obtained was collected by filtration and dried under vacuum to give N-methyl-1-(1,2-oxazol-5-yl)cyclopropan-1-amine hydrochloride (210.0 mg, 1.2 mmol, 63.1% yield) as crystalline solid.

### Synthesis of N-methyl-1-(1,2-oxazol-3-yl)cyclopropan-1-amine

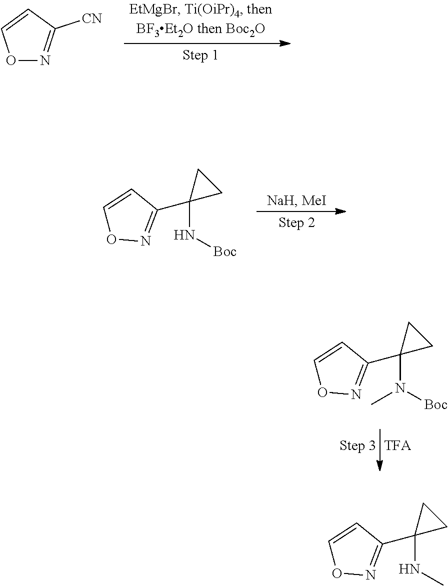

Step 1: To a cooled (−70° C.) to solution of 1,2-oxazole-3-carbonitrile (4.0 g, 42.5 mmol) and titanium tetraisopropoxide (13.3 g, 46.8 mmol) in Et2O (200 mL) was added ethylmagnesium bromide (11.9 g, 89.3 mmol, 26.3 mL). The resulting yellow solution was stirred for 10 min at −70° C. then slowly warmed to r.t. Boron trifluoride-diethyl etherate (12.1 g, 85.1 mmol, 10.7 mL) was then added. After stirring for 1 h, 1N HCl (100 mL) and diethyl ether (200 mL) were added. NaOH (10% aq, 200 mL) was added to the resulting mixture, followed by addition of di-tert-butyl dicarbonate (46.4 g, 212 mmol, 48.9 mL). The resulting biphasic mixture was stirred vigorously overnight. The phases were separated, and the aqueous phase was extracted with diethyl ether (3×100 mL). The combined organic extracts were dried over Na₂SO₄, filtered and concentrated under reduced pressure to give viscous yellow oil, which mainly consisted of desired product and Boc₂O. This oil was dissolved in 50 mL of dioxane. To this solution was added dropwise a solution of 2-aminoacetic acid (15.96 g, 212.66 mmol) and sodium carbonate (22.54 g, 212.66 mmol) in 100 mL of water. The mixture was left to stir overnight then concentrated under reduced pressure. The residue was partitioned between 300 mL of water and 150 mL of MTBE. The organic phase was washed with 5 mL of water, brine, dried over Na₂SO₄ and concentrated under reduced pressure to give tert-butyl N-[1-(1,2-oxazol-3-yl)cyclopropyl]carbamate (6.0 g, 26.8 mmol, 62.9% yield) as light yellow oil.

Step 2: Sodium hydride (67 mg, 2.81 mmol) was suspended in 10 mL of dry THF. A solution of tert-butyl N-[1-(1,2-oxazol-3-yl)cyclopropyl]carbamate (524 mg, 2.34 mmol) in 2 mL of dry THF was then added dropwise (water bath cooling). The resulting mixture was stirred until gas evolution ceased and then cooled to 0° C. Iodomethane (498 mg, 3.51 mmol, 220 μL) was added dropwise and the resulting mixture was warmed to r.t. and then stirred overnight. The reaction mixture was poured into saturated aq. ammonium chloride solution. The resulting mixture was extracted EtOAc (2×10 mL). The combined organic extracts were combined, dried over sodium sulfate and concentrated under reduced pressure giving crude tert-butyl N-methyl-N-[1-(1,2-oxazol-3-yl)cyclopropyl]carbamate (537 mg, 2.25 mmol, 96.4% yield) which was used in next step without purification.

Step 3: tert-Butyl N-methyl-N-[1-(1,2-oxazol-3-yl)cyclopropyl]carbamate (536 mg, 2.25 mmol) was dissolved in 50 ml of dry DCM. 2,2,2-Trifluoroacetic acid (770 mg, 6.75 mmol, 520 μl) was added in one portion and the resulting mixture was stirred at r.t. overnight. The reaction mixture was concentrated under reduced pressure to obtain N-methyl-1-(1,2-oxazol-3-yl)cyclopropan-1-amine (64 mg, 463 μmol, 20.6% yield).

### Synthesis of N-methyl-1-(3-methyl-1,2,4-oxadiazol-5-yl)cyclopropan-1-amine

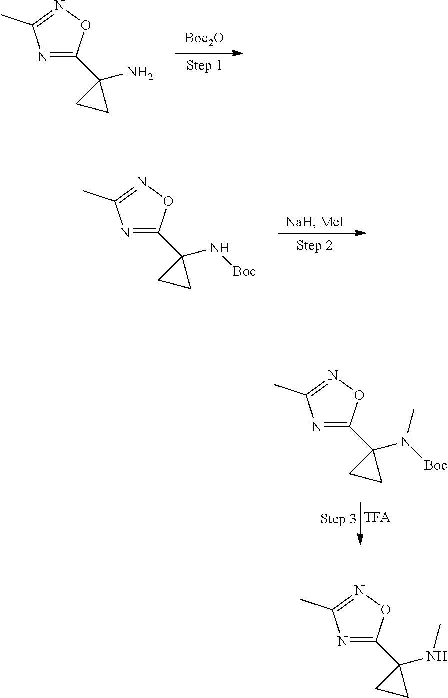

Step 1: 1-(3-Methyl-1,2,4-oxadiazol-5-yl)cyclopropan-1-amine hydrochloride (1.5 g, 8.54 mmol) and di-tert-butyl dicarbonate (2.05 g, 9.39 mmol, 2.16 mL) were mixed in dichloromethane (50 mL), and triethylamine (949.0 mg, 9.38 mmol, 1.31 mL) was added dropwise at 0° C. The reaction mixture was stirred at ambient temperature overnight then washed with water (2×10 mL), dried over sodium sulfate and evaporated in vacuo to give tert-butyl N-[1-(3-methyl-1,2,4-oxadiazol-5-yl)cyclopropyl]carbamate (1.61 g, 6.72 mmol, 78.9% yield).

Step 2: Sodium hydride (209.7 mg, 8.74 mmol) was suspended in dry THF (10 mL). A solution of tert-butyl N-[1-(3-methyl-1,2,4-oxadiazol-5-yl)cyclopropyl]carbamate (1.61 g, 6.72 mmol) in dry THF (10 mL) was added dropwise (water bath cooling). The resulting mixture was stirred until gas release was complete, and then cooled to 0° C. Iodomethane (1.05 g, 7.4 mmol, 460.0 μL) was added dropwise. The resulting mixture was warmed to r.t. and then stirred overnight.

The reaction mixture was poured into saturated aq. ammonium chloride solution and extracted twice with 20 mL of CH₂Cl₂. The combined organic extracts were dried over sodium sulfate and concentrated. The residue (1.56 g) was purified by column chromatography on silica gel using hexane/MTBE (gradient 100/0 to 50/50) as eluent to obtain tert-butyl N-methyl-N-[1-(3-methyl-1,2,4-oxadiazol-5-yl)cyclopropyl]carbamate (914.0 mg, 3.61 mmol, 53.7% yield) as colorless oil.

Step 3: tert-Butyl N-methyl-N-[1-(3-methyl-1,2,4-oxadiazol-5-yl)cyclopropyl]carbamate (914.0 mg, 3.61 mmol) was dissolved in 50 mL of dry DCM. 2,2,2-Trifluoroacetic acid (2.06 g, 18.04 mmol, 1.39 mL) was added in one portion and the resulting mixture was stirred at r.t. overnight. The reaction mixture was concentrated giving N-methyl-1-(3-methyl-1,2,4-oxadiazol-5-yl)cyclopropan-1-amine trifluoroacetate (522.0 mg, 1.95 mmol, 54.1% yield) Synthesis of 1-amino-N-methylcyclopropane-1-carboxamide

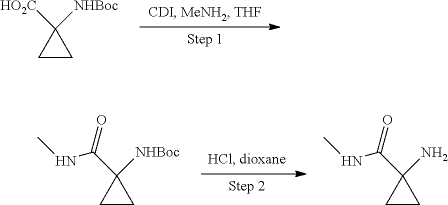

Step 1: 1-(1H-imidazole-1-carbonyl)-1H-imidazole (2.42 g, 14.9 mmol) was added to a solution of 1-((tert-butoxycarbonyl)amino)cyclopropanecarboxylic acid (2.0 g, 9.94 mmol) in 10 mL of dry THF at r.t. When the gas release completed (˜20 min), a solution of methanamine (50 mL, 20% solution in methanol) was added dropwise. The resulting solution was was stirred overnight. The solvent was evaporated in vacuo and the residue was partitioned between DCM (30 mL) and water (10 mL). The organic phase was separated, washed with water, brine, dried over sodium sulfate and concentrated under reduced pressure to obtain tert-butyl N-[1-(methylcarbamoyl)cyclopropyl]carbamate (1.9 g, 8.89 mmol, 89.4% yield) as a white solid.

Step 2: Tert-butyl N-[1-(methylcarbamoyl)cyclopropyl]carbamate (1.9 g, 8.89 mmol) was dissolved in 25 mL of 4M HCl in dioxane. and the resulting mixture was stirred overnight.

The mixture was concentrated under reduced pressure to obtain 1-amino-N-methylcyclopropane-1-carboxamide hydrochloride (1.29 g, 8.58 mmol, 96.4% yield) as a white solid.

### Synthesis of tert-butyl 3-(1-[3-(methoxycarbonyl)phenyl]cyclopropyl(methyl) carbamoyl)-4H,5H,6H,7H-pyrazolo[1,5-a]pyrazine-5-carboxylate

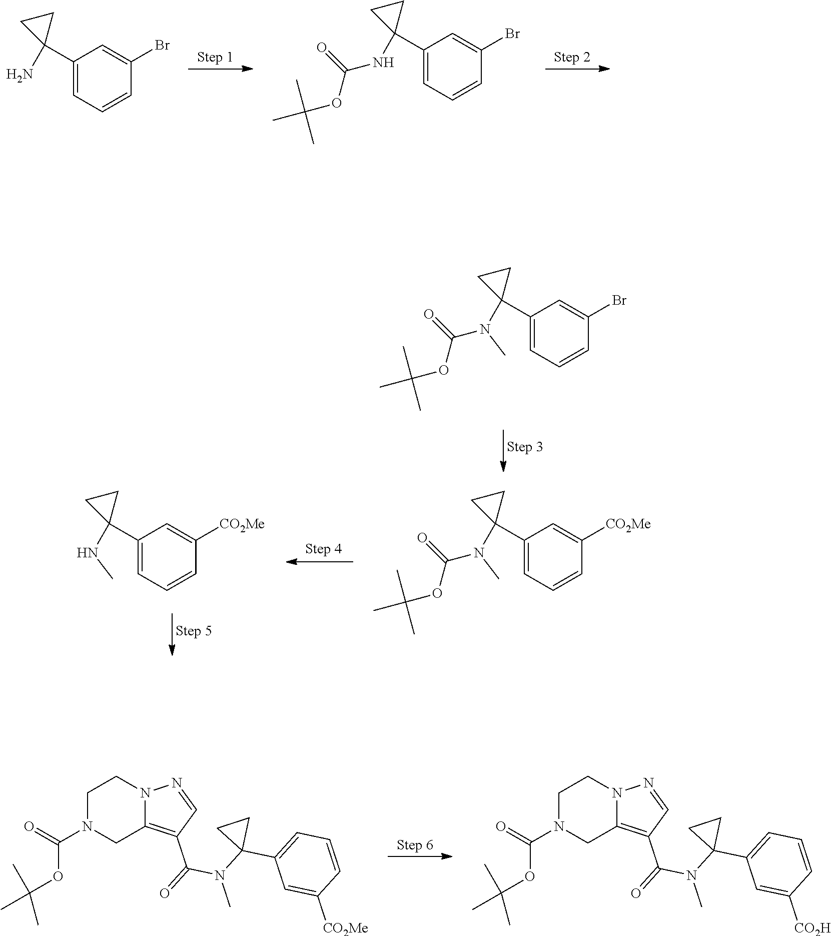

Step 1: To a cooled (0° C.) suspension of 1-(3-bromophenyl)cyclopropan-1-amine hydrochloride (1.01 g, 4.05 mmol) in dry DCM (10 mL) was added di-tert-butyl dicarbonate (882.91 mg, 4.05 mmol) and triethylamine (450.12 mg, 4.45 mmol, 620.0 μl). The reaction mixture was stirred overnight at r.t., and then diluted with water (5 mL). The organic phase was separated, washed with 10% aq. H₃PO₄ and water, dried over Na₂SO₄, filtered and concentrated to afford tert-butyl N-[1-(3-bromophenyl)cyclopropyl]carbamate (1.1 g, 3.52 mmol, 87.1% yield) as a brown oil.

Step 2: To a cooled (0° C.) suspension of sodium hydride (212.04 mg, 8.84 mmol, 1) in dry THF (5 ml) under Ar was added dropwise a solution of tert-butyl N-[1-(3-bromophenyl)cyclopropyl]carbamate (1.1 g, 3.53 mmol) in THF (2 ml). The reaction mixture was stirred for 1 h at r.t. and then cooled to 0° C. Iodomethane (752.4 mg, 5.3 mmol, 330.0 μl) was added dropwise and the reaction mixture was stirred at r.t. overnight. The mixture was diluted with brine (10 mL) and extracted with EtOAc (2*10 mL). The combined organic phases were washed with brine, dried over Na₂SO₄, filtered and concentrated to afford tert-butyl N-[1-(3-bromophenyl)cyclopropyl]-N-methylcarbamate (700.0 mg, 2.15 mmol, 60.7% yield) as yellow oil.

Step 3: To a solution of tert-butyl N-[1-(3-bromophenyl)cyclopropyl]-N-methylcarbamate (701.88 mg, 2.15 mmol) in MeOH (30 mL) was added [1,1′-Bis(diphenylphosphino)ferrocene]dichloropalladium(II), complex with dichloromethane (175.7 mg, 215.15 μmol) and triethylamine (261.36 mg, 2.58 mmol, 360.0 μl). The reaction mixture was carbonylated (CO atmosphere) at 135° C. and 40 atm pressure overnight. The mixture was cooled and concentrated to dryness. The residue was purified with column chromatography on silica (hexane-EtOAc 3:1 as eluent) to afford methyl 3-(1-[(tert-butoxy)carbonyl](methyl)aminocyclopropyl)benzoate (380.0 mg, 1.24 mmol, 57.8% yield) as colorless oil.

Step 4: To a stirred solution of methyl 3-(1-[(tert-butoxy)carbonyl](methyl)aminocyclopropyl)benzoate (380.0 mg, 1.24 mmol) in dry DCM (5 mL) was added dioxane/HCl (2 mL, 4M). The reaction mixture was stirred at r.t. for 5 h. The mixture was concentrated, the residue was triturated with hexane, and product collected by filtration to afford methyl 3-[1-(methylamino)cyclopropyl]benzoate hydrochloride (290.0 mg, 1.2 mmol, 96.4% yield) as white solid.

Step 5: To a cooled (0° C.) solution of 5-[(tert-butoxy)carbonyl]-4H,5H,6H,7H-pyrazolo[1,5-a]pyrazine-3-carboxylic acid (210.94 mg, 789.21 μmol) and [(dimethylamino)(3H-[1,2,3]triazolo[4,5-b]pyridin-3-yloxy)methylidene]dimethylazanium; hexafluoro-lambda5-phosphanuide (300.08 mg, 789.21 μmol) in DMF (0.8 mL) were added successively methyl 3-[1-(methylamino)cyclopropyl]benzoate hydrochloride (190.76 mg, 789.21 μmol) and triethylamine (319.44 mg, 3.16 mmol, 440.0 μl). The reaction mixture was stirred at r.t. overnight and diluted with brine. The mixture was extracted with EtOAc (2*20 mL). The combined organic phases was washed with brine, dried over Na₂SO₄, filtered and concentrated to afford tert-butyl 3-(1-[3-(methoxycarbonyl)phenyl]cyclopropyl(methyl)carbamoyl)-4H,5H,6H,7H-pyrazolo[1,5-a]pyrazine-5-carboxylate (270.0 mg, 594.03 μmol, 75.3% yield) as brown oil.

Step 6: To a solution of tert-butyl 3-(1-[3-(methoxycarbonyl)phenyl]cyclopropyl(methyl)carbamoyl)-4H,5H,6H,7H-pyrazolo[1,5-a]pyrazine-5-carboxylate (270.34 mg, 594.79 μmol) in THF/water/MeOH (2 mL/2 mL/1 mL) lithium hydroxide monohydrate (74.88 mg, 1.78 mmol) was added and the reaction mixture was stirred overnight at r.t. The mixture was concentrated, the residue was dissolved in water (5 mL) and the mixture was extracted with MTBE (3 mL). The aqueous phase was separated and acidified with 5% aq. HCl to pH 4. The product was extracted with EtOAc (2*5 mL). The combined organic phases was dried over Na₂SO₄, filtered and concentrated to afford 3-(1-N-methyl-5-[(tert-butoxy)carbonyl]-4H,5H,6H,7H-pyrazolo[1,5-a]pyrazine-3-amidocyclopropyl)benzoic acid (220.0 mg, 499.44 μmol, 84% yield) as yellow solid.

Rt (Method G) 1.23 mins, m/z 441 [M+H]⁺

1H NMR (400 MHz, DMSO-d₆) δ 12.99 (br.s, 1H), δ 7.81 (d, J=7.0 Hz, 1H), 7.63 (s, 1H), 7.50 (m, 1H), 7.30 (d, J=7.9 Hz, 1H), 6.94 (s, 1H), 4.75 (m, 2H), 4.05 (s, 2H), 3.78 (m, 2H), 3.06 (s, 3H), 1.58 (m, 2H), 1.44 (m, 11H).

### Synthesis of 4-(1-N-methyl-5-[(tert-butoxy)carbonyl]-4H,5H,6H,7H-pyrazolo[1,5-a]pyrazine-3-amidocyclopropyl)benzoic acid

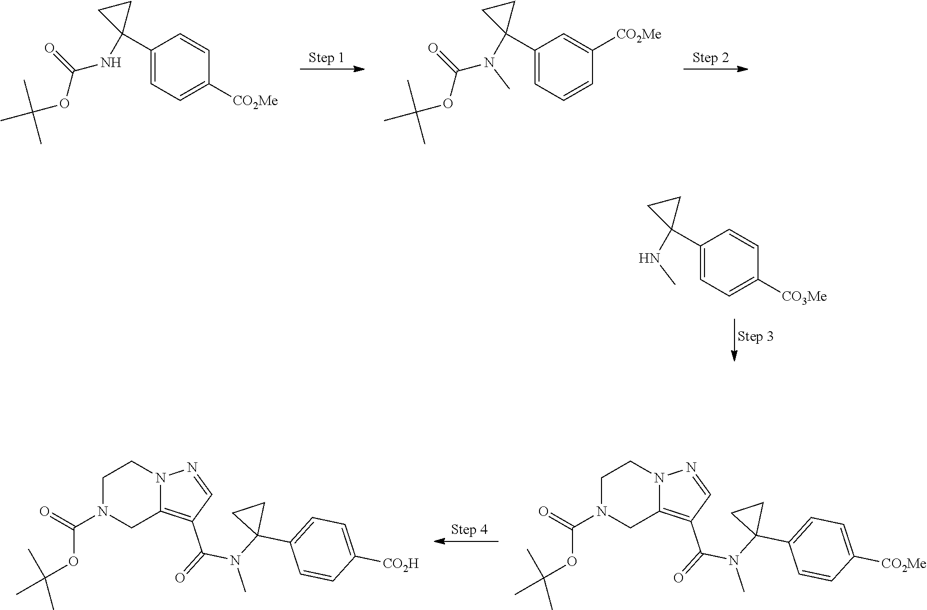

Step 1:Sodium hydride (123.54 mg, 5.15 mmol) was suspended in dry DMF (10 mL). A solution of methyl 4-(1-[(tert-butoxy)carbonyl]aminocyclopropyl)benzoate (999.86 mg, 3.43 mmol) in dry DMF (1 mL) was added dropwise (water bath cooling). The resulting mixture was stirred until gas evolution ceased and then cooled to 0° C. Iodomethane (2.44 g, 17.16 mmol) was added dropwise at that temperature; the resulting mixture was warmed to r.t. and then stirred overnight. The reaction mixture was poured into saturated aq. ammonium chloride solution. The resulting mixture was extracted twice with EtOAc (2×10 mL). The combined organic extracts were dried over Na₂SO₄ and concentrated to give methyl 4-(1-[(tert-butoxy)carbonyl](methyl)aminocyclopropyl)benzoate (900.0 mg, 2.95 mmol, 85.9% yield).

Step 2: Methyl 4-(1-[(tert-butoxy)carbonyl](methyl)aminocyclopropyl)benzoate (800.0 mg, 2.62 mmol) was dissolved in dioxane/HCl (10 mL, 4M solution) and the resulting mixture was stirred at r.t. After consumption of the starting material the resulting solution was evaporated to dryness to obtain crude methyl 4-[1-(methylamino)cyclopropyl]benzoate hydrochloride (600.0 mg, 2.48 mmol, 94.8% yield) which was used in next step without purification.

Step 3:Methyl 4-[1-(methylamino)cyclopropyl]benzoate hydrochloride (650.0 mg, 2.69 mmol), [(dimethylamino)(3H-[1,2,3]triazolo[4,5-b]pyridin-3-yloxy)methylidene]dimethylazanium; hexafluoro-lambda5-phosphanuide (1.12 g, 2.96 mmol) and triethylamine (680.14 mg, 6.72 mmol, 940.0 μl) were dissolved in dry DMF (5 mL) and the resulting mixture was stirred for 10 minutes. 5-[(Tert-butoxy)carbonyl]-4H,5H,6H,7H-pyrazolo[1,5-a]pyrazine-3-carboxylic acid (718.6 mg, 2.69 mmol) was added thereto and the resulting mixture was stirred at r.t. overnight. The resulting mixture was diluted with water (50 mL). The resulting precipitate was collected by filtration. The filter cake was redissolved in EtOAc (20 mL), dried over Na₂SO₄ and concentrated to give tert-butyl 3-(1-[4-(methoxycarbonyl)phenyl]cyclopropyl(methyl)carbamoyl)-4H,5H,6H,7H-pyrazolo[1,5-a]pyrazine-5-carboxylate (1.0 g, 2.2 mmol, 81.8% yield) which was used in next step without purification.

Step 4: Tert-butyl 3-(1-[4-(methoxycarbonyl)phenyl]cyclopropyl(methyl)carbamoyl)-4H,5H,6H,7H-pyrazolo[1,5-a]pyrazine-5-carboxylate (899.77 mg, 1.98 mmol) was mixed with sodium hydroxide (237.54 mg, 5.94 mmol) in methanol (10 mL) and the resulting mixture was stirred at r.t. overnight. After consumption of the starting material (1H NMR control) the resulting mixture was evaporated to dryness. The residue was partitioned between water (5 mL) and EtOAc (5 mL). The aqueous layer was collected and acidified with a solution of sodium hydrogen sulfate (713.02 mg, 5.94 mmol) in 5 mL of water. The precipitate was collected by filtration, then re-dissolved in EtOAc (10 mL), dried over Na₂SO₄ and evaporated to dryness.

The residue was purified by HPLC to obtain 4-(1-N-methyl-5-[(tert-butoxy)carbonyl]-4H,5H,6H,7H-pyrazolo[1,5-a]pyrazine-3-amidocyclopropyl)benzoic acid (366.0 mg, 830.89 μmol, 42% yield).

Rt (Method G) 1.23 mins, m/z 441 [M+H]⁺

1H NMR (400 MHz, DMSO-d₆) δ 12.88 (br.s, 1H), 7.92 (d, J=7.9 Hz, 2H), 7.17 (d, J=8.1 Hz, 2H), 6.93 (s, 1H), 4.76 (m, 2H), 4.05 (s, 2H), 3.77 (m, 2H), 3.04 (s, 3H), 1.64 (m, 2H), 1.43 (m, 11H).

### Synthesis of 2-(1-{N-methyl-5-[(tert-butoxy)carbonyl]-4H,5H,6H,7H-pyrazolo[1,5-a]pyrazine-3-amido}cyclopropyl)pyrimidine-5-carboxylic acid

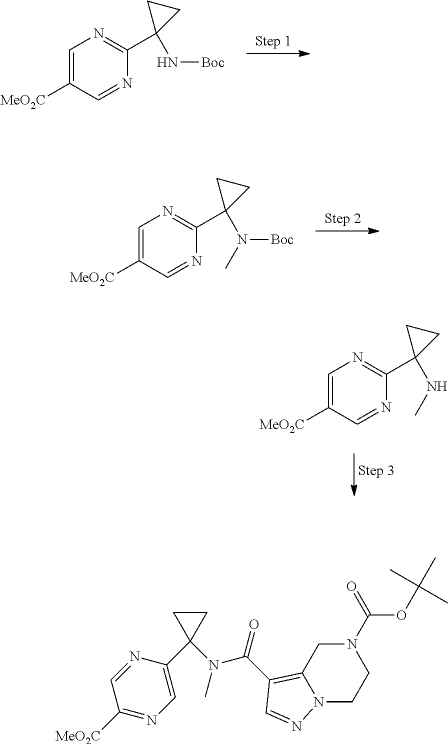 [SMILES: COC(=O)c1cnc(C2(NC(=O)OC(C)(C)C)CC2)nc1 ; COC(=O)c1cnc(C2(N(C)C(=O)OC(C)(C)C)CC2)nc1 ; CNC1(c2ncc(C(=O)OC)cn2)CC1 ; COC(=O)c1cnc(C2(N(C)C(=O)c3cnn4c3CN(C(=O)OC(C)(C)C)CC4)CC2)cn1]

Step 1: To a cooled (0° C.) suspension of sodium hydride (278.12 mg, 11.59 mmol) in dry DMF (20 mL) was added dropwise methyl 2-(1-[(tert-butoxy)carbonyl]aminocyclopropyl)pyrimidine-5-carboxylate (1.7 g, 5.8 mmol). The mixture was stirred until gas evolution ceased. Iodomethane (1.07 g, 7.53 mmol) was then added dropwise. The resulting mixture was warmed to r.t., stirred overnight, and then poured into water. The resulting mixture was extracted with EtOAc (2×50 mL). The organic phases were combined, washed with water, dried over sodium sulfate and concentrated to give methyl 2-(1-[(tert-butoxy)carbonyl](methyl)aminocyclopropyl)pyrimidine-5-carboxylate (700.0 mg, 99.0% purity, 2.25 mmol, 38.9% yield) that was used in the next step without further purification.

Step 2: Methyl 2-(1-[(tert-butoxy)carbonyl](methyl)aminocyclopropyl)pyrimidine-5-carboxylate (700.0 mg, 2.28 mmol) was dissolved in 4M HCl in dioxane (30 mL). The resulting mixture was stirred overnight then evaporated to dryness to give 1-[5-(methoxycarbonyl)pyrimidin-2-yl]-N-methylcyclopropan-1-aminium chloride (440.0 mg, 95.0% purity, 1.72 mmol, 75.3% yield) as a solid that was used in the next step without purification.

Step 3: To a stirred solution of methyl 2-[1-(methylamino)cyclopropyl]pyrimidine-5-carboxylate hydrochloride (439.34 mg, 1.8 mmol) and 5-[(tert-butoxy)carbonyl]-4H,5H,6H,7H-pyrazolo[1,5-a]pyrazine-3-carboxylic acid (481.87 mg, 1.8 mmol) in dry DMF (7 mL) were added [(dimethylamino)(3H-[1,2,3]triazolo[4,5-b]pyridin-3-yloxy)methylidene]dimethylazanium; hexafluoro-lambda5-phosphanuide (891.16 mg, 2.34 mmol) and triethylamine (638.88 mg, 6.31 mmol, 880.0 pVL, 3.5 equiv.). The mixture was stirred overnight then poured into water (50 mL) and extracted with EtOAc (2×50 mL). The combined organic extracts were washed with water (3×20 mL), dried (sodium sulfate), and concentrated.

The residue was purified by HPLC to give methyl 2-(1-N-methyl-5-[(tert-butoxy)carbonyl]-4H,5H,6H,7H-pyrazolo[1,5-a]pyrazine-3-amidocyclopropyl)pyrimidine-5-carboxylate (111.0 mg, 98.0% purity, 238.29 μmol, 13.2% yield) as white semi-solid.

### Synthesis of 6-(1-{5-[(tert-butoxy)carbonyl]-4H,5H,6H,7H-pyrazolo[1,5-a]pyrazine-3-amido}cyclopropyl)pyridine-3-carboxylic acid

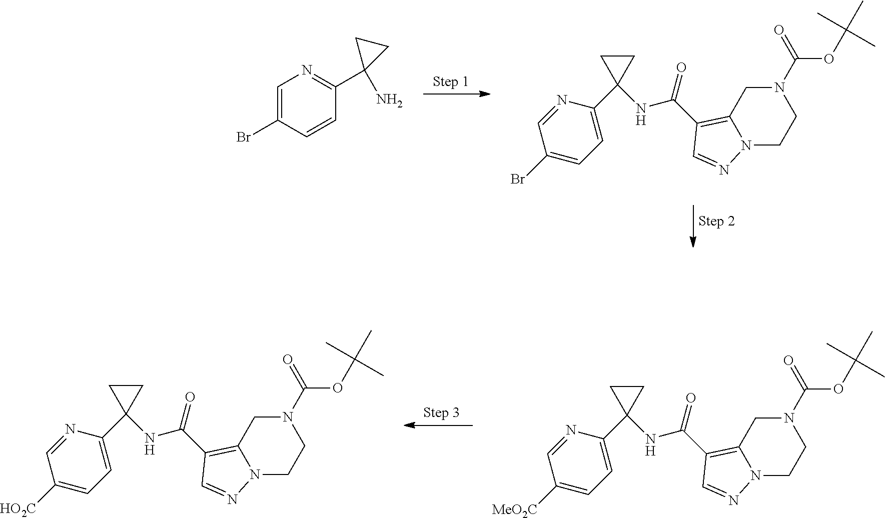 [SMILES: NC1(c2ccc(Br)cn2)CC1 ; CC(C)(C)OC(=O)N1CCn2ncc(C(=O)NC3(c4ccc(Br)cn4)CC3)c2C1 ; CC(C)(C)OC(=O)N1CCn2ncc(C(=O)NC3(c4ccc(C(=O)O)cn4)CC3)c2C1 ; COC(=O)c1ccc(C2(NC(=O)c3cnn4c3CN(C(=O)OC(C)(C)C)CC4)CC2)nc1]

Step 1: To a solution of 1-(5-bromopyridin-2-yl)cyclopropan-1-amine dihydrochloride (600.65 mg, 2.1 mmol) in DMF (5 mL) were added 5-[(tert-butoxy)carbonyl]-4H,5H,6H,7H-pyrazolo[1,5-a]pyrazine-3-carboxylic acid (561.34 mg, 2.1 mmol), HATU (798.55 mg, 2.1 mmol) and DIPEA (1.36 g, 10.51 mmol, 1.83 mL, 5.0 equiv.). The reaction mixture was stirred overnight at room temperature. The resulting mixture was diluted with water (10 mL) and extracted with EtOAc (3×20 mL). The combined organic extracts were washed with brine, dried over sodium sulfate, filtered, and concentrated. The residue was purified by HPLC to afford tert-butyl 3-[1-(5-bromopyridin-2-yl)cyclopropyl]carbamoyl-4H,5H,6H,7H-pyrazolo[1,5-a]pyrazine-5-carboxylate (400.0 mg, 865.16 μmol, 41.2% yield) as white solid.

Step 2: To a solution of tert-butyl 3-[1-(5-bromopyridin-2-yl)cyclopropyl]carbamoyl-4H,5H,6H,7H-pyrazolo[1,5-a]pyrazine-5-carboxylate (400.0 mg, 865.16 μmol) in MeOH (20 mL) were added Pd(dppf)C₂.DCM complex (35.33 mg, 43.26 μmol), and triethylamine (105.07 mg, 1.04 mmol, 140.0 μL, 1.2 equiv.). The mixture was carbonylated at 125° C. and 40 atm overnight. The mixture was cooled to room temperature and concentrated to dryness. The residue was dissolved in EtOAc (10 mL), washed with water (5 mL), dried over sodium sulfate, filtered, and concentrated to afford methyl 6-(1-5-[(tert-butoxy)carbonyl]-4H,5H,6H,7H-pyrazolo[1,5-a]pyrazine-3-amidocyclopropyl)pyridine-3-carboxylate (390.0 mg, 70.0% purity, 618.37 μmol, 71.5% yield) as brown solid, that was used in the next step without further purification.

Step 3: To a solution of methyl 6-(1-5-[(tert-butoxy)carbonyl]-4H,5H,6H,7H-pyrazolo[1,5-a]pyrazine-3-amidocyclopropyl)pyridine-3-carboxylate (390.0 mg, 883.39 μmol) in THF/water/MeOH (2 mL/2 mL/1 mL) was added lithium hydroxide monohydrate (148.43 mg, 3.54 mmol). The reaction mixture was stirred overnight at room temperature then concentrated under reduced pressure. The residue was purified by HPLC to give 6-(1-{5-[(tert-butoxy)carbonyl]-4H,5H,6H,7H-pyrazolo[1,5-a]pyrazine-3-amido}cyclopropyl)pyridine-3-carboxylic acid.

### Synthesis of 2-(1-{5-[(tert-butoxy)carbonyl]-4H,5H,6H,7H-pyrazolo[1,5-a]pyrazine-3-amido}cyclopropyl)pyrimidine-5-carboxylic acid

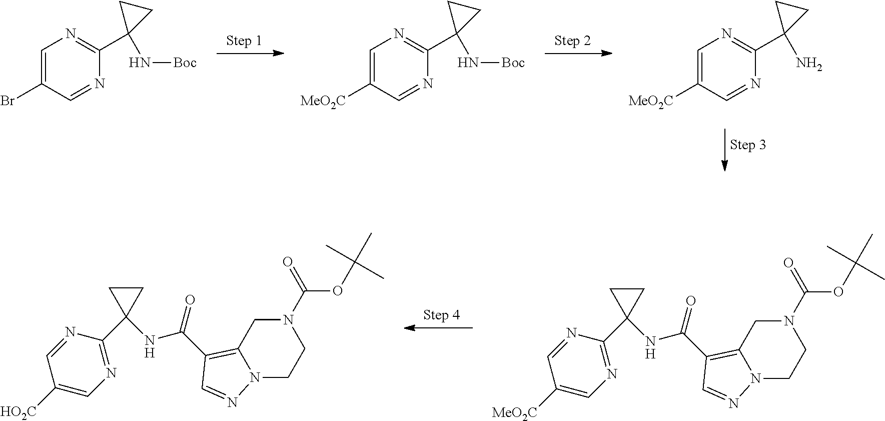 [SMILES: CC(C)(C)OC(=O)NC1(c2ncc(Br)cn2)CC1 ; COC(=O)c1cnc(C2(NC(=O)OC(C)(C)C)CC2)nc1 ; COC(=O)c1cnc(C2(N)CC2)nc1 ; CC(C)(C)OC(=O)N1CCn2ncc(C(=O)NC3(c4ncc(C(=O)O)cn4)CC3)c2C1 ; COC(=O)c1cnc(C2(NC(=O)c3cnn4c3CN(C(=O)OC(C)(C)C)CC4)CC2)nc1]

Step 1: Tert-butyl N-[1-(5-bromopyrimidin-2-yl)cyclopropyl]carbamate (3.0 g, 9.55 mmol), triethylamine (1.16 g, 11.46 mmol) and Pd(dppf)C₂.DCM complex (3 mol %) were dissolved in methanol (100 mL). The reaction mixture was heated at 120° C. in a high pressure vessel at 40 atm CO pressure for 18 h, then cooled to room temperature. Solvent was removed in vacuo and water (100 mL) was added. The mixture was stirred at room temperature for 1 hour and product was collected by filtration. The solid was washed with water (100 mL) and air-dried to give methyl 2-(1-[(tert-butoxy)carbonyl]aminocyclopropyl)pyrimidine-5-carboxylate (2.5 g, 98.0% purity, 8.35 mmol, 87.5% yield) as an orange solid.

Step 2: To methyl 2-(1-[(tert-butoxy)carbonyl]aminocyclopropyl)pyrimidine-5-carboxylate (800.0 mg, 2.73 mmol) was added 4M HCl in dioxane (40 mL, 160 mmol). The resulting mixture was stirred overnight at room temperature. The product was collected by filtration and washed with MTBE (20 mL), and air-dried to obtain 1-[5-(methoxycarbonyl)pyrimidin-2-yl]cyclopropan-1-aminium chloride (400.0 mg, 98.0% purity, 1.71 mmol, 62.6% yield) as white solid.

Step 3: To a stirred solution of methyl 2-(1-aminocyclopropyl)pyrimidine-5-carboxylate hydrochloride (400.19 mg, 1.74 mmol) and 5-[(tert-butoxy)carbonyl]-4H,5H,6H,7H-pyrazolo[1,5-a]pyrazine-3-carboxylic acid (465.74 mg, 1.74 mmol) in DMF (7 mL) were added [(dimethylamino)(3H-[1,2,3]triazolo[4,5-b]pyridin-3-yloxy)methylidene]dimethylazanium; hexafluoro-lambda5-phosphanuide (861.31 mg, 2.27 mmol) and triethylamine (617.1 mg, 6.1 mmol, 850.0 μL, 3.5 equiv.). The mixture was stirred overnight at room temperature and then poured into water (50 mL) and extracted with MTBE (2×50 mL). The combined organic extracts were washed with water (3×20 mL), and dried over anhydrous sodium sulfate. The solvent was removed under vacuum to yield methyl 2-(1-5-[(tert-butoxy)carbonyl]-4H,5H,6H,7H-pyrazolo[1,5-a]pyrazine-3-amidocyclopropyl)pyrimidine-5-carboxylate (700.0 mg, 91.0% purity, 1.44 mmol, 82.6% yield).

Step 4: To a solution of methyl 2-(1-5-[(tert-butoxy)carbonyl]-4H,5H,6H,7H-pyrazolo[1,5-a]pyrazine-3-amidocyclopropyl)pyrimidine-5-carboxylate (700.2 mg, 1.58 mmol) in MeOH/THF/H₂O (4:4:1) (27 mL) was added lithium hydroxide monohydrate (265.63 mg, 6.33 mmol). The mixture was stirred for 18 h, and then concentrated. Water (200 mL) was added and the resulting solution was cooled to (0-5° C.) and adjusted to pH 3-4 with 1M NaHSO₄. The suspension was stirred for 30 minutes and the product was collected by filtration. The filter cake was washed with water, then dried to afford 2-(1-{5-[(tert-butoxy)carbonyl]-4H,5H,6H,7H-pyrazolo[1,5-a]pyrazine-3-amido}cyclopropyl)pyrimidine-5-carboxylic acid (310.0 mg, 98.0% purity, 709.08 μmol, 44.8% yield) as pale yellow solid.

### Synthesis of tert-butyl 3-((1-(5-hydroxypyridin-2-yl)cyclopropyl)(methyl)carbamoyl)-6,7-dihydro-1H-pyrazolo[4,3-c]pyridine-5(4H)-carboxylate

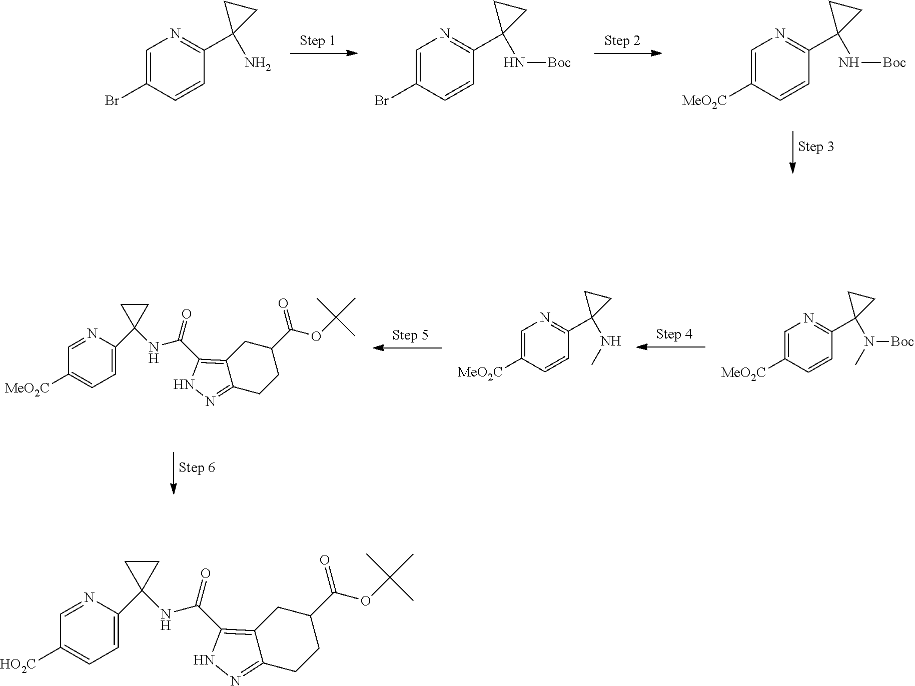

Step 1: To a solution of 1-(5-bromopyridin-2-yl)cyclopropan-1-amine dihydrochloride (4.0 g, 13.98 mmol) in DCM (50 mL) was added di-tert-butyl dicarbonate (3.2 g, 14.67 mmol, 3.37 mL, 1.05 equiv.). The resulting mixture was stirred for 5 mins then triethylamine (3.54 g, 34.94 mmol, 4.87 mL, 2.5 equiv.) was added dropwise. The resulting mixture was stirred at r.t. for 12 hours, then transferred to a separating funnel. The organic phase was washed with water (20 mL), and brine, then dried over sodium sulfate to obtain tert-butyl N-[1-(5-bromopyridin-2-yl)cyclopropyl]carbamate (4.2 g, 13.41 mmol, 96% yield).

Step 2: Tert-butyl (1-(5-bromopyridin-2-yl)cyclopropyl)carbamate (4.2 g, 13.41 mmol) was carbonylated in MeOH (100 mL) at 130° C. and 50 atm. CO pressure with Pd(dppf)C₂.DCM complex as catalyst. Once reaction was complete, the mixture was concentrated and the residue was partitioned between water (100 mL) and EtOAc (100 mL). The organic layer was collected, dried over sodium sulfate and concentrated to obtain methyl 6-(1-[(tert-butoxy)carbonyl]aminocyclopropyl)pyridine-3-carboxylate (4.6 g, 15.74 mmol, 117.3% yield) which was used in the next step without further purification.

Step 3: To a cooled (water bath) suspension of sodium hydride (106.92 mg, 4.46 mmol) in dry DMF (15 mL) was added dropwise a solution of methyl 6-(1-[(tert-butoxy)carbonyl]aminocyclopropyl)pyridine-3-carboxylate (1.0 g, 3.43 mmol) in dry DMF (5 mL). The resulting mixture was stirred until gas evolution ceased. The mixture was cooled to 0° C. followed by the dropwise addition of iodomethane (729.6 mg, 5.14 mmol, 320.0 μL, 1.5 equiv.). The resulting mixture was warmed to r.t. and then stirred overnight. The mixture was poured into saturated aq. ammonium chloride solution, and the product was extracted with EtOAc (2×40 mL). The organic phases were combined, dried over sodium sulfate and concentrated to give methyl 6-(1-[(tert-butoxy)carbonyl](methyl)aminocyclopropyl)pyridine-3-carboxylate (800.0 mg, 2.61 mmol, 76.2% yield).

Step 4: To methyl 6-(1-[(tert-butoxy)carbonyl](methyl)aminocyclopropyl)pyridine-3-carboxylate (800.0 mg, 2.61 mmol) was added 4M HCl in dioxane (50 mL, 200 mmol). The resulting mixture was stirred at r.t. for 12 hours then evaporated to dryness to obtain methyl 6-[1-(methylamino)cyclopropyl]pyridine-3-carboxylate dihydrochloride (700.0 mg, 2.51 mmol, 96% yield) that was used in the next step without further purification.

Step 5: 5-[(tert-Butoxy)carbonyl]-1H,4H,5H,6H,7H-pyrazolo[4,3-c]pyridine-3-carboxylic acid (670.1 mg, 2.51 mmol), [(dimethylamino)(3H-[1,2,3]triazolo[4,5-b]pyridin-3-yloxy)methylidene]dimethylazanium; hexafluoro-lambda5-phosphanuide (1.05 g, 2.76 mmol) and triethylamine (887.93 mg, 8.77 mmol) were mixed in dry DMF (10 mL). The resulting mixture was stirred at r.t. for 10 minutes, followed by the addition of methyl 6-[1-(methylamino)cyclopropyl]pyridine-3-carboxylate dihydrochloride (700.0 mg, 2.51 mmol).

The resulting mixture was stirred at r.t. overnight. Then, the reaction mixture was poured into H₂O (60 mL). The product was collected by filtration, washed with H₂O (2×10 mL) and air-dried to obtain methyl 6-(1-N-methyl-5-[(tert-butoxy)carbonyl]-1H,4H,5H,6H,7H-pyrazolo[4,3-c]pyridine-3-amidocyclopropyl)pyridine-3-carboxylate (350.0 mg, 768.37 μmol, 30.6% yield) which was used in next step without further purification.

Step 6: To a solution of methyl 6-(1-N-methyl-5-[(tert-butoxy)carbonyl]-1H,4H,5H,6H,7H-pyrazolo[4,3-c]pyridine-3-amidocyclopropyl)pyridine-3-carboxylate (349.77 mg, 767.87 μmol) in MeOH (20 mL) was added lithium hydroxide monohydrate (322.23 mg, 7.68 mmol).

The reaction mixture was stirred at 50° C. overnight, then concentrated and partitioned between water (10 mL) and EtOAc (10 mL). The aqueous layer was collected and acidified with NaHSO₄ (15% aq. sol). The resulting mixture was extracted with EtOAc (2×20 mL). The combined organic extracts were dried over sodium sulfate and concentrated to give tert-butyl 3-((1-(5-hydroxypyridin-2-yl)cyclopropyl)(methyl)carbamoyl)-6,7-dihydro-1H-pyrazolo[4,3-c]pyridine-5(4H)-carboxylate.

### Synthesis of 6-(1-{5-[(tert-butoxy)carbonyl]-1H,4H,5H,6H,7H-pyrazolo[4,3-c]pyridine-3-amido}cyclopropyl)pyridine-3-carboxylic acid

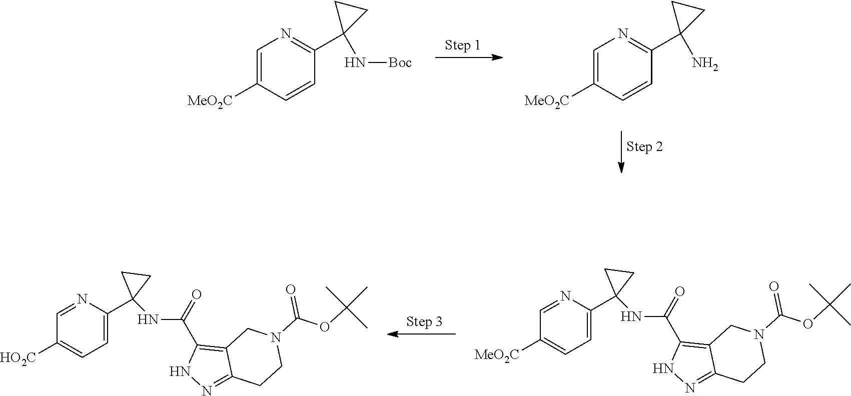 [SMILES: COC(=O)c1ccc(C2(NC(=O)OC(C)(C)C)CC2)nc1 ; COC(=O)c1ccc(C2(N)CC2)nc1 ; CC(C)(C)OC(=O)N1CCc2n[nH]c(C(=O)NC3(c4ccc(C(=O)O)cn4)CC3)c2C1 ; COC(=O)c1ccc(C2(NC(=O)c3[nH]nc4c3CN(C(=O)OC(C)(C)C)CC4)CC2)nc1]

Step 1: To methyl 6-(1-[(tert-butoxy)carbonyl]aminocyclopropyl)pyridine-3-carboxylate (2.0 g, 6.84 mmol) was added 4M HCl in dioxane (50 mL, 200 mmol). The resulting mixture was stirred at r.t. for 12 hours, then concentrated to dryness to give methyl 6-(1-aminocyclopropyl)pyridine-3-carboxylate dihydrochloride (2.0 g, 7.54 mmol, 110.3% yield) that was used in the next step without further purification.

Step 2: 5-[(tert-butoxy)carbonyl]-1H,4H,5H,6H,7H-pyrazolo[4,3-c]pyridine-3-carboxylic acid (1.01 g, 3.77 mmol), [(dimethylamino)(3H-[1,2,3]triazolo[4,5-b]pyridin-3-yloxy)methylidene]dimethylazanium; hexafluoro-lambda-5-phosphanuide (1.58 g, 4.15 mmol) and triethylamine (1.34 g, 13.2 mmol, 1.84 mL, 3.5 equiv.) were mixed in dry DMF (10 mL).

The resulting mixture was stirred at r.t. for 10 minutes, followed by the addition of methyl 6-(1-aminocyclopropyl)pyridine-3-carboxylate dihydrochloride (999.94 mg, 3.77 mmol). The reaction mixture was stirred at r.t. overnight. Then, the mixture was poured into water (60 mL).

The precipitate was collected by filtration, washed with water (2×10 mL) and dried to obtain crude methyl 6-(1-5-[(tert-butoxy)carbonyl]-1H,4H,5H,6H,7H-pyrazolo[4,3-c]pyridine-3-amidocyclopropyl)pyridine-3-carboxylate (1.1 g, 2.49 mmol, 66.1% yield) which was used in next step without further purification.

Step 3: To a solution of methyl 6-(1-5-[(tert-butoxy)carbonyl]-1H,4H,5H,6H,7H-pyrazolo[4,3-c]pyridine-3-amidocyclopropyl)pyridine-3-carboxylate (500.0 mg, 1.13 mmol) in MeOH (20 mL) was added lithium hydroxide monohydrate (475.15 mg, 11.32 mmol). The reaction mixture was heated at 50° C. overnight. The resulting mixture was cooled and concentrated under reduced pressure. The residue was partitioned between water (10 mL) and EtOAc (10 mL). The aqueous layer was collected and acidified with NaHSO₄ (15% aq. sol). The resulting mixture was extracted with EtOAc (2×20 mL). The combined organic layer was dried over sodium sulfate and concentrated to give 6-(1-{5-[(tert-butoxy)carbonyl]-1H,4H,5H,6H,7H-pyrazolo[4,3-c]pyridine-3-amido}cyclopropyl)pyridine-3-carboxylic acid.

### Synthesis of 2-(1-{N-methyl-5-[(tert-butoxy)carbonyl]-1H,4H,5H,6H,7H-pyrazolo[4,3-c]pyridine-3-amido}cyclopropyl)pyrimidine-5-carboxylic acid

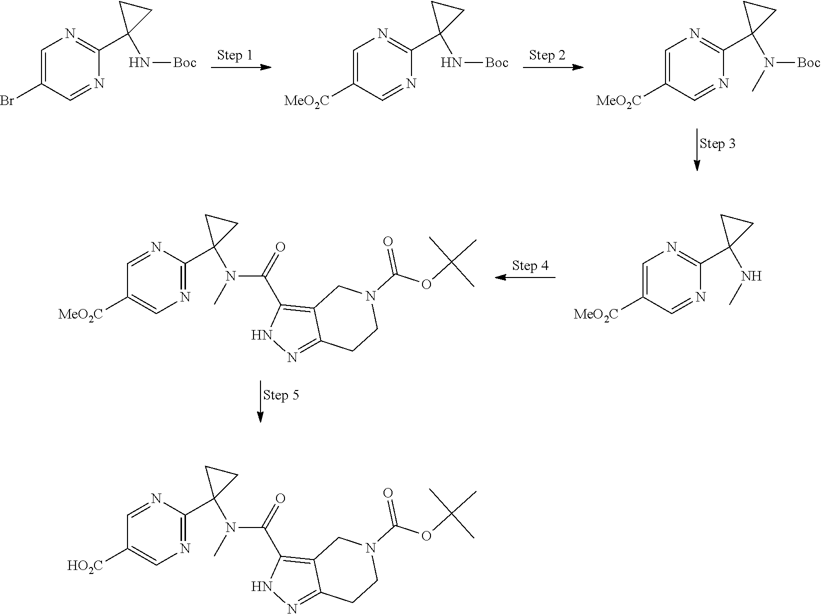 [SMILES: CC(C)(C)OC(=O)NC1(c2ncc(Br)cn2)CC1 ; COC(=O)c1cnc(C2(NC(=O)OC(C)(C)C)CC2)nc1 ; COC(=O)c1cnc(C2(N(C)C(=O)OC(C)(C)C)CC2)nc1 ; COC(=O)c1cnc(C2(N(C)C(=O)c3[nH]nc4c3CN(C(=O)OC(C)(C)C)CC4)CC2)nc1 ; CNC1(c2ncc(C(=O)OC)cn2)CC1 ; CN(C(=O)c1[nH]nc2c1CN(C(=O)OC(C)(C)C)CC2)C1(c2ncc(C(=O)O)cn2)CC1]

Step 1: A solution of tert-butyl N-[1-(5-bromopyrimidin-2-yl)cyclopropyl]carbamate (3.0 g, 9.55 mmol), Pd(dppf)Cl2.DCM complex (139.75 mg, 190.99 μmol) and triethylamine (2.9 g, 28.65 mmol) in MeOH (100 mL) was heated overnight at 120° C. in a steel bomb under CO pressure at 25 bar. After cooling to r.t. the solution was concentrated and the residue was purified by HPLC to give methyl 2-(1-[(tert-butoxy)carbonyl]aminocyclopropyl)pyrimidine-5-carboxylate (2.6 g, 8.86 mmol, 92.8% yield).

Step 2: To a cooled (water bath) solution of methyl 2-(1-[(tert-butoxy)carbonyl]aminocyclopropyl)pyrimidine-5-carboxylate (725.0 mg, 2.47 mmol) in DMF (50 mL) was added sodium hydride (118.68 mg, 4.95 mmol) portionwise, maintaining the temperature below 25° C. After gas evolution ceased, iodomethane (526.48 mg, 3.71 mmol, 230.0 μL, 1.5 equiv.) was added dropwise. The resulting mixture was stirred overnight at room temperature. The reaction mixture was poured into water (400 mL) and extracted with EtOAc (200 mL). The organic phase was washed with water (2×100 mL), brine, dried over sodium sulfate, and concentrated to give methyl 2-(1-[(tert-butoxy)carbonyl](methyl)aminocyclopropyl)pyrimidine-5-carboxylate (550.0 mg, 1.79 mmol, 72.4% yield).

Step 3: To methyl 2-(1-[(tert-butoxy)carbonyl](methyl)aminocyclopropyl)pyrimidine-5-carboxylate (550.0 mg, 1.79 mmol) was added 4M HCl in dioxane (15 mL, 60 mmol). The reaction mixture was stirred at room temperature overnight. Product was collected by filtration, washed with MTBE, then dried to afford methyl 2-[1-(methylamino)cyclopropyl]pyrimidine-5-carboxylate hydrochloride (200.0 mg, 820.71 μmol, 45.9% yield).

Step 4: To a solution of 5-[(tert-butoxy)carbonyl]-1H,4H,5H,6H,7H-pyrazolo[4,3-c]pyridine-3-carboxylic acid (76.7 mg, 286.97 μmol) and triethylamine (87.12 mg, 860.91 μmol, 120.0 μL, 3.0 equiv.) in dry DMF (20 mL) was added (1H-1,2,3-benzotriazol-1-yloxy)tris(dimethylamino)phosphonium hexafluorophosphate (139.61 mg, 315.67 μmol). The resulting mixture was stirred for 10 mins, followed by the addition of methyl 2-[1-(methylamino)cyclopropyl]pyrimidine-5-carboxylate hydrochloride (70.0 mg, 287.25 μmol).

The reaction mixture was stirred overnight at room temperature. Then, the mixture was partitioned between EtOAc (100 mL) and water (200 mL). The organic phase was washed with water (50 mL), brine, dried over sodium sulfate, and concentrated under reduced pressure. The residue was purified by HPLC to afford methyl 2-(1-N-methyl-5-[(tert-butoxy)carbonyl]-1H,4H,5H,6H,7H-pyrazolo[4,3-c]pyridine-3-amidocyclopropyl)-pyrimidine-5-carboxylate (100.0 mg, 219.06 μmol, 76.3% yield).

Step 5: To a solution of methyl 2-(1-N-methyl-5-[(tert-butoxy)carbonyl]-1H,4H,5H,6H,7H-pyrazolo[4,3-c]pyridine-3-amidocyclopropyl)pyrimidine-5-carboxylate (100.0 mg, 219.06 μmol) in MeOH (3 mL), was added a solution of sodium hydroxide (19.27 mg, 481.8 μmol) in water (0.5 mL). The resulting mixture was stirred overnight at room temperature. The reaction mixture was concentrated under reduced pressure and the residue was taken up in water (10 mL). The resulting solution was acidified with NaHSO₄ and extracted with MTBE (2×10 mL).

The combined organic extracts were dried over sodium sulfate and concentrated to give 2-(1-N-methyl-5-[(tert-butoxy)carbonyl]-1H,4H,5H,6H,7H-pyrazolo[4,3-c]pyridine-3-amidocyclopropyl)pyrimidine-5-carboxylic acid (60.0 mg, 135.6 μmol, 61.9% yield).

### Synthesis of 2-(1-{5-[(tert-butoxy)carbonyl]-1H,4H,5H,6H,7H-pyrazolo[4,3-c]pyridine-3-amido}cyclopropyl)pyrimidine-5-carboxylic acid

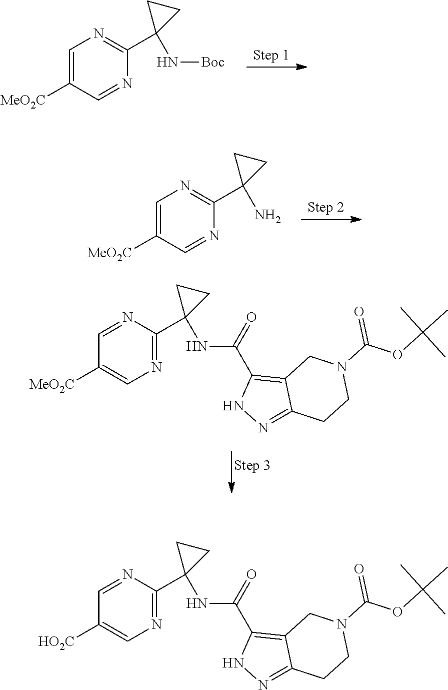

Step 1: To methyl 2-(1-[(tert-butoxy)carbonyl]aminocyclopropyl)pyrimidine-5-carboxylate (710.0 mg, 2.42 mmol) was added 4M HCl in dioxane (20 mL, 80 mmol). The mixture was stirred at room temperature overnight. The precipitate was collected by filtration and washed MTBE, then dried to give methyl 2-(1-aminocyclopropyl)pyrimidine-5-carboxylate hydrochloride (540.0 mg, 2.35 mmol, 97.1% yield) as pale pink powder.

Step 2: To a solution of 5-[(tert-butoxy)carbonyl]-1H,4H,5H,6H,7H-pyrazolo[4,3-c]pyridine-3-carboxylic acid (628.21 mg, 2.35 mmol) and triethylamine (832.42 mg, 8.23 mmol, 1.15 mL, 3.5 equiv.) in dry DMF (20 mL) was added (1H-1,2,3-benzotriazol-1-yloxy)tris(dimethylamino)phosphonium hexafluorophosphate (1.14 g, 2.59 mmol). The resulting mixture was stirred for 10 mins, then methyl 2-(1-aminocyclopropyl)pyrimidine-5-carboxylate hydrochloride (540.0 mg, 2.35 mmol) was added and the stirring was continued overnight. The reaction mixture was partitioned between EtOAc (50 mL) and water (50 mL).

The organic phase was washed with brine, dried over sodium sulfate, concentrated under reduced pressure then purified by HPLC to give methyl 2-(1-5-[(tert-butoxy)carbonyl]-1H,4H,5H,6H,7H-pyrazolo[4,3-c]pyridine-3-amidocyclopropyl)pyrimidine-5-carboxylate (70.0 mg, 158.2 μmol, 7% yield).

Step 3: To a solution of methyl 2-(1-5-[(tert-butoxy)carbonyl]-1H,4H,5H,6H,7H-pyrazolo[4,3-c]pyridine-3-amidocyclopropyl)pyrimidine-5-carboxylate (70.0 mg, 158.2 μmol) in MeOH (3 mL) was added a solution of sodium hydroxide (22.15 mg, 553.87 μmol) in water (0.2 mL).

The resulting mixture was stirred overnight at room temperature then concentrated under reduced pressure. The residue was taken up in water (15 mL), washed with EtOAc (10 mL), then acidified with aq. HCl (1N) to pH-3 and extracted with EtOAc (2×50 mL). The combined organic extracts were dried over sodium sulfate and concentrated give 2-(1-{5-[(tert-butoxy)carbonyl]-1H,4H,5H,6H,7H-pyrazolo[4,3-c]pyridine-3-amido}cyclopropyl)pyrimidine-5-carboxylic acid (36.0 mg, 84.03 μmol, 53.1% yield) as white powder.

### Synthesis of tert-butyl 3-(1-[4-(methoxycarbonyl)phenyl]cyclopropylcarbamoyl)-4H,5H,6H,7H-pyrazolo[1,5-a]pyrazine-5-carboxylate

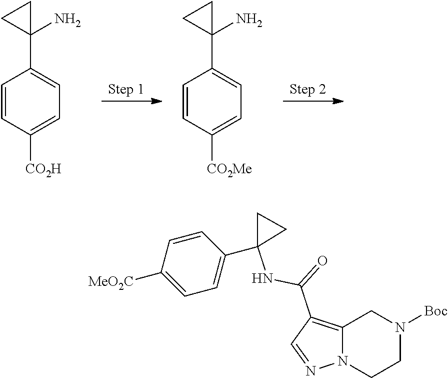 [SMILES: NC1(c2ccc(C(=O)O)cc2)CC1 ; COC(=O)c1ccc(C2(N)CC2)cc1 ; COC(=O)c1ccc(C2(NC(=O)c3cnn4c3CN(C(=O)OC(C)(C)C)CC4)CC2)cc1]

Step 1: To a solution of 4-(1-aminocyclopropyl)benzoic acid hydrochloride (490.78 mg, 2.3 mmol) in dry methanol (30 mL) was added thionyl chloride (410.0 mg, 3.45 mmol, 250.0 μL, 1.5 equiv.) The mixture was heated at reflux overnight, then cooled to room temperature and evaporated to dryness to give methyl 4-(1-aminocyclopropyl)benzoate hydrochloride (500.0 mg, 2.2 mmol, 95.6% yield).

Step 2: 5-[(tert-butoxy)carbonyl]-4H,5H,6H,7H-pyrazolo[1,5-a]pyrazine-3-carboxylic acid (254.85 mg, 953.48 μmol), HATU (398.8 mg, 1.05 mmol) and triethylamine (241.21 mg, 2.38 mmol, 330.0 μL, 2.5 equiv.) were mixed in dry DMF (5 mL) at room temperature. The resulting mixture was stirred for 10 mins, followed by the addition of methyl 4-(1-aminocyclopropyl)benzoate (182.33 mg, 953.48 μmol). The reaction mixture was stirred at room temperature overnight. The resulting mixture was concentrated then purified directly by HPLC to obtain tert-butyl 3-(1-[4-(methoxycarbonyl)phenyl]cyclopropylcarbamoyl)-4H,5H,6H,7H-pyrazolo[1,5-a]pyrazine-5-carboxylate (527.0 mg, 1.2 mmol, 125.5% yield).

### Synthesis of tert-butyl 3-(1-[3-(methoxycarbonyl)phenyl]cyclopropylcarbamoyl)-4H,5H,6H,7H-pyrazolo[1,5-a]pyrazine-5-carboxylate

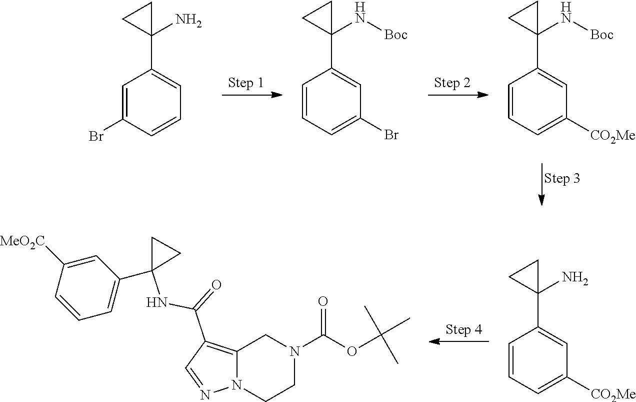 [SMILES: NC1(c2cccc(Br)c2)CC1 ; CC(C)(C)OC(=O)NC1(c2cccc(Br)c2)CC1 ; COC(=O)c1cccc(C2(NC(=O)OC(C)(C)C)CC2)c1 ; COC(=O)c1cccc(C2(NC(=O)c3cnn4c3CN(C(=O)OC(C)(C)C)CC4)CC2)c1 ; COC(=O)c1cccc(C2(N)CC2)c1]

Step 1: To a cooled (0° C.) suspension of 1-(3-bromophenyl)cyclopropan-1-amine hydrochloride (2.0 g, 8.05 mmol) in dry DCM (15 mL) were added di-tert-butyl dicarbonate (1.76 g, 8.05 mmol) and triethylamine (977.02 mg, 9.66 mmol). The reaction mixture was stirred at room temperature for 4 h. Water (5 mL) was added, the organic phase was separated and washed with 5% aq. HCl, water, dried over sodium sulfate, filtered, and concentrated to give tert-butyl N-[1-(3-bromophenyl)cyclopropyl]carbamate (2.2 g, 7.05 mmol, 87.6% yield) as white solid.

Step 2: To a solution of tert-butyl N-[1-(3-bromophenyl)cyclopropyl]carbamate (2.2 g, 7.05 mmol) in MeOH (80 mL) were added Pd(dppf)Cl₂.DCM complex (575.46 mg, 704.67 μmol) and triethylamine (855.67 mg, 8.46 mmol). The mixture was carbonylated at 125° C. and 40 atm for 20 h. The resulting mixture was cooled and concentrated to dryness. The residue was dissolved in EtOAc (20 mL) and the solution was washed with water (5 mL), dried over sodium sulfate, filtered, and concentrated. The residue was purified by flash column chromatography on silica (hexane-EtOAc 3:1 as eluent) to afford methyl 3-(1-[(tert-butoxy)carbonyl]aminocyclopropyl)benzoate (1.3 g, 4.46 mmol, 63.3% yield) as brown oil.

Step 3: To a solution of methyl 3-(1-[(tert-butoxy)carbonyl]aminocyclopropyl)benzoate (1.3 g, 4.46 mmol) in DCM (10 mL) was added 4M HCl in dioxane (7.8 mL, 31.2 mmol). The reaction mixture was stirred at room temperature for 8 h. The precipitate was collected by filtration and washed with dry EtOAc, then air-dried to afford methyl 3-(1-aminocyclopropyl)benzoate hydrochloride (900.0 mg, 3.95 mmol, 88.6% yield) as white solid.

Step 4: To a solution of 5-[(tert-butoxy)carbonyl]-4H,5H,6H,7H-pyrazolo[1,5-a]pyrazine-3-carboxylic acid (586.75 mg, 2.2 mmol) in dry DMF (5 mL) was added HATU (834.71 mg, 2.2 mmol). The resulting mixture was stirred for 10 mins, then methyl 3-(1-aminocyclopropyl)benzoate hydrochloride (500.0 mg, 2.2 mmol) and triethylamine (888.56 mg, 8.78 mmol) were added. The reaction mixture was stirred overnight, then partitioned between EtOAc (20 mL) and water (30 mL). The organic phase was washed with water (3×10 mL), sat. aq. NaHCO₃, and brine, then dried over sodium sulfate, and concentrated under reduced pressure to give tert-butyl 3-(1-[3-(methoxycarbonyl)phenyl]cyclopropylcarbamoyl)-4H,5H,6H,7H-pyrazolo[1,5-a]pyrazine-5-carboxylate (710.0 mg, 1.61 mmol, 73.4% yield) as colorless solid.

### Synthesis of tert-butyl 3-[(1-[4-(methoxycarbonyl)phenyl]methylcyclopropyl)(methyl)carbamoyl]-4H,5H,6H,7H-pyrazolo[1,5-a]pyrazine-5-carboxylate

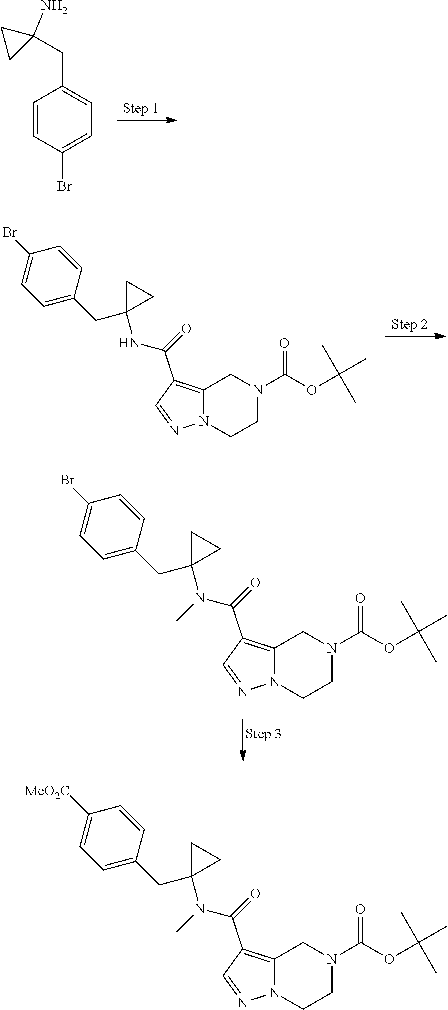 [SMILES: NC1(Cc2ccc(Br)cc2)CC1 ; CC(C)(C)OC(=O)N1CCn2ncc(C(=O)NC3(Cc4ccc(Br)cc4)CC3)c2C1 ; CN(C(=O)c1cnn2c1CN(C(=O)OC(C)(C)C)CC2)C1(Cc2ccc(Br)cc2)CC1 ; COC(=O)c1ccc(CC2(N(C)C(=O)c3cnn4c3CN(C(=O)OC(C)(C)C)CC4)CC2)cc1]

Step 1: To a solution of 5-[(tert-butoxy)carbonyl]-4H,5H,6H,7H-pyrazolo[1,5-a]pyrazine-3-carboxylic acid (1.12 g, 4.19 mmol) and triethylamine (963.2 mg, 9.52 mmol, 1.33 mL, 2.5 equiv.) in dry DMF (40 mL) was added (1H-1,2,3-benzotriazol-1-yloxy)tris(dimethylamino)phosphonium hexafluorophosphate (1.85 g, 4.19 mmol. The resulting mixture was stirred for 10 mins, then 1-[(4-bromophenyl)methyl]cyclopropan-1-amine hydrochloride (1.0 g, 3.81 mmol) was added and the stirring was continued overnight.

The reaction mixture was partitioned between EtOAc (50 mL) and water (150 mL). The organic phase was washed with water (50 mL), brine, dried over sodium sulfate, and concentrated under reduced pressure to give tert-butyl 3-(1-[(4-bromophenyl)methyl]cyclopropylcarbamoyl)-4H,5H,6H,7H-pyrazolo[1,5-a]pyrazine-5-carboxylate (2.0 g, 90.0% purity, 3.79 mmol, 99.4% yield).

Step 2: To a cooled (water bath) solution of tert-butyl 3-(1-[(4-bromophenyl)methyl]cyclopropylcarbamoyl)-4H,5H,6H,7H-pyrazolo[l1,5-a]pyrazine-5-carboxylate (2.0 g, 4.21 mmol) in DMF (50 mL), was added sodium hydride (201.92 mg, 8.41 mmol) portionwise, maintaining the temperature below 25° C. After gas evolution ceased, iodomethane (895.74 mg, 6.31 mmol, 390.0 μL, 1.5 equiv.) was added dropwise and the resulting mixture was left to stir overnight at room temperature. The reaction mixture was poured into water (400 mL) and extracted with EtOAc (200 mL). The organic phase was washed with water (2×100 mL), brine, dried over sodium sulfate, and concentrated to afford tert-butyl 3-(1-[(4-bromophenyl)methyl]cyclopropyl(methyl)carbamoyl)-4H,5H,6H,7H-pyrazolo[1,5-a]pyrazine-5-carboxylate (1.8 g, 3.68 mmol, 87.4% yield).

Step 3: A solution of tert-butyl 3-(1-[(4-bromophenyl)methyl]cyclopropyl(methyl)carbamoyl)-4H,5H,6H,7H-pyrazolo[1,5-a]pyrazine-5-carboxylate (1.5 g, 3.06 mmol), Pd(dppf)C₂.DCM complex (44.85 mg, 61.3 μmol), and triethylamine (930.38 mg, 9.19 mmol) in MeOH (100 mL) was heated overnight at 120° C. in a steel bomb under CO pressure at 25 bar. After cooling to room temperature, the solution was concentrated and the residue was purified by HPLC to afford tert-butyl 3-[(1-[4-(methoxycarbonyl)phenyl]methylcyclopropyl)(methyl)carbamoyl]-4H,5H,6H,7H-pyrazolo[1,5-a]pyrazine-5-carboxylate (245.0 mg, 522.9 μmol, 17.1% yield).

### Synthesis of 4-[(1-{5-[(tert-butoxy)carbonyl]-4H,5H,6H,7H-pyrazolo[1,5-a]pyrazin-3-yl}-N-methylformamido)methyl]benzoic acid

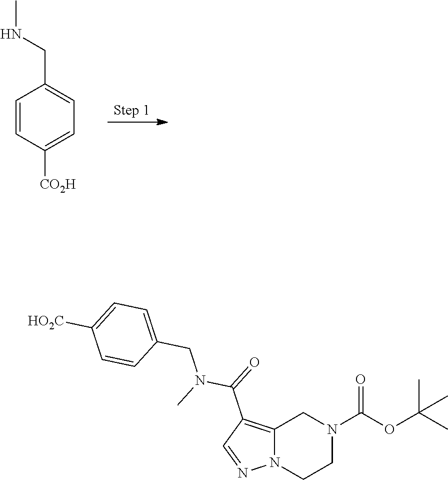 [SMILES: CNCc1ccc(C(=O)O)cc1 ; CN(Cc1ccc(C(=O)O)cc1)C(=O)c1cnn2c1CN(C(=O)OC(C)(C)C)CC2]

Step 1: 5-[(tert-Butoxy)carbonyl]-4H,5H,6H,7H-pyrazolo[1,5-a]pyrazine-3-carboxylic acid (142.52 mg, 533.23 μmol), HATU (202.75 mg, 533.23 μmol) and triethylamine (188.76 mg, 1.87 mmol, 260.0 μL, 3.5 equiv.) were mixed in dry DMF (5 mL) at room temperature. The mixture was stirred for 10 mins, then 4-[(methylamino)methyl]benzoic acid hydrochloride (107.53 mg, 533.23 μmol) was added. The reaction mixture was stirred at room temperature overnight, then concentrated. The residue was purified directly by HPLC to give 4-[(1-5-[(tert-butoxy)carbonyl]-4H,5H,6H,7H-pyrazolo[1,5-a]pyrazin-3-yl-N-methylformamido)methyl]benzoic acid (70.0 mg, 168.9 μmol, 31.7% yield).

### Synthesis of methyl 6-(1-N-methyl-5-[(tert-butoxy)carbonyl]-4H,5H,6H,7H-pyrazolo[1,5-a]pyrazine-3-amidocyclopropyl)pyrimidine-4-carboxylate

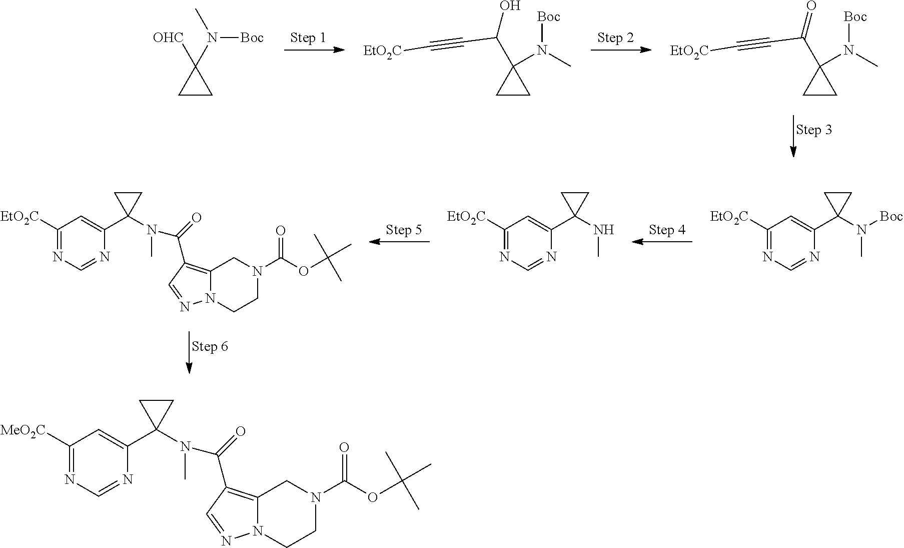

Step 1: To a cooled (−78° C.) solution of ethyl prop-2-ynoate (2.43 g, 24.75 mmol) in dry THF (50 mL) was added N-butyllithium (1.57 g, 24.54 mmol, 10.05 mL, 1.19 equiv.). The resulting solution was stirred for 1 h, then a solution of tert-butyl N-(1-formylcyclopropyl)-N-methylcarbamate (4.11 g, 20.62 mmol) in dry THF (20 mL) was added dropwise over 20 mins.

The reaction mixture was stirred for 3 h at −78° C., then quenched by addition of NH₄Cl solution (sat. aq., 150 mL). The suspension obtained was warmed to room temperature and the layers were separated. The aqueous layer was extracted with ethyl acetate (2×100 mL). The combined organic extracts were washed with brine (100 mL), dried (sodium sulfate), and concentrated to afford crude ethyl 4-(1-[(tert-butoxy)carbonyl](methyl)aminocyclopropyl)-4-hydroxybut-2-ynoate (5.5 g, 18.5 mmol, 89.7% yield) as yellow oil, that was used in the next step without further purification.

Step 2: To a solution of ethyl 4-(1-[(tert-butoxy)carbonyl](methyl)aminocyclopropyl)-4-hydroxybut-2-ynoate (5.5 g, 18.5 mmol) in dry DCM (80 mL) was added 1,1-bis(acetyloxy)-3-oxo-3H-llambda5,2-benziodaoxol-1-yl acetate (7.85 g, 18.5 mmol). The reaction mixture was stirred at room temperature for 2 h. The mixture was cooled to 0° C. and sat. aq. solution of sodium bicarbonate was added dropwise. The mixture was stirred for 1 h and the organic layer was separated, washed with sat. aq. solution of sodium bicarbonate, water, dried over sodium sulfate, filtered, and concentrated to afford crude ethyl 4-(1-[(tert-butoxy)carbonyl](methyl)aminocyclopropyl)-4-oxobut-2-ynoate (4.67 g, 15.81 mmol, 85.5% yield) as a yellow oil, that was used in the next step without further purification.

Step 3: To a solution of ethyl 4-(1-[(tert-butoxy)carbonyl](methyl)aminocyclopropyl)-4-oxobut-2-ynoate (4.67 g, 15.81 mmol) in acetonitrile (50 mL) and water (cat.), were added methanimidamide acetic salt (2.47 g, 23.72 mmol) and sodium carbonate (5.03 g, 47.44 mmol).

The reaction mixture was heated at reflux for 8 h. The mixture was concentrated under reduced pressure, and the residue obtained was dissolved in EtOAc (100 mL). The solution was washed with water (2×30 mL), dried over sodium sulfate, filtered, and concentrated. The residue was purified by column chromatography on silica (EtOAc-hexane 1:5 as eluent) to afford ethyl 6-(1-[(tert-butoxy)carbonyl](methyl)aminocyclopropyl)pyrimidine-4-carboxylate (1.3 g, 4.05 mmol, 25.6% yield) as yellow solid.

Step 4: To a solution of ethyl 6-(1-[(tert-butoxy)carbonyl](methyl)aminocyclopropyl)pyrimidine-4-carboxylate (1.3 g, 4.05 mmol) in dry DCM (10 mL) was added 4M HCl in dioxane (7.15 mL). The reaction mixture was stirred at room temperature for 8 h. The reaction mixture was concentrated under reduced pressure and the residue was dried under vacuum to afford crude ethyl 6-[1-(methylamino)cyclopropyl]pyrimidine-4-carboxylate hydrochloride (1.0 g, 3.88 mmol, 95.9% yield) as brown solid, that was used in the next step without further purification.

Step 5: To a solution of 5-[(tert-butoxy)carbonyl]-4H,5H,6H,7H-pyrazolo[1,5-a]pyrazine-3-carboxylic acid (517.5 mg, 1.94 mmol) in dry DMF (5 mL) was added HATU (736.18 mg, 1.94 mmol). The resulting mixture was stirred for 10 mins, then ethyl 6-[1-(methylamino)cyclopropyl]pyrimidine-4-carboxylate hydrochloride (498.98 mg, 1.94 mmol) and triethylamine (784.08 mg, 7.75 mmol, 1.08 mL, 4.0 equiv.) were added. The mixture was stirred overnight, then partitioned between EtOAc (50 mL) and water (50 mL). The organic phase was washed with water (3×10 mL), brine, dried over sodium sulfate, and concentrated.

The residue was purified by HPLC to afford crude ethyl 6-(1-N-methyl-5-[(tert-butoxy)carbonyl]-4H,5H,6H,7H-pyrazolo[1,5-a]pyrazine-3-amidocyclopropyl)pyrimidine-4-carboxylate (190.0 mg, 92.0% purity, 371.5 μmol, 19.2% yield) as brown oil.

Step 6: To a solution of ethyl 6-(1-N-methyl-5-[(tert-butoxy)carbonyl]-4H,5H,6H,7H-pyrazolo[1,5-a]pyrazine-3-amidocyclopropyl)pyrimidine-4-carboxylate (190.35 mg, 404.55 μmol) in THF/water (1 mL/1 mL) was added lithium hydroxide monohydrate (50.93 mg, 1.21 mmol). The reaction mixture was stirred at room temperature for 5 h. The mixture was concentrated, the residue was dissolved in water (5 mL), and the solution was extracted with MTBE (2×2 mL). The aqueous phase was concentrated to dryness; the residue was dried on vacuum and dissolved in dry DMF (1 mL). The solution was cooled to 0° C. and iodomethane (229.69 mg, 1.62 mmol) was added. The mixture was stirred at r.t. for 10 h and concentrated to dryness. The residue was purified directly by HPLC to afford methyl 6-(1-N-methyl-5-[(tert-butoxy)carbonyl]-4H,5H,6H,7H-pyrazolo[1,5-a]pyrazine-3-amidocyclopropyl)pyrimidine-4-carboxylate (55.9 mg, 122.45 μmol, 31.2% yield) as a pale yellow solid.

### Synthesis of ethyl 2-(1-N-methyl-5-[(tert-butoxy)carbonyl]-4H,5H,6H,7H-pyrazolo[1,5-a]pyrazine-3-amidocyclopropyl)pyrimidine-4-carboxylate

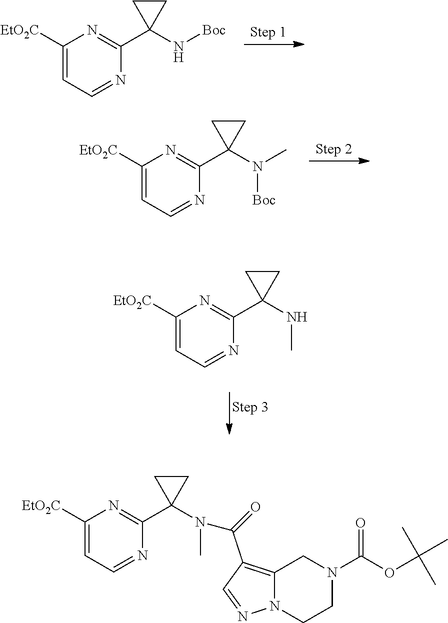 [SMILES: CCOC(=O)c1ccnc(C2(NC(=O)OC(C)(C)C)CC2)n1 ; CCOC(=O)c1ccnc(C2(N(C)C(=O)OC(C)(C)C)CC2)n1 ; CCOC(=O)c1ccnc(C2(NC)CC2)n1 ; CCOC(=O)c1ccnc(C2(N(C)C(=O)c3cnn4c3CN(C(=O)OC(C)(C)C)CC4)CC2)n1]

Step 1: To a suspension of sodium hydride (170.42 mg, 7.1 mmol) in dry DMF (20 mL) was added ethyl 2-(1-[(tert-butoxy)carbonyl]aminocyclopropyl)pyrimidine-4-carboxylate (1.0 g, 3.25 mmol) in one portion. The obtained mixture was stirred until gas evolution ceased (approx. 2 h, at room temperature). The mixture was cooled (10° C.), then iodomethane (831.57 mg, 5.86 mmol, 360.0 μL, 1.8 equiv.) was added dropwise. The resulting mixture was warmed to room temperature and stirred overnight (18 h). The reaction mixture was poured into water (100 mL), and product extracted with EtOAc (2×100 mL). The combined organic extracts were washed with water (20 mL), dried over sodium sulfate, and concentrated to give ethyl 2-(1-[(tert-butoxy)carbonyl](methyl)aminocyclopropyl)pyrimidine-4-carboxylate (800.0 mg, 90.0% purity, 2.24 mmol, 68.8% yield) (mixture of Me and Et—esters) that was used in the next step without further purification.

Step 2: To ethyl 2-(1-[(tert-butoxy)carbonyl](methyl)aminocyclopropyl)pyrimidine-4-carboxylate (800.0 mg, 2.49 mmol) was added 4M HCl in dioxane (30 mL). The resulting mixture was stirred overnight at room temperature then evaporated to dryness to give ethyl 2-[1-(methylamino)cyclopropyl]pyrimidine-4-carboxylate hydrochloride (600.0 mg, 90.0% purity, 2.1 mmol, 84.1% yield) as a solid that was used in the next step without further purification.

Step 3: To a solution of 5-[(tert-butoxy)carbonyl]-4H,5H,6H,7H-pyrazolo[1,5-a]pyrazine-3-carboxylic acid (622.02 mg, 2.33 mmol) and HATU (1.06 g, 2.79 mmol) in DMF (25 mL) was added DIPEA (1.05 g, 8.15 mmol, 3.5 equiv.). The reaction mixture was stirred for 15 mins at room temperature, then ethyl 2-[1-(methylamino)cyclopropyl]pyrimidine-4-carboxylate hydrochloride (600.0 mg, 2.33 mmol) was added. The mixture was stirred overnight, then the mixture was poured into water (100 mL) and extracted with EtOAc (3×100 mL). The combined organic extracts were washed with water (3×30 mL), dried over anhydrous sodium sulfate, and concentrated to yield crude product (800 mg) which was purified by HPLC to give ethyl 2-(1-N-methyl-5-[(tert-butoxy)carbonyl]-4H,5H,6H,7H-pyrazolo[1,5-a]pyrazine-3-amidocyclopropyl)pyrimidine-4-carboxylate (297.0 mg, 97.0% purity, 612.28 μmol, 26.3% yield) as semi-solid.

### Synthesis of methyl 3-[1-(methylamino)cyclopropyl]-1,2-oxazole-5-carboxylate hydrochloride

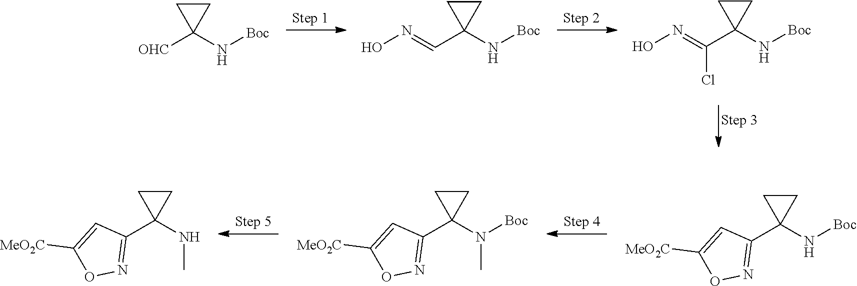 [SMILES: CC(C)(C)OC(=O)NC1(C=O)CC1 ; CC(C)(C)OC(=O)NC1(/C=N/O)CC1 ; CC(C)(C)OC(=O)NC1(/C(Cl)=N/O)CC1 ; CNC1(c2cc(C(=O)OC)on2)CC1 ; COC(=O)c1cc(C2(N(C)C(=O)OC(C)(C)C)CC2)no1 ; COC(=O)c1cc(C2(NC(=O)OC(C)(C)C)CC2)no1]

Step 1: To a stirred solution of tert-butyl N-(1-formylcyclopropyl)carbamate (1.03 g, 5.56 mmol) and hydroxylamine hydrochloride (773.22 mg, 11.13 mmol) in EtOH (10 mL), was added pyridine (880.0 mg, 11.13 mmol, 900.0 μL, 2.0 equiv.). The reaction mixture was stirred at room temperature for 18 h then concentrated in vacuo. The residue was partitioned between water (20 mL) and MTBE (70 mL). The organic layer was washed with 0.1N HCl (10 mL), water (10 mL), brine (10 mL), dried over anhydrous sodium sulfate, and filtered. The filtrate was concentrated to give tert-butyl N-1-[(E)-(hydroxyimino)methyl]cyclopropylcarbamate (800.0 mg, 95.0% purity, 3.8 mmol, 68.2% yield) that was used in the next step without further purification.

Step 2: To a cooled (0° C.), stirred solution of tert-butyl N-1-[(1E)-(hydroxyimino)methyl]cyclopropylcarbamate (800.33 mg, 4.0 mmol) in DMF (8 mL) was added 1-chloropyrrolidine-2,5-dione (560.41 mg, 4.2 mmol). The reaction mixture was stirred for 18 h at room temperature. Then, the obtained solution was used in the next step without an additional work-up.

Step 3: The solution obtained in Step 2 was cooled (0° C.) then copper(II) acetate hydrate (79.14 mg, 396.4 μmol) was added. The reaction mixture was stirred for 5 mins, then methyl prop-2-ynoate (399.92 mg, 4.76 mmol) and sodium hydrogen carbonate (499.5 mg, 5.95 mmol) were added. The mixture was stirred for 24 h at room temperature then concentrated in vacuo. The obtained residue poured into water (50 mL) and extracted with EtOAc (3×50 mL). The combined organic fractions were washed with water (30 mL), dried over anhydrous sodium sulfate, and concentrated to give methyl 3-(1-[(tert-butoxy)carbonyl]aminocyclopropyl)-1,2-oxazole-5-carboxylate (1.0 g, 98.0% purity, 3.47 mmol, 87.6% yield).

Step 4: To a suspension of sodium hydride (185.53 mg, 7.73 mmol) in DMF (8 mL) was added a solution of methyl 3-(1-[(tert-butoxy)carbonyl]aminocyclopropyl)-1,2-oxazole-5-carboxylate (1.0 g, 3.54 mmol) in DMF (2 mL). The obtained mixture was stirred until gas evolution ceased (˜2 h), the solution was cooled (10° C.), then iodomethane (855.03 mg, 6.02 mmol) was added.

The reaction mixture was warmed to room temperature and stirred overnight. The resulting mixture was poured into water (50 mL) and product was extracted with MTBE (2×50 mL).

Organic phases were combined, washed with water (2×30 mL), dried over sodium sulfate, and concentrated. The product was purified by column chromatography (silica, hexane:MTBE 2:1) to give methyl 3-(1-[(tert-butoxy)carbonyl](methyl)aminocyclopropyl)-1,2-oxazole-5-carboxylate (420.0 mg, 96.0% purity, 1.36 mmol, 38.4% yield).

Step 5: To methyl 3-(1-[(tert-butoxy)carbonyl](methyl)aminocyclopropyl)-1,2-oxazole-5-carboxylate (400.0 mg, 1.35 mmol) was added 4M HCl in dioxane (20 mL, 80 mmol). The resulting mixture was stirred overnight, then evaporated to dryness to give methyl 3-[1-(methylamino)cyclopropyl]-1,2-oxazole-5-carboxylate hydrochloride (270.0 mg, 95.0% purity, 1.1 mmol, 81.7% yield) as a solid.

### Synthesis of tert-butyl 3-((1-(4-(methoxycarbonyl)phenyl)cyclopropyl)(methyl)carbamoyl)-6,7-dihydropyrazolo[1,5-a]pyrazine-5(4H)-carboxylate

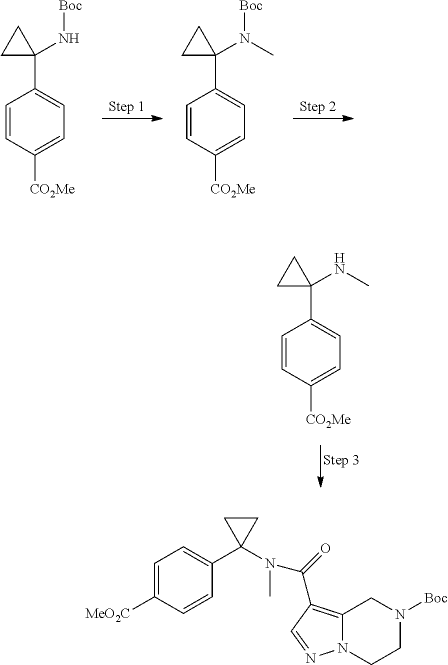 [SMILES: COC(=O)c1ccc(C2(NC(=O)OC(C)(C)C)CC2)cc1 ; COC(=O)c1ccc(C2(N(C)C(=O)OC(C)(C)C)CC2)cc1 ; CNC1(c2ccc(C(=O)OC)cc2)CC1 ; COC(=O)c1ccc(C2(N(C)C(=O)c3cnn4c3CN(C(=O)OC(C)(C)C)CC4)CC2)cc1]

Step 1: To a cooled (0° C.) suspension of sodium hydride (321.2 mg, 13.38 mmol) in dry DMF (15 mL) was added dropwise a solution of 4-(1-[(tert-butoxy)carbonyl]aminocyclopropyl)benzoate (3.0 g, 10.3 mmol) in dry DMF (5 mL). The resulting mixture was stirred until gas evolution ceased, then iodomethane (2.19 g, 15.44 mmol) was added dropwise. The resulting mixture was warmed to room temperature and then stirred overnight. The reaction mixture was poured into saturated aq. ammonium chloride solution and extracted with EtOAc (2×40 mL). The organic phases were combined, dried over sodium sulfate, and concentrated to give methyl 4-(1-[(tert-butoxy)carbonyl](methyl)aminocyclopropyl)benzoate (3.0 g, 9.82 mmol, 95.4% yield).

Step 2: To methyl 4-(1-[(tert-butoxy)carbonyl](methyl)aminocyclopropyl)benzoate (3.0 g, 9.82 mmol) was added 4M HCl in dioxane (50 mL). The reaction mixture was stirred at r.t. for 12 hours then evaporated to dryness to give methyl 4-[1-(methylamino)cyclopropyl]benzoate hydrochloride (1.5 g, 6.21 mmol, 63.2% yield).

Step 3: Methyl 4-[1-(methylamino)cyclopropyl]benzoate hydrochloride (531.8 mg, 2.2 mmol), HATU (920.21 mg, 2.42 mmol) and triethylamine (556.58 mg, 5.5 mmol) were mixed in dry DMF (5 mL). The mixture was stirred for 10 mins, followed by the addition of 5-[(tert-butoxy)carbonyl]-4H,5H,6H,7H-pyrazolo[1,5-a]pyrazine-3-carboxylic acid (588.05 mg, 2.2 mmol). The resulting mixture was stirred at overnight then partitioned between water (50 mL) and EtOAc (50 mL). The organic phase was separated, dried over sodium sulfate and concentrated. The residue was purified by HPLC to give tert-butyl 3-(1-[4-(methoxycarbonyl)phenyl]cyclopropyl(methyl)carbamoyl)-4H,5H,6H,7H-pyrazolo[1,5-a]pyrazine-5-carboxylate (158.5 mg, 348.72 μmol, 15.9% yield) as white solid.

### Synthesis of tert-butyl 3-({1-[3-(methoxycarbonyl)phenyl]cyclopropyl}(methyl)carbamoyl)-4H,5H,6H,7H-pyrazolo[1,5-a]pyrazine-5-carboxylate

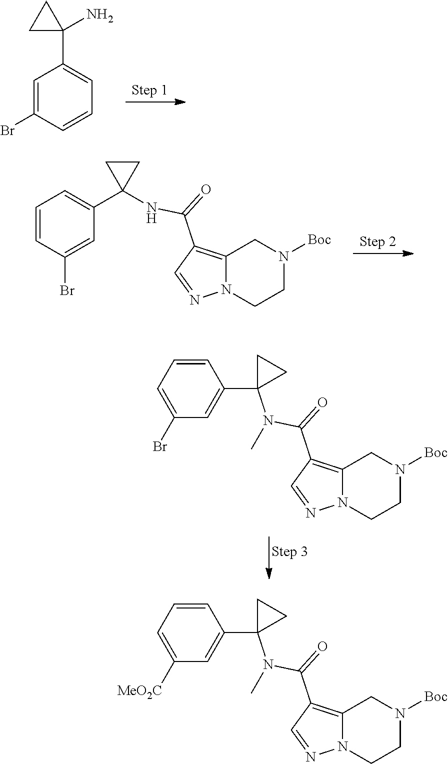 [SMILES: NC1(c2cccc(Br)c2)CC1 ; CC(C)(C)OC(=O)N1CCn2ncc(C(=O)NC3(c4cccc(Br)c4)CC3)c2C1 ; CN(C(=O)c1cnn2c1CN(C(=O)OC(C)(C)C)CC2)C1(c2cccc(Br)c2)CC1 ; COC(=O)c1cccc(C2(N(C)C(=O)c3cnn4c3CN(C(=O)OC(C)(C)C)CC4)CC2)c1]

Step 1: To a solution of 5-[(tert-butoxy)carbonyl]-4H,5H,6H,7H-pyrazolo[1,5-a]pyrazine-3-carboxylic acid (1.61 g, 6.03 mmol) in dry DMF (15 mL) was added HATU (2.29 g, 6.03 mmol). The resulting mixture was stirred for 10 mins, followed by addition of 1-(3-bromophenyl)cyclopropan-1-amine hydrochloride (1.5 g, 6.03 mmol) and triethylamine (2.44 g, 24.11 mmol, 3.36 mL, 4.0 equiv.). The reaction mixture was stirred at room temperature overnight, then partitioned between EtOAc (100 mL) and water (50 mL). The organic fraction was washed with water (3×50 mL), brine, dried over sodium sulfate, and concentrated to afford tert-butyl 3-[1-(3-bromophenyl)cyclopropyl]carbamoyl-4H,5H,6H,7H-pyrazolo[1,5-a]pyrazine-5-carboxylate (2.3 g, 4.99 mmol, 82.7% yield) as beige solid.

Step 2: To a cooled (0° C.) solution of tert-butyl 3-[1-(3-bromophenyl)cyclopropyl]carbamoyl-4H,5H,6H,7H-pyrazolo[1,5-a]pyrazine-5-carboxylate (2.3 g, 4.98 mmol) in dry DMF (20 mL) was added sodium hydride (298.72 mg, 12.45 mmol). The mixture was stirred for 30 mins, then iodomethane (1.41 g, 9.96 mmol, 620.0 μL, 2.0 equiv.) was added dropwise. The reaction mixture was stirred at r.t. overnight. The mixture was diluted with brine (50 mL) and extracted with EtOAc (3×50 mL). The combined organic extracts were washed with brine, dried over sodium sulfate, filtered, and concentrated to give tert-butyl 3-[1-(3-bromophenyl)cyclopropyl](methyl)carbamoyl-4H,5H,6H,7H-pyrazolo[1,5-a]pyrazine-5-carboxylate (2.3 g, 4.84 mmol, 97.2% yield) as a beige foam.

Step 3: To a solution of tert-butyl 3-[1-(3-bromophenyl)cyclopropyl](methyl)carbamoyl-4H,5H,6H,7H-pyrazolo[1,5-a]pyrazine-5-carboxylate (2.3 g, 4.84 mmol) in MeOH (100 mL) was added Pd(dppf)C₂.DCM complex (395.1 mg, 483.81 μmol) and triethylamine (587.48 mg, 5.81 mmol). The mixture was carbonylated at 125° C. and 40 atm for 20 h. The resulting mixture was cooled and concentrated to dryness. The residue was dissolved in EtOAc (100 mL) and the solution was washed with water (20 mL), dried over sodium sulfate, filtered, and concentrated.

The residue was re-dissolved in chloroform (50 mL) and di-tert-butyl dicarbonate (316.77 mg, 1.45 mmol) was added. The reaction mixture was stirred at r.t. for 5 h and concentrated. The residue was purified by column chromatography (silica, EtOAc-hexane 1:1 to EtOAc) to afford tert-butyl 3-(1-[3-(methoxycarbonyl)phenyl]cyclopropyl(methyl)carbamoyl)-4H,5H,6H,7H-pyrazolo[1,5-a]pyrazine-5-carboxylate (1.0 g, 2.2 mmol, 45.5% yield) as yellow solid.

### Synthesis of tert-butyl 1-({1-[4-(methoxycarbonyl)phenyl]cyclopropyl}(methyl)carbamoyl)-5H,6H,7H,8H-imidazo[1,5-a]pyrazine-7-carboxylate

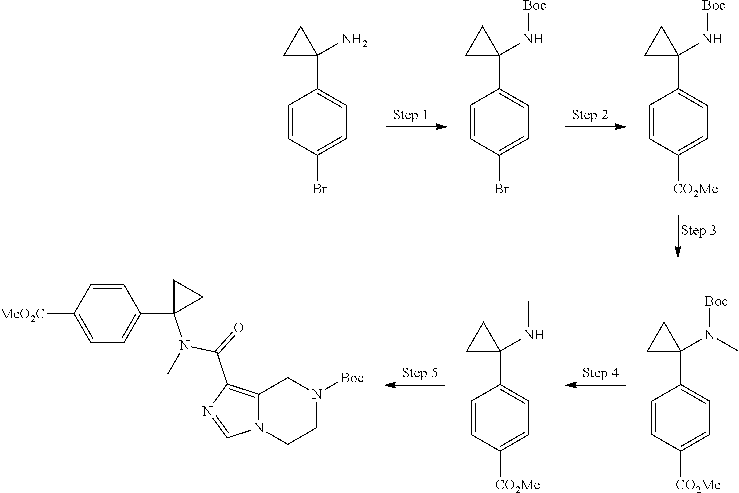 [SMILES: NC1(c2ccc(Br)cc2)CC1 ; CC(C)(C)OC(=O)NC1(c2ccc(Br)cc2)CC1 ; COC(=O)c1ccc(C2(NC(=O)OC(C)(C)C)CC2)cc1 ; COC(=O)c1ccc(C2(N(C)C(=O)c3ncn4c3CN(C(=O)OC(C)(C)C)CC4)CC2)cc1 ; CNC1(c2ccc(C(=O)OC)cc2)CC1 ; COC(=O)c1ccc(C2(N(C)C(=O)OC(C)(C)C)CC2)cc1]

Step 1: Triethylamine (4.48 g, 44.27 mmol, 6.17 mL, 1.1 equiv.) was added portionwise to a mixture of 1-(4-bromophenyl)cyclopropan-1-amine hydrochloride (10.0 g, 40.24 mmol) and di-tert-butyl dicarbonate (9.66 g, 44.27 mmol, 10.18 mL, 1.1 equiv.) in DCM (100 mL). The resulting mixture was stirred overnight at room temperature, then washed with water (70 mL), dried over sodium sulfate, and concentrated in vacuo to give tert-butyl N-[1-(4-bromophenyl)cyclopropyl]carbamate (10.5 g, 33.63 mmol, 83.6% yield).

Step 2: 1-(N-boc-amino)-1-(4-bromophenyl)cyclopropane (10.5 g, 33.63 mmol) was carbonylated in MeOH (100 mL) at 130° C. and 50 atm CO pressure with Pd(dppf)C2.DCM complex as catalyst. After consumption of the starting material, the resulting mixture was concentrated and the residue was partitioned between water (100 mL) and EtOAc (200 mL).

The organic layer was collected, dried over sodium sulfate and concentrated to give methyl 4-(1-[(tert-butoxy)carbonyl]aminocyclopropyl)benzoate (9.5 g, 32.61 mmol, 97% yield) which was used in the next step without further purification.

Step 3: To a cooled (0° C.) suspension of sodium hydride (616.74 mg, 25.7 mmol) in dry DMF (20 mL) was added dropwise a solution of methyl 4-(1-[(tert-butoxy)carbonyl]aminocyclopropyl)benzoate (4.99 g, 17.13 mmol) in dry DMF (20 mL). The resulting mixture was stirred until gas evolution ceased, then iodomethane (3.65 g, 25.7 mmol, 1.6 mL, 1.5 equiv.) was added dropwise. The resulting mixture was warmed to r.t. and stirred overnight. The reaction mixture was poured into saturated aq. NH₄Cl solution. The resulting mixture was extracted with EtOAc (2×50 mL) The organic phases were combined, dried over sodium sulfate and concentrated to give methyl 4-(1-[(tert-butoxy)carbonyl](methyl)aminocyclopropyl)benzoate (3.0 g, 9.82 mmol, 57.3% yield).

Step 4: To methyl 4-(1-[(tert-butoxy)carbonyl](methyl)aminocyclopropyl)benzoate (3.0 g, 9.82 mmol) was added 4M HCl in dioxane (20 mL). The resulting mixture was stirred overnight, then evaporated to dryness. The residue was triturated with MTBE, filtered and dried to give methyl 4-[1-(methylamino)cyclopropyl]benzoate hydrochloride (1.1 g, 4.55 mmol, 46.3% yield) as solid residue.

Step 5: Methyl 4-[1-(methylamino)cyclopropyl]benzoate hydrochloride (200.0 mg, 827.42 μmol), HATU (346.0 mg, 909.97 μmol) and triethylamine (209.27 mg, 2.07 mmol, 290.0 μL, 2.5 equiv.) were mixed in dry DMF (5 mL) at room temperature. The resulting mixture was stirred for 10 minutes followed by the addition of 7-[(tert-butoxy)carbonyl]-5H,6H,7H,8H-imidazo[1,5-a]pyrazine-1-carboxylic acid (221.11 mg, 827.25 μmol). The reaction mixture was stirred at room temperature overnight, then partitioned between water (50 mL) and EtOAc (50 mL). The organic phase was separated, dried over sodium sulfate, and concentrated. The residue was purified by HPLC to give tert-butyl 1-(1-[4-(methoxycarbonyl)phenyl]cyclopropyl(methyl)carbamoyl)-5H,6H,7H,8H-imidazo [1,5-a]pyrazine-7-carboxylate (45.5 mg, 100.11 μmol, 12.1% yield) as white solid.

### Synthesis of tert-butyl 1-({1-[3-(methoxycarbonyl)phenyl]cyclopropyl}(methyl)carbamoyl)-5H,6H,7H,8H-imidazo[1,5-a]pyrazine-7-carboxylate

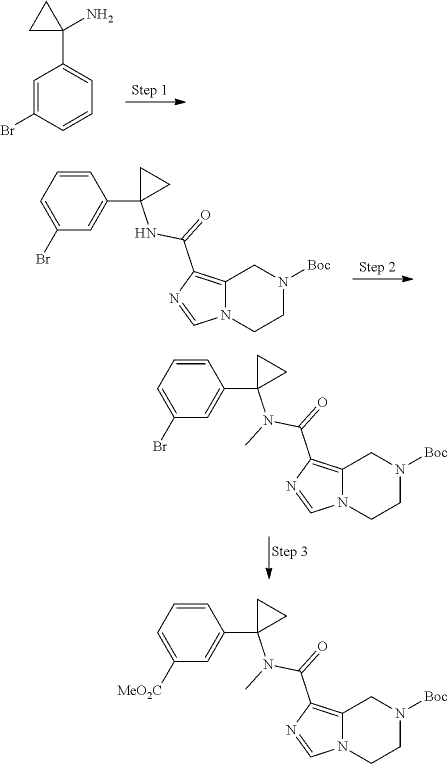

Step 1: To a solution of 7-[(tert-butoxy)carbonyl]-5H,6H,7H,8H-imidazo[1,5-a]pyrazine-1-carboxylic acid (630.0 mg, 2.36 mmol) in dry DMF (5 mL) was added HATU (895.87 mg, 2.36 mmol). The resulting mixture was stirred for 30 mins followed by the addition of 1-(3-bromophenyl)cyclopropan-1-amine hydrochloride (585.61 mg, 2.36 mmol) and triethylamine (953.66 mg, 9.42 mmol, 1.31 mL, 4.0 equiv.). The reaction mixture was stirred at room temperature overnight then partitioned between EtOAc (50 mL) and water (30 mL). The organic phase was washed with water (2×20 mL), brine, dried over sodium sulfate, and concentrated under reduced pressure to give crude tert-butyl 1-[1-(3-bromophenyl)cyclopropyl]carbamoyl-5H,6H,7H,8H-imidazo[1,5-a]pyrazine-7-carboxylate (1.0 g, 85.0% purity, 1.84 mmol, 78.2% yield) as yellow solid, that was used in the next step without further purification.

Step 2: To a cooled (0° C.) solution of tert-butyl 1-[1-(3-bromophenyl)cyclopropyl]carbamoyl-5H,6H,7H,8H-imidazo[1,5-a]pyrazine-7-carboxylate (1.0 g, 2.17 mmol) in dry DMF (10 mL) was added sodium hydride (130.12 mg, 5.42 mmol). The mixture was stirred for 30 mins, then iodomethane (615.6 mg, 4.34 mmol, 270.0 μL, 2.0 equiv.) was added dropwise. The reaction mixture was stirred at r.t. overnight then diluted with brine (50 mL) and extracted with EtOAc (3×30 mL). The combined organic extracts were washed with brine, dried over sodium sulfate, filtered, and concentrated to give tert-butyl 1-[1-(3-bromophenyl)cyclopropyl](methyl)carbamoyl-5H,6H,7H,8H-imidazo[1,5-a]pyrazine-7-carboxylate (1.0 g, 2.1 mmol, 97% yield).

Step 3: To a solution of tert-butyl 1-[1-(3-bromophenyl)cyclopropyl](methyl)carbamoyl-5H,6H,7H,8H-imidazo[1,5-a]pyrazine-7-carboxylate (999.87 mg, 2.1 mmol) in MeOH (50 mL) were added Pd(dppf)C₂.DCM complex (171.77 mg, 210.33 μmol) and triethylamine (255.4 mg, 2.52 mmol). The mixture was carbonylated at 120° C. and 40 atm for 40 h. The mixture was cooled to room temperature and concentrated to dryness. The residue was re-dissolved in EtOAc (50 mL) and washed with water (25 mL), dried over sodium sulfate, filtered, and concentrated. The residue was purified by HPLC to give tert-butyl 1-(1-[3-(methoxycarbonyl)phenyl]cyclopropyl(methyl)carbamoyl)-5H,6H,7H,8H-imidazo [1,5-a]pyrazine-7-carboxylate (115.3 mg, 253.67 μmol, 12.1% yield) as brown solid.

### Synthesis of tert-butyl 3-({1-[4-(methoxycarbonyl)phenyl]cyclopropyl}(methyl)carbamoyl)-6-methyl-4H,5H,6H,7H-pyrazolo[1,5-a]pyrazine-5-carboxylate

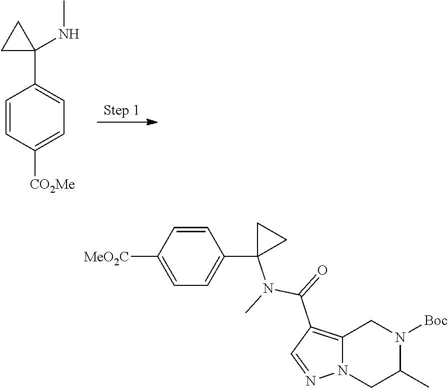 [SMILES: CNC1(c2ccc(C(=O)OC)cc2)CC1 ; COC(=O)c1ccc(C2(N(C)C(=O)c3cnn4c3CN(C(=O)OC(C)(C)C)C(C)C4)CC2)cc1]

Step 1: Methyl 4-[1-(methylamino)cyclopropyl]benzoate hydrochloride (200.0 mg, 827.42 μmol), HATU (346.35 mg, 910.91 μmol) and triethylamine (209.49 mg, 2.07 mmol, 290.0 μL, 2.5 equiv.) were mixed in dry DMF (5 mL) at room temperature. The resulting mixture was stirred for 10 mins then 5-[(tert-butoxy)carbonyl]-6-methyl-4H,5H,6H,7H-pyrazolo[1,5-a]pyrazine-3-carboxylic acid (232.95 mg, 828.1 μmol) was added. The resulting mixture was stirred at room temperature overnight then partitioned between water (50 mL) and EtOAc (50 mL). The organic phase was separated, dried over sodium sulfate, and concentrated. The residue was purified by HPLC to give tert-butyl 3-(1-[4-(methoxycarbonyl)phenyl]cyclopropyl(methyl)carbamoyl)-6-methyl-4H,5H,6H,7H-pyrazolo[1,5-a]pyrazine-5-carboxylate (206.5 mg, 440.73 μmol, 53.2% yield) as white solid.

### Synthesis of tert-butyl 3-({1-[3-(methoxycarbonyl)phenyl]cyclopropyl}(methyl)carbamoyl)-6-methyl-4H,5H,6H,7H-pyrazolo[1,5-a]pyrazine-5-carboxylate

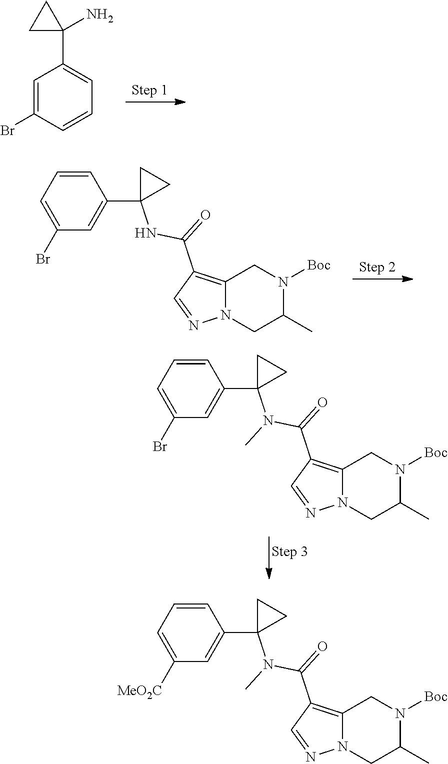

Step 1: To a solution of 5-[(tert-butoxy)carbonyl]-6-methyl-4H,5H,6H,7H-pyrazolo[1,5-a]pyrazine-3-carboxylic acid (690.0 mg, 2.45 mmol) in dry DMF (5 mL) was added HATU (932.62 mg, 2.45 mmol). The resulting mixture was stirred for 10 mins then 1-(3-bromophenyl)cyclopropan-1-amine hydrochloride (609.63 mg, 2.45 mmol) and triethylamine (992.79 mg, 9.81 mmol) were added. The resulting mixture was stirred at room temperature overnight then partitioned between EtOAc (50 mL) and water (30 mL). The organic phase was washed with water (2×20 mL), brine, then dried over sodium sulfate, and concentrated under reduced pressure to give tert-butyl 3-[1-(3-bromophenyl)cyclopropyl]carbamoyl-6-methyl-4H,5H,6H,7H-pyrazolo[1,5-a]pyrazine-5-carboxylate (1.15 g, 2.42 mmol, 98.6% yield) as brown solid.

Step 2: To a cooled (0° C.) solution of tert-butyl 3-[1-(3-bromophenyl)cyclopropyl]carbamoyl-6-methyl-4H,5H,6H,7H-pyrazolo[1,5-a]pyrazine-5-carboxylate (1.15 g, 2.42 mmol) in dry DMF (10 mL), was added sodium hydride (145.14 mg, 6.05 mmol). The mixture was stirred for 30 mins, then iodomethane (686.78 mg, 4.84 mmol) was added dropwise. The reaction mixture was stirred at r.t. overnight. The mixture was diluted with brine (50 mL) and extracted with EtOAc (3×30 mL). The combined organic extracts were washed with brine, dried over sodium sulfate, filtered, and concentrated to afford tert-butyl 3-[1-(3-bromophenyl)cyclopropyl](methyl)carbamoyl-6-methyl-4H,5H,6H,7H-pyrazolo [1,5-a]pyrazine-5-carboxylate (1.0 g, 2.04 mmol, 84.5% yield) as brown solid.

Step 3: To a solution of tert-butyl 3-[1-(3-bromophenyl)cyclopropyl](methyl)carbamoyl-6-methyl-4H,5H,6H,7H-pyrazolo[1,5-a]pyrazine-5-carboxylate (994.38 mg, 2.03 mmol) in MeOH (60 mL) were added Pd(dppf)Cl₂.DCM complex (165.93 mg, 203.18 μmol) and triethylamine (246.84 mg, 2.44 mmol, 340.0 μL, 1.2 equiv.) were added. The resulting mixture was carbonylated at 125° C. and 40 atm for 36 h. The mixture was cooled to room temperature and concentrated to dryness. The residue was dissolved in EtOAc (50 mL). The solution was washed with water (20 mL), dried over sodium sulfate, filtered, and concentrated. The residue was purified by HPLC to afford tert-butyl 3-(1-[3-(methoxycarbonyl)phenyl]cyclopropyl(methyl)carbamoyl)-6-methyl-4H,5H,6H,7H-pyrazolo[1,5-a]pyrazine-5-carboxylate (413.7 mg, 882.95 μmol, 43.5% yield) as brown solid.

### Synthesis of methyl 4-[1-(methylamino)cyclopropyl]benzoate hydrochloride

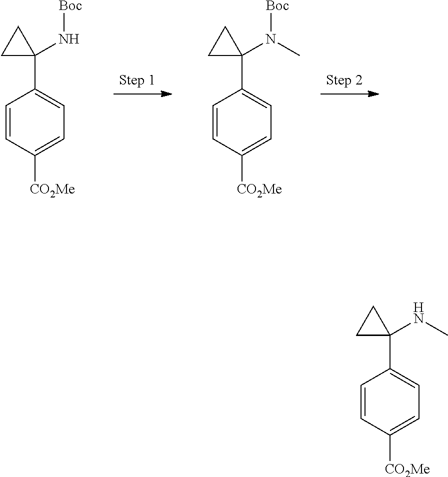 [SMILES: COC(=O)c1ccc(C2(NC(=O)OC(C)(C)C)CC2)cc1 ; COC(=O)c1ccc(C2(N(C)C(=O)OC(C)(C)C)CC2)cc1 ; CNC1(c2ccc(C(=O)OC)cc2)CC1]

Step 1: To a cooled (0° C.) suspension of sodium hydride (98.83 mg, 4.12 mmol) in dry DMF (10 mL) was added dropwise a solution of methyl 4-(1-[(tert-butoxy)carbonyl]aminocyclopropyl)benzoate (1.0 g, 3.43 mmol) in dry DMF (5 mL). The resulting mixture was stirred until gas evolution ceased (approx. 20 mins). Iodomethane (730.68 mg, 5.15 mmol) was added dropwise, and the resulting mixture warmed to r.t. and stirred overnight. The mixture was poured into saturated aq. NH₄Cl solution, and extracted with EtOAc (2×50 mL) The combined organic extracts were dried over sodium sulfate and concentrated to give methyl 4-(1-[(tert-butoxy)carbonyl](methyl)aminocyclopropyl)benzoate (900.0 mg, 2.95 mmol, 85.9% yield).

Step 2: To methyl 4-(1-[(tert-butoxy)carbonyl](methyl)aminocyclopropyl)benzoate (900.0 mg, 2.95 mmol) was added 4M HCl in dioxane (20 mL, 80 mmol). The reaction mixture was stirred overnight then evaporated to dryness. The residue was triturated with MTBE, filtered, and air-dried to give methyl 4-[1-(methylamino)cyclopropyl]benzoate hydrochloride (500.0 mg, 2.07 mmol, 70.2% yield) as solid.

### Synthesis of methyl 3-[1-(methylamino)cyclopropyl]benzoate hydrochloride

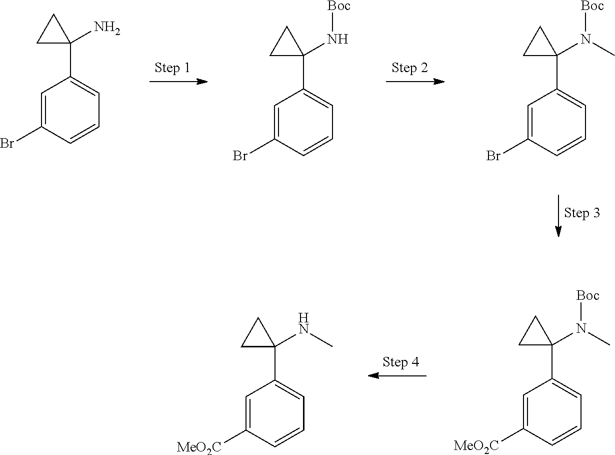 [SMILES: NC1(c2cccc(Br)c2)CC1 ; CC(C)(C)OC(=O)NC1(c2cccc(Br)c2)CC1 ; CN(C(=O)OC(C)(C)C)C1(c2cccc(Br)c2)CC1 ; CNC1(c2cccc(C(=O)OC)c2)CC1 ; COC(=O)c1cccc(C2(N(C)C(=O)OC(C)(C)C)CC2)c1]

Step 1: To a cooled (0° C.) solution of 1-(3-bromophenyl)cyclopropan-1-amine hydrochloride (4.4 g, 17.7 mmol) in DCM (50 mL) was added di-tert-butyl dicarbonate (3.86 g, 17.7 mmol) Triethylamine (2.15 g, 21.24 mmol) was added dropwise, the reaction mixture was warmed to room temperature, then stirred for 5 h. The mixture was diluted with water (25 mL). The organic phase was separated, dried over sodium sulfate, filtered, and concentrated to afford tert-butyl N-[1-(3-bromophenyl)cyclopropyl]carbamate (4.8 g, 15.37 mmol, 86.8% yield) as white solid.

Step 2: To a cooled (0° C.) solution of tert-butyl N-[1-(3-bromophenyl)cyclopropyl]carbamate (4.8 g, 15.38 mmol) in dry DMF (30 mL) under an atmosphere of argon was added sodium hydride (922.45 mg, 38.44 mmol) portionwise. The mixture was stirred for 30 mins followed by the dropwise addition of iodomethane (4.36 g, 30.75 mmol). The reaction mixture was stirred at r.t. overnight. The mixture was diluted with brine (50 mL) and extracted with EtOAc (3×30 mL). The combined organic extracts were washed with brine, dried over sodium sulfate, filtered, and concentrated to afford tert-butyl N-[1-(3-bromophenyl)cyclopropyl]-N-methylcarbamate (4.3 g, 13.18 mmol, 85.7% yield).

Step 3: To a solution of tert-butyl N-[1-(3-bromophenyl)cyclopropyl]-N-methylcarbamate (4.3 g, 13.18 mmol) in MeOH (150 mL) were added Pd(dppf)C₂.DCM complex (1.08 g, 1.32 mmol) and triethylamine (1.6 g, 15.82 mmol). The mixture was carbonylated at 135° C. and 40 atm for 28 h. The resulting mixture was cooled and evaporated to dryness. The residue was dissolved in EtOAc (50 mL). The solution was washed with water (25 mL), dried over sodium sulfate, filtered, and concentrated. The residue was purified by flash column chromatography on silica (hexane-EtOAc 4:1) to give methyl 3-(1-[(tert-butoxy)carbonyl](methyl)aminocyclopropyl)benzoate (3.24 g, 90.0% purity, 9.55 mmol, 72.4% yield) as yellow oil.

Step 4: To a solution of methyl 3-(1-[(tert-butoxy)carbonyl](methyl)aminocyclopropyl)benzoate (3.24 g, 10.61 mmol) in dry DCM (20 mL) was added 4M HCl in dioxane (18.7 mL). The mixture was stirred for 10 h at room temperature then concentrated under reduced pressure. The residue was triturated with dry EtOAc. The solid was collected by filtration and air-dried to afford methyl 3-[1-(methylamino)cyclopropyl]benzoate hydrochloride (2.1 g, 8.69 mmol, 81.9% yield) as pink solid.

### Synthesis of tert-butyl 3-({1-[4-(methoxycarbonyl)phenyl]cyclopropyl}(methyl)carbamoyl)-1-{[2-(trimethylsilyl)ethoxy]methyl}-1H,4H,5H,6H,7H-pyrazolo[4,3-c]pyridine-5-carboxylate

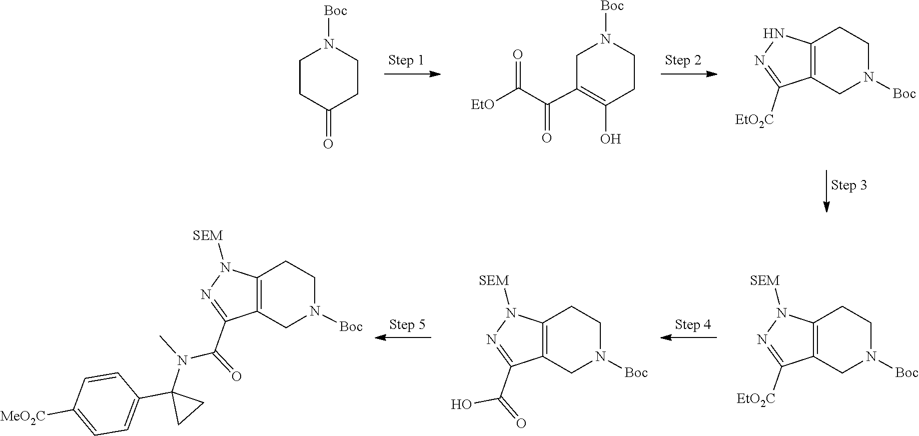 [SMILES: CC(C)(C)OC(=O)N1CCC(=O)CC1 ; CCOC(=O)C(=O)C1=C(O)CCN(C(=O)OC(C)(C)C)C1 ; CCOC(=O)c1n[nH]c2c1CN(C(=O)OC(C)(C)C)CC2 ; COC(=O)c1ccc(C2(N(C)C(=O)c3nn(COCC[Si](C)(C)C)c4c3CN(C(=O)OC(C)(C)C)CC4)CC2)cc1 ; CC(C)(C)OC(=O)N1CCc2c(c(C(=O)O)nn2COCC[Si](C)(C)C)C1 ; CCOC(=O)c1nn(COCC[Si](C)(C)C)c2c1CN(C(=O)OC(C)(C)C)CC2]

Step 1: Lithium bis(trimethylsilyl)azanide (27.72 g, 165.66 mmol, 165.66 mL, 1.1 equiv.) was dissolved in dry diethyl ether (150 mL) and cooled to −78° C. (dry-ice/acetone). To the cooled mixture under argon atmosphere was added a solution of tert-butyl 4-oxopiperidine-1-carboxylate (30.01 g, 150.6 mmol) in dry diethyl ether/dry THF (3:1) (200 mL) (over 15 min). The mixture was stirred for 30 mins, then a solution of diethyl oxalate (24.21 g, 165.66 mmol, 22.5 mL, 1.1 equiv.) in dry diethyl ether (50 mL) was added. The resulting mixture was stirred for 30 mins at −78° C. after which the cooling was removed. When the mixture reached 0° C., a yellow suspension formed. The mixture was poured into 1M KHSO₄ (200 mL) and the layers were separated. The aqueous phase was extracted with EtOAc (2×100 mL). The combined organic extracts were washed with water, dried (sodium sulfate), filtered, and concentrated to give crude tert-butyl 5-(2-ethoxy-2-oxoacetyl)-4-hydroxy-1,2,3,6-tetrahydropyridine-1-carboxylate (49.0 g, 90.0% purity, 147.33 mmol, 97.8% yield) as orange oil, which was used in the next step without further purification.

Step 2: To a stirred solution of tert-butyl 3-(2-ethoxy-2-oxoacetyl)-4-oxopiperidine-1-carboxylate (49.02 g, 163.76 mmol) in absolute EtOH (250 mL) were added acetic acid (14.16 g, 235.81 mmol, 13.62 mL, 1.6 equiv.) and hydrazine hydrate (7.38 g, 147.38 mmol, 12.3 mL, 1.0 equiv.). The mixture was stirred for 5 h then the mixture was concentrated. The residue was diluted with saturated aqueous solution of NaHCO₃ and the product was extracted with EtOAc (3×100 mL). The combined organic phase was dried (sodium sulfate), filtered, and concentrated. The residue was triturated with hexane, and the obtained solid was collected by filtration to afford 5-tert-butyl 3-ethyl 1H,4H,5H,6H,7H-pyrazolo[4,3-c]pyridine-3,5-dicarboxylate (41.6 g, 140.86 mmol, 95.6% yield) as light yellow solid.

Step 3: To a cooled (0° C.) suspension of sodium hydride (1.02 g, 42.38 mmol) in dry THF (50 mL) under an argon atmosphere was added dropwise a solution of 5-tert-butyl 3-ethyl 1H,4H,5H,6H,7H-pyrazolo[4,3-c]pyridine-3,5-dicarboxylate (5.01 g, 16.95 mmol) in dry THF (20 mL). The resulting mixture was stirred for 30 mins then [2-(chloromethoxy)ethyl]trimethylsilane (3.67 g, 22.04 mmol, 3.9 mL, 1.3 equiv.) was added dropwise. The reaction mixture was stirred for 30 mins then warmed to room temperature. The resulting mixture was poured in water (100 mL), the product was extracted with EtOAc (3×50 mL). The combined organic extracts were washed with brine, dried over sodium sulfate, and concentrated to afford 5-tert-butyl 3-ethyl 1-[2-(trimethylsilyl)ethoxy]methyl-1H,4H,5H,6H,7H-pyrazolo[4,3-c]pyridine-3,5-dicarboxylate (6.7 g, 15.74 mmol, 92.9% yield) as colorless solid.

Step 4: To a stirred solution of 5-tert-butyl 3-ethyl 1-[2-(trimethylsilyl)ethoxy]methyl-1H,4H,5H,6H,7H-pyrazolo[4,3-c]pyridine-3,5-dicarboxylate (6.7 g, 15.74 mmol) in THF (50 mL) and water (25 mL) was added lithium hydroxide monohydrate (2.31 g, 55.1 mmol). The reaction mixture was stirred at 50° C. for 3 h then concentrated under reduced pressure; the residue was carefully acidified with sat. aq. solution of KHSO₄ to pH 4-5. The product was extracted with EtOAc (2×50 mL). The organic phase was separated, dried with sodium sulfate, filtered, and concentrated. The residue was triturated with hexane, the product was collected by filtration and dried to afford 5-[(tert-butoxy)carbonyl]-1-[2-(trimethylsilyl)ethoxy]methyl-1H,4H,5H,6H,7H-pyrazolo[4,3-c]pyridine-3-carboxylic acid (4.6 g, 11.57 mmol, 73.5% yield) as light yellow solid.

Step 5: To a solution of 5-[(tert-butoxy)carbonyl]-1-[2-(trimethylsilyl)ethoxy]methyl-1H,4H,5H,6H,7H-pyrazolo[4,3-c]pyridine-3-carboxylic acid (600.0 mg, 1.51 mmol) in dry DMF (5 mL) was added HATU (574.14 mg, 1.51 mmol). The resulting mixture was stirred for 30 mins, followed by addition of methyl 4-[1-(methylamino)cyclopropyl]benzoate hydrochloride (364.98 mg, 1.51 mmol) and triethylamine (611.18 mg, 6.04 mmol, 840.0 μL, 4.0 equiv.). The resulting mixture was stirred overnight, then partitioned between EtOAc (50 mL) and water (30 mL). The organic phase was washed with water (2×20 mL), brine, dried over sodium sulfate, and concentrated under reduced pressure. The residue was purified by HPLC to afford tert-butyl 3-(1-[4-(methoxycarbonyl)phenyl]cyclopropyl(methyl)carbamoyl)-1-[2-(trimethylsilyl)ethoxy]methyl-1H,4H,5H,6H,7H-pyrazolo[4,3-c]pyridine-5-carboxylate (470.0 mg, 803.72 μmol, 53.2% yield) as brown solid.

### Synthesis of tert-butyl 3-({1-[4-(methoxycarbonyl)phenyl]cyclopropyl}(methyl)carbamoyl)-6-methyl-1-{[2-(trimethylsilyl)ethoxy]methyl}-1H,4H,5H,6H,7H-pyrazolo[4,3-c]pyridine-5-carboxylate

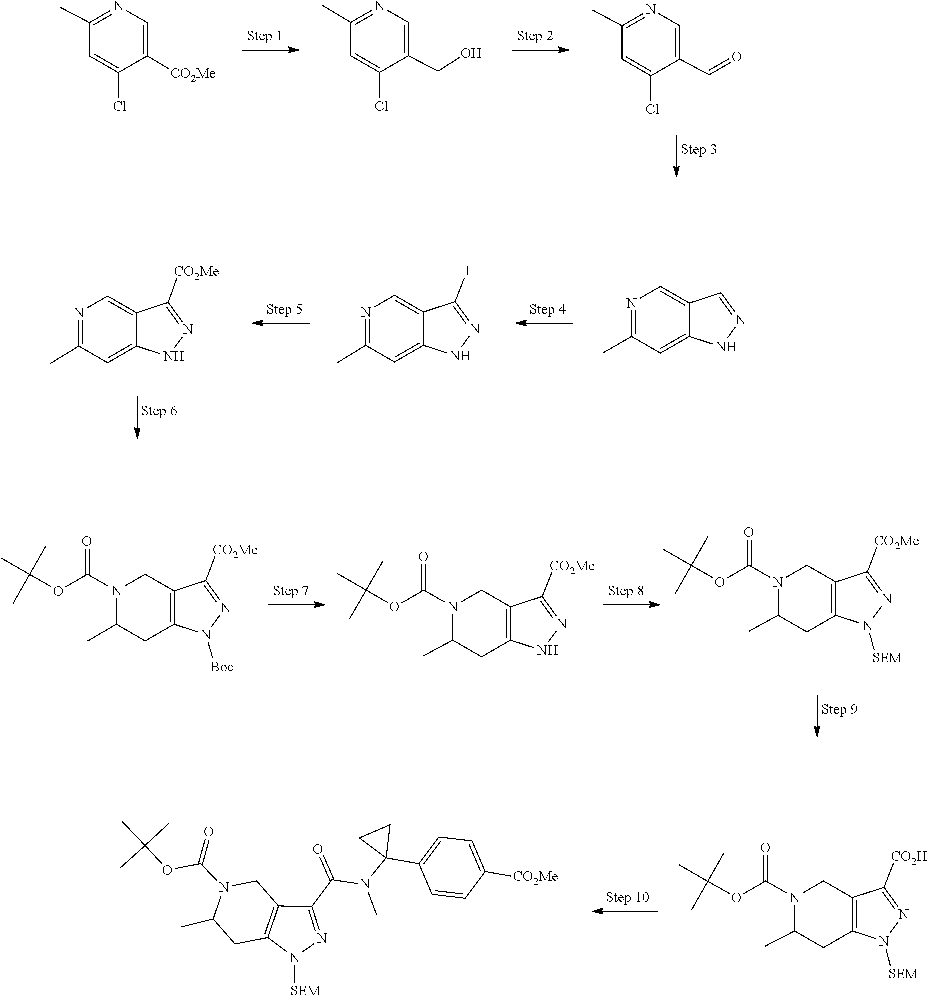

Step 1: To a suspension of lithium aluminum hydride (5.7 g) in THF (500 mL) at −25° C., a solution of methyl 4-chloro-6-methylnicotinate (30.0 g, 161.63 mmol) in tetrahydrofuran (100 mL) was added dropwise. The resulting mixture was stirred at 0° C. for 1.5 hours. Then, water (6 mL in 50 ml of THF), NaOH (6 mL, 15% aqueous solution) and water (18 mL) were dropped successively to the reaction mixture (0-5° C.). The obtained mixture was stirred for 30 minutes at room temperature then filtered. The filtercake was washed by THF (2×200 mL). The filtrate was concentrated to give (4-chloro-6-methylpyridin-3-yl)methanol (20.0 g, 93.0% purity, 118.02 mmol, 73% yield) as an yellow solid. The crude (93% purity) product was used without purification.

Step 2: To a solution of (4-chloro-6-methylpyridin-3-yl)methanol (42.5 g, 269.67 mmol) in CH₂Cl₂ (1300 mL) was added 1,1-bis(acetyloxy)-3-oxo-3H-llambda5,2-benziodaoxol-1-yl acetate (131.54 g, 310.12 mmol) in few portions (over ˜10 mins), maintaining temperature below 5° C. with water/ice bath cooling. After reaction was complete the mixture was poured into a saturated aqueous solution of sodium hydrogen carbonate (113.27 g, 1.35 mol), Na₂S₂O₃.5H₂O (100.39 g, 0.404 mol) and stirred until organic phase became transparent (about 18 h, at 10-20° C.). The layers were separated and the aqueous layer extracted with DCM (300 mL). The combined organic extracts were washed with brine (200 mL), dried over sodium sulfate, and concentrated under reduced pressure to give 4-chloro-6-methylpyridine-3-carbaldehyde (37.0 g, 98.0% purity, 233.06 mmol, 86.4% yield) as yellow solid.

Step 3: To a suspension of 4-chloro-6-methylpyridine-3-carbaldehyde (31.0 g, 199.26 mmol) (1 equiv.) in 1,4-dioxane (1100 mL) under nitrogen was added hydrazine hydrate (279.3 g, 5.58 mol, 279.3 mL, 28.0 equiv.). The mixture was refluxed for 48 h then cooled. The layers were separated and the organic layer was concentrated under reduced pressure. Then, water (200 mL) was added to the obtained residue. The suspension was stirred at room temperature for 1 hour, filtered, the solid was washed with water (100 mL), and air-dried to give 6-methyl-1H-pyrazolo[4,3-c]pyridine (3.7 g, 95.0% purity, 26.4 mmol, 13.2% yield) as a yellow solid.

Step 4: To a cooled (water bath) suspension of 6-methyl-1H-pyrazolo[4,3-c]pyridine (5.0 g, 37.55 mmol) (1.00 equiv.) and potassium hydroxide (7.58 g, 135.19 mmol) (3.60 equiv.) in DMF (80 mL), was added iodine (19.06 g, 75.11 mmol) (2.00 equiv.). The reaction mixture was stirred for 1 h then, the mixture was quenched by addition of a saturated aqueous solution of Na₂S203, extracted with ethyl acetate (3×200 mL), dried over anhydrous sodium sulfate, and concentrated under reduced pressure to give 3-iodo-6-methyl-1H-pyrazolo[4,3-c]pyridine (5.2 g, 98.0% purity, 19.67 mmol, 52.4% yield) as a yellow solid.

Step 5: 3-iodo-6-methyl-1H-pyrazolo[4,3-c]pyridine (5.2 g, 20.07 mmol), triethylamine (2.44 g, 24.09 mmol) and Pd(dppf)Cl₂.DCM complex (50 mg, 3 mol %) were dissolved in MeOH (200 mL). The reaction mixture was heated at 120° C. in high pressure vessel at 40 atm pressure in CO atmosphere for 24 h. Then, the solvent was evaporated in vacuo. The residue was re-dissolved in water (100 mL). The mixture was stirred at room temperature for 1 hour and filtered. The solid obtained was washed with water (100 mL) and air-dried to give crude product as an orange solid. The obtained solid was purified by flash chromatography (MeOH:DCM 1:30) to give methyl 6-methyl-1H-pyrazolo[4,3-c]pyridine-3-carboxylate (1.2 g, 98.0% purity, 6.15 mmol, 30.6% yield).

Step 6: To a suspension of methyl 6-methyl-1H-pyrazolo[4,3-c]pyridine-3-carboxylate (1.2 g, 6.28 mmol) and di-tert-butyl dicarbonate (2.81 g, 12.87 mmol) in methanol (50 mL), was added Pd(OH)₂ (20% on activated carbon, 0.1 mmol). The mixture was hydrogenated in an autoclave at 45 atm H₂ at room temperature for 48 h. Then, the reaction mixture was filtered through a pad of silica and the pad was washed with methanol (50 mL). The filtrate was concentrated under reduced pressure to give 1,5-di-tert-butyl 3-methyl 6-methyl-1H,4H,5H,6H,7H-pyrazolo[4,3-c]pyridine-1,3,5-tricarboxylate (2.2 g, 90.0% purity, 5.01 mmol, 79.8% yield) as an oil (mixture of mono- and di-Boc product) which was used to the next step without further purification.

Step 7: MeOH (70 mL) and saturated aqueous solution of NaHCO₃ (15 mL) were added to 1,5-di-tert-butyl 3-methyl 6-methyl-1H,4H,5H,6H,7H-pyrazolo[4,3-c]pyridine-1,3,5-tricarboxylate (2.2 g, 5.56 mmol). The mixture was stirred at room temperature for 18 h, then the solvent was evaporated in vacuo. The residue was mixed with water (25 mL). The obtained suspension was extracted with MTBE (2×50 mL), dried over anhydrous sodium sulfate, and concentrated under reduced pressure to give crude 5-tert-butyl 3-methyl 6-methyl-1H,4H,5H,6H,7H-pyrazolo[4,3-c]pyridine-3,5-dicarboxylate (1.7 g, 90.0% purity, 5.18 mmol, 93.1% yield) as a yellow semi-solid which was used to the next step without further purification.

Step 8: To a cooled (0° C.) solution of 5-tert-butyl 3-methyl 6-methyl-1H,4H,5H,6H,7H-pyrazolo[4,3-c]pyridine-3,5-dicarboxylate (1.7 g, 5.76 mmol) (1 eq.) in THF (75 mL) was added portionwise sodium hydride (334.06 mg, 13.92 mmol). The mixture was stirred at room temperature for 30 mins followed by the dropwise addition of [2-(chloromethoxy)ethyl]trimethylsilane (1.28 g, 7.66 mmol). The resulting mixture was stirred at room temperature for an additional 16 h, then quenched with water and extracted with EtOAc (3×50 mL). The combined organic extracts were dried over anhydrous sodium sulfate, filtered, concentrated and purified by fish column chromatography (hexane:MTBE 2:1) to yield 5-tert-butyl 3-methyl 6-methyl-1-[2-(trimethylsilyl)ethoxy]methyl-1H,4H,5H,6H,7H-pyrazolo[4,3-c]pyridine-3,5-dicarboxylate (1.6 g, 97.0% purity, 3.65 mmol, 63.3% yield) as yellow oil.

Step 9: 5-tert-butyl 3-methyl 6-methyl-1-[2-(trimethylsilyl)ethoxy]methyl-1H,4H,5H,6H,7H-pyrazolo[4,3-c]pyridine-3,5-dicarboxylate (1.6 g, 3.76 mmol) and lithium hydroxide monohydrate (473.2 mg, 11.28 mmol) were stirred in a mixture of THF:H₂O:methanol (v/v 3:1: 1, 50 mL) at 25° C. for 18 h.Then, the reaction mixture was concentrated under reduced pressure. The residue was acidified with saturated solution of citric acid to pH 4. The mixture was extracted with EtOAc (3×30 mL). The combined organic extracts were dried over anhydrous sodium sulfate, filtered, and concentrated under reduced pressure. The obtained residue was purified by HPLC to give 5-[(tert-butoxy)carbonyl]-6-methyl-1-[2-(trimethylsilyl)ethoxy]methyl-1H,4H,5H,6H,7H-pyrazolo[4,3-c]pyridine-3-carboxylic acid (1.1 g, 97.0% purity, 2.59 mmol, 69% yield) as white semi-solid.

Step 10: 5-(tert-butoxycarbonyl)-6-methyl-1-((2-(trimethylsilyl)ethoxy)methyl)-4,5,6,7-tetrahydro-1H-pyrazolo[4,3-c]pyridine-3-carboxylic acid (402.77 mg, 978.61 μmol) and HATU (427.91 mg, 1.13 mmol) were mixed in DMF (5 mL). The resulting mixture was stirred for 15 mins at room temperature, then methyl 4-[1-(methylamino)cyclopropyl]benzoate hydrochloride (236.54 mg, 978.61 μmol) and triethylamine (326.7 mg, 3.23 mmol, 450.0 μL, 3.3 equiv.) were added. The reaction mixture was stirred overnight (18 h) at room temperature.

Then, the mixture was poured into water (50 mL) and extracted with MTBE (3×50 mL). The combined organic extracts were washed with water (3×30 mL), dried over anhydrous sodium sulfate, and the solvent was removed in vacuo. The residue obtained was purified by flash column chromatography (hexane:MTBE) to afford tert-butyl 3-(1-[4-(methoxycarbonyl)phenyl]cyclopropyl(methyl)carbamoyl)-6-methyl-1-[2-(trimethylsilyl)ethoxy]methyl-1H,4H,5H,6H,7H-pyrazolo[4,3-c]pyridine-5-carboxylate (265.0 mg, 98.0% purity, 433.7 μmol, 44.3% yield) as semi-solid.

### Synthesis of 5-tert-butyl 3-ethyl 4H,5H,6H,7H-[1,2]oxazolo[4,3-c]pyridine-3,5-dicarboxylate

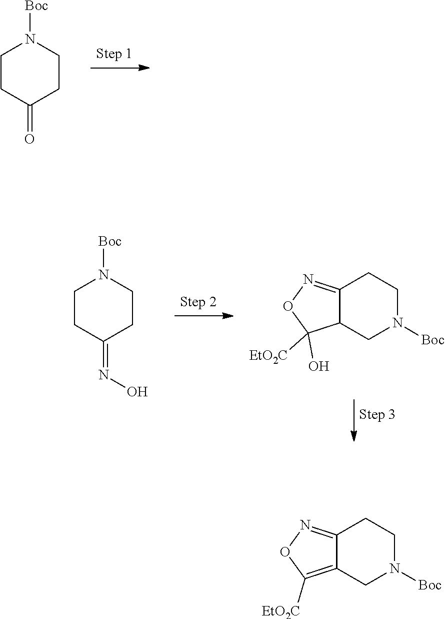 [SMILES: CC(C)(C)OC(=O)N1CCC(=O)CC1 ; CC(C)(C)OC(=O)N1CCC(=NO)CC1 ; CCOC(=O)C1(O)ON=C2CCN(C(=O)OC(C)(C)C)CC21 ; CCOC(=O)c1onc2c1CN(C(=O)OC(C)(C)C)CC2]

Step 1: To a solution of hydroxylamine hydrochloride (10.7 g, 153.95 mmol) in ethanol (100 mL) and water (25 mL) were added tert-butyl 4-oxopiperidine-1-carboxylate (20.45 g, 102.64 mmol) and potassium acetate (16.12 g, 164.22 mmol). The white suspension was stirred under reflux for 3 h, then cooled and filtered. The filtrate was concentrated under reduced pressure.

The residue was partitioned between water (200 mL) and DCM (250 mL). The layers were separated and the organic layer was extracted with DCM (50 mL). The combined organic extracts were dried (sodium sulfate) and concentrated to afford tert-butyl 4-(hydroxyimino)piperidine-1-carboxylate (20.2 g, 94.28 mmol, 91.9% yield) as beige solid.

Step 2: To a cooled (−78° C.) solution of tert-butyl 4-(hydroxyimino)piperidine-1-carboxylate (35.2 g, 164.28 mmol) in THF (300 mL) under argon was added dropwise a solution of sec-butyllithium (31.57 g, 492.85 mmol, 352.04 mL, 3.0 equiv.). The mixture was stirred for 1 h, then diethyl oxalate (33.61 g, 230.0 mmol) was added dropwise. The mixture was stirred for 15 mins then warmed to room temperature and stirred for a further 1 h. The reaction was quenched by addition of sat. aq. NH₄Cl (1000 mL) and extracted with EtOAc (3×300 mL). The combined organic extracts were dried over sodium sulfate and concentrated to yield crude 5-tert-butyl 3-ethyl 3-hydroxy-3H,3aH,4H,5H,6H,7H-[1,2]oxazolo[4,3-c]pyridine-3,5-dicarboxylate (43.2 g, 137.43 mmol, 83.7% yield) as brown oil, that was used in the next step without further purification.

Step 3: To a cooled (0° C.) solution of 5-tert-butyl 3-ethyl 3-hydroxy-3H,3aH,4H,5H,6H,7H-[1,2]oxazolo[4,3-c]pyridine-3,5-dicarboxylate (6.0 g, 19.09 mmol) and triethylamine (5.79 g, 57.26 mmol, 7.98 mL, 3.0 equiv.) in THF (40 mL) was added methanesulfonyl chloride (2.84 g, 24.81 mmol, 1.92 mL, 1.3 equiv.). The cooling bath was removed and the mixture was stirred for 1 h. The solution was concentrated under reduced pressure then diluted with EtOAc (100 mL), and washed with saturated aqueous NH₄Cl (50 mL). The water layer was extracted with EtOAc (10 mL). The combined organic extracts were dried over sodium sulfate and concentrated under reduced pressure. The residue was purified by column chromatography (silica, hexane-EtOAc gradient) to afford 5-tert-butyl 3-ethyl 4H,5H,6H,7H-[1,2]oxazolo[4,3-c]pyridine-3,5-dicarboxylate (1.0 g, 3.37 mmol, 17.7% yield) as yellow oil.

### Synthesis of 4,6-dichloro-5-fluoro-1H-indole-2-carboxylic acid

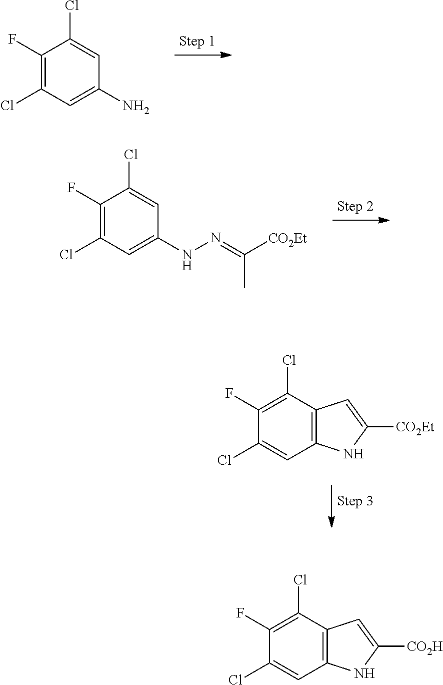 [SMILES: Nc1cc(Cl)c(F)c(Cl)c1 ; CCOC(=O)/C(C)=N/Nc1cc(Cl)c(F)c(Cl)c1 ; CCOC(=O)c1cc2c(Cl)c(F)c(Cl)cc2[nH]1 ; O=C(O)c1cc2c(Cl)c(F)c(Cl)cc2[nH]1]

Step 1: To a cooled (0° C.) solution of sodium nitrite (2.49 g, 36.11 mmol) in EtOH/H₂O (25 mL/25 mL) was added dropwise a solution of 3,5-dichloro-4-fluoroaniline (5.0 g, 27.78 mmol) in HCl (conc., 11 mL), H₂O (10 mL) and EtOH (25 mL). After 5 mins at 0° C., ethyl 2-methyl-3-oxobutanoate (4.41 g, 30.55 mmol) was added in one portion. The resulting mixture was then added over 3 minutes to a cooled (−10° C.), stirred mixture of EtOH (100 mL) and aqueous KOH (50%, 21 mL), maintaining the internal temperature between −10° C. and −5° C. After addition was complete, the mixture was warmed to 5° C. (over ˜15 mins) and then poured into stirring saturated aqueous NH₄Cl solution (400 mL). The precipitate was collected by filtration, washed with water (100 mL) and re-disolved in DCM (200 mL). The resulting solution was dried over anhydrous sodium sulfate and concentrated under reduced pressure. The residue was purified by flash column chromatography (silica, petroleum ether/MTBE gradient, MTBE from 10-25%) to provide ethyl (2Z)-2-[2-(3,5-dichloro-4-fluorophenyl)hydrazin-1-ylidene]propanoate (1.2 g, 4.09 mmol, 14.7% yield).

Step 2: To a solution of ethyl (2Z)-2-[2-(3,5-dichloro-4-fluorophenyl)hydrazin-1-ylidene]propanoate (1.2 g, 4.09 mmol) in benzene (70 mL) was added 4-methylbenzene-1-sulfonic acid (1.76 g, 10.23 mmol). The mixture was refluxed overnight. After cooling to r.t., the reaction mixture was diluted with EtOAc (50 mL), washed with water, and aq Na₂CO₃. The mixture was dried over sodium sulfate and concentrated under reduced pressure. The residue was purified by flash column chromatography to afford ethyl 4,6-dichloro-5-fluoro-1H-indole-2-carboxylate (140.0 mg, 507.08 μmol, 12.4% yield) as light yellow powder.

Step 3: To a solution of ethyl 4,6-dichloro-5-fluoro-1H-indole-2-carboxylate (140.0 mg, 507.08 μmol) in EtOH (3 mL) was added a solution of sodium hydroxide (60.95 mg, 1.52 mmol) in H₂O (1 mL). The resulting solution was stirred overnight, then concentrated. The residue was partitioned between water (20 mL) and EtOAc (10 mL). The aqueous phase was separated, acidified with NaHSO₄ and extracted with EtOAc (20 mL). The organic phase was washed with brine, dried over sodium sulfate and concentrated in vacuum to give 4,6-dichloro-5-fluoro-1H-indole-2-carboxylic acid (45.0 mg, 181.42 μmol, 35.7% yield).

### Synthesis of 2-(1-{N-methyl-5-[(tert-butoxy)carbonyl]-4H,5H,6H,7H-pyrazolo[1,5-a]pyrazine-3-amido}cyclopropyl)benzoic acid

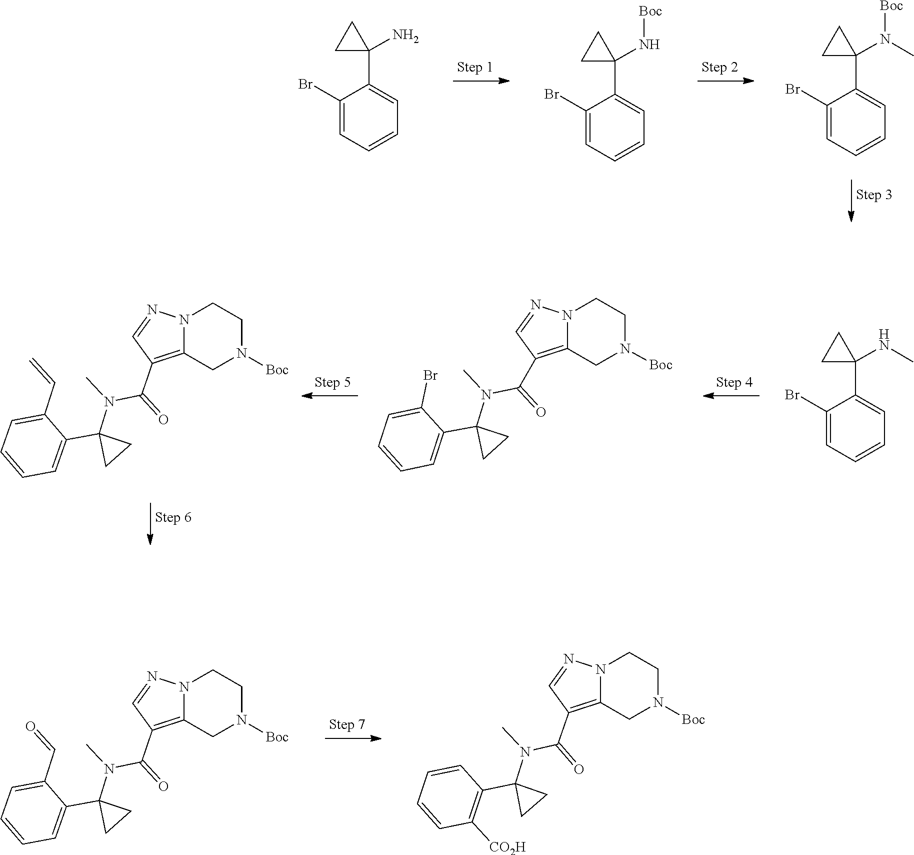

Step 1: To a cooled (0° C.), stirred suspension of 1-(2-bromophenyl)cyclopropan-1-amine hydrochloride (3.75 g, 15.1 mmol) in DCM (50 mL) were added di-tert-butyl dicarbonate (3.3 g, 15.1 mmol) and triethylamine (1.76 g, 17.36 mmol, 2.42 mL, 1.15 equiv.). The reaction mixture was stirred overnight and diluted with water (10 mL). The organic phase was separated, washed with water, dried over sodium sulfate, filtered and concentrated to afford tert-butyl N-[1-(2-bromophenyl)cyclopropyl]carbamate (3.8 g, 12.17 mmol, 80.6% yield) as yellow oil. Step 2: To a cooled (0° C.) suspension of sodium hydride (730.28 mg, 30.43 mmol) in dry DMF (20 mL) was added dropwise a solution of tert-butyl N-[1-(2-bromophenyl)cyclopropyl]carbamate (3.8 g, 12.17 mmol) in dry DMF (10 mL). The reaction mixture was stirred at room temperature for 30 mins. The resulting mixture was cooled (0° C.) and iodomethane (3.46 g, 24.34 mmol, 1.52 mL, 2.0 equiv.) was added. The reaction mixture was stirred at room temperature overnight. The obtained suspension was poured onto ice water and the product was extracted with ethyl acetate (3×20 mL). The combined organic extracts were washed with water and brine, dried over sodium sulfate, and concentrated in vacuo to afford tert-butyl N-[1-(2-bromophenyl)cyclopropyl]-N-methylcarbamate (3.03 g, 9.29 mmol, 76.3% yield) as yellow oil.

Step 3: To a stirred solution of tert-butyl N-[1-(2-bromophenyl)cyclopropyl]-N-methylcarbamate (1.3 g, 3.98 mmol) in dry DCM (10 mL) was added 4M HCl in dioxane (726.24 mg, 19.92 mmol, 6.05 mL, 5.0 equiv.) was added. The reaction mixture was stirred at room temperature for 10 h, then concentrated under reduced pressure. The residue was triturated with hexane, filtered, and dried to afford 1-(2-bromophenyl)-N-methylcyclopropan-1-amine hydrochloride (970.0 mg, 3.69 mmol, 92.7% yield) as white solid.

Step 4: To a cooled (0° C.), stirred solution of HATU (1.4 g, 3.69 mmol) and 5-[(tert-butoxy)carbonyl]-4H,5H,6H,7H-pyrazolo[1,5-a]pyrazine-3-carboxylic acid (987.31 mg, 3.69 mmol) in DMF (10 mL) were added 1-(2-bromophenyl)-N-methylcyclopropan-1-amine hydrochloride (969.92 mg, 3.69 mmol) and triethylamine (1.5 g, 14.78 mmol). The reaction mixture was stirred for 1 h, warmed to room temperature, and stirred overnight. The mixture was poured into water (20 mL) and product was extracted with EtOAc (3×10 mL). The combined organic extracts were washed with water, aq. sodium bicarbonate, dried over sodium sulfate, filtered, and concentrated under reduced pressure to afford tert-butyl 3-[1-(2-bromophenyl)cyclopropyl](methyl)carbamoyl-4H,5H,6H,7H-pyrazolo[1,5-a]pyrazine-5-carboxylate (1.8 g, 89.0% purity, 3.37 mmol, 91.2% yield) as brown solid.

Step 5: To a degassed solution of tert-butyl 3-[1-(2-bromophenyl)cyclopropyl](methyl)carbamoyl-4H,5H,6H,7H-pyrazolo[1,5-a]pyrazine-5-carboxylate (1.8 g, 3.79 mmol) in EtOH (20 mL) were added potassium ethenyltrifluoroboranuide (1.02 g, 7.58 mmol), Pd(dppf)Cl₂.DCM complex (309.44 mg, 378.92 μmol) and triethylamine (3.83 g, 37.88 mmol, 5.28 mL, 10.0 equiv.). The reaction mixture was stirred at 85° C. for 30 h. The mixture was cooled to room temperature and concentrated under reduced pressure. The residue was dissolved in EtOAc (10 mL), filtered through a silica pad, and concentrated. The residue was purified by column chromatography on silica (from MTBE-hexane 1:3 to MTBE-hexane 9:1 as eluent) to afford tert-butyl 3-[1-(2-ethenylphenyl)cyclopropyl](methyl)carbamoyl-4H,5H,6H,7H-pyrazolo[1,5-a]pyrazine-5-carboxylate (900.0 mg, 2.13 mmol, 56.2% yield) as yellow foam.

Step 6: To a solution of tert-butyl 3-[1-(2-ethenylphenyl)cyclopropyl](methyl)carbamoyl-4H,5H,6H,7H-pyrazolo[1,5-a]pyrazine-5-carboxylate (900.0 mg, 2.13 mmol) in EtOAc (10 mL) and water (5 mL) was added ruthenium (IV) oxide (14.17 mg, 106.48 μmol). The reaction mixture was stirred for 30 mins, then sodium periodate (1.82 g, 8.52 mmol) was added. The reaction mixture was stirred for 20 h. The organic phase was separated, dried over sodium sulfate, filtered, and concentrated under reduced pressure. The residue was purified by HPLC to afford tert-butyl 3-[1-(2-formylphenyl)cyclopropyl](methyl)carbamoyl-4H,5H,6H,7H-pyrazolo[1,5-a]pyrazine-5-carboxylate (170.0 mg, 400.48 μmol, 18.8% yield) as yellow solid.

Step 7: To a cooled (0° C.) solution of tert-butyl 3-[1-(2-formylphenyl)cyclopropyl](methyl)carbamoyl-4H,5H,6H,7H-pyrazolo[1,5-a]pyrazine-5-carboxylate (170.0 mg, 400.48 μmol) in tert-butanol (2 mL) and 2-methyl-2-butene (1 mL) was slowly added solution of sodium chlorite (46.98 mg, 519.42 μmol) and sodium dihydrogen phosphate (95.87 mg, 799.11 μmol) in water (2 mL). The reaction mixture was stirred at room temperature overnight then concentrated. The residue was dissolved in water (5 mL) and acidified to pH 3 with 5% aq. HCl. The mixture was extracted with EtOAc (2×5 mL). The combined organic extracts were washed with water (5 mL), dried over sodium sulfate, filtered, and concentrated under reduced pressure to afford 2-(1-N-methyl-5-[(tert-butoxy)carbonyl]-4H,5H,6H,7H-pyrazolo[1,5-a]pyrazine-3-amidocyclopropyl)benzoic acid (157.0 mg, 356.42 μmol, 89.2% yield) as white foam.

### Synthesis of 2-(1-{N-methyl-5-[(tert-butoxy)carbonyl]-4H,5H,6H,7H-pyrazolo[1,5-a]pyrazine-3-amido}cyclopropyl)pyridine-3-carboxylic acid

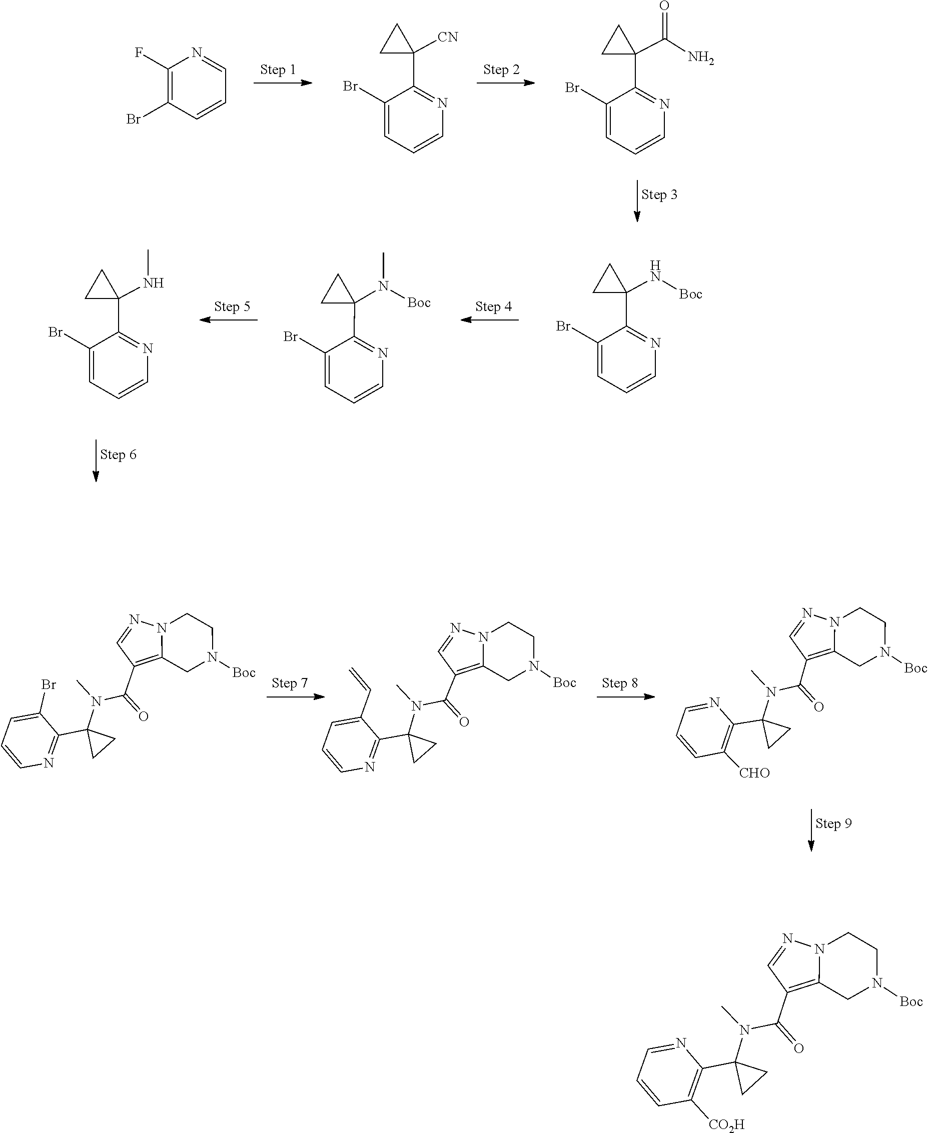

Step 1: Lithium bis(trimethylsilyl)azanide (29.47 g, 176.14 mmol, 176.14 mL, 3.1 equiv.) was added dropwise to a cooled (−5° C.) mixture of 3-bromo-2-fluoropyridine (10.0 g, 56.82 mmol), cyclopropanecarbonitrile (11.44 g, 170.46 mmol, 12.55 mL, 3.0 equiv.), 4 Angstrom molecular sieves, and toluene (100 mL). The reaction mixture was allowed to warm to room temperature, stirred for 1 h, then poured into water, and filtered. The mixture was extracted with EtOAc (2×15 mL). The combined organic extracts were washed with brine, dried over sodium sulfate, filtered, and concentrated. The residue was purified with column chromatography on silica (hexane-MTBE 4:1 as eluent) to afford 1-(3-bromopyridin-2-yl)cyclopropane-1-carbonitrile (6.5 g, 29.14 mmol, 51.3% yield) as light yellow solid.

Step 2: A mixture of 1-(3-bromopyridin-2-yl)cyclopropane-1-carbonitrile (5.7 g, 25.55 mmol) and sulfuric acid (90%, 12 mL) was stirred at room temperature overnight. The mixture was poured into a cold aqueous solution of NH₃ (25%) and the mixture was concentrated to dryness.

The residue was triturated with dry MeOH (100 mL) and filtered. The filtrate was concentrated, the residue was dried in vacuo to afford 1-(3-bromopyridin-2-yl)cyclopropane-1-carboxamide (6.0 g, 24.89 mmol, 97.4% yield) as yellow solid.

Step 3: 1-(3-bromopyridin-2-yl)cyclopropane-1-carboxamide (1.5 g, 6.22 mmol) was dissolved in dry t-BuOH (20 mL per mmol) with a few drops of pyridine and flushed with argon. Lead tetraacetate (6.07 g, 13.69 mmol) was added, and the reaction mixture was heated at reflux for 2 h. The mixture was cooled to room temperature, concentrated under reduced pressure, and the residue diluted with sat. aq. NaHCO₃ (to pH 8) and EtOAc (30 mL). The biphasic mixture was filtered. The filtrate was transferred to a separatory funnel. The organic phase was separated and the water phase was extracted with EtOAc (2×15 mL). The combined organic extracts were dried over sodium sulfate, filtered, and concentrated. The residue was purified by column chromatography (silica, EtOAc-hexane 5:1) to afford tert-butyl N-[1-(3-bromopyridin-2-yl)cyclopropyl]carbamate (330.0 mg, 1.05 mmol, 16.9% yield) as yellow solid Step 4: To a cooled (0° C.), stirred solution of tert-butyl N-[1-(3-bromopyridin-2-yl)cyclopropyl]carbamate (330.21 mg, 1.05 mmol) in dry DMF (3 mL) under argon was added sodium hydride (63.25 mg, 2.64 mmol). The mixture was stirred for 1 h then iodomethane (224.48 mg, 1.58 mmol) was added. The mixture was stirred at 0° C. for 1 h, warmed to room temperature, and stirred overnight. The mixture was poured into water (10 mL) and extracted with EtOAc (3×10 mL). The combined organic extracts were washed with water, brine, dried over sodium sulfate, filtered, and concentrated to afford crude tert-butyl N-[1-(3-bromopyridin-2-yl)cyclopropyl]-N-methylcarbamate (240.0 mg, 733.46 μmol, 69.6% yield) as yellow oil. The obtained product was used in the next step without further purification.

Step 5: To a solution of tert-butyl N-[1-(3-bromopyridin-2-yl)cyclopropyl]-N-methylcarbamate (239.94 mg, 733.28 μmol) in MeOH (1 mL) was added conc. HCl (0.2 mL).

The reaction mixture was stirred at room temperature overnight. The mixture was concentrated under reduced pressure. The residue was dried under vacuum to afford 1-(3-bromopyridin-2-yl)-N-methylcyclopropan-1-amine dihydrochloride (210.0 mg, 699.95 μmol, 95.5% yield) as brown solid.

Step 6: To a solution of 5-[(tert-butoxy)carbonyl]-4H,5H,6H,7H-pyrazolo[1,5-a]pyrazine-3-carboxylic acid (186.84 mg, 699.04 μmol) in DMF (1 mL) was added HATU (265.8 mg, 699.04 pmol). The reaction mixture was stirred for 10 mins, then 1-(3-bromopyridin-2-yl)-N-methylcyclopropan-1-amine dihydrochloride (209.73 mg, 699.04 μmol) and triethylamine (353.68 mg, 3.5 mmol) were added. The resulting mixture was stirred for 5 h, then poured into water (3 mL) and extracted with EtOAc (2×5 mL). The combined organic extracts were washed with brine, dried with sodium sulfate, filtered, and concentrated to afford crude tert-butyl 3-[1-(3-bromopyridin-2-yl)cyclopropyl](methyl)carbamoyl-4H,5H,6H,7H-pyrazolo [1,5-a]pyrazine-5-carboxylate (300.0 mg, 629.77 μmol, 90.1% yield) as brown solid. The obtained product was used in the next step without further purification.

Step 7: To a solution of tert-butyl 3-[1-(3-bromopyridin-2-yl)cyclopropyl](methyl)carbamoyl-4H,5H,6H,7H-pyrazolo[1,5-a]pyrazine-5-carboxylate (300.0 mg, 629.77 μmol) in EtOH (5 mL) under argon were added potassium ethenyltrifluoroboranuide (168.9 mg, 1.26 mmol), Pd(dppf)Cl₂.DCM complex (51.48 mg, 63.04 μmol), and triethylamine (637.95 mg, 6.3 mmol, 880.0 μL, 10.0 equiv.). The reaction mixture was stirred at 85° C. for 30 h then cooled to room temperature and concentrated under reduced pressure. The residue was dissolved in EtOAc (10 mL), filtered through a silica pad, and concentrated. The residue was purified by column chromatography on silica (from MTBE-hexane 1:3 to MTBE-hexane 9:1 as eluent) to afford tert-butyl 3-[1-(3-ethenylpyridin-2-yl)cyclopropyl](methyl)carbamoyl-4H,5H,6H,7H-pyrazolo[1,5-a]pyrazine-5-carboxylate (160.0 mg, 377.8 μmol, 59.9% yield) as yellow solid.

Step 8: To a solution of tert-butyl 3-[1-(3-ethenylpyridin-2-yl)cyclopropyl](methyl)carbamoyl-4H,5H,6H,7H-pyrazolo[1,5-a]pyrazine-5-carboxylate (160.0 mg, 377.8 μmol) in EtOAc (1 mL) and water (1 mL) were added ruthenium (IV) oxide (2.52 mg, 18.92 μmol) and sodium periodate (323.74 mg, 1.51 mmol). The mixture was stirred at room temperature for 24 h. The organic phase was separated, and the aqueous phase was extracted with EtOAc (1 mL). The combined organic phases was dried over sodium sulfate, filtered and concentrated. The residue was purified by HPLC to give tert-butyl 3-[1-(3-formylpyridin-2-yl)cyclopropyl](methyl)carbamoyl-4H,5H,6H,7H-pyrazolo[1,5-a]pyrazine-5-carboxylate (43.0 mg, 86.0% purity, 86.91 μmol, 23% yield) as colorless foam.

Step 9: tert-Butyl 3-[1-(3-formylpyridin-2-yl)cyclopropyl](methyl)carbamoyl-4H,5H,6H,7H-pyrazolo[1,5-a]pyrazine-5-carboxylate (42.98 mg, 101.02 μmol) was dissolved in tert butanol (1 mL) and 2-methyl-2-butene (0.5 mL). The resulting mixture was cooled to 0° C. and a solution of sodium chlorite (11.88 mg, 131.33 μmol) and sodium dihydrogen phosphate (24.24 mg, 202.05 μmol) in water (1 mL) was added slowly. The reaction mixture was stirred at room temperature overnight. The mixture was concentrated, the residue dissolved in water (5 mL) and acidified to pH 3 with 5% aq. HCl. The mixture was extracted with EtOAc (2×5 mL). The combined organic extracts were washed with water (5 mL), dried over sodium sulfate, filtered, and concentrated. The residue was purified by HPLC to afford 2-(1-N-methyl-5-[(tert-butoxy)carbonyl]-4H,5H,6H,7H-pyrazolo[1,5-a]pyrazine-3-amidocyclopropyl)pyridine-3-carboxylic acid (11.0 mg, 24.92 μmol, 24.7% yield) as white foam.

### Synthesis of 2-[4-(1-{N-methyl-5-[(tert-butoxy)carbonyl]-4H,5H,6H,7H-pyrazolo[1,5-a]pyrazine-3-amido}cyclopropyl)phenyl]acetic acid

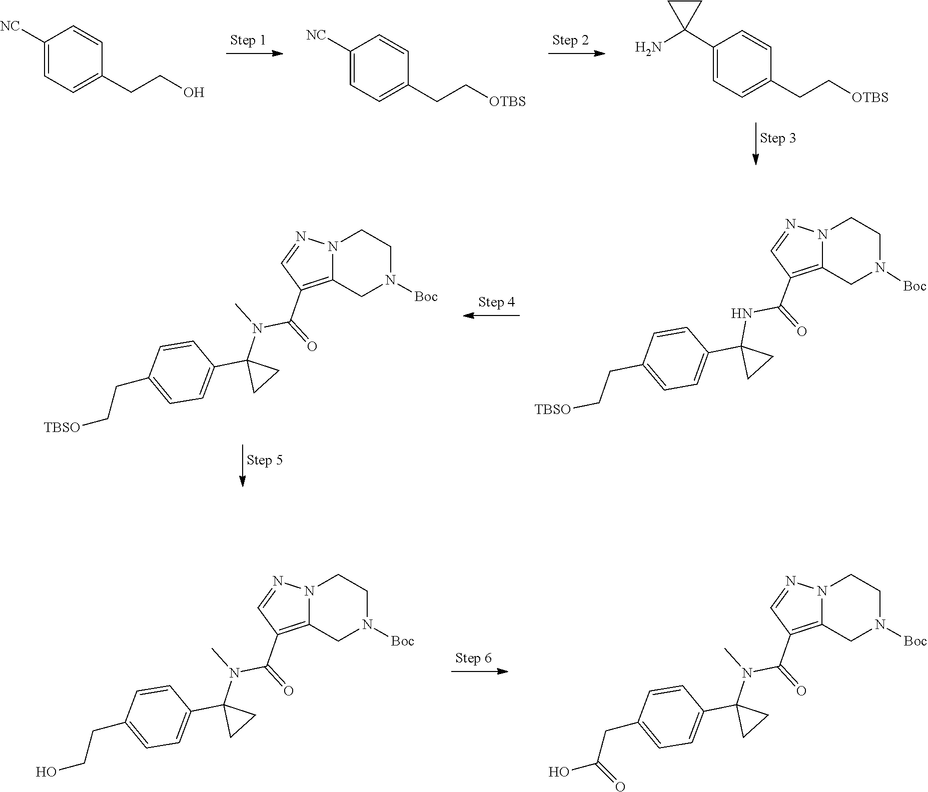

Step 1: To a stirred solution of 4-(2-hydroxyethyl)benzonitrile (7.5 g, 50.96 mmol), tert-butyl(chloro)dimethylsilane (9.99 g, 66.25 mmol), and triethylamine (10.31 g, 101.92 mmol, 14.21 mL, 2.0 equiv.) in DCM (100 mL) was added DMAP (124.52 mg, 1.02 mmol). The mixture was stirred overnight. The reaction mixture was washed with water (2×100 mL), dried over sodium sulfate, and concentrated under reduced pressure to give 4-2-[(tert-butyldimethylsilyl)oxy]ethylbenzonitrile (12.8 g, 48.96 mmol, 96.1% yield) as light brown oil.

Step 2: To a cooled (−70° C.) solution of 4-2-[(tert-butyldimethylsilyl)oxy]ethylbenzonitrile (999.99 mg, 3.82 mmol) and tetrakis(propan-2-yloxy)titanium (1.2 g, 4.21 mmol, 1.25 mL, 1.1 equiv.) in dry Et₂O (30 mL) was added ethylmagnesium bromide (1.07 g, 8.03 mmol, 2.36 mL, 2.1 equiv.). The solution was stirred for 10 mins, warmed to room temperature, then BF₃.OEt₂ (1.09 g, 7.65 mmol, 970.0 μL, 2.0 equiv.) was added. The mixture was stirred for 1 h, then 1N HCl (10 mL) and ether (20 mL) were added. Na₂CO₃ (10% aq, 20 mL) was added to the resulting two clear phases, followed by MTBE (100 mL). After 10 mins vigorous stirring, the organic phase was separated, washed with brine, dried over sodium sulfate and concentrated under reduced pressure. The residue was purified by flash column chromatography (40 g silica, MTBE/methanol with methanol from 0-15%) to give 1-(4-2-[(tert-butyldimethylsilyl)oxy]ethylphenyl)cyclopropan-1-amine (370.0 mg, 1.27 mmol, 33.2% yield) as pale yellow oil.

Step 3: To a solution of 5-[(tert-butoxy)carbonyl]-4H,5H,6H,7H-pyrazolo[1,5-a]pyrazine-3-carboxylic acid (366.74 mg, 1.37 mmol) and triethylamine (277.69 mg, 2.74 mmol, 380.0 μL, 2.0 equiv.) in dry DMF (20 mL) was added HATU (573.89 mg, 1.51 mmol). The resulting mixture was stirred for 10 mins, then 1-(4-2-[(tert-butyldimethylsilyl)oxy]ethylphenyl)cyclopropan-1-amine (200.0 mg, 686.1 μmol) was added.

The reaction mixture was stirred overnight at room temperature. The mixture was partitioned between EtOAc (50 mL) and water (150 mL). The combined organic extracts were washed with water (2×30 mL), brine, dried over sodium sulfate, and concentrated under reduced pressure.

The residue was purified by HPLC to afford tert-butyl 3-[1-(4-2-[(tert-butyldimethylsilyl)oxy]ethylphenyl) cyclopropyl]carbamoyl-4H,5H,6H,7H-pyrazolo[1,5-a]pyrazine-5-carboxylate (150.0 mg, 277.38 μmol, 20.2% yield).

Step 4: To a solution of tert-butyl 3-[1-(4-2-[(tert-butyldimethylsilyl)oxy]ethylphenyl) cyclopropyl]carbamoyl-4H,5H,6H,7H-pyrazolo[1,5-a]pyrazine-5-carboxylate (150.11 mg, 277.58 μmol) in dry DMF (5 mL) was added sodium hydride (16.65 mg, 693.95 μmol). After gas evolution ceased, iodomethane (98.5 mg, 693.95 μmol, 40.0 μL, 2.5 equiv.) was added dropwise. The resulting mixture was stirred overnight at room temperature. The reaction mixture was poured into water (50 mL) and extracted with EtOAc (30 mL). The combined organic extracts were washed with water (2×10 mL), brine, dried over sodium sulfate, and concentrated in vacuo to give tert-butyl 3-[1-(4-2-[(tert-butyldimethylsilyl)oxy]ethylphenyl)cyclopropyl](methyl)carbamoyl-4H,5H,6H,7H-pyrazolo[1,5-a]pyrazine-5-carboxylate (160.0 mg, 90.0% purity, 259.55 μmol, 93.5% yield).

Step 5: To a solution of tert-butyl 3-[1-(4-2-[(tert-butyldimethylsilyl)oxy]ethylphenyl)cyclopropyl](methyl) carbamoyl-4H,5H,6H,7H-pyrazolo[1,5-a]pyrazine-5-carboxylate (150.0 mg, 270.37 μmol) in THF (10 mL) was added tetrabutyl ammonium fluoride (141.26 mg, 540.25 μmol, 540.0 μL, 2.0 equiv.). The mixture was stirred at room temperature overnight then partitioned between EtOAc (20 mL) and water (50 mL). The organic phase was washed with water (2×10 mL), dried over sodium sulfate, and concentrated under reduced pressure to afford tert-butyl 3-(1-[4-(2-hydroxyethyl)phenyl]cyclopropyl(methyl)carbamoyl)-4H,5H,6H,7H-pyrazolo[1,5-a]pyrazine-5-carboxylate (100.0 mg, 227.0 μmol, 84% yield).

Step 6: A mixture of tert-butyl 3-(1-[4-(2-hydroxyethyl)phenyl]cyclopropyl(methyl)carbamoyl)-4H,5H,6H,7H-pyrazolo[1,5-a]pyrazine-5-carboxylate (69.98 mg, 158.85 μmol), (2,2,6,6-tetramethylpiperidin-1-yl)oxidanyl (1.74 mg, 11.12 μmol), MeCN (20 mL), sodium dihydrogen phosphate (76.23 mg, 635.4 μmol), water (15 mL) and sodium hydroxide (25.4 mg, 635.05 μmol, 250.0 μL, 4.0 equiv.) was heated to 35° C. Then, sodium chlorite (28.73 mg, 317.7 μmol) in water (2 mL) and dilute bleach (NaClO, 1 mL, 0.5%) were added simultaneously over 2 min. The mixture was stirred at 35° C. overnight.

The mixture was allowed to cool to room temperature and water (30 mL) was added. The pH was adjusted to 8.0 with 2N NaOH solution. The reaction was quenched by pouring into cold (0° C.) Na₂SO₃ solution. After stirring for 30 mins at room temperature, MTBE (20 mL) was added. The organic layer was separated and discarded. More MTBE (30 mL) was added, and the aqueous layer was acidified with NaHSO₄. The organic layer was separated, washed with water (10 mL) and brine (150 mL), and then concentrated to give the crude product, which was purified by HPLC to give 2-[4-(1-N-methyl-5-[(tert-butoxy)carbonyl]-4H,5H,6H,7H-pyrazolo[1,5-a]pyrazine-3-amidocyclopropyl)phenyl]acetic acid (15.0 mg, 33.0 μmol, 20.8% yield) as white solid.

### Synthesis of tert-butyl 3-(1-[4-(methoxycarbonyl)phenyl]cyclopropyl(methyl)carbamoyl)-4H,5H,6H,7H-[1,2]oxazolo[4,5-c]pyridine-5-carboxylate

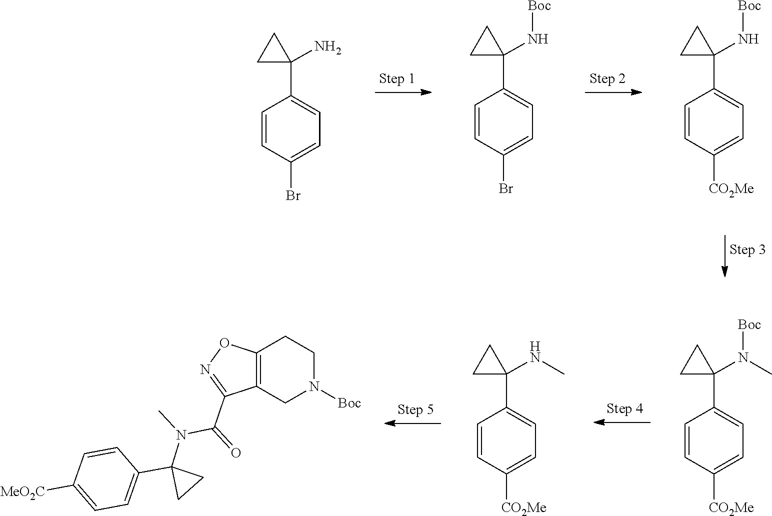 [SMILES: NC1(c2ccc(Br)cc2)CC1 ; CC(C)(C)OC(=O)NC1(c2ccc(Br)cc2)CC1 ; COC(=O)c1ccc(C2(NC(=O)OC(C)(C)C)CC2)cc1 ; COC(=O)c1ccc(C2(N(C)C(=O)c3noc4c3CN(C(=O)OC(C)(C)C)CC4)CC2)cc1 ; CNC1(c2ccc(C(=O)OC)cc2)CC1 ; COC(=O)c1ccc(C2(N(C)C(=O)OC(C)(C)C)CC2)cc1]

Step 1: To a solution of 1-(4-bromophenyl)cyclopropan-1-amine hydrochloride (2.0 g, 8.05 mmol) and di-tert-butyl dicarbonate (1.93 g, 8.85 mmol) in DCM (50 mL) was added dropwise triethylamine (895.6 mg, 8.85 mmol). The resulting mixture was stirred at room temperature for 12 h then the mixture was transferred to a separatory funnel. The organic phase was washed with water (20 mL), brine, dried over sodium sulfate and concentrated to give tert-butyl N-[1-(4-bromophenyl)cyclopropyl]carbamate (2.0 g, 6.41 mmol, 79.6% yield).

Step 2: 1-(N-boc-amino)-1-(4-bromophenyl)cyclopropane (2.0 g, 6.41 mmol) was carbonylated in MeOH (100 mL) at 130° C. and 50 atm CO pressure with Pd(dppf)C₂.DCM complex (100 mg) as catalyst for 18 hours. The resulting mixture was cooled and concentrated and the residue partitioned between water (100 mL) and EtOAc (100 mL). The organic layer was collected, dried over sodium sulfate, and concentrated to give methyl 4-(1-[(tert-butoxy)carbonyl]aminocyclopropyl)benzoate (1.5 g, 5.15 mmol, 80.4% yield) which was used in the next step without additional purification.

Step 3: To a cooled (0° C.) suspension of sodium hydride (148.24 mg, 6.18 mmol) in dry DMF (15 mL), was added dropwise a solution of methyl 4-(1-[(tert-butoxy)carbonyl]aminocyclopropyl)benzoate (1.5 g, 5.15 mmol) in dry DMF (5 mL). The resulting mixture was stirred until gas evolution ceased, then iodomethane (1.1 g, 7.72 mmol) was added dropwise. The resulting mixture was warmed to room temperature, stirred overnight then poured into saturated aq. ammonium chloride solution. The product was extracted with EtOAc (2×40 mL). The combined organic extracts were dried over sodium sulfate and concentrated to give methyl 4-(1-[(tert-butoxy)carbonyl](methyl)aminocyclopropyl)benzoate (1.2 g, 3.93 mmol, 76.3% yield).

Step 4: To methyl 4-(1-[(tert-butoxy)carbonyl](methyl)aminocyclopropyl)benzoate (1.2 g, 3.93 mmol) was added 4M HCl in dioxane (20 mL, 80 mmol). The resulting mixture was stirred at room temperature overnight, then evaporated to dryness to give methyl 4-[1-(methylamino)cyclopropyl]benzoate hydrochloride (850.0 mg, 3.52 mmol, 89.5% yield).

Step 5: 5-[(tert-butoxy)carbonyl]-4H,5H,6H,7H-[1,2]oxazolo[4,5-c]pyridine-3-carboxylic acid (500.6 mg, 1.87 mmol), HATU (780.49 mg, 2.05 mmol) and triethylamine (471.9 mg, 4.66 mmol, 650.0 μL, 2.5 equiv.) were mixed in dry DMF (5 mL) at room temperature. The resulting mixture was stirred for 10 mins, then methyl 4-[1-(methylamino)cyclopropyl]benzoate hydrochloride (451.05 mg, 1.87 mmol) was added. The reaction mixture was stirred at room temperature overnight then partitioned between water (50 mL) and EtOAc (50 mL). The organic phase was separated, dried over sodium sulfate, and concentrated. The residue was purified by HPLC to give tert-butyl 3-(1-[4-(methoxycarbonyl)phenyl]cyclopropyl(methyl)carbamoyl)-4H,5H,6H,7H-[1,2]oxazolo[4,5-c]pyridine-5-carboxylate (486.0 mg, 1.07 mmol, 57.2% yield) as white solid.

### Synthesis of 3-(1-{N-methyl-5-[(tert-butoxy)carbonyl]-4H,5H,6H,7H-pyrazolo[1,5-a]pyrazine-3-amido}cyclopropyl)benzoic acid

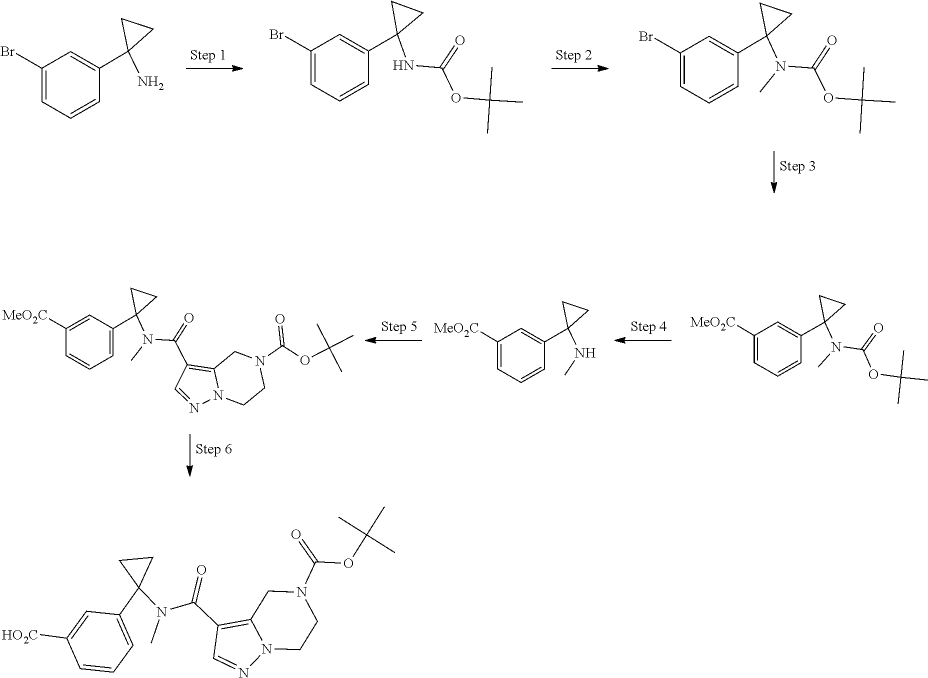

Step 1: To a cooled (0° C.) suspension of 1-(3-bromophenyl)cyclopropan-1-amine hydrochloride (1.01 g, 4.05 mmol) in dry DCM (10 mL) were added di-tert-butyl dicarbonate (882.91 mg, 4.05 mmol) and triethylamine (450.12 mg, 4.45 mmol, 620.0 μL, 1.1 equiv.). The reaction mixture was stirred overnight then diluted with water (5 mL). The organic phase was separated, washed with 10% aqueous solution of H₃PO₄ and water, dried over sodium sulfate, filtered and concentrated under reduced pressure to afford tert-butyl N-[1-(3-bromophenyl)cyclopropyl]carbamate (1.1 g, 3.52 mmol, 87.1% yield) as brown oil.

Step 2: To a cooled (0° C.) suspension of sodium hydride (212.04 mg, 8.84 mmol, 1.5 equiv.) in dry THF (5 mL) under argon, was added dropwise a solution of tert-butyl N-[1-(3-bromophenyl)cyclopropyl]carbamate (1.1 g, 3.53 mmol) in THF (2 mL). The reaction mixture was warmed to room temperature and stirred for 1 h, then re-cooled to 0° C. Iodomethane (752.4 mg, 5.3 mmol, 330.0 μL, 1.5 equiv.) was added dropwise and the reaction mixture was stirred at room temperature overnight. The mixture was diluted with brine (10 mL) and extracted with EtOAc (2×10 mL). The combined organic extracts were washed with brine, dried over sodium sulfate, filtered, and concentrated to give tert-butyl N-[1-(3-bromophenyl)cyclopropyl]-N-methylcarbamate (700.0 mg, 2.15 mmol, 60.7% yield) as yellow oil.

Step 3: To a solution of tert-butyl N-[1-(3-bromophenyl)cyclopropyl]-N-methylcarbamate (701.88 mg, 2.15 mmol) in MeOH (30 mL) were added Pd(dppf)Cl₂.DCM complex (175.7 mg, 215.15 μmol) and triethylamine (261.36 mg, 2.58 mmol, 360.0 μL, 1.2 equiv.). The reaction mixture was carbonylated at 135° C. and 40 atm overnight. The resulting mixture was cooled and concentrated to dryness. The residue was purified by column chromatography on silica (hexane:EtOAc 3:1 as eluent) to afford methyl 3-(1-[(tert-butoxy)carbonyl](methyl)aminocyclopropyl)benzoate (380.0 mg, 1.24 mmol, 57.8% yield) as a colorless oil.

Step 4: To a stirred solution of methyl 3-(1-[(tert-butoxy)carbonyl](methyl)aminocyclopropyl)benzoate (380.0 mg, 1.24 mmol) in dry DCM (5 mL) was added 4M HCl in dioxane (2 mL, 8 mmol) was added. The reaction mixture was stirred at room temperature for 5 h, and then concentrated under reduced pressure. The residue was triturated with hexane, product was collected by filtration and air-dried to afford methyl 3-[1-(methylamino)cyclopropyl]benzoate hydrochloride (290.0 mg, 1.2 mmol, 96.4% yield) as white solid.

Step 5: To a cooled (0° C.) solution of 5-[(tert-butoxy)carbonyl]-4H,5H,6H,7H-pyrazolo[1,5-a]pyrazine-3-carboxylic acid (210.94 mg, 789.21 μmol) in DMF (0.8 mL) was added HATU (300.08 mg, 789.21 μmol). The resulting mixture was stirred for 5 mins at room temperature, then methyl 3-[1-(methylamino)cyclopropyl]benzoate hydrochloride (190.76 mg, 789.21 μmol) and triethylamine (319.44 mg, 3.16 mmol, 440.0 μL, 4.0 equiv.) were added. The reaction mixture was stirred at room temperature overnight, and then diluted with brine. The mixture was extracted with EtOAc (2×20 mL). The combined organic extracts were washed with brine, dried over sodium sulfate, filtered, and concentrated to give tert-butyl 3-(1-[3-(methoxycarbonyl)phenyl]cyclopropyl(methyl)carbamoyl)-4H,5H,6H,7H-pyrazolo[1,5-a]pyrazine-5-carboxylate (270.0 mg, 594.03 μmol, 75.3% yield) as brown oil.

Step 6: To a solution of tert-butyl 3-(1-[3-(methoxycarbonyl)phenyl]cyclopropyl(methyl)carbamoyl)-4H,5H,6H,7H-pyrazolo[1,5-a]pyrazine-5-carboxylate (270.34 mg, 594.79 μmol) in THF/water/MeOH (2 mL/2 mL/1 mL), was added lithium hydroxide monohydrate (74.88 mg, 1.78 mmol). The reaction mixture was stirred overnight at room temperature and then concentrated. The residue was dissolved in water (5 mL) and the mixture was extracted with MTBE (3 mL). The aqueous phase was separated and acidified with 5% aq. HCl to pH 4. The product was extracted with EtOAc (2×5 mL). The combined organic extracts were dried over sodium sulfate, filtered and concentrated to afford 3-(1-N-methyl-5-[(tert-butoxy)carbonyl]-4H,5H,6H,7H-pyrazolo[1,5-a]pyrazine-3-amidocyclopropyl)benzoic acid (220.0 mg, 499.44 μmol, 84% yield) as yellow solid.

### Synthesis of 4-(1-{N-methyl-5-[(tert-butoxy)carbonyl]-4H,5H,6H,7H-pyrazolo[1,5-a]pyrazine-3-amido}cyclopropyl)benzoic acid

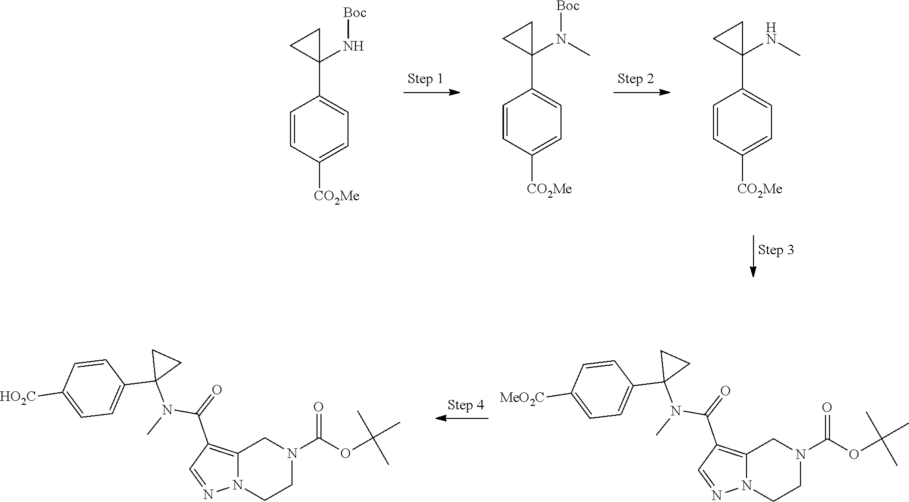 [SMILES: COC(=O)c1ccc(C2(NC(=O)OC(C)(C)C)CC2)cc1 ; COC(=O)c1ccc(C2(N(C)C(=O)OC(C)(C)C)CC2)cc1 ; CNC1(c2ccc(C(=O)OC)cc2)CC1 ; CN(C(=O)c1cnn2c1CN(C(=O)OC(C)(C)C)CC2)C1(c2ccc(C(=O)O)cc2)CC1 ; COC(=O)c1ccc(C2(N(C)C(=O)c3cnn4c3CN(C(=O)OC(C)(C)C)CC4)CC2)cc1]

Step 1: To a cooled (0° C.) suspension of sodium hydride (123.54 mg, 5.15 mmol) in dry DMF (10 mL) was added dropwise a solution of methyl 4-(1-[(tert-butoxy)carbonyl]aminocyclopropyl)benzoate (999.86 mg, 3.43 mmol) in dry DMF (1 mL). The resulting mixture was stirred until gas evolution ceased. Iodomethane (2.44 g, 17.16 mmol) was added dropwise. The resulting mixture was warmed to r.t. and stirred overnight. The reaction mixture was then poured into saturated aq. ammonium chloride solution. The product was extracted twice with EtOAc (10 mL). The organic phases were combined, dried over sodium sulfate and concentrated in vacuo to give methyl 4-(1-[(tert-butoxy)carbonyl](methyl)aminocyclopropyl)benzoate (900.0 mg, 2.95 mmol, 85.9% yield).

Step 2: To methyl 4-(1-[(tert-butoxy)carbonyl](methyl)aminocyclopropyl)benzoate (800.0 mg, 2.62 mmol) was added 4M HCl in dioxane (10 mL, 40 mmol). The resulting mixture was stirred at r.t. overnight and then concentrated to give methyl 4-[1-(methylamino)cyclopropyl]benzoate hydrochloride (600.0 mg, 2.48 mmol, 94.8% yield), which was used in next step without further purification.

Step 3: Methyl 4-[1-(methylamino)cyclopropyl]benzoate hydrochloride (650.0 mg, 2.69 mmol), HATU (1.12 g, 2.96 mmol) and triethylamine (680.14 mg, 6.72 mmol, 940.0 μL, 2.5 equiv.) were mixed in dry DMF (5 mL) at room temperature. The resulting mixture was stirred for 10 minutes followed by the addition of 5-[(tert-butoxy)carbonyl]-4H,5H,6H,7H-pyrazolo[1,5-a]pyrazine-3-carboxylic acid (718.6 mg, 2.69 mmol). The reaction mixture was stirred at room temperature overnight. The resulting mixture was diluted with water (50 mL).

The precipitate was collected by filtration. The filtercake was re-dissolved in EtOAc (20 mL), dried over sodium sulfate and concentrated to give tert-butyl 3-(1-[4-(methoxycarbonyl)phenyl]cyclopropyl(methyl)carbamoyl)-4H,5H,6H,7H-pyrazolo[1,5-a]pyrazine-5-carboxylate (1.0 g, 2.2 mmol, 81.8% yield) which was used in next step without further purification.

Step 4: To a solution of tert-butyl 3-(1-[4-(methoxycarbonyl)phenyl]cyclopropyl(methyl)carbamoyl)-4H,5H,6H,7H-pyrazolo[1,5-a]pyrazine-5-carboxylate (899.77 mg, 1.98 mmol) in methanol (10 mL) was added sodium hydroxide (237.54 mg, 5.94 mmol). The resulting mixture was stirred at r.t. overnight and then evaporated to dryness. The residue was partitioned between water (5 mL) and EtOAc (5 mL).

The aqueous layer was acidified with a solution of sodium hydrogen sulfate (713.02 mg, 5.94 mmol) in water (5 mL). The precipitate was collected by filtration, dissolved in EtOAc (10 mL), dried over sodium sulfate, filtered, and concentrated to dryness. The residue was purified by HPLC to give 4-(1-N-methyl-5-[(tert-butoxy)carbonyl]-4H,5H,6H,7H-pyrazolo[1,5-a]pyrazine-3-amidocyclopropyl)benzoic acid (366.0 mg, 830.89 μmol, 42% yield).

### Synthesis of 6-(1-{N-methyl-5-[(tert-butoxy)carbonyl]-4H,5H,6H,7H-pyrazolo[1,5-a]pyrazine-3-amido}cyclopropyl)pyridine-3-carboxylic acid

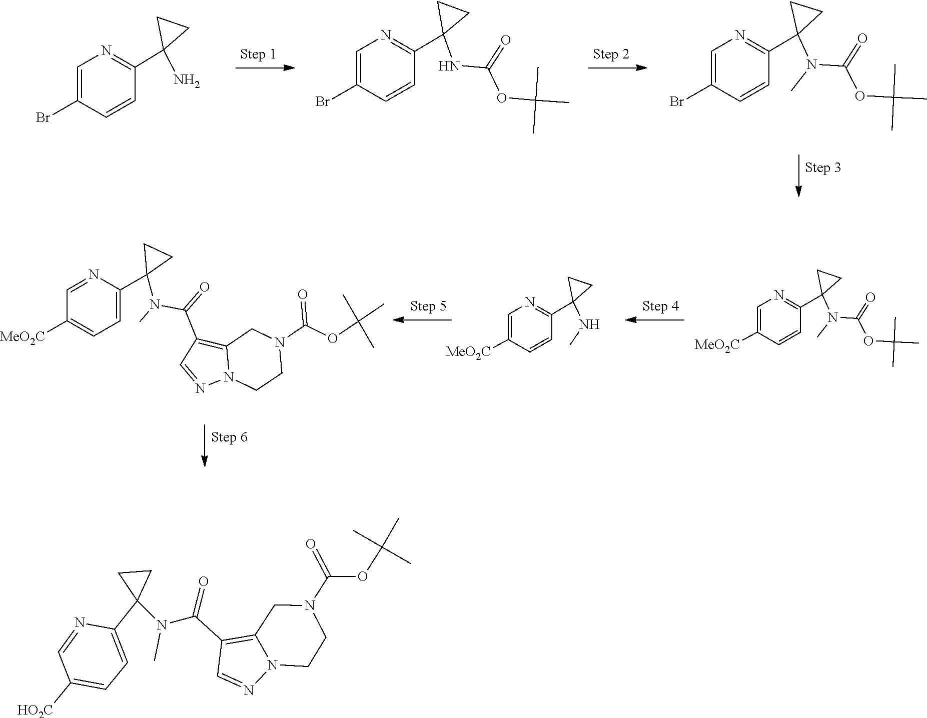

Step 1: To a cooled (0° C.) solution of 1-(5-bromopyridin-2-yl)cyclopropan-1-amine dihydrochloride (1.0 g, 3.5 mmol) in DCM (10 mL), was added di-tert-butyl dicarbonate (763.05 mg, 3.5 mmol). Triethylamine (778.33 mg, 7.69 mmol, 1.07 mL, 2.2 equiv.) was added dropwise and the mixture was stirred at room temperature overnight. The resulting mixture was diluted with water and the organic phase was separated. The organic layer was washed with water, dried over sodium sulfate, filtered and concentrated under reduced pressure to give tert-butyl N-[1-(5-bromopyridin-2-yl)cyclopropyl]carbamate (930.0 mg, 2.97 mmol, 84.9% yield).

Step 2: To a cooled (0° C.) solution of tert-butyl (1-(5-bromopyridin-2-yl)cyclopropyl)carbamate (930.0 mg, 2.97 mmol) in dry DMF (5 mL), was added sodium hydride (154.45 mg, 6.44 mmol). The mixture was stirred for 30 min, then iodomethane (632.45 mg, 4.46 mmol) was added dropwise. The reaction mixture was stirred at r.t. overnight. The resulting mixture was diluted with brine (10 mL) and extracted with EtOAc (3×10 mL). The combined organic extracts were washed with brine, dried over sodium sulfate, filtered and concentrated to give tert-butyl N-[1-(5-bromopyridin-2-yl)cyclopropyl]-N-methylcarbamate (1.0 g, 90.0% purity, 2.75 mmol, 92.6% yield) as yellow solid.

Step 3: To a solution of tert-butyl N-[1-(5-bromopyridin-2-yl)cyclopropyl]-N-methylcarbamate (997.6 mg, 3.05 mmol) in MeOH (50 mL) were added [1,1′-bis(diphenylphosphino)ferrocene]dichloropalladium(II), complex with dichloromethane (248.97 mg, 304.87 μmol) and triethylamine (370.26 mg, 3.66 mmol, 510.0 μL, 1.2 equiv.). The mixture was carbonylated at 135° C. and 40 atm for 20 h. The resulting mixture was cooled and concentrated to dryness. The residue was dissolved in EtOAc (20 mL) and the solution was washed with water (5 mL), dried over sodium sulfate, filtered and concentrated to give methyl 6-(1-[(tert-butoxy)carbonyl](methyl)aminocyclopropyl)pyridine-3-carboxylate (800.0 mg, 90.0% purity, 2.35 mmol, 77.1% yield) as brown solid, that was used in the next step without further purification.

Step 4: To a solution of methyl 6-(1-[(tert-butoxy)carbonyl](methyl)aminocyclopropyl)pyridine-3-carboxylate (800.28 mg, 2.61 mmol) in dry DCM (5 mL) was added 4M HCl in dioxane (4.5 ml, 10 mmol) was added. The reaction mixture was stirred overnight. The resulting mixture was concentrated under reduced pressure.

The obtained solid was used in the next step without additional purification.

Step 5: To a solution of 5-[(tert-butoxy)carbonyl]-4H,5H,6H,7H-pyrazolo[1,5-a]pyrazine-3-carboxylic acid (606.14 mg, 2.27 mmol) in dry DMF (3 mL) was added HATU (948.52 mg, 2.49 mmol). The resulting mixture was stirred for 10 mins, followed by the addition of methyl 6-[1-(methylamino)cyclopropyl]pyridine-3-carboxylate hydrochloride (550.4 mg, 2.27 mmol) and triethylamine (252.43 mg, 2.49 mmol, 350.0 μL, 1.1 equiv.). The reaction mixture was stirred overnight. The resulting mixture was partitioned between EtOAc (30 mL) and water (10 mL). The organic phase was washed with water (2×10 mL), brine, dried over sodium sulfate and concentrated. The residue was purified by HPLC to give methyl 6-(1-N-methyl-5-[(tert-butoxy)carbonyl]-4H,5H,6H,7H-pyrazolo[1,5-a]pyrazine-3-amidocyclopropyl)pyridine-3-carboxylate (320.0 mg, 702.51 μmol, 31% yield) as brown foam.

Step 6: To a solution of methyl 6-(1-N-methyl-5-[(tert-butoxy)carbonyl]-4H,5H,6H,7H-pyrazolo[1,5-a]pyrazine-3-amidocyclopropyl)pyridine-3-carboxylate (320.0 mg, 702.51 μmol) in THF-water (5 mL/1 mL) was added lithium hydroxide monohydrate (117.86 mg, 2.81 mmol). The mixture was stirred at r.t. overnight then concentrated under reduced pressure. The residue was dissolved in water (5 mL) and acidified with 5% aq. HCl to pH 3. The obtained precipitate was collected by filtration and air-dried to afford 6-(1-N-methyl-5-[(tert-butoxy)carbonyl]-4H,5H,6H,7H-pyrazolo[1,5-a]pyrazine-3-amidocyclopropyl)pyridine-3-carboxylic acid (195.0 mg, 441.7 μmol, 62.9% yield) as light brown solid.

then filtrate concentrated under reduced pressure to obtain 6,6-difluoro-4-azaspiro[2.4]heptane (0.8 g, 6.01 mmol, 50% yield).

### Synthesis of tert-butyl 3-{[(2R)-1,1,1-trifluoropropan-2-yl]carbamoyl}-4H,5H,6H,7H-pyrazolo[1,5-a]pyrazine-5-carboxylate

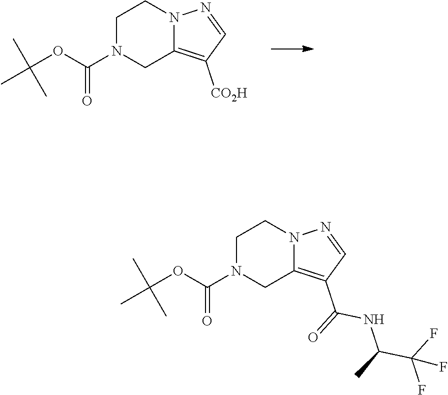 [SMILES: CC(C)(C)OC(=O)N1CCn2ncc(C(=O)O)c2C1 ; C[C@@H](NC(=O)c1cnn2c1CN(C(=O)OC(C)(C)C)CC2)C(F)(F)F]

To a solution of 5-[(tert-butoxy)carbonyl]-4H,5H,6H,7H-pyrazolo[1,5-a]pyrazine-3-carboxylic acid (804.39 mg, 3.01 mmol) and triethylamine (609.07 mg, 6.02 mmol, 840.0 μL) in dry DMF (30 mL) was added HATU (1.22 g, 3.21 mmol). The resulting mixture was stirred for 10 mins then (2R)-1,1,1-trifluoropropan-2-amine hydrochloride (300.0 mg, 2.01 mmol) was added and the stirring was continued overnight. The reaction mixture was partitioned between EtOAc (50 mL) and H₂O (300 mL). The organic phase was washed with H₂O (2×50 mL), brine, dried over sodium sulfate and concentrated under reduced pressure to give a viscous brown residue, which was purified by HPLC to give tert-butyl 3-[(2R)-1,1,1-trifluoropropan-2-yl]carbamoyl-4H,5H,6H,7H-pyrazolo[1,5-a]pyrazine-5-carboxylate (353.2 mg, 974.76 μmol, 48.6% yield).

¹H NMR (500 MHz, CDCl₃) δ 1.40 (d, 3H), 1.50 (s, 9H), 3.86 (m, 1H), 3.94 (m, 1H), 4.19 (m, 2H), 4.92 (m, 3H), 5.85 (m, 1H), 7.70 (s, 1H).

LCMS: m/z 363.4

### Example 1

2-(3-{4-azaspiro[2.4]heptane-4-carbonyl}-4H,5H,6H,7H-[1,2]oxazolo[4,5-c]pyridine-5-carbonyl)-1H-indole

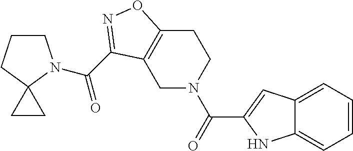 [SMILES: O=C(c1cc2ccccc2[nH]1)N1CCc2onc(C(=O)N3CCCC34CC4)c2C1]

Step 1: 5-(tert-butoxycarbonyl)-4,5,6,7-tetrahydroisoxazolo[4,5-c]pyridine-3-carboxylic acid (25 mg, 0.093 mmol) and HATU (42.5 mg, 0.112 mmol) were stirred in dry N,N-dimethylformamide (1 mL) for 10 minutes. This was added to a solution of 4-azaspiro[2.4]heptane hydrochloride (12.45 mg, 0.093 mmol) and triethylamine (0.065 mL, 0.466 mmol) in dry N,N-dimethylformamide (1 mL). The mixture was stirred at room temperature for 4 hours. The reaction was quenched by the addition of water (0.2 mL). The mixture was diluted with water (35 mL) and EtOAc (35 mL). The water layer was extracted with EtOAc (1×35 mL). The combined organic layer was washed with water (2×20 mL) and brine (20 mL). The organic layer was dried over Na₂SO₄ and concentrated in vacuo to give tert-butyl 3-(4-azaspiro[2.4]heptane-4-carbonyl)-6,7-dihydroisoxazolo[4,5-c]pyridine-5(4H)-carboxylate (0.033 g, 0.095 mmol, 102% yield).

Step 2: Tert-butyl 3-(4-azaspiro[2.4]heptane-4-carbonyl)-6,7-dihydroisoxazolo[4,5-c]pyridine-5(4H)-carboxylate (0.033 g, 0.095 mmol) was stirred in hydrochloric acid (4M in dioxane, 5 mL, 20.00 mmol). The mixture was stirred at room temperature for 2 hours. Solvents were evaporated in vacuo. The residue was stripped with CH₂Cl₂ (twice) to give (4-azaspiro[2.4]heptan-4-yl)(4,5,6,7-tetrahydroisoxazolo[4,5-c]pyridin-3-yl)methanone hydrochloride.

Step 3: 1H-indole-2-carboxylic acid (0.015 g, 0.095 mmol) and HATU (0.043 g, 0.114 mmol) were stirred in dry N,N-dimethylformamide (1 mL) for 10 minutes. In a separate vial (4-azaspiro[2.4]heptan-4-yl)(4,5,6,7-tetrahydroisoxazolo[4,5-c]pyridin-3-yl)methanone hydrochloride (0.027 g, 0.095 mmol) was dissolved in dry N,N-dimethylformamide (1 mL). To this triethylamine (0.066 mL, 0.476 mmol) was added. After 5 minutes the solution of acid was added. The mixture turns clear. The mixture was stirred at room temperature for 16 hours. The reaction was quenched with water (0.25 mL), filtered over a nylon filter and purified directly to give (5-(1H-indole-2-carbonyl)-4,5,6,7-tetrahydroisoxazolo[4,5-c]pyridin-3-yl)(4-azaspiro[2.4]heptan-4-yl)methanone (0.022 g, 0.056 mmol, 59.2% yield) as a white solid Rt (Method A) 3.51 mins, m/z 391 [M+H]⁺

1H NMR (400 MHz, DMSO-d6) δ 11.67 (s, 1H), 7.65 (d, J=7.9 Hz, 1H), 7.43 (d, J=8.1 Hz, 1H), 7.25-7.17 (m, 1H), 7.07 (t, J=7.5 Hz, 1H), 6.92 (s, 1H), 4.98-4.46 (m, 2H), 4.18-3.94 (m, 2H), 3.90-3.84 (m, 2H), 3.20-2.86 (m, 2H), 1.97-1.83 (m, 6H), 0.61-0.51 (m, 2H).

### Example 2

### 5-(1H-indole-2-carbonyl)-N-methyl-N-[1-(1,3-oxazol-4-yl)cyclopropyl]-4H,5H,6H,7H-[1,2]oxazolo[4,5-c]pyridine-3-carboxamide

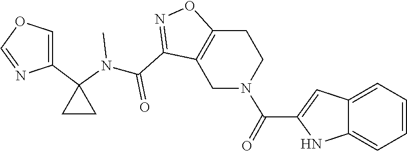 [SMILES: CN(C(=O)c1noc2c1CN(C(=O)c1cc3ccccc3[nH]1)CC2)C1(c2cocn2)CC1]

Step 1: 5-(tert-butoxycarbonyl)-4,5,6,7-tetrahydroisoxazolo[4,5-c]pyridine-3-carboxylic acid (25 mg, 0.093 mmol) and HATU (42.5 mg, 0.112 mmol) were stirred in dry N,N-dimethylformamide (1 mL) for 10 minutes. This was added to a solution of N-methyl-1-(oxazol-4-yl)cyclopropan-1-amine hydrochloride (17.90 mg, 0.103 mmol) and triethylamine (0.065 mL, 0.466 mmol) in dry N,N-dimethylformamide (1 mL). The mixture was stirred at room temperature for 16 hours. The reaction was quenched by the addition of water (0.2 mL) and diluted with water (35 mL) and EtOAc (35 mL). The water layer was extracted with EtOAc (35 mL). The combined organic extracts were washed with water (2×20 mL) and brine (20 mL).

The organic layer was dried over Na₂SO₄ and concentrated in vacuo to obtain tert-butyl 3-(methyl(1-(oxazol-4-yl)cyclopropyl)carbamoyl)-6,7-dihydroisoxazolo[4,5-c]pyridine-5(4H)-carboxylate (0.035 g, 0.090 mmol, 97% yield).

Step 2: Tert-butyl 3-(methyl(1-(oxazol-4-yl)cyclopropyl)carbamoyl)-6,7-dihydroisoxazolo[4,5-c]pyridine-5(4H)-carboxylate (0.036 g, 0.093 mmol) was stirred in hydrochloric acid (4M in dioxane, 5 mL, 20.00 mmol). The mixture was stirred at room temperature for 2 hours. Solvents were evaporated in vacuo. The residue was stripped with CH₂Cl₂ (twice) to give Nmethyl-N-(1-(oxazol-4-yl)cyclopropyl)-4,5,6,7-tetrahydroisoxazolo[4,5-c]pyridine-3-carboxamide hydrochloride that was used without further purification.

Step 3: In a vial 1H-indole-2-carboxylic acid (0.015 g, 0.092 mmol) and HATU (0.042 g, 0.111 mmol) were stirred in dry N,N-dimethylformamide (1 mL) for 10 minutes. In a separate vial N-methyl-N-(1-(oxazol-4-yl)cyclopropyl)-4,5,6,7-tetrahydroisoxazolo[4,5-c]pyridine-3-carboxamide hydrochloride (0.030 g, 0.092 mmol) was dissolved in dry N,N-dimethylformamide (1 mL). To this triethylamine (0.064 mL, 0.462 mmol) was added. After 5 minutes the solution of acid was added. The mixture turns clear. The mixture was stirred at room temperature for 16 hours. The reaction was quenched with water (0.25 mL), filtered over a nylon filter and purified by HPLC to give 5-(1H-indole-2-carbonyl)-N-methyl-N-(1-(oxazol-4-yl)cyclopropyl)-4,5,6,7-tetrahydroisoxazolo[4,5-c]pyridine-3-carboxamide (0.035 g, 0.081 mmol, 88% yield) as a white solid.

Rt (Method A) 3.17 mins, m/z 432 [M+H]⁺

1H NMR (400 MHz, DMSO-d6) δ 11.71-11.52 (m, 1H), 8.35-8.11 (m, 1H), 8.09-7.92 (m, 1H), 7.69-7.59 (m, 1H), 7.47-7.39 (m, 1H), 7.25-7.16 (m, 1H), 7.11-7.01 (m, 1H), 6.95-6.86 (m, 1H), 5.15-4.33 (m, 2H), 4.22-3.86 (m, 2H), 3.32-3.28 (m, 1H), 3.20-2.90 (m, 4H), 1.41-1.12 (m, 4H).

### Example 3

### 2-(3-{6,6-difluoro-4-azaspiro[2.4]heptane-4-carbonyl}-4H,5H,6H,7H-[1,2]oxazolo[4,5-c]pyridine-5-carbonyl)-1H-indole

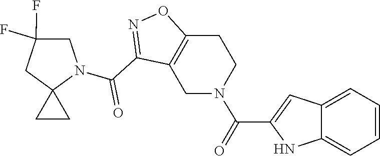 [SMILES: O=C(c1cc2ccccc2[nH]1)N1CCc2onc(C(=O)N3CC(F)(F)CC34CC4)c2C1]

Step 1: 5-(tert-butoxycarbonyl)-4,5,6,7-tetrahydroisoxazolo[4,5-c]pyridine-3-carboxylic acid (25 mg, 0.093 mmol) and HATU (42.5 mg, 0.112 mmol) were stirred in dry N,N-dimethylformamide (1 mL) for 10 minutes. This was then added to a solution of 6,6-difluoro-4-azaspiro[2.4]heptane hydrochloride (17.39 mg, 0.103 mmol) and triethylamine (0.065 mL, 0.466 mmol) in dry N,N-dimethylformamide (1 mL). The mixture was stirred at room temperature for 16 hours, then quenched by the addition of water (0.2 mL). The mixture was diluted with water (35 mL) and EtOAc (35 mL). The water layer was extracted with EtOAc (35 mL). The combined organic extracts were washed with water (2×20 mL) and brine (20 mL).

The organic layer was dried over Na₂SO₄ and concentrated in vacuo to give tert-butyl 3-(6,6-difluoro-4-azaspiro[2.4]heptane-4-carbonyl)-6,7-dihydroisoxazolo[4,5-c]pyridine-5(4H)-carboxylate (0.034 g, 0.089 mmol, 95% yield).

Step 2: Tert-butyl 3-(6,6-difluoro-4-azaspiro[2.4]heptane-4-carbonyl)-6,7-dihydroisoxazolo[4,5-c]pyridine-5(4H)-carboxylate (0.034 g, 0.089 mmol) was stirred in hydrochloric acid (4M in dioxane, 5 mL, 20.00 mmol). The mixture was stirred at room temperature for 2 hours. Solvents were evaporated in vacuo. The residue was stripped with CH₂Cl₂ (twice) to give (6,6-difluoro-4-azaspiro[2.4]heptan-4-yl)(4,5,6,7-tetrahydroisoxazolo[4,5-c]pyridin-3-yl)methanone hydrochloride that was used in the next step without further purification.

Step 3: 1H-indole-2-carboxylic acid (0.014 g, 0.088 mmol) and HATU (0.040 g, 0.105 mmol) were stirred in dry N,N-dimethylformamide (1 mL) for 10 minutes. In a separate vial (6,6-difluoro-4-azaspiro[2.4]heptan-4-yl)(4,5,6,7-tetrahydroisoxazolo[4,5-c]pyridin-3-yl)methanone hydrochloride (0.028 g, 0.088 mmol) was dissolved in dry N,N-dimethylformamide (1 mL). To this was added triethylamine (0.061 mL, 0.438 mmol) was added. After 5 minutes the solution of acid was added. The mixture was stirred at room temperature for 4 hours, then quenched with water (0.25 mL), filtered over a nylon filter. The product was purified directly by HPLC to give (5-(1H-indole-2-carbonyl)-4,5,6,7-tetrahydroisoxazolo[4,5-c]pyridin-3-yl)(6,6-difluoro-4-azaspiro[2.4]heptan-4-yl)methanone (0.017 g, 0.040 mmol, 45.5% yield) as a white solid.

Rt (Method A) 3.6 mins, m/z 427 [M+H]⁺

1H NMR (400 MHz, DMSO-d6) δ 11.66 (s, 1H), 7.65 (d, J=7.8 Hz, 1H), 7.43 (d, J=8.2 Hz, 1H), 7.20 (ddd, J=8.1, 6.9, 1.2 Hz, 1H), 7.06 (dd, J=8.0, 6.7 Hz, 1H), 6.92 (s, 1H), 4.93-4.56 (m, 2H), 4.36 (t, J=12.9 Hz, 2H), 4.13-3.93 (m, 2H), 3.15-2.93 (m, 2H), 2.61-2.53 (m, 2H), 2.05-1.82 (m, 2H), 0.77-0.67 (m, 2H).

### Example 4

### 5-(4-ethyl-6-fluoro-1H-indole-2-carbonyl)-N-[1-(methoxymethyl)cyclopropyl]-N-methyl-4H,5H,6H,7H-[1,2]oxazolo[4,5-c]pyridine-3-carboxamide

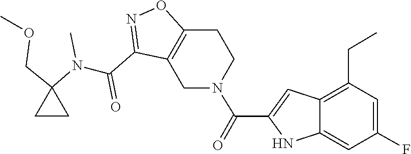 [SMILES: CCc1cc(F)cc2[nH]c(C(=O)N3CCc4onc(C(=O)N(C)C5(COC)CC5)c4C3)cc12]

Step 1: 5-(tert-butoxycarbonyl)-4,5,6,7-tetrahydroisoxazolo[4,5-c]pyridine-3-carboxylic acid (0.2 g, 0.746 mmol) and HATU (0.340 g, 0.895 mmol) were stirred in N,N-dry dimethylformamide (1 mL) for 10 minutes. This mixture was then added to a solution of 1-(methoxymethyl)-N-methylcyclopropan-1-amine hydrochloride (0.124 g, 0.820 mmol) and triethylamine (0.520 mL, 3.73 mmol) in dry N,N-dimethylformamide (1 mL). The mixture was stirred at room temperature for 16 hours then quenched by the addition of water (0.2 mL). The product was purified directly by HPLC to give tert-butyl 3-((1-(methoxymethyl)cyclopropyl)(methyl)carbamoyl)-6,7-dihydroisoxazolo[4,5-c]pyridine-5(4H)-carboxylate (0.211 g, 0.577 mmol, 77% yield).

Step 2: tert-butyl 3-((1-(methoxymethyl)cyclopropyl)(methyl)carbamoyl)-6,7-dihydroisoxazolo[4,5-c]pyridine-5(4H)-carboxylate (0.211 g, 0.577 mmol) was stirred in hydrochloric acid, 4N in dioxane (5 mL, 20.00 mmol). The mixture was stirred at room temperature for 2 hours. Solvents were evaporated in vacuo. The residue was stripped with CH₂Cl₂ (twice) to obtain N-(1-(methoxymethyl)cyclopropyl)-N-methyl-4,5,6,7-tetrahydroisoxazolo[4,5-c]pyridine-3-carboxamide hydrochloride that was used in the next step without further purification.

Step 3: 4-ethyl-6-fluoro-1H-indole-2-carboxylic acid (0.024 g, 0.116 mmol) and HATU dr(0.053 g, 0.139 mmol) were stirred in dry dry N,N-dimethylformamide (1 mL) for 10 minutes.

In a separate vial N-(1-(methoxymethyl)cyclopropyl)-N-methyl-4,5,6,7-tetrahydroisoxazolo[4,5-c]pyridine-3-carboxamide hydrochloride (0.035 g, 0.116 mmol) was dissolved in dry N,N-dimethylformamide (1 mL). To this was added triethylamine (0.081 mL, 0.580 mmol). After 5 minutes the solution of acid was added. The mixture was stirred at room temperature for 2 hours. The reaction was quenched with water (0.25 mL). The product was purified by directly by HPLC to give 5-(4-ethyl-6-fluoro-1H-indole-2-carbonyl)-N-(1-(methoxymethyl)cyclopropyl)-N-methyl-4,5,6,7-tetrahydroisoxazolo[4,5-c]pyridine-3-carboxamide (0.029 g, 0.064 mmol, 55.0% yield).

Rt (Method A) 3.61 mins, m/z 455 [M+H]⁺

1H NMR (400 MHz, DMSO-d6) δ 11.71 (s, 1H), 7.02-6.93 (m, 2H), 6.78 (dd, J=10.8, 2.3 Hz, 1H), 4.97-4.35 (m, 2H), 4.17-3.79 (m, 2H), 3.28-3.15 (m, 4H), 3.12-2.99 (m, 4H), 2.89 (q, J=7.5 Hz, 2H), 1.31-1.24 (m, 3H), 0.95-0.64 (m, 4H).

### Example 5

### 5-(4-chloro-1H-indole-2-carbonyl)-N-[1-(methoxymethyl)cyclopropyl]-N-methyl-4H,5H,6H,7H-[1,2]oxazolo[4,5-c]pyridine-3-carboxamide

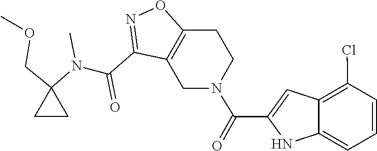 [SMILES: COCC1(N(C)C(=O)c2noc3c2CN(C(=O)c2cc4c(Cl)cccc4[nH]2)CC3)CC1]

Rt (Method A) 3.49 mins, m/z 443/445 [M+H]⁺

1H NMR (400 MHz, DMSO-d6) δ 12.06 (s, 1H), 7.41 (dd, J=8.0, 2.7 Hz, 1H), 7.24-7.12 (m, 2H), 6.89 (s, 1H), 4.99-4.48 (m, 2H), 4.19-3.92 (m, 2H), 3.28-3.16 (m, 4H), 3.05 (s, 4H), 0.94-0.71 (m, 4H). 4-chloro-1H-indole-2-carboxylic acid (0.023 g, 0.116 mmol) and HATU (0.053 g, 0.139 mmol) were stirred in dry N,N-dimethylformamide (1 mL) for 10 minutes. In a separate vial N-(1-(methoxymethyl)cyclopropyl)-N-methyl-4,5,6,7-tetrahydroisoxazolo[4,5-c]pyridine-3-carboxamide hydrochloride (0.035 g, 0.116 mmol) was dissolved in dry N,N-dimethylformamide (1 mL). To this was added triethylamine (0.081 mL, 0.580 mmol). After 5 minutes the solution of acid was added. The mixture was stirred at room temperature for 2 hours. The reaction was quenched with water (0.25 mL) and purified directly by HPLC to obtain 5-(4-chloro-1H-indole-2-carbonyl)-N-(1-(methoxymethyl)cyclopropyl)-N-methyl-4,5,6,7-tetrahydroisoxazolo[4,5-c]pyridine-3-carboxamide (0.031 g, 0.070 mmol, 60.3% yield).

### Example 6

### 5-(4,6-difluoro-1H-indole-2-carbonyl)-N-[1-(methoxymethyl)cyclopropyl]-N-methyl-4H,5H,6H,7H-[1,2]oxazolo[4,5-c]pyridine-3-carboxamide

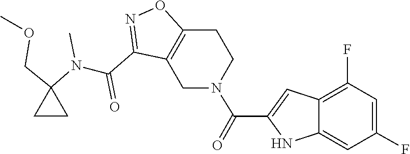 [SMILES: COCC1(N(C)C(=O)c2noc3c2CN(C(=O)c2cc4c(F)cc(F)cc4[nH]2)CC3)CC1]

4,6-difluoro-1H-indole-2-carboxylic acid (0.023 g, 0.116 mmol) and HATU (0.053 g, 0.139 mmol) were stirred in dry N,N-dimethylformamide (1 mL) for 10 minutes. In a separate vial N-(1-(methoxymethyl)cyclopropyl)-N-methyl-4,5,6,7-tetrahydroisoxazolo[4,5-c]pyridine-3-carboxamide hydrochloride (0.035 g, 0.116 mmol) was dissolved in dry N,N-dimethylformamide (1 mL). To this was added triethylamine (0.081 mL, 0.580 mmol). After 5 minutes the solution of acid was added. The mixture was stirred at room temperature for 2 hours. The reaction was quenched with water (0.25 mL) and purified directly by HPLC to give 5-(4,6-difluoro-1H-indole-2-carbonyl)-N-(1-(methoxymethyl)cyclopropyl)-N-methyl-4,5,6,7-tetrahydroisoxazolo[4,5-c]pyridine-3-carboxamide (0.036 g, 0.081 mmol, 69.8% yield).

Rt (Method A) 3.44 mins, m/z 445 [M+H]⁺

1H NMR (400 MHz, DMSO-d6) δ 12.02 (s, 1H), 7.07-6.97 (m, 2H), 6.92 (td, J=10.4, 2.1 Hz, 1H), 5.06-4.32 (m, 2H), 4.27-3.81 (m, 2H), 3.28-3.17 (m, 4H), 3.12-2.96 (m, 4H), 1.02-0.64 (m, 4H).

### Example 7

### 5-(1H-indole-2-carbonyl)-N-[1-(methoxymethyl)cyclopropyl]-N-methyl-4H,5H,6H,7H-[1,2]oxazolo[4,5-c]pyridine-3-carboxamide

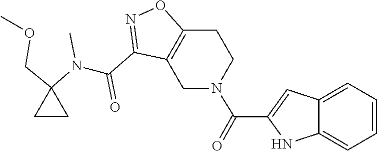 [SMILES: COCC1(N(C)C(=O)c2noc3c2CN(C(=O)c2cc4ccccc4[nH]2)CC3)CC1]

1H-indole-2-carboxylic acid (0.019 g, 0.116 mmol) and HATU (0.053 g, 0.139 mmol) were stirred in dry N,N-dimethylformamide (1 mL) for 10 minutes. In a separate vial N-(1-(methoxymethyl)cyclopropyl)-N-methyl-4,5,6,7-tetrahydroisoxazolo[4,5-c]pyridine-3-carboxamide hydrochloride (0.035 g, 0.116 mmol) was dissolved in dry N,N-dimethylformamide (1 mL). To thiswas added triethylamine (0.081 mL, 0.580 mmol). After 5 minutes the solution of acid was added. The mixture was stirred at room temperature for 2 hours. The reaction was quenched with water (0.25 mL) and purified directly by HPLC to give 5-(1H-indole-2-carbonyl)-N-(1-(methoxymethyl)cyclopropyl)-N-methyl-4,5,6,7-tetrahydroisoxazolo[4,5-c]pyridine-3-carboxamide (0.037 g, 0.091 mmol, 78% yield).

Rt (Method A) 3.28 mins, m/z 409 [M+H]⁺

1H NMR (400 MHz, DMSO-d6) δ 11.66 (s, 1H), 7.64 (d, J=8.0 Hz, 1H), 7.43 (d, J=8.3 Hz, 1H), 7.20 (t, J=7.6 Hz, 1H), 7.06 (t, J=7.5 Hz, 1H), 6.92 (s, 1H), 5.03-4.37 (m, 2H), 4.16-3.95 (m, 2H), 3.29-3.15 (m, 4H), 3.13-2.92 (m, 4H), 0.94-0.69 (m, 4H).

### Example 8

### N-[I1-(hydroxymethyl)cyclopropyl]-5-(1H-indole-2-carbonyl)-N-methyl-4H,5H,6H,7H-[1,2]oxazolo[4,5-c]pyridine-3-carboxamide

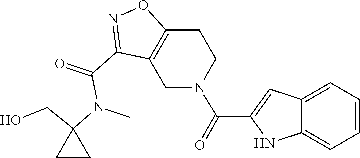 [SMILES: CN(C(=O)c1noc2c1CN(C(=O)c1cc3ccccc3[nH]1)CC2)C1(CO)CC1]

Step 1: 5-(tert-butoxycarbonyl)-4,5,6,7-tetrahydroisoxazolo[4,5-c]pyridine-3-carboxylic acid (40 mg, 0.149 mmol) was dissolved in dimethyl sulfoxide (dry) (0.5 mL) and HATU (62.4 mg, 0.164 mmol) was added. The mixture was stirred for 10 min. In a separate vial, (1-(methylamino)cyclopropyl)methyl benzoate hydrochloride (36.0 mg, 0.149 mmol) was dissolved in dimethyl sulfoxide (dry) (0.500 mL) and triethylamine (0.104 mL, 0.746 mmol) was added. The mixtures were combined and stirred for 1 h. The reaction mixture was partitioned between 10 mL EtOAc and 10 mL water. NaCl and some brine was added to separate the layers. The layers were separated and the aqueous layer was extracted with EtOAc (10 mL). The combined organic layers were washed with brine (4×10 mL), dried with Na₂SO₄ and concentrated to give tert-butyl 3-((1-((benzoyloxy)methyl)cyclopropyl)(methyl)carbamoyl)-6,7-dihydroisoxazolo[4,5-c]pyridine-5(4H)-carboxylate (64 mg, 0.124 mmol, 83% yield).

Step 2: Tert-butyl 3-((1-((benzoyloxy)methyl)cyclopropyl)(methyl)carbamoyl)-6,7-dihydroisoxazolo[4,5-c]pyridine-5(4H)-carboxylate (64 mg, 0.124 mmol) was dissolved in HCl (4 M in dioxane) (1 mL, 4.00 mmol) and the mixture was stirred for 1 h. The reaction mixture was concentrated and stripped with DCM to give (1-(N-methyl-4,5,6,7-tetrahydroisoxazolo[4,5-c]pyridine-3-carboxamido)cyclopropyl)methyl benzoate hydrochloride that was used in the next step without further purification.

Step 3: Indole-2-carboxylic acid (19.74 mg, 0.122 mmol) was dissolved in dimethyl sulfoxide (dry) (0.5 mL) and HATU (51.2 mg, 0.135 mmol) was added. In a separate vial, (1-(N-methyl-4,5,6,7-tetrahydroisoxazolo[4,5-c]pyridine-3-carboxamido)cyclopropyl)methyl benzoate hydrochloride (48 mg, 0.122 mmol) was dissolved in dimethyl sulfoxide (dry) (0.500 mL) and triethylamine (0.085 mL, 0.612 mmol) was added. A drop of water were added and the mixture was purified by HPLC to give (1-(5-(1H-indole-2-carbonyl)-N-methyl-4,5,6,7-tetrahydroisoxazolo[4,5-c]pyridine-3-carboxamido)cyclopropyl)methyl benzoate (30 mg, 0.060 mmol, 49.1% yield).

Step 4: (1-(5-(1H-indole-2-carbonyl)-N-methyl-4,5,6,7-tetrahydroisoxazolo[4,5-c]pyridine-3-carboxamido)cyclopropyl)methyl benzoate (20.5 mg, 0.041 mmol) was dissolved in tetrahydrofuran (0.5 mL) and a solution of lithium hydroxide monohydrate (6.90 mg, 0.164 mmol) in water (0.500 mL) was added. The mixture was stirred at 60° C. for 2.5 h. The reaction mixture was neutralized with 1M HCl (0.15 mL) and purified by HPLC to give N-(1-(hydroxymethyl)cyclopropyl)-5-(1H-indole-2-carbonyl)-N-methyl-4,5,6,7-tetrahydroisoxazolo[4,5-c]pyridine-3-carboxamide (4.1 mg, 10.39 μmol, 25.3% yield) Rt (Method A) 2.94 mins, m/z 395 [M+H]⁺

1H NMR (400 MHz, DMSO-d6) δ 11.66 (s, 1H), 7.70-7.59 (m, 1H), 7.43 (d, J=8.2 Hz, 1H), 7.21 (t, J=7.6 Hz, 1H), 7.06 (t, J=7.5 Hz, 1H), 6.98-6.89 (m, 1H), 5.06-3.51 (m, 7H), 3.24-3.18 (m, 1H), 3.16-2.94 (m, 4H), 0.92-0.62 (m, 4H).

### Example 9

### {1-[N-methyl-5-(1H-indole-2-carbonyl)-4H,5H,6H,7H-[1,2]oxazolo[4,5-c]pyridine-3-amido]cyclopropyl}methyl benzoate

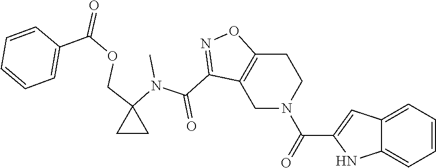 [SMILES: CN(C(=O)c1noc2c1CN(C(=O)c1cc3ccccc3[nH]1)CC2)C1(COC(=O)c2ccccc2)CC1]

Indole-2-carboxylic acid (19.74 mg, 0.122 mmol) was dissolved in dimethyl sulfoxide (dry) (0.5 mL) and HATU (51.2 mg, 0.135 mmol) was added. In a separate vial, (1-(N-methyl-4,5,6,7-tetrahydroisoxazolo[4,5-c]pyridine-3-carboxamido)cyclopropyl)methyl benzoate hydrochloride (48 mg, 0.122 mmol) was dissolved in dimethyl sulfoxide (dry) (0.500 mL) and triethylamine (0.085 mL, 0.612 mmol) was added. A drop of water were added and the mixture was purified by HPLC to give (1-(5-(1H-indole-2-carbonyl)-N-methyl-4,5,6,7-tetrahydroisoxazolo[4,5-c]pyridine-3-carboxamido)cyclopropyl)methyl benzoate (30 mg, 0.060 mmol, 49.1% yield).

Rt (Method A) 3.72 mins, m/z 399 [M+H]⁺

1H NMR (400 MHz, DMSO-d6) δ 11.66 (s, 1H), 8.02-7.95 (m, 2H), 7.72-7.59 (m, 2H), 7.58-7.48 (m, 2H), 7.43 (d, J=8.2 Hz, 1H), 7.24-7.17 (m, 1H), 7.10-7.03 (m, 1H), 6.96-6.85 (m, 1H), 5.28-3.85 (m, 6H), 3.27-3.24 (m, 1H), 3.17-2.95 (m, 4H), 1.16-0.85 (m, 4H).

### Example 10

### N-cyclopropyl-5-(1H-indole-2-carbonyl)-4H,5H,6H,7H-[1,2]oxazolo[4,5-c]pyridine-3-carboxamide

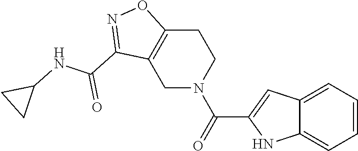 [SMILES: O=C(NC1CC1)c1noc2c1CN(C(=O)c1cc3ccccc3[nH]1)CC2]

Step 1: 5-(tert-butoxycarbonyl)-4,5,6,7-tetrahydroisoxazolo[4,5-c]pyridine-3-carboxylic acid (0.025 g, 0.093 mmol) and HATU (0.043 g, 0.112 mmol) were stirred in dry N,N-dimethylformamide (1 mL) for 10 minutes. This mixture was then added to a solution of cyclopropylamine (6.46 μL, 0.093 mmol) and triethylamine (0.065 mL, 0.466 mmol) in dry N,N-dimethylformamide (1 mL). The mixture was stirred at room temperature for 16 hours.

Additional cyclopropylamine (5.32 mg, 0.093 mmol) was added. The mixture was stirred for a further 1 hour. The reaction was quenched by the addition of water (0.2 mL) and purified directly by HPLC to give tert-butyl 3-(cyclopropylcarbamoyl)-6,7-dihydroisoxazolo[4,5-c]pyridine-5(4H)-carboxylate (0.028 g, 0.091 mmol, 98% yield).

Step 2: Tert-butyl 3-(cyclopropylcarbamoyl)-6,7-dihydroisoxazolo[4,5-c]pyridine-5(4H)-carboxylate (0.028 g, 0.091 mmol) was stirred in hydrochloric acid (4M in dioxane, 5 mL, 20.00 mmol). The mixture was stirred at room temperature for 2 hours. Solvents were evaporated in vacuo. The residue was stripped with CH₂Cl₂ (twice) to give N-cyclopropyl-4,5,6,7-tetrahydroisoxazolo[4,5-c]pyridine-3-carboxamide hydrochloride that was usewd in the next step without further purification.

Step 3: 1H-indole-2-carboxylic acid (0.015 g, 0.090 mmol) and HATU (0.041 g, 0.108 mmol) were stirred in dry N,N-dimethylformamide (1 mL) for 10 minutes. In a separate vial N-cyclopropyl-4,5,6,7-tetrahydroisoxazolo[4,5-c]pyridine-3-carboxamide hydrochloride (0.022 g, 0.090 mmol) was dissolved in dry N,N-dimethylformamide (1 mL). To this was added triethylamine (0.063 mL, 0.451 mmol). After 5 minutes the solution of acid was added. The mixture was stirred at room temperature for 16 hours. The reaction was quenched with water (0.25 mL). The product was purified by directly by HPLC to give N-cyclopropyl-5-(1H-indole-2-carbonyl)-4,5,6,7-tetrahydroisoxazolo[4,5-c]pyridine-3-carboxamide (0.013 g, 0.037 mmol, 41.1% yield).

Rt (Method A) 3.1 mins, m/z 351 [M+H]⁺

1H NMR (400 MHz, DMSO-d6) δ 11.67 (s, 1H), 8.89 (d, J=4.0 Hz, 1H), 7.65 (d, J=7.8 Hz, 1H), 7.43 (d, J=8.3 Hz, 1H), 7.21 (t, J=7.6 Hz, 1H), 7.07 (t, J=7.4 Hz, 1H), 6.93 (s, 1H), 5.04-4.67 (m, 2H), 4.20-3.89 (m, 2H), 3.18-2.76 (m, 3H), 0.88-0.44 (m, 4H).

### Example 11

### N-cyclopropyl-5-(1H-indole-2-carbonyl)-N-methyl-4H,5H,6H,7H-[1,2]oxazolo[4,5-c]pyridine-3-carboxamide

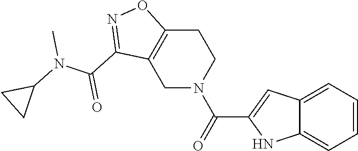 [SMILES: CN(C(=O)c1noc2c1CN(C(=O)c1cc3ccccc3[nH]1)CC2)C1CC1]

Step 1: 5-(tert-butoxycarbonyl)-4,5,6,7-tetrahydroisoxazolo[4,5-c]pyridine-3-carboxylic acid (0.025 g, 0.093 mmol) and HATU (0.043 g, 0.112 mmol) were stirred in dry N,N-dimethylformamide (1 mL) for 10 minutes. This solution was then added to a solution of N-cyclopropyl-methylamine hydrochloride (10.03 mg, 0.093 mmol) and triethylamine (0.065 mL, 0.466 mmol) in N,N-dimethylformamide (dry) (1 mL). The mixture was stirred at room temperature for 16 hours, then quenched by the addition of water (0.2 mL). The product was purified directly by HPLC to give tert-butyl 3-(cyclopropyl(methyl)carbamoyl)-6,7-dihydroisoxazolo[4,5-c]pyridine-5(4H)-carboxylate (0.033 g, 0.103 mmol, 110% yield).

Step 2: Tert-butyl 3-(cyclopropyl(methyl)carbamoyl)-6,7-dihydroisoxazolo[4,5-c]pyridine-5(4H)-carboxylate (0.033 g, 0.103 mmol) was stirred in hydrochloric acid (4M in dioxane, 5 mL, 20.00 mmol). The mixture was stirred at room temperature for 2 hours. Solvents were evaporated in vacuo. The residue was stripped with CH₂Cl₂ (twice) to give N-cyclopropyl-N-methyl-4,5,6,7-tetrahydroisoxazolo[4,5-c]pyridine-3-carboxamidehydrochloride that was used in the next step without further purification.

Step 3: 1H-indole-2-carboxylic acid (0.016 g, 0.101 mmol) and HATU (0.046 g, 0.121 mmol) were stirred in dry N,N-dimethylformamide (1 mL) for 10 minutes. In a separate vial N-cyclopropyl-N-methyl-4,5,6,7-tetrahydroisoxazolo[4,5-c]pyridine-3-carboxamide hydrochloride (0.026 g, 0.101 mmol) was dissolved in dry N,N-dimethylformamide (1 mL). To this was added triethylamine (0.070 mL, 0.504 mmol). After 5 minutes the solution of acid was added. The mixture was stirred at room temperature for 16 hours, and then quenched with water (0.25 mL). The product was purified directly by HPLC to give N-cyclopropyl-5-(1H-indole-2-carbonyl)-N-methyl-4,5,6,7-tetrahydroisoxazolo[4,5-c]pyridine-3-carboxamide (0.029 g, 0.080 mmol, 79% yield).

Rt (Method A) 3.16 mins, m/z 365 [M+H]⁺

1H NMR (400 MHz, DMSO-d6) δ 11.66 (s, 1H), 7.64 (d, J=8.1 Hz, 1H), 7.43 (d, J=8.2 Hz, 1H), 7.21 (t, J=7.5 Hz, 1H), 7.07 (t, J=7.4 Hz, 1H), 6.93 (s, 1H), 4.92-4.53 (m, 2H), 4.17-3.95 (m, 2H), 3.15-2.80 (m, 6H), 0.85-0.48 (m, 4H).

### Example 12

### 4-{1-[N-methyl-5-(1H-indole-2-carbonyl)-4H,5H,6H,7H-pyrazolo[1,5-a]pyrazine-3-amido]cyclopropyl}benzoic acid

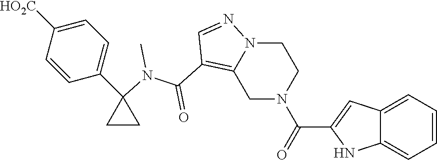 [SMILES: CN(C(=O)c1cnn2c1CN(C(=O)c1cc3ccccc3[nH]1)CC2)C1(c2ccc(C(=O)O)cc2)CC1]

Step 1: 4-(1-(5-(tert-butoxycarbonyl)-N-methyl-4,5,6,7-tetrahydropyrazolo[1,5-a]pyrazine-3-carboxamido)cyclopropyl)benzoic acid (100 mg, 0.227 mmol) was dissolved in HCl (4M in dioxane) (1.419 mL, 5.68 mmol) and the resulting light brown solution was stirred at rt. LCMS after 1 h. Further dioxane (0.3 mL) was added and the mixture was stirred for a further 1 h. The reaction mixture was diluted with dioxane (6 mL) and concentrated. Co-evaporation with toluene (2×6 mL) gave 4-(1-(N-methyl-4,5,6,7-tetrahydropyrazolo[1,5-a]pyrazine-3-carboxamido)cyclopropyl)benzoic acid hydrochloride as an off-white solid that was used in the next step without further purification.

Step 2: To a solution of 1H-indole-2-carboxylic acid (20.53 mg, 0.127 mmol) in dimethyl sulfoxide (0.6 mL) was added HATU (53.3 mg, 0.140 mmol). Tthe resulting solution was stirred at r.t. for 45 min. A mixture of 4-(1-(N-methyl-4,5,6,7-tetrahydropyrazolo[1,5-a]pyrazine-3-carboxamido)cyclopropyl)benzoic acid hydrochloride (48 mg, 0.127 mmol) and triethylamine (0.089 mL, 0.637 mmol) in dimethyl sulfoxide (0.6 mL) was then added and the mixture stirred at r.t. for five days. The reaction mixture was then filtered and purified directly by HPLC to give 4-(1-(5-(1H-indole-2-carbonyl)-N-methyl-4,5,6,7-tetrahydropyrazolo[1,5-a]pyrazine-3-carboxamido)cyclopropyl)benzoic acid (0.015 g, 24% yield) as a white solid.

Rt (Method A) 2.45 mins, m/z 484 [M+H]⁺

1H NMR (400 MHz, DMSO-d6) δ 11.70 (d, J=2.2 Hz, 1H), 7.92 (d, J=8.0 Hz, 2H), 7.66 (d, J=8.0 Hz, 1H), 7.44 (d, J=8.2 Hz, 1H), 7.26-7.19 (m, 1H), 7.15 (d, J=8.1 Hz, 2H), 7.07 (t, J=7.5 Hz, 1H), 6.95 (s, 2H), 5.22 (s, 2H), 4.27 (m, 3H), 4.09 (s, 1H), 3.57 (s, 1H), 3.04 (s, 2H), 1.62 (m, 2H), 1.42 (m, 2H).

### Example 13

### 3-{1-[N-methyl-5-(1H-indole-2-carbonyl)-4H,5H,6H,7H-pyrazolo[1,5-a]pyrazine-3-amido]cyclopropyl}benzoic acid

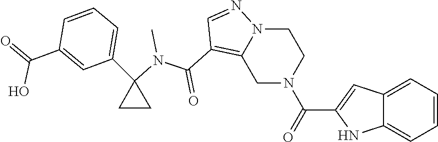 [SMILES: CN(C(=O)c1cnn2c1CN(C(=O)c1cc3ccccc3[nH]1)CC2)C1(c2cccc(C(=O)O)c2)CC1]

Step 1: 3-(1-(5-(tert-butoxycarbonyl)-N-methyl-4,5,6,7-tetrahydropyrazolo[1,5-a]pyrazine-3-carboxamido)cyclopropyl)benzoic acid (112 mg, 0.254 mmol) was dissolved in 4M HCl in dioxane (1.6 mL, 6.40 mmol) and the resulting solution was stirred at r.t. for 4 h. The reaction mixture was diluted with dioxane (4 mL) and concentrated. The residue was co-evaporated with toluene (2×10 mL) to give 3-(1-(N-methyl-4,5,6,7-tetrahydropyrazolo[1,5-a]pyrazine-3-carboxamido)cyclopropyl)benzoic acid hydrochloride (0.085 g, 89% yield) as an off-white solid.

Step 2: To a solution of 1H-indole-2-carboxylic acid (18.18 mg, 0.113 mmol) in dimethyl sulfoxide (0.6 mL) was added HATU (47.2 mg, 0.124 mmol). The resulting solution was stirred at r.t. for 45 min. A solution of 3-(1-(N-methyl-4,5,6,7-tetrahydropyrazolo[1,5-a]pyrazine-3-carboxamido)cyclopropyl)benzoic acid hydrochloride (42.5 mg, 0.113 mmol) in dimethyl sulfoxide (0.7 mL) was added dropwise, followed by triethylamine (0.079 mL, 0.564 mmol). The resulting mixture was stirred at r.t. for 20 h. The reaction mixture was filtered and purified directly by HPLC to give 3-(1-(5-(1H-indole-2-carbonyl)-N-methyl-4,5,6,7-tetrahydropyrazolo[1,5-a]pyrazine-3-carboxamido)cyclopropyl)benzoic acid (0.0105 g, 19% yield).

Rt (Method A) 2.5 mins, m/z 484 [M+H]⁺

1H NMR (400 MHz, DMSO-d6) δ 11.71 (s, 1H), 7.79 (d, J=7.6 Hz, 1H), 7.73-7.54 (m, 2H), 7.54-7.35 (m, 2H), 7.33-7.14 (m, 2H), 7.14-6.85 (m, 3H), 5.44-4.93 (m, 2H), 4.47-3.94 (m, 4H), 3.16-2.94 (m, 3H), 1.70-1.22 (m, 4H).

### Example 14

### 12′-(1H-indole-2-carbonyl)-4′-methyl-4′,7′,8′,12′-tetraazaspiro[cyclopropane-1,5′-tricyclo[7.4.0.0²′7]tridecane]-1′,8′-dien-3′-one

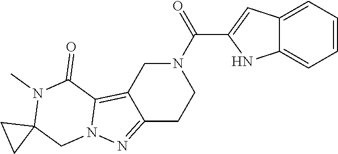 [SMILES: CN1C(=O)c2c3c(nn2CC12CC2)CCN(C(=O)c1cc2ccccc2[nH]1)C3]

Step 1: Tert-butyl (1-(hydroxymethyl)cyclopropyl)(methyl)carbamate (0.739 g, 3.67 mmol) was dissolved in dichloromethane (25 mL). To this was added triethylamine (0.768 mL, 5.51 mmol) and DMAP (0.045 g, 0.367 mmol). The mixture was cooled to 0° C. and benzoyl chloride (0.511 mL, 4.41 mmol) was added. The mixture was stirred at 0° C. for 30 minutes, and at room temperature for 1 hour. The mixture was quenched with saturated aqueous NH₄Cl solution. The aqueous layer was extracted with CH₂Cl₂. The combined organic extracts were washed with brine. The organic layer was dried over Na₂SO₄ concentrated in vacuo, then purified by column chromatography to give (1-((tertbutoxycarbonyl)(methyl)amino)cyclopropyl)methyl benzoate (0.982 g, 3.22 mmol, 88% yield).

Step 2: (1-((tert-butoxycarbonyl)(methyl)amino)cyclopropyl)methyl benzoate (0.982 g, 3.22 mmol) was dissolved in dry 1,4-dioxane (25 mL). To this was added HCl (4M in dioxane, 25 mL, 100 mmol). The mixture was stirred at room temperature for 3 hours. Solvents were evaporated in vacuo. The residue was stripped with CH₂Cl₂, toluene and CH₂Cl₂ to give (1-(methylamino)cyclopropyl)methyl benzoate hydrochloride (0.761 g, 3.15 mmol, 98% yield) as a white solid that bwas used in the next step without further purification.

Step 3: 5-(tert-butoxycarbonyl)-1-((2-(trimethylsilyl)ethoxy)methyl)-4,5,6,7-tetrahydro-1Hpyrazolo[4,3-c]pyridine-3-carboxylic acid (1.252 g, 3.15 mmol) and (1-(methylamino)cyclopropyl)methyl benzoate hydrochloride (0.761 g, 3.15 mmol) were dissolved in pyridine (20 mL). The mixture was cooled with salt/ice bath to—12° C. To this was added POCl3 (0.587 mL, 6.30 mmol). The mixture was stirred for 3 hours. Solvents were evaporated in vacuo. The residue was stripped with heptane (twice). The solids were dissolved in CH₂Cl₂ and washed with 1M KHSO₄ (twice), and brine. The organic layer was dried over Na₂SO₄ and concentrated in vacuo. The product was purified by column chromatography to give tert-butyl 3-((1-((benzoyloxy)methyl)cyclopropyl)(methyl)carbamoyl)-1-((2-(trimethylsilyl)ethoxy)methyl)-1,4,6,7-tetrahydro-5H-pyrazolo [4,3-c]pyridine-5-carboxylate (1.335 g, 2.283 mmol, 72.5% yield) as a colourless oil.

Step 4: Tert-butyl 3-((1-((benzoyloxy)methyl)cyclopropyl)(methyl)carbamoyl)-1-((2-(trimethylsilyl)ethoxy)methyl)-1,4,6,7-tetrahydro-5H-pyrazolo [4,3-c]pyridine-5-carboxylate (1.335 g, 2.283 mmol) was dissolved in 4M HCl in dioxane (20 mL, 80 mmol) and stirred for 16 hours. Solvents were evaporated in vacuo. The residue was stripped with CH₂Cl₂ (twice) to obtain (1-(N-methyl-4,5,6,7-tetrahydro-1H-pyrazolo[4,3-c]pyridine-3-carboxamido)cyclopropyl)methyl benzoate dihydrochloride that was used in the next step without further purification.

Step 5: (1-(N-methyl-4,5,6,7-tetrahydro-1H-pyrazolo[4,3-c]pyridine-3-carboxamido)cyclopropyl)methyl benzoate dihydrochloride (0.976 g, 2.284 mmol) was suspended in dichloromethane (30 mL). To this was added triethylamine (0.700 mL, 5.02 mmol). To this was added Boc-anhydride (0.583 mL, 2.51 mmol) was added. The mixture was stirred at room temperature for 1.5 hours. The reaction was quenched with saturated aqueous.

NH₄Cl solution, and product extracted with CH₂Cl₂. The combined organic extracts were washed with brine, dried over Na₂SO₄ and concentrated in vacuo. The product was purified by column chromatography to give tert-butyl 3-((1-((benzoyloxy)methyl)cyclopropyl)(methyl)carbamoyl)-1,4,6,7-tetrahydro-5H-pyrazolo[4,3-c]pyridine-5-carboxylate (0.846 g, 1.861 mmol, 81% yield) as a white foam.

Step 6: Tert-butyl 3-((1-((benzoyloxy)methyl)cyclopropyl)(methyl)carbamoyl)-1,4,6,7-tetrahydro-5H-pyrazolo[4,3-c]pyridine-5-carboxylate (0.846 g, 1.861 mmol) was dissolved in tetrahydrofuran (15 mL). To this was added water (15 mL), followed by lithium hydroxide monohydrate (0.234 g, 5.58 mmol). The mixture was stirred at room temperature for 16 hours. The mixture was acidified with 1M HCl, (5.58 mL, 5.58 mmol), then concentrated under vacuum. The residue was stripped with toluene, then purified by HPLC to give tert-butyl 3-((1-(hydroxymethyl)cyclopropyl)(methyl)carbamoyl)-1,4,6,7-tetrahydro-5H-pyrazolo[4,3-c]pyridine-5-carboxylate (0.523 g, 1.492 mmol, 80% yield).

Step 7: Tert-butyl 3-((1-(hydroxymethyl)cyclopropyl)(methyl)carbamoyl)-1,4,6,7-tetrahydro-5Hpyrazolo[4,3-c]pyridine-5-carboxylate (0.523 g, 1.492 mmol) was dissolved in dry tetrahydrofuran (60 mL). To this was added triphenylphosphine (0.509 g, 1.940 mmol). A solution of DIAD (0.377 mL, 1.940 mmol) in dry tetrahydrofuran (20 mL) was added dropwise. The mixture was then stirred at 80° C. for 2 hours. The mixture was poured in water (20 mL) and extracted with EtOAc (2×20 mL). The combined organic extracts were washed with brine (30 mL). The organic layer was dried over Na₂SO₄ and concentrated in vacuo to give tert-butyl 9′-methyl-10′-oxo-3′,4′,9′,10′-tetrahydro-7′Hspiro[cyclopropane-1,8′-pyrido[4′,3′:3,4]pyrazolo[1,5-a]pyrazine]-2′(1′H)-carboxylate that was used in the next step without further purification.

Step 8: Tert-butyl 9′-methyl-10′-oxo-3′,4′,9′,10′-tetrahydro-7′H-spiro[cyclopropane-1,8′-pyrido[4′,3′:3,4]pyrazolo[1,5-a]pyrazine]-2′(1′H)-carboxylate (0.496 g, 1.492 mmol) was dissolved in 4M HCl in dioxane (20 mL, 80 mmol). The mixture was stirred at roomtemperature for 16 hours. Solvents were evaporated in vacuo. The residue was suspended in CH₂Cl₂. Solids were filtered, and washed with CH₂Cl₂ (twice) and EtOAc (removal of residual TPPO). The solids were dried in vacuo to give 9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one hydrochloride (0.366 g, 1.362 mmol, 91% yield) as a white solid.

Step 9: 9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one hydrochloride (0.030 g, 0.112 mmol) was dissolved in dry N,N-dimethylformamide (1 mL). To this was added triethylamine (0.078 mL, 0.558 mmol). In a separate vial HATU (0.051 g, 0.134 mmol) and 1H-indole-2-carboxylic acid (0.018 g, 0.112 mmol) were stirred in dry N,N-dimethylformamide (1 mL) for 10 minutes.

This solution was added to the former solution. The mixture was stirred at room temperature for 16 hours. The mixture was quenched with water (0.250 mL). The solution was filtered and the filter rinsed with DMSO (0.2 mL). The product was purified by HPLC to give 2′-(1H-indole-2-carbonyl)-9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one (0.040 g, 0.107 mmol, 95% yield).

Rt (Method A) 2.9 mins, m/z 376 [M+H]⁺

1H NMR (400 MHz, DMSO-d6) δ 11.60 (d, J=2.2 Hz, 1H), 7.64 (d, J=8.0 Hz, 1H), 7.43 (d, J=8.1 Hz, 1H), 7.19 (ddd, J=8.1, 6.9, 1.2 Hz, 1H), 7.09-7.01 (m, 1H), 6.88 (s, 1H), 5.10-4.72 (m, 2H), 4.27-4.12 (m, 2H), 4.10-3.87 (m, 2H), 2.95-2.68 (m, 5H), 1.24-1.11 (m, 2H), 0.95-0.82 (m, 2H).

### Example 15

### 12′-(6-chloro-5-fluoro-1H-indole-2-carbonyl)-4′-methyl-4′,7′,8′,12′-tetraazaspirol[cyclopropane-1,5′-tricyclo[7.4.0.0²′7]tridecane]-1′,8′-dien-3′-one

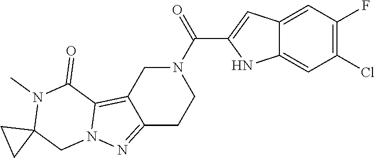 [SMILES: CN1C(=O)c2c3c(nn2CC12CC2)CCN(C(=O)c1cc2cc(F)c(Cl)cc2[nH]1)C3]

Step 1: Tert-butyl 9′-methyl-10′-oxo-3′,4′,9′,10′-tetrahydro-7′H-spiro[cyclopropane-1,8′-pyrido[4′,3′:3,4]pyrazolo[1,5-a]pyrazine]-2′(1′H)-carboxylate (0.020 g, 0.060 mmol) was dissolved in 4M HCl in dioxane (5 mL, 20.00 mmol). The mixture was stirred at room temperature for 2 hours. Solvents were then removed in vacuo. The residue was stripped with CH₂Cl₂ (twice) to give 9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one hydrochloride that was used in the next step without further purification.

Step 2: 9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one hydrochloride (0.016 g, 0.060 mmol) was dissolved in dry N,N-dimethylformamide (1 mL). To this was added triethylamine (0.041 mL, 0.298 mmol). In a separate vial HATU (0.027 g, 0.071 mmol) and 6-chloro-5-fluoro-1Hindole-2-carboxylic acid (0.013 g, 0.060 mmol) were stirred in N,N-Dimethylformamide (dry) (1 mL) for 10 minutes. This solution was then added to the former solution, and the mixture stirred at room temperature for 3 hours. The mixture was quenched with water (0.250 mL). The solution was filtered and the filter rinsed with DMSO (1 mL). The product was purified directly by HPLC to give 2′-(6-chloro-5-fluoro-1H-indole-2-carbonyl)-9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one (0.005 g, 0.012 mmol, 19.63% yield).

Rt (Method A) 3.19 mins, m/z 428/430 [M+H]⁺

1H NMR (400 MHz, DMSO-d6) δ 11.90 (s, 1H), 7.66 (d, J=10.0 Hz, 1H), 7.55 (d, J=6.5 Hz, 1H), 6.92 (s, 1H), 5.19-4.67 (m, 2H), 4.28-4.15 (m, 2H), 4.10-3.87 (m, 2H), 2.96-2.71 (m, 5H), 1.27-1.10 (m, 2H), 0.98-0.82 (m, 2H).

### Example 16

### 4′-methyl-12′-(4-methyl-1H-indole-2-carbonyl)-4′,7′,8′,12′-tetraazaspiro[cyclopropane-1,5′-tricyclo[7.4.0.0²′7]tridecane]-1′,8′-dien-3′-one

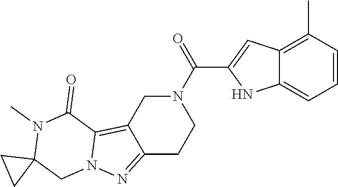 [SMILES: Cc1cccc2[nH]c(C(=O)N3CCc4nn5c(c4C3)C(=O)N(C)C3(CC3)C5)cc12]

9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one hydrochloride (0.030 g, 0.112 mmol) was dissolved in N,N-Dimethylformamide (dry) (1 mL). To this was added triethylamine (0.078 mL, 0.558 mmol).

In a separate vial HATU (0.051 g, 0.134 mmol) and 4-methyl-1H-indole-2-carboxylic acid (0.020 g, 0.112 mmol) were stirred in dry N,N-dimethylformamide (1 mL) for 10 minutes. This solution was then added to the former solution. The mixture was stirred at room temperature for 16 hours, then quenched with water (0.250 mL). DMSO (1 mL) was added and the product purified by HPLC to give 9′-methyl-2′-(4-methyl-1H-indole-2-carbonyl)-1′,2′,3′,4′-tetrahydro-7′H-spiro[cyclopropane-1,8′-pyrido[4′,3′:3,4]pyrazolo[1,5-a]pyrazin]-10′(9′H)-one (0.037 g, 0.095 mmol, 85% yield).

Rt (Method A) 3.02 mins, m/z 390 [M+H]⁺

1H NMR (400 MHz, DMSO-d6) δ 11.57 (d, J=2.3 Hz, 1H), 7.24 (d, J=8.3 Hz, 1H), 7.08 (dd, J=8.3, 7.0 Hz, 1H), 6.91-6.81 (m, 2H), 5.05-4.81 (m, 2H), 4.24-4.16 (m, 2H), 4.07-3.93 (m, 2H), 2.94-2.69 (m, 5H), 1.23-1.12 (m, 2H), 0.94-0.85 (m, 2H). One signal (3H) coincides with DMSO.

### Example 17

### 12′-(4-chloro-1H-indole-2-carbonyl)-4′-methyl-4′,7′,8′,12′-tetraazaspiro[cyclopropane-1,5′-tricyclo[7.4.0.0²′7]tridecane]-1′,8′-dien-3′-one

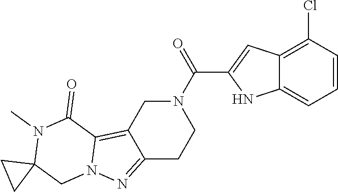 [SMILES: CN1C(=O)c2c3c(nn2CC12CC2)CCN(C(=O)c1cc2c(Cl)cccc2[nH]1)C3]

9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one hydrochloride (0.030 g, 0.112 mmol) was dissolved in dry N,N-dimethylformamide (1 mL). To this was added triethylamine (0.078 mL, 0.558 mmol). In a separate vial HATU (0.051 g, 0.134 mmol) and 4-chloro-1H-indole-2-carboxylic acid (0.022 g, 0.112 mmol) were stirred in dry N,N-dimethylformamide (1 mL) for 10 minutes. This solution was then added to the former solution. The mixture was stirred at room temperature for 16 hours, then quenched with water (0.250 mL). The solution was filtered and flushed with DMSO (1 mL). The product was purified directly by HPLC to give 2′-(4-chloro-1H-indole-2-carbonyl)-9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one (0.040 g, 0.098 mmol, 87% yield).

Rt (Method A) 3.12 mins, m/z 410/412 [M+H]⁺

1H NMR (400 MHz, DMSO-d6) δ 12.02 (s, 1H), 7.41 (d, J=8.1 Hz, 1H), 7.24-7.12 (m, 2H), 6.85 (s, 1H), 5.14-4.76 (m, 2H), 4.26-4.13 (m, 2H), 4.07-3.90 (m, 2H), 2.93-2.68 (m, 5H), 1.24-1.11 (m, 2H), 0.94-0.83 (m, 2H).

### Example 18

### 12′-(5-fluoro-4-methyl-1H-indole-2-carbonyl)-4′-methyl-4′,7′,8′,12′-tetraazaspiro[cyclopropane-1,5′-tricyclo[7.4.0.0²′7]tridecane]-1′,8′-dien-3′-one

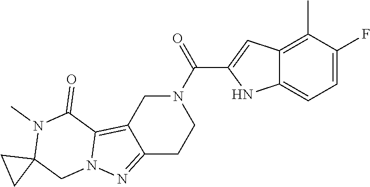 [SMILES: Cc1c(F)ccc2[nH]c(C(=O)N3CCc4nn5c(c4C3)C(=O)N(C)C3(CC3)C5)cc12]

9′-methyl-1′,2′,3′,4′-tetrahydro-7′H-spiro[cyclopropane-1,8′-pyrido[4′,3′:3,4]pyrazolo[1,5-a]pyrazin]-10′(9′H)-one hydrochloride (0.030 g, 0.112 mmol) was dissolved in dry N,N-dimethylformamide (1 mL). To this was added triethylamine (0.078 mL, 0.558 mmol). In a separate vial HATU (0.051 g, 0.134 mmol) and 5-fluoro-4-methyl-1Hindole-2-carboxylic acid (0.010 g, 0.052 mmol) were stirred in dry N,N-dimethylformamide (1 mL) for 10 minutes. This solution was then added to the former solution. The mixture was stirred at room temperature for 16 hours. The mixture was then quenched with water (0.250 mL), and the solution filtered and flushed with DMSO (1 mL). The product was purified directly by HPLC to give 2′-(5-fluoro-4-methyl-1H-indole-2-carbonyl)-9′-methyl-1′,2′,3′,4′-tetrahydro-7′H-spiro[cyclopropane-1,8′pyrido[4′,3′:3,4]pyrazolo[1,5-a]pyrazin]-10′(9′H)-one (0.015 g, 0.037 mmol, 33.0% yield).

Rt (Method A) 3.08 mins, m/z 408 [M+H]⁺

1H NMR (400 MHz, DMSO-d6) δ 11.67 (d, J=2.0 Hz, 1H), 7.24 (dd, J=8.8, 4.2 Hz, 1H), 7.01 (dd, J=10.2, 8.9 Hz, 1H), 6.92 (d, J=2.1 Hz, 1H), 5.01-4.82 (m, 2H), 4.24-4.15 (m, 2H), 4.05-3.94 (m, 2H), 2.92-2.69 (m, 5H), 2.43-2.37 (m, 3H), 1.23-1.14 (m, 2H), 0.93-0.84 (m, 2H).

### Example 19

### 6-{1-[5-(1H-indole-2-carbonyl)-4H,5H,6H,7H-pyrazolo[1,5-a]pyrazine-3-amido]cyclopropyl}pyridine-3-carboxylic acid

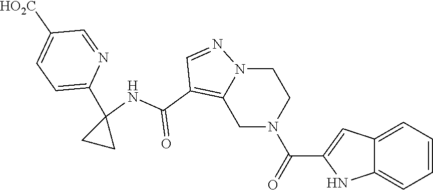 [SMILES: O=C(O)c1ccc(C2(NC(=O)c3cnn4c3CN(C(=O)c3cc5ccccc5[nH]3)CC4)CC2)nc1]

A solution of 1H-indole-2-carboxylic acid (18.86 mg, 0.117 mmol) and HATU (44.5 mg, 0.117 mmol) in dimethyl sulfoxide (0.6 mL) was stirred at rt for 1 h, then triethylamine (0.082 mL, 0.585 mmol) was added, followed by a solution of 6-(1-(4,5,6,7-tetrahydropyrazolo[1,5-a]pyrazine-3-carboxamido)cyclopropyl)nicotinic acid hydrochloride (42.6 mg, 0.117 mmol) in dimethyl sulfoxide (0.6 mL). The reaction mixture was stirred overnight, then filtered and purified directly by basic prep HPLC to give the desired product as an off-white fluffy solid (36 mg, 65% yield).

Rt (Method A2) 2.47 mins, m/z 471 [M+H]⁺

1H NMR (400 MHz, DMSO-d6) δ 11.70 (s, 1H), 8.98 (s, 1H), 8.89 (d, J=2.1 Hz, 1H), 8.26-7.91 (m, 2H), 7.63 (d, J=7.9 Hz, 1H), 7.54-7.32 (m, 2H), 7.20 (t, J=7.6 Hz, 1H), 7.05 (t, J=7.5 Hz, 1H), 6.94 (s, 1H), 5.39-4.96 (m, 2H), 4.43-4.08 (m, 4H), 1.67-1.49 (m, 2H), 1.37-1.18 (m, 2H).

### Example 20

### 2-{1-[5-(1H-indole-2-carbonyl)-4H,5H,6H,7H-pyrazolo[1,5-a]pyrazine-3-amido]cyclopropyl}pyrimidine-5-carboxylic acid

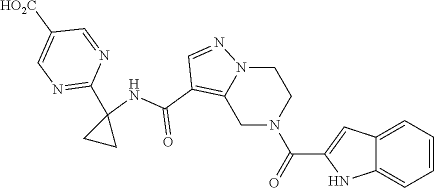 [SMILES: O=C(O)c1cnc(C2(NC(=O)c3cnn4c3CN(C(=O)c3cc5ccccc5[nH]3)CC4)CC2)nc1]

Step 1: Tert-butyl 3-((1-(5-(methoxycarbonyl)pyrimidin-2-yl)cyclopropyl)(methyl)carbamoyl)-6,7-dihydropyrazolo[1,5-a]pyrazine-5(4H)-carboxylate (73 mg, 0.160 mmol) was suspended in tetrahydrofuran (1 mL) and a solution of lithium hydroxide monohydrate (42.0 mg, 1 mmol) in water (1 mL) was added and the mixture was stirred at 60° C. for 1 h. After cooling to r.t., 1 M HCl (2 mL) was added followed by the addition of water (10 mL) and the mixture was extracted with EtOAc. The organic layer was washed with brine, dried over sodium sulfate to afford 2-(1-(5-(tert-butoxycarbonyl)-N-methyl-4,5,6,7-tetrahydropyrazolo[1,5-a]pyrazine-3-carboxamido)cyclopropyl)pyrimidine-5-carboxylic acid as a white solid (60 mg, 85% yield).

Step 2: 2-(1-(5-(tert-butoxycarbonyl)-N-methyl-4,5,6,7-tetrahydropyrazolo[1,5-a]pyrazine-3-carboxamido)cyclopropyl)pyrimidine-5-carboxylic acid (60 mg, 0.136 mmol) was dissolved in 4M HCl in dioxane (1 mL, 4.00 mmol) and the mixture was stirred overnight. The suspension was concentrated and stripped with dichloromethane to afford 2-(1-(N-methyl-4,5,6,7-tetrahydropyrazolo[1,5-a]pyrazine-3-carboxamido)cyclopropyl)pyrimidine-5-carboxylic acid hydrochloride as an off white solid (43 mg, 85% yield).

Step 3: Indole-2-carboxylic acid (11.93 mg, 0.074 mmol) was dissolved in DMSO (400 μL) and Et₃N (25.8 μL, 0.185 mmol) was added followed by the addition of HATU (28.1 mg, 0.074 mmol). The mixture was stirred for 1 h. In a separate vial. 2-(1-(N-methyl-4,5,6,7-tetrahydropyrazolo[1,5-a]pyrazine-3-carboxamido)cyclopropyl)pyrimidine-5-carboxylic acid hydrochloride (28.0 mg, 0.074 mmol) was dissolved in DMSO (400 μL) and Et₃N (25.8 μL, 0.185 mmol) was added. The reaction mixture was stirred overnight, then filtered, flushed with MeOH, and purified by using preparative HPLC to afford 2-(1-(5-(1H-indole-2-carbonyl)-N-methyl-4,5,6,7-tetrahydropyrazolo1[1,5-a]pyrazine-3-carboxamido)cyclopropyl)pyrimidine-5-carboxylic acid as a white solid (6.7 mg, 18% yield).

Rt (Method A2) 2.45 mins, m/z 472 [M+H]⁺

1H NMR (400 MHz, DMSO-d6) δ 11.70 (s, 1H), 8.96 (d, J=32.6 Hz, 3H), 8.12 (s, 1H), 7.63 (d, J=8.0 Hz, 1H), 7.43 (d, J=8.1 Hz, 1H), 7.28-7.00 (m, 3H), 6.93 (s, 1H), 5.38-4.95 (m, 2H), 4.27 (d, J=28.2 Hz, 4H), 1.73-1.55 (m, 2H), 1.41-1.27 (m, 2H).

### Example 21

### 5-(4-chloro-1H-indole-2-carbonyl)-N-(2-hydroxyethyl)-N-methyl-4H,5H,6H,7H-[1,2]oxazolo[4,5-c]pyridine-3-carboxamide

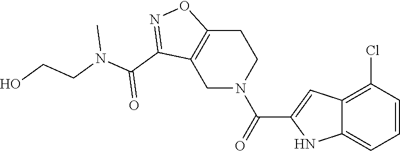 [SMILES: CN(CCO)C(=O)c1noc2c1CN(C(=O)c1cc3c(Cl)cccc3[nH]1)CC2]

Rt 3.00 mins (Method A) [M+H]=403.1/405.1

1H NMR (400 MHz, DMSO-d6) δ 12.06 (s, 1H), 7.41 (d, J=7.9 Hz, 1H), 7.24-7.13 (m, 2H), 6.89 (s, 1H), 4.90-4.60 (m, 3H), 4.15-3.96 (m, 2H), 3.65-3.45 (m, 4H), 3.23 (s, 1H), 3.13-2.91 (m, 4H).

### Example 22

### N-(2-hydroxyethyl)-5-(1H-indole-2-carbonyl)-N-methyl-4H,5H,6H,7H-[1,2]oxazolo[4,5-c]pyridine-3-carboxamide

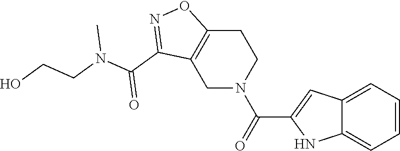 [SMILES: CN(CCO)C(=O)c1noc2c1CN(C(=O)c1cc3ccccc3[nH]1)CC2]

Rt 2.80 mins (Method A) [M+H]=369.1

1H NMR (400 MHz, DMSO-d6) δ 11.66 (s, 1H), 7.64 (d, J=8.1 Hz, 1H), 7.43 (d, J=8.0 Hz, 1H), 7.21 (t, J=7.5 Hz, 1H), 7.06 (t, J=7.5 Hz, 1H), 6.92 (s, 1H), 4.87-4.59 (m, 3H), 4.16-3.97 (m, 2H), 3.68-3.42 (m, 4H), 3.23 (s, 1H), 3.13-2.91 (m, 4H).

### Example 23

### 5-(4,6-difluoro-1H-indole-2-carbonyl)-N-(2-hydroxyethyl)-N-methyl-4H,5H,6H,7H-[1,2]oxazolo[4,5-c]pyridine-3-carboxamide

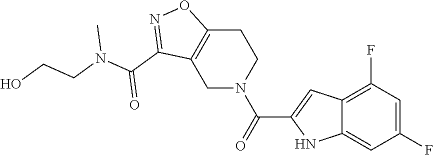 [SMILES: CN(CCO)C(=O)c1noc2c1CN(C(=O)c1cc3c(F)cc(F)cc3[nH]1)CC2]

Rt 2.96 mins (Method A) [M+H]=405.1

1H NMR (400 MHz, DMSO-d6) δ 12.10 (s, 1H), 7.08-6.85 (m, 3H), 4.91-4.55 (m, 3H), 4.17-3.92 (m, 2H), 3.69-3.42 (m, 4H), 3.23 (s, 1H), 3.15-2.87 (m, 4H).

### Example 24

### 2-({1-[5-(1H-indole-2-carbonyl)-2H,4H,5H,6H,7H-pyrazolo[4,3-c]pyridin-3-yl]-N-methylformamido}methyl)benzoic acid

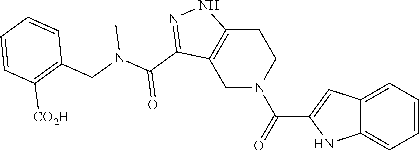 [SMILES: CN(Cc1ccccc1C(=O)O)C(=O)c1n[nH]c2c1CN(C(=O)c1cc3ccccc3[nH]1)CC2]

Rt 3.01 mins (Method B) [M+H]=458.2

1H NMR (400 MHz, DMSO-d6) δ 13.06 (m, 2H), 11.64 (s, 1H), 7.89 (d, J=7.4 Hz, 1H), 7.63 (d, J=8.9 Hz, 1H), 7.31 (m, 5H), 7.07 (m, 1H), 6.89 (s, 1H), 5.50 (m, 1H), 5.00 (m, 3H), 4.01 (m, 3H), 3.39 (m, 1H), 2.92 (m, 4H).

### Example 25

### 2-[3-(3,3-difluoropyrrolidine-1-carbonyl)-4H,5H,6H,7H-[1,2]oxazolo[4,5-c]pyridine-5-carbonyl]-1H-indole

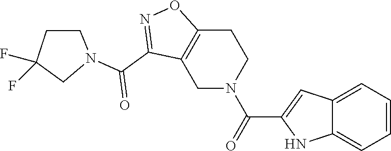 [SMILES: O=C(c1cc2ccccc2[nH]1)N1CCc2onc(C(=O)N3CCC(F)(F)C3)c2C1]

Rt 3.36 mins (Method A) [M+H]=401.1

1H NMR (400 MHz, DMSO-d6) δ 11.66 (s, 1H), 7.65 (d, J=7.9 Hz, 1H), 7.43 (d, J=8.1 Hz, 1H), 7.21 (ddd, J=8.0, 6.9, 1.2 Hz, 1H), 7.07 (ddd, J=8.1, 7.0, 1.0 Hz, 1H), 6.92 (s, 1H), 5.13-4.51 (m, 2H), 4.32-3.65 (m, 6H), 3.19-2.94 (m, 2H), 2.49-2.36 (m, 2H).

### Example 26

### 5-(1H-indole-2-carbonyl)-N-methyl-N-[(pyridin-2-yl)methyl]-4H,5H,6H,7H-[1,2]oxazolo[4,5-c]pyridine-3-carboxamide

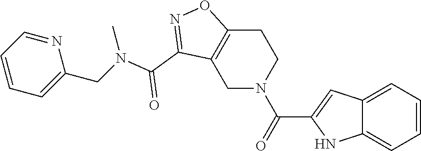 [SMILES: CN(Cc1ccccn1)C(=O)c1noc2c1CN(C(=O)c1cc3ccccc3[nH]1)CC2]

### Example 27—Intentionally Left Blank

### Example 28 —Intentionally Left Blank

### Example 29

### 2-{1-[5-(1H-indole-2-carbonyl)-4H,5H,6H,7H-pyrazolo[1,5-a]pyrazine-3-amido]cyclopropyl}pyrimidine-5-carboxylic acid

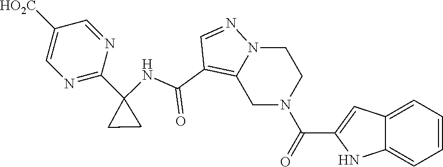 [SMILES: O=C(O)c1cnc(C2(NC(=O)c3cnn4c3CN(C(=O)c3cc5ccccc5[nH]3)CC4)CC2)nc1]

Rt 2.45 mins (Method A2) [M+H]⁺ 472.2

1H NMR (400 MHz, DMSO-d6) δ 11.70 (s, 1H), 8.96 (d, J=32.6 Hz, 3H), 8.12 (s, 1H), 7.63 (d, J=8.0 Hz, 1H), 7.43 (d, J=8.1 Hz, 1H), 7.28-7.00 (m, 3H), 6.93 (s, 1H), 5.38-4.95 (m, 2H), 4.27 (d, J=28.2 Hz, 4H), 1.73-1.55 (m, 2H), 1.41-1.27 (m, 2H).

### Example 30

### 2-{1-[N-methyl-5-(1H-indole-2-carbonyl)-4H,5H,6H,7H-pyrazolo[1,5-a]pyrazine-3-amido]cyclopropyl}benzoic acid

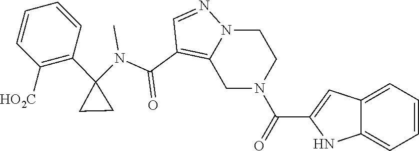 [SMILES: CN(C(=O)c1cnn2c1CN(C(=O)c1cc3ccccc3[nH]1)CC2)C1(c2ccccc2C(=O)O)CC1]

Rt 2.54 mins (Method A2) [M+H]⁺ 484.1

1H NMR (400 MHz, DMSO-d6) δ 11.70 (s, 1H), 8.07-7.15 (m, 7H), 7.08 (t, J=7.5 Hz, 1H), 6.95 (s, 1H), 5.37-4.76 (m, 2H), 4.43-4.05 (m, 4H), 3.19 (s, 3H), 1.64-0.99 (m, 4H).

### Example 31

### 6-{1-[N-methyl-5-(1H-indole-2-carbonyl)-4H,5H,6H,7H-pyrazolo[1,5-a]pyrazine-3-amido]cyclopropyl}pyridine-3-carboxylic acid

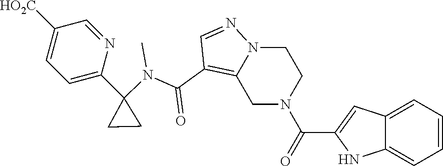 [SMILES: CN(C(=O)c1cnn2c1CN(C(=O)c1cc3ccccc3[nH]1)CC2)C1(c2ccc(C(=O)O)cn2)CC1]

Step 1: 6-(1-(5-(tert-butoxycarbonyl)-N-methyl-4,5,6,7-tetrahydropyrazolo[1,5-a]pyrazine-3-carboxamido)cyclopropyl)nicotinic acid (100 mg, 0.227 mmol) was dissolved in 4M HCl in dioxane (2 mL, 8.00 mmol) and the resulting brown suspension was stirred at r.t. for 1 h. The reaction mixture was evaporated, and the residue was co-evaporated with toluene (2×10 mL) to give 6-(1-(N-methyl-4,5,6,7-tetrahydropyrazolo[1,5-a]pyrazine-3-carboxamido)cyclopropyl)nicotinic acid hydrochloride as a light brown solid (86 mg, quant. yield).

Step 2: To a solution of 1H-indole-2-carboxylic acid (18 mg, 0.114 mmol) in DMSO (0.5 mL) was added HATU (43.3 mg, 0.114 mmol) and the resulting light brown solution was stirred at r.t. After 1 h, Et₃N (0.079 mL, 0.569 mmol) was added, followed by the addition of solution of 6-(1-(N-methyl-4,5,6,7-tetrahydropyrazolo [1,5-a]pyrazine-3-carboxamido)cyclopropyl)nicotinic acid hydrochloride (43 mg, 0.114 mmol) in DMSO (0.6 mL). The reaction mixture was stirred for 1 h, then filtered through a micro filter and purified directly using preparative HPLC to give the pMBATroduct as a solid (22 mg, 40% yield).

Rt 2.57 mins (Method A2) [M+H]⁺ 485.1

1H NMR (400 MHz, DMSO-d6) δ 11.72 (s, 1H), 9.06-8.89 (m, 1H), 8.27-8.03 (m, 1H), 7.74-7.57 (m, 1H), 7.53-7.14 (m, 3H), 7.08 (t, J=7.5 Hz, 1H), 6.95 (s, 1H), 6.85 (s, 1H), 5.41-4.93 (m, 2H), 4.44-3.96 (m, 4H), 3.07 (s, 3H), 1.99-1.77 (m, 1H), 1.77-1.20 (m, 3H).

### Example 32

### 3-{1-[5-(1H-indole-2-carbonyl)-4H,5H,6H,7H-pyrazolo[1,5-a]pyrazine-3-amido]cyclopropyl}benzoic acid

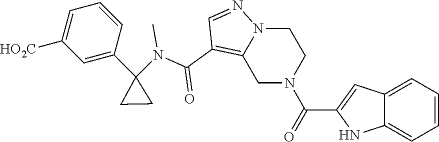 [SMILES: CN(C(=O)c1cnn2c1CN(C(=O)c1cc3ccccc3[nH]1)CC2)C1(c2cccc(C(=O)O)c2)CC1]

Rt 3.35 mins (Method B2) [M+H]=470.2

1H NMR (400 MHz, DMSO-d6) δ 11.69 (s, 1H), 8.91 (s, 1H), 8.12 (s, 1H), 7.77 (s, 1H), 7.74-7.67 (m, 1H), 7.64 (d, J=8.0 Hz, 1H), 7.43 (d, J=8.2 Hz, 1H), 7.39-7.26 (m, 2H), 7.24-7.16 (m, 1H), 7.05 (t, J=7.5 Hz, 1H), 6.93 (s, 1H), 5.41-4.92 (m, 2H), 4.41-4.09 (m, 4H), 1.24 (s, 4H). One signal (1H) coincides with water signal.

### Example 33

### 3-{1-[N-methyl-5-(6-chloro-5-fluoro-1H-indole-2-carbonyl)-4H,5H,6H,7H-pyrazolo[1,5-a]pyrazine-3-amido]cyclopropyl}benzoic acid

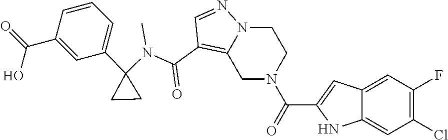 [SMILES: CN(C(=O)c1cnn2c1CN(C(=O)c1cc3cc(F)c(Cl)cc3[nH]1)CC2)C1(c2cccc(C(=O)O)c2)CC1]

Step 1: 3-(1-(5-(tert-butoxycarbonyl)-N-methyl-4,5,6,7-tetrahydropyrazolo[1,5-a]pyrazine-3-carboxamido)cyclopropyl)benzoic acid (0.050 g, 0.114 mmol) was suspended in 4M HCl in dioxane (1 mL, 4.00 mmol) and the resulting white suspension was stirred at r.t overnight. The reaction mixture was concentrated and co-evaporated with MeOH (2×5 mL) and dichloromethane (2×5 mL) to obtain 3-(1-(N-methyl-4,5,6,7-tetrahydropyrazolo[1,5-a]pyrazine-3-carboxamido)cyclopropyl)benzoic acid hydrochloride as a yellow solid (45 mg, quant. yield).

Step 2: 6-chloro-5-fluoro-1H-indole-2-carboxylic acid (0.024 g, 0.114 mmol) was dissolved in N,N-dimethylformamide (0.75 mL) and HATU (0.046 g, 0.120 mmol) was added. The mixture was stirred for 30 mins. The resulting solution was added to a suspension of 3-(1-(N-methyl-4,5,6,7-tetrahydropyrazolo[1,5-a]pyrazine-3-carboxamido)cyclopropyl)benzoic acid hydrochloride (0.043 g, 0.114 mmol) and Et₃N (0.079 mL, 0.570 mmol) in N,N-dimethylformamide (0.75 mL) and the mixture was stirred overnight at r.t. The reaction mixture was filtered through a micro filter and purified by preparative HPLC to afford a white fluffy solid (5 mg, 7% yield).

Rt 2.91 mins (Method A2) [M+H]⁺ 536.0/538.0

1H NMR (400 MHz, DMSO-d6) δ 11.94 (s, 1H), 7.83-7.75 (m, 1H), 7.74-7.64 (m, 1H), 7.62 (s, 1H), 7.57 (d, J=6.4 Hz, 1H), 7.53-7.41 (m, 1H), 7.32-7.15 (m, 1H), 7.06-6.85 (m, 2H), 5.51-4.88 (m, 2H), 4.42-3.92 (m, 4H), 3.04 (s, 3H), 1.71-1.29 (m, 4H)—proton from carboxylic acid not observed.

### Example 34

### 4-({1-[N-methyl-5-(1H-indole-2-carbonyl)-4H,5H,6H,7H-pyrazolo[1,5-a]pyrazine-3-amido]cyclopropyl}methyl)benzoic acid

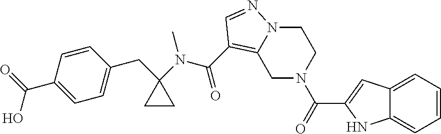 [SMILES: CN(C(=O)c1cnn2c1CN(C(=O)c1cc3ccccc3[nH]1)CC2)C1(Cc2ccc(C(=O)O)cc2)CC1]

Rt 2.66 mins (Method A2) [M+H]⁺ 498.1

1H NMR (400 MHz, DMSO-d6) δ 11.74 (s, 1H), 7.94-7.59 (m, 4H), 7.45 (d, J=8.2 Hz, 1H), 7.39-7.14 (m, 3H), 7.07 (t, J=7.5 Hz, 1H), 6.96 (s, 1H), 5.39-4.85 (m, 2H), 4.42-4.05 (m, 4H), 2.98-2.54 (m, 5H), 1.30-0.64 (m, 4H).

### Example 35

### 3-{1-[N-methyl-5-(5-fluoro-4-methyl-1H-indole-2-carbonyl)-4H,5H,6H,7H-pyrazolo[1,5-a]pyrazine-3-amido]cyclopropyl}benzoic acid

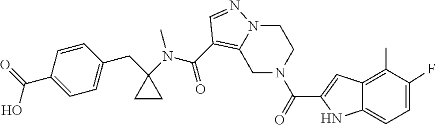 [SMILES: Cc1c(F)ccc2[nH]c(C(=O)N3CCn4ncc(C(=O)N(C)C5(Cc6ccc(C(=O)O)cc6)CC5)c4C3)cc12]

Rt 2.84 mins (Method A2) [M+H]⁺ 516.1

1H NMR (400 MHz, DMSO-d6) δ 13.69-12.22 (m, 1H), 11.95-11.53 (m, 1H), 7.86-7.75 (m, 1H), 7.63 (s, 1H), 7.56-7.39 (m, 1H), 7.26 (dd, J=8.9, 4.3 Hz, 2H), 7.13-6.85 (m, 3H), 5.41-4.93 (m, 2H), 4.45-3.98 (m, 4H), 3.04 (s, 3H), 2.42 (s, 3H), 1.67-1.30 (m, 4H)—proton of carboxylic acid hardly observed.

### Example 36

### 2-{1-[N-methyl-5-(1H-indole-2-carbonyl)-4H,5H,6H,7H-pyrazolo[1,5-a]pyrazine-3-amido]cyclopropyl}pyrimidine-4-carboxylic acid

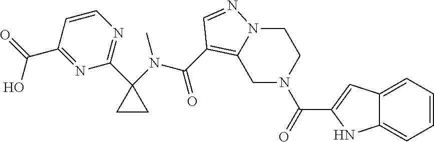 [SMILES: CN(C(=O)c1cnn2c1CN(C(=O)c1cc3ccccc3[nH]1)CC2)C1(c2nccc(C(=O)O)n2)CC1]

Rt 2.49 mins (Method A2) [M+H]⁺ 486.1

1H NMR (400 MHz, DMSO-d6) δ 11.48 (s, 1H), 8.73 (s, 1H), 7.72-7.36 (m, 3H), 7.28-6.78 (m, 4H), 5.24-5.06 (m, 2H), 4.35-3.93 (m, 4H), 1.92-1.39 (m, 4H)—mixture of conformers observed.

### Example 37

### 12′-(4-fluoro-1H-indole-2-carbonyl)-4′-methyl-4′,7′,8′,12′-tetraazaspiro[cyclopropane-1,5′-tricyclo[7.4.0.0², □]tridecane]-1′,8′-dien-3′-one

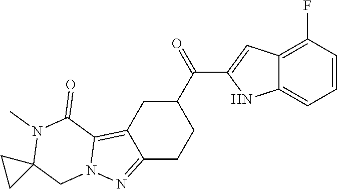 [SMILES: CN1C(=O)c2c3c(nn2CC12CC2)CCC(C(=O)c1cc2c(F)cccc2[nH]1)C3]

Rt 3.34 mins (Method B2) [M+H]⁺ 394.1

1H NMR (400 MHz, DMSO-d6) δ 12.01 (s, 1H), 7.27 (d, J=8.2 Hz, 1H), 7.18 (td, J=8.0, 5.2 Hz, 1H), 6.92 (s, 1H), 6.85 (dd, J=10.8, 7.6 Hz, 1H), 5.31-4.62 (m, 2H), 4.29-4.15 (m, 2H), 4.11-3.88 (m, 2H), 3.00-2.71 (m, 5H), 1.25-1.15 (m, 2H), 0.94-0.86 (m, 2H).

### Example 38

### 5-(1H-indole-2-carbonyl)-N-[1-(methoxymethyl)cyclopropyl]-N-methyl-4H,5H,6H,7H-[1,2]oxazolo[4,3-c]pyridine-3-carboxamide

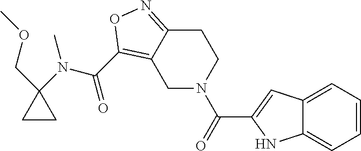 [SMILES: COCC1(N(C)C(=O)c2onc3c2CN(C(=O)c2cc4ccccc4[nH]2)CC3)CC1]

Rt 3.54 mins (Method B2) [M+H]⁺ 409.1

1H NMR (400 MHz, DMSO-d6) δ 11.67 (s, 1H), 7.65 (d, J=8.0 Hz, 1H), 7.43 (d, J=8.2 Hz, 1H), 7.24-7.16 (m, 1H), 7.10-7.02 (m, 1H), 6.96-6.88 (m, 1H), 5.25-4.64 (m, 2H), 4.26-3.79 (m, 3H), 3.57-3.39 (m, 1H), 3.30-3.20 (m, 4H), 3.09-2.95 (m, 4H), 1.02-0.73 (m, 4H).

### Example 39

### N-cyclopropyl-5-(1H-indole-2-carbonyl)-N-methyl-4,5,6,7-tetrahydroisoxazolo[4,3-c]pyridine-3-carboxamide

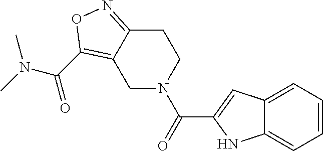 [SMILES: CN(C)C(=O)c1onc2c1CN(C(=O)c1cc3ccccc3[nH]1)CC2]

Step 1: Ethyl 5-(1H-indole-2-carbonyl)-4,5,6,7-tetrahydroisoxazolo[4,3-c]pyridine-3-carboxylate (58 mg, 0.171 mmol) was suspended in tetrahydrofuran (1 mL) and a solution of lithium hydroxide monohydrate (42 mg, 1.001 mmol) in water (1.000 mL) was added. After stirring the mixture 1 h, 1M HCl (2 mL) and water (5 mL) were added and the resulting suspension was stirred for 30 mins. The suspension was filtered and the solids were washed with water and Et₂O to yield 5-(1H-indole-2-carbonyl)-4,5,6,7-tetrahydroisoxazolo[4,3-c]pyridine-3-carboxylic acid as an off-white solid (41.6 mg, 78% yield).

Step 2: 5-(1H-indole-2-carbonyl)-4,5,6,7-tetrahydroisoxazolo[4,3-c]pyridine-3-carboxylic acid (21 mg, 0.067 mmol) was dissolved in DMSO (400 μL) and HATU (28.2 mg, 0.074 mmol) was added. After 10 mins, Et₃N (47.0 μL, 0.337 mmol) was added immediately followed by a solution of N-methylcyclopropanamine hydrochloride (7.98 mg, 0.074 mmol) in DMSO (400 μL) and the mixture was stirred for 1 h. A drop of water was added and the reaction mixture was filtered, flushed with acetonitrile and water, and purified using preparative HPLC to afford N-cyclopropyl-5-(1H-indole-2-carbonyl)-N-methyl-4,5,6,7-tetrahydroisoxazolo[4,3-c]pyridine-3-carboxamide as a white solid (15.9 mg, 64% yield).

Rt 3.46 mins (Method B2) [M+H]⁺ 365.1

1H NMR (400 MHz, DMSO-d6) δ 11.67 (s, 1H), 7.65 (d, J=8.0 Hz, 1H), 7.43 (d, J=8.2 Hz, 1H), 7.24-7.17 (m, 1H), 7.10-7.03 (m, 1H), 6.92 (s, 1H), 5.22-4.63 (m, 2H), 4.15-3.88 (m, 2H), 3.17-2.86 (m, 6H), 0.79-0.54 (m, 4H).

### Example 40

### 5-(1H-indole-2-carbonyl)-N-[(2R)-1,1,1-trifluoropropan-2-yl]-4H,5H,6H,7H-[1,2]oxazolo[4,3-c]pyridine-3-carboxamide

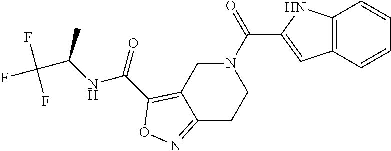 [SMILES: C[C@@H](NC(=O)c1onc2c1CN(C(=O)c1cc3ccccc3[nH]1)CC2)C(F)(F)F]

Rt 3.68 mins (Method B2) [M−H] 405.0

1H NMR (400 MHz, DMSO-d6) δ 11.68 (s, 1H), 9.59 (d, J=8.4 Hz, 1H), 7.65 (d, J=8.0 Hz, 1H), 7.43 (d, J=8.2 Hz, 1H), 7.24-7.17 (m, 1H), 7.10-7.03 (m, 1H), 6.93 (s, 1H), 5.14-4.89 (m, 2H), 4.83-4.72 (m, 1H), 4.19-3.84 (m, 2H), 3.17-2.91 (m, 2H), 1.36 (d, J=7.1 Hz, 3H).

### Example 41

### 3-{1-[N-methyl-7-(1H-indole-2-carbonyl)-6-methyl-5H,6H,7H,8H-imidazo[1,5-a]pyrazine-1-amido]cyclopropyl}benzoic acid

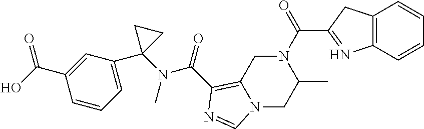 [SMILES: CC1Cn2cnc(C(=O)N(C)C3(c4cccc(C(=O)O)c4)CC3)c2CN1C(=O)C1=Nc2ccccc2C1]

Step 1: Tert-butyl 1-((1-(3-(methoxycarbonyl)phenyl)cyclopropyl)(methyl)carbamoyl)-6-methyl-5,6-dihydroimidazo[1,5-a]pyrazine-7(8H)-carboxylate (100 mg, 0.213 mmol) is suspended in 4M HCl in dioxane (2.03 mL, 8.11 mmol). After stirring for 2 h the reaction mixture was concentrated in vacuo and was stripped with dichloromethane to afford methyl 3-(1-(N,6-dimethyl-5,6,7,8-tetrahydroimidazo[1,5-a]pyrazine-1-carboxamido)cyclopropyl)benzoate hydrochloride as beige solid (85.1 mg, 98% yield).

Step 2: To a mixture of 1H-indole-2-carboxylic acid (16.72 mg, 0.104 mmol) and HATU (41.4 mg, 0.109 mmol) in dichloromethane (0.5 mL) was added Et₃N (0.101 mL, 0.726 mmol). After stirring for 30 mins at r.t. a solution of methyl 3-(1-(N,6-dimethyl-5,6,7,8-tetrahydroimidazo[1,5-a]pyrazine-1-carboxamido)cyclopropyl)benzoate hydrochloride (42 mg, 0.104 mmol) in dichloromethane (0.500 mL) was added. The resulting reaction mixture was stirred for 3 days. To the reaction mixture a solution of 1H-indole-2-carboxylic acid (8.36 mg, 0.052 mmol) and HATU (19.72 mg, 0.052 mmol) in N,N-dimethylformamide (0.5 mL) was added. After stirring overnight the reaction mixture was concentrated in vacuo. The resulting solid was dissolved in DMSO and purified by preparative HPLC to afford methyl 3-(1-(7-(1H-indole-2-carbonyl)-N,6-dimethyl-5,6,7,8-tetrahydroimidazo[1,5-a]pyrazine-1-carboxamido)cyclopropyl)benzoate as beige solid (25.4 mg, 48% yield).

Step 3: Methyl 3-(1-(7-(1H-indole-2-carbonyl)-N,6-dimethyl-5,6,7,8-tetrahydroimidazo[1,5-a]pyrazine-1-carboxamido)cyclopropyl)benzoate (24.6 mg, 0.048 mmol) was dissolved in tetrahydrofuran (5.8 mL). To this water (0.58 mL) was added, followed by lithium hydroxide monohydrate (12.11 mg, 0.289 mmol). The mixture was stirred at r.t. for three days. The reaction mixture was diluted with water (3 mL) and was acidified with 1M HCl solution to pH 3. The product was extracted with EtOAc (3×4 mL). The combined organic layers were washed with brine (4 mL), dried over sodium sulfate and concentrated in vacuo. The resulting solid was dissolved in DMSO and purified by preparative HPLC to afford the product as a white solid (23.4 mg, 98% yield).

Rt 3.02 mins (Method B2) [M+H]⁺ 498.4

1H NMR (400 MHz, DMSO-d6) δ 13.10 (s, 1H), 11.70 (s, 1H), 7.81 (d, J=7.4 Hz, 1H), 7.71-7.61 (m, 2H), 7.56-7.42 (m, 2H), 7.35-7.17 (m, 2H), 7.08 (t, J=7.5 Hz, 1H), 7.04-6.90 (m, 2H), 5.84-5.47 (m, 1H), 5.40-5.21 (m, 1H), 5.08-4.59 (m, 1H), 4.49-4.02 (m, 2H), 3.05 (s, 3H), 1.70-1.18 (m, 7H).

### Example 42

### 3-{1-[N-methyl-5-(4,5-difluoro-1H-indole-2-carbonyl)-4H,5H,6H,7H-pyrazolo[1,5-a]pyrazine-3-amido]cyclopropyl}benzoic acid

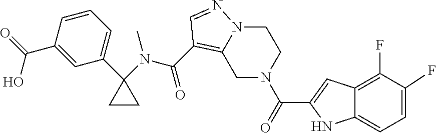 [SMILES: CN(C(=O)c1cnn2c1CN(C(=O)c1cc3c(F)c(F)ccc3[nH]1)CC2)C1(c2cccc(C(=O)O)c2)CC1]

Rt 2.80 mins (Method A2) [M+H]⁺ 520.2

1H NMR (400 MHz, DMSO-d6) δ 12.13 (bs, 1H), 7.80 (d, J=7.6 Hz, 1H), 7.62 (s, 1H), 7.54-7.38 (m, 1H), 7.33-7.19 (m, 3H), 7.07 (s, 1H), 6.97 (s, 1H), 5.50-4.84 (m, 2H), 4.43-3.96 (m, 4H), 3.16-2.99 (m, 3H), 1.68-1.28 (m, 4H)—proton of carboxylic acid not observed.

### Example 43

### 12′-(1H-indole-2-carbonyl)-4′,7′,8′,12′-tetraazaspiro[cyclopropane-1,5′-tricyclo[7.4.0.0², D]tridecane]-1′,8′-dien-3′-one

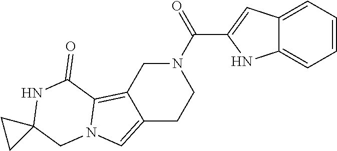 [SMILES: O=C1NC2(CC2)Cn2cc3c(c21)CN(C(=O)c1cc2ccccc2[nH]1)CC3]

Rt 3.05 mins (Method A2) [M+H]⁺ 362.2

1H NMR (400 MHz, DMSO-d6) δ 11.66 (s, 1H), 8.40 (s, 1H), 7.65 (d, J=8.0 Hz, 1H), 7.43 (d, J=8.3 Hz, 1H), 7.25-7.17 (m, 1H), 7.10-7.03 (m, 1H), 6.89 (s, 1H), 5.33-4.55 (m, 2H), 4.20 (s, 2H), 4.13-3.83 (m, 2H), 2.99-2.77 (m, 2H), 0.90-0.76 (m, 4H).

### Example 44

### 4-{1-[N-methyl-5-(1H-indole-2-carbonyl)-2H,4H,5H,6H,7H-pyrazolo[4,3-c]pyridine-3-amido]cyclopropyl}benzoic acid

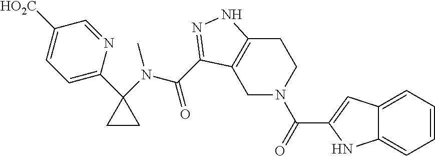 [SMILES: CN(C(=O)c1n[nH]c2c1CN(C(=O)c1cc3ccccc3[nH]1)CC2)C1(c2ccc(C(=O)O)cn2)CC1]

Step 1: To tert-butyl 3-((1-(4-(methoxycarbonyl)phenyl)cyclopropyl)(methyl)carbamoyl)-1-((2-(trimethylsilyl)ethoxy)methyl)-1,4,6,7-tetrahydro-5H-pyrazolo[4,3-c]pyridine-5-carboxylate (0.050 g, 0.086 mmol) was added 4M HCl in dioxane (1 mL, 4.00 mmol) and the resulting clear solution was stirred at r.t. overnight. The reaction mixture was concentrated in vacuo and co-evaporated with dichloromethane (3×5 mL) to obtain methyl 4-(1-(N-methyl-4,5,6,7-tetrahydro-2H-pyrazolo[4,3-c]pyridine-3-carboxamido)cyclopropyl)benzoate dihydrochloride as a white solid (37 mg, quant. yield).

Step 2: 1H-indole-2-carboxylic acid (0.014 g, 0.087 mmol) and Et₃N (0.060 mL, 0.433 mmol) were dissolved in N,N-dimethylformamide (0.5 mL) and HATU (0.035 g, 0.091 mmol) was added. After stirring for 15 mins, the reaction mixture was added to a suspension of methyl 4-(1-(N-methyl-4,5,6,7-tetrahydro-2H-pyrazolo[4,3-c]pyridine-3-carboxamido)cyclopropyl)benzoate dihydrochloride (0.037 g, 0.087 mmol) in N,N-dimethylformamide (0.5 mL) and the resulting reaction mixture was stirred for 1 h. The reaction mixture was poured out into water (30 mL) and extracted with EtOAc (3×30 mL). The organic layers were combined, washed with brine (5×20 mL), dried over anhydrous sodium sulfate, filtered and concentrated to obtain methyl 4-(1-(5-(1H-indole-2-carbonyl)-N-methyl-4,5,6,7-tetrahydro-2H-pyrazolo[4,3-c]pyridine-3-carboxamido)cyclopropyl)benzoate as a brown oil (43 mg, quant. yield).

Step 3: Methyl 4-(1-(5-(1H-indole-2-carbonyl)-N-methyl-4,5,6,7-tetrahydro-2H-pyrazolo[4,3-c]pyridine-3-carboxamido)cyclopropyl)benzoate (0.043 g, 0.086 mmol) was dissolved in tetrahydrofuran (2 mL). A solution of lithium hydroxide monohydrate (0.084 g, 2 mmol) in water (2 mL) was added and the resulting clear solution was stirred for 2 h. The reaction mixture was acidified with 6 M aqueous HCl (0.35 mL) and purified by preparative HPLC to afford 4-(1-(5-(1H-indole-2-carbonyl)-N-methyl-4,5,6,7-tetrahydro-2H-pyrazolo [4,3-c]pyridine-3-carboxamido)cyclopropyl)benzoic acid as a white fluffy solid (42 mg, 27% yield).

Rt 2.61 mins (Method A2) [M+H]⁺ 484.1

1H NMR (400 MHz, DMSO-d6) δ 13.52-12.71 (m, 1H), 11.71 (s, 1H), 7.90 (d, J=8.0 Hz, 2H), 7.70 (d, J=7.9 Hz, 1H), 7.49 (d, J=8.3 Hz, 1H), 7.25 (t, J=7.7 Hz, 1H), 7.22-7.06 (m, 3H), 6.93 (s, 1H), 5.31-4.60 (m, 2H), 4.21-3.86 (m, 2H), 3.24-3.03 (m, 3H), 3.03-2.77 (m, 2H), 1.65-1.27 (m, 4H)—proton of carboxylic acid not observed.

### Example 45

### 2-{1-[N-methyl-5-(4-chloro-1H-indole-2-carbonyl)-4H,5H,6H,7H-pyrazolo[1,5-a]pyrazine-3-amido]cyclopropyl}pyrimidine-5-carboxylic acid

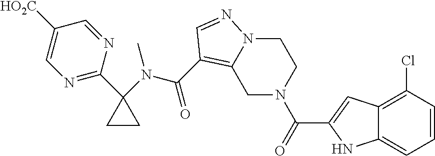 [SMILES: CN(C(=O)c1cnn2c1CN(C(=O)c1cc3c(Cl)cccc3[nH]1)CC2)C1(c2ncc(C(=O)O)cn2)CC1]

Rt 3.44 mins (Method B2) [M+H]⁺ 520.1/522.0

1H NMR (400 MHz, DMSO-d6) δ 12.11 (s, 1H), 9.14-8.99 (m, 2H), 7.46-7.39 (m, 1H), 7.33-7.01 (m, 3H), 6.93 (s, 1H), 6.80 (s, 1H), 5.70-4.70 (m, 2H), 4.46-3.98 (m, 4H), 3.16-3.01 (m, 3H), 2.01-1.85 (m, 1H), 1.72-1.38 (m, 3H).

### Example 46

### 5-(1H-indole-2-carbonyl)-N-[(2R)-1,1,1-trifluoropropan-2-yl]-4H,5H,6H,7H-[1,2]oxazolo[4,5-c]pyridine-3-carboxamide

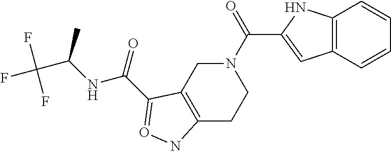

Rt 3.65 mins (Method A2) [M+H]⁺ 407.1

1H NMR (400 MHz, DMSO-d6) δ 11.68 (s, 1H), 9.60 (d, J=7.9 Hz, 1H), 7.65 (d, J=7.9 Hz, 1H), 7.43 (d, J=8.2 Hz, 1H), 7.25-7.18 (m, 1H), 7.07 (t, J=7.4 Hz, 1H), 6.93 (s, 1H), 5.42-4.61 (m, 3H), 4.21-3.78 (m, 2H), 3.15-2.89 (m, 2H), 1.36 (d, J=7.0 Hz, 3H).

### Example 47

### 4-({1-[N-methyl-5-(1H-indole-2-carbonyl)-4H,5H,6H,7H-pyrazolo[1,5-a]pyrazine-3-amido]cyclopropyl}methoxy)benzoic acid

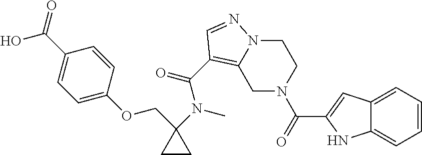 [SMILES: CN(C(=O)c1cnn2c1CN(C(=O)c1cc3ccccc3[nH]1)CC2)C1(COc2ccc(C(=O)O)cc2)CC1]

Step 1: A cooled (0° C.) solution of tert-butyl (1-(hydroxymethyl)cyclopropyl)(methyl)carbamate (50 mg, 0.248 mmol) in tetrahydrofuran (2 mL) was brought under a nitrogen atmosphere, and methyl 4-hydroxybenzoate (45.4 mg, 0.298 mmol) and triphenylphosphine (78 mg, 0.298 mmol) were added. The mixture was stirred for 5 mins, after which a solution of diisopropyl azodicarboxylate (0.058 mL, 0.298 mmol) in tetrahydrofuran (1 mL) was added dropwise. The reaction was allowed to warm up to r.t. under a nitrogen atmosphere. After stirring overnight the reaction mixture was concentrated in vacuo and the residue was taken up in EtOAc (5 mL). The organic layer was washed with 1M aqueous NaOH (5 mL), brine (5 mL), dried over sodium sulfate, filtered, and concentrated in vacuo. The residue was dissolved in a minimal amount of EtOAc and dichloromethane (˜1:1) and purified using column chromatography (EtOAc in heptanes 20% to 50%) to give methyl 4-((1-((tert-butoxycarbonyl)(methyl)amino)cyclopropyl)methoxy)benzoate as a sticky oil (83 mg, quant. yield).

Step 2: Methyl 4-((1-((tert-butoxycarbonyl)(methyl)amino)cyclopropyl)methoxy)benzoate (83 mg, 0.247 mmol) was dissolved in 4M HCl in dioxane (2 mL, 8.00 mmol) and the resulting clear solution was stirred at r.t. overnight. The reaction mixture was evaporated and the residue was stripped with toluene (2×10 mL) to give methyl 4-((1-(methylamino)cyclopropyl)methoxy)benzoate as a white solid (44 mg, 76% yield).

Step 3: To a solution of 5-(1H-indole-2-carbonyl)-4,5,6,7-tetrahydropyrazolo[1,5-a]pyrazine-3-carboxylic acid (58.0 mg, 0.187 mmol) in N,N-dimethylformamide (0.5 mL) was added HATU (71.1 mg, 0.187 mmol) and the resulting suspension was stirred at r.t. during 30 mins. Then, Et₃N (0.130 mL, 0.935 mmol) was added, followed by a solution of methyl 4-((1-(methylamino)cyclopropyl)methoxy)benzoate (44 mg, 0.187 mmol) in N,N-dimethylformamide (0.6 mL). The reaction mixture was filtered through a micro filter and purified by using preparative HPLC to give methyl 4-((1-(5-(1H-indole-2-carbonyl)-N-methyl-4,5,6,7-tetrahydropyrazolo[1,5-a]pyrazine-3-carboxamido)cyclopropyl)methoxy)benzoate as a light yellow solid (58 mg, 58% yield).

Step 4: To the yellow solution of methyl 4-((1-(5-(1H-indole-2-carbonyl)-N-methyl-4,5,6,7-tetrahydropyrazolo[1,5-a]pyrazine-3-carboxamido)cyclopropyl)methoxy)benzoate (58 mg, 0.110 mmol) in tetrahydrofuran (2 mL) was added 0.5 M lithium hydroxide in water (2.199 mL, 1.099 mmol) and the resulting solution was stirred at r.t. overnight. The reaction mixture was brought to neutral pH by addition of 1M aqueous HCl (1 mL), concentrated in vacuo and the residue was co-evaporated with MeCN (5 mL) to give 4-((1-(N-methyl-4,5,6,7-tetrahydropyrazolo[1,5-a]pyrazine-3-carboxamido)cyclopropyl)methoxy)benzoic acid as a yellow solid (40.7 mg, quant. yield).

Step 5: HATU (41.8 mg, 0.110 mmol) was added to a solution of 1H-indole-2-carboxylic acid (17.73 mg, 0.110 mmol) in DMSO (0.5 mL) and the resulting brown solution was stirred at r.t. during 45 mins. Then, Et₃N (0.077 mL, 0.550 mmol) was added, followed by a solution of 4-((1-(N-methyl-4,5,6,7-tetrahydropyrazolo[1,5-a]pyrazine-3-carboxamido)cyclopropyl)methoxy)benzoic acid (40.7 mg, 0.110 mmol) in DMSO (1 mL) and the mixture was stirred overnight. The reaction mixture was filtered through a micro filter and purified by using preparative HPLC to give the product as a solid (17 mg, 30% yield).

Rt 2.68 mins (method A2) [M+H]⁺ 514.2

1H NMR (400 MHz, DMSO-d6) δ 11.70 (s, 1H), 8.28-7.75 (m, 3H), 7.65 (d, J=8.0 Hz, 1H), 7.44 (d, J=8.2 Hz, 1H), 7.21 (t, J=7.6 Hz, 1H), 7.16-6.70 (m, 4H), 5.78-4.85 (m, 2H), 4.56-3.86 (m, 6H), 3.20-2.90 (m, 3H), 1.52-0.71 (m, 4H)—proton of carboxylic acid not observed.

### Example 48

### 4′-methyl-12′-[4-(trifluoromethyl)-1H-indole-2-carbonyl]-4′,7′,8′,12′-tetraazaspiro[cyclopropane-1,5′-tricyclo1[7.4.0.0²,]tridecane]-1′,8′-dien-3′-one

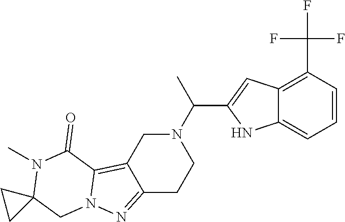 [SMILES: CC(c1cc2c(C(F)(F)F)cccc2[nH]1)N1CCc2nn3c(c2C1)C(=O)N(C)C1(CC1)C3]

Rt 3.59 mins (Method A2) [M+H]⁺ 444.1

1H NMR (400 MHz, DMSO-d6) δ 12.26 (s, 1H), 7.73 (d, J=8.3 Hz, 1H), 7.47 (d, J=7.5 Hz, 1H), 7.37 (t, J=7.8 Hz, 1H), 6.87 (s, 1H), 5.17-4.66 (m, 2H), 4.22 (s, 2H), 4.07-3.91 (m, 2H), 2.91-2.70 (m, 5H), 1.21-1.12 (m, 2H), 0.94-0.85 (m, 2H).

### Example 49

### 4-{1-[N-methyl-5-(4-chloro-1H-indole-2-carbonyl)-2H,4H,5H,6H,7H-pyrazolo[4,3-c]pyridine-3-amido]cyclopropyl}benzoic acid

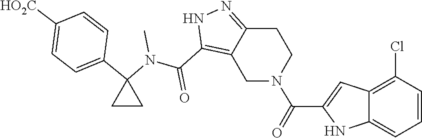 [SMILES: CN(C(=O)c1[nH]nc2c1CN(C(=O)c1cc3c(Cl)cccc3[nH]1)CC2)C1(c2ccc(C(=O)O)cc2)CC1]

Rt 2.79 mins (Method A2) [M+H]⁺ 518.1/520.1

1H NMR (400 MHz, DMSO-d6) δ 13.59-12.62 (m, 1H), 12.06 (s, 1H), 7.85 (d, J=8.1 Hz, 2H), 7.42 (d, J=8.0 Hz, 1H), 7.26-7.06 (m, 4H), 6.85 (s, 1H), 5.28-4.60 (m, 2H), 4.12-3.80 (m, 2H), 3.18-2.99 (m, 3H), 2.97-2.71 (m, 2H), 1.57-1.21 (m, 4H)—proton of carboxylic acid not observed.

### Example 50

### 4-{1-[N-methyl-5-(6-chloro-5-fluoro-1H-indole-2-carbonyl)-4H,5H,6H,7H-pyrazolo[1,5-a]pyrazine-3-amido]cyclopropyl}benzoic acid

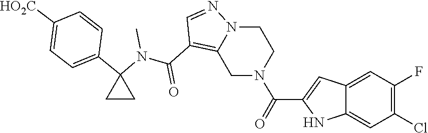 [SMILES: CN(C(=O)c1cnn2c1CN(C(=O)c1cc3cc(F)c(Cl)cc3[nH]1)CC2)C1(c2ccc(C(=O)O)cc2)CC1]

Rt 3.62 mins (Method B2) [M+H]⁺ 536.1/538.0

### Example 51

### 4-{1-[N-methyl-5-(4-chloro-1H-indole-2-carbonyl)-4H,5H,6H,7H-pyrazolo[1,5-a]pyrazine-3-amido]cyclopropyl}benzoic acid

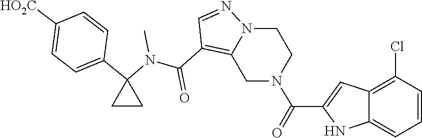 [SMILES: CN(C(=O)c1cnn2c1CN(C(=O)c1cc3c(Cl)cccc3[nH]1)CC2)C1(c2ccc(C(=O)O)cc2)CC1]

Rt 3.57 mins (Method B2) [M+H]⁺ 518.1/520.1

### Example 52

### 4-{1-[N-methyl-5-(4,5-difluoro-1H-indole-2-carbonyl)-4H,5H,6H,7H-pyrazolo[1,5-a]pyrazine-3-amido]cyclopropyl}benzoic acid

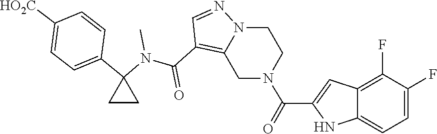 [SMILES: CN(C(=O)c1cnn2c1CN(C(=O)c1cc3c(F)c(F)ccc3[nH]1)CC2)C1(c2ccc(C(=O)O)cc2)CC1]

Rt 3.51 mins (Method B2) [M+H]⁺ 520.1

1H NMR (400 MHz, DMSO-d6) δ 13.76-12.31 (m, 1H), 12.15 (s, 1H), 8.00-7.77 (m, 2H), 7.32-7.23 (m, 2H), 7.20-7.11 (m, 2H), 7.07 (s, 1H), 6.96 (s, 1H), 5.62-4.76 (m, 2H), 4.51-3.95 (m, 4H), 3.04 (s, 3H), 1.75-1.27 (m, 4H).

### Example 53

### 4-{1-[N-methyl-5-(5-fluoro-4-methyl-1H-indole-2-carbonyl)-4H,5H,6H,7H-pyrazolo[1,5-a]pyrazine-3-amido]cyclopropyl}benzoic acid

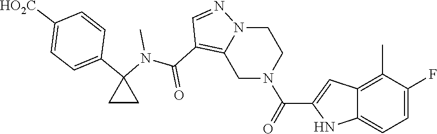 [SMILES: Cc1c(F)ccc2[nH]c(C(=O)N3CCn4ncc(C(=O)N(C)C5(c6ccc(C(=O)O)cc6)CC5)c4C3)cc12]

Rt 3.54 mins (Method B2) [M+H]⁺ 516.1

### Example 54

### 4-{1-[N-methyl-5-(6-fluoro-4-methyl-1H-indole-2-carbonyl)-4H,5H,6H,7H-pyrazolo[1,5-a]pyrazine-3-amido]cyclopropyl}benzoic acid

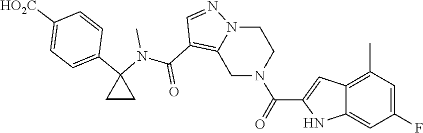 [SMILES: Cc1cc(F)cc2[nH]c(C(=O)N3CCn4ncc(C(=O)N(C)C5(c6ccc(C(=O)O)cc6)CC5)c4C3)cc12]

Rt 3.55 mins (Method B2) [M+H]⁺ 516.1

### Example 55

### 4-{1-[N-methyl-5-(6-chloro-5-fluoro-1H-indole-2-carbonyl)-2H,4H,5H,6H,7H-pyrazolo[4,3-c]pyridine-3-amido]cyclopropyl}benzoic acid

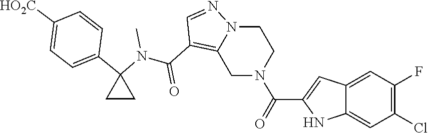 [SMILES: CN(C(=O)c1cnn2c1CN(C(=O)c1cc3cc(F)c(Cl)cc3[nH]1)CC2)C1(c2ccc(C(=O)O)cc2)CC1]

Rt 3.59 mins (Method B2) [M+H]⁺ 536.1/538.0

1H NMR (400 MHz, DMSO-d6) δ 13.32-12.77 (m, 1H), 11.89 (s, 1H), 7.83 (d, J=7.8 Hz, 2H), 7.63 (d, J=10.0 Hz, 1H), 7.54 (d, J=6.4 Hz, 1H), 7.11 (s, 2H), 6.90 (s, 1H), 5.14-4.60 (m, 2H), 4.10-3.73 (m, 2H), 3.41 (s, 2H), 3.08-2.71 (m, 3H), 1.51-1.20 (m, 4H)—proton of the carboxylic acid is not observed

### Example 56

### 2-{1-[N-methyl-5-(1H-indole-2-carbonyl)-2H,4H,5H,6H,7H-pyrazolo[4,3-c]pyridine-3-amido]cyclopropyl}pyrimidine-5-carboxylic acid

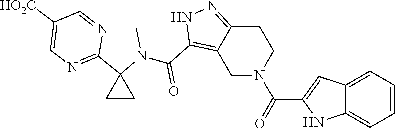 [SMILES: CN(C(=O)c1[nH]nc2c1CN(C(=O)c1cc3ccccc3[nH]1)CC2)C1(c2ncc(C(=O)O)cn2)CC1]

Rt 2.52 mins (Method A2) [M+H]⁺ 486.2

1H NMR (400 MHz, DMSO-d6) δ 12.93 (d, J=154.7 Hz, 1H), 11.63 (s, 1H), 9.07 (s, 2H), 7.71-7.54 (m, 1H), 7.48-7.35 (m, 1H), 7.24-7.13 (m, 1H), 7.11-6.98 (m, 1H), 6.91-6.80 (m, 1H), 5.27-4.46 (m, 2H), 4.19-3.66 (m, 2H), 3.52-3.04 (m, 3H), 3.00-2.69 (m, 2H), 1.97-1.82 (m, 1H), 1.68-1.27 (m, 3H)—proton of carboxylic acid not observed.

### Example 57

### 2-{1-[N-methyl-5-(6-chloro-5-fluoro-1H-indole-2-carbonyl)-4H,5H,6H,7H-pyrazolo[1,5-a]pyrazine-3-amido]cyclopropyl}pyrimidine-5-carboxylic acid

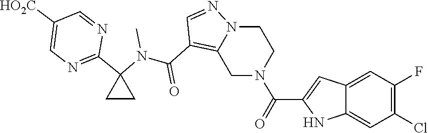 [SMILES: CN(C(=O)c1cnn2c1CN(C(=O)c1cc3cc(F)c(Cl)cc3[nH]1)CC2)C1(c2ncc(C(=O)O)cn2)CC1]

Rt 2.78 mins (Method A2) [M+H]⁺ 538.1/540.1

1H NMR (400 MHz, DMSO-d6) δ 11.95 (s, 1H), 9.19-8.96 (m, 2H), 7.80-7.46 (m, 2H), 7.32-6.71 (m, 2H), 5.50-4.75 (m, 2H), 4.50-3.90 (m, 4H), 3.08 (s, 3H), 2.01-1.33 (m, 4H).

### Example 58

### 2-{1-[N-methyl-5-(4,5-difluoro-1H-indole-2-carbonyl)-4H,5H,6H,7H-pyrazolo[1,5-a]pyrazine-3-amido]cyclopropyl}pyrimidine-5-carboxylic acid

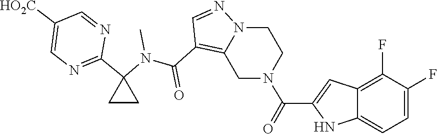 [SMILES: CN(C(=O)c1cnn2c1CN(C(=O)c1cc3c(F)c(F)ccc3[nH]1)CC2)C1(c2ncc(C(=O)O)cn2)CC1]

Rt 2.68 mins (Method A2) [M+H]⁺ 522.1

1H NMR (400 MHz, DMSO-d6) δ 12.14 (s, 1H), 9.20-8.74 (m, 2H), 7.34-6.93 (m, 3H), 6.80 (s, 1H), 5.43-4.77 (m, 2H), 4.52-3.89 (m, 4H), 3.08 (s, 3H), 2.03-1.29 (m, 4H).

### Example 59

### 2-{1-[N-methyl-5-(5-fluoro-4-methyl-1H-indole-2-carbonyl)-4H,5H,6H,7H-pyrazolo[1,5-a]pyrazine-3-amido]cyclopropyl}pyrimidine-5-carboxylic acid

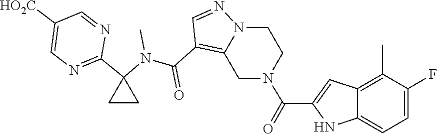 [SMILES: Cc1c(F)ccc2[nH]c(C(=O)N3CCn4ncc(C(=O)N(C)C5(c6ncc(C(=O)O)cn6)CC5)c4C3)cc12]

Rt 2.71 mins (Method A2) [M+H]⁺ 518.2

1H NMR (400 MHz, DMSO-d6) δ 11.77 (d, J=2.3 Hz, 1H), 9.07 (d, J=28.3 Hz, 2H), 7.35-6.69 (m, 4H), 5.14 (s, 2H), 4.18 (d, J=53.3 Hz, 4H), 3.08 (s, 3H), 2.45-2.29 (m, 3H), 2.02-1.37 (m, 4H).

### Example 60

### 2-{1-[N-methyl-5-(6-fluoro-4-methyl-1H-indole-2-carbonyl)-4H,5H,6H,7H-pyrazolo[1,5-a]pyrazine-3-amido]cyclopropyl}pyrimidine-5-carboxylic acid

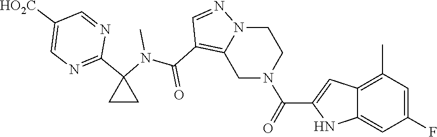 [SMILES: Cc1cc(F)cc2[nH]c(C(=O)N3CCn4ncc(C(=O)N(C)C5(c6ncc(C(=O)O)cn6)CC5)c4C3)cc12]

Rt 2.71 mins (Method A2) [M+H]⁺ 518.1

1H NMR (400 MHz, DMSO-d6) δ 11.76 (d, J=2.4 Hz, 1H), 9.20-8.95 (m, 2H), 7.30-6.67 (m, 4H), 5.40-4.80 (m, 2H), 4.46-3.97 (m, 4H), 3.08 (s, 3H), 2.52 (s, 3H), 2.02-1.84 (m, 1H), 1.78-1.39 (m, 3H).

### Example 61

### 2-({1-[N-methyl-5-(1H-indole-2-carbonyl)-4H,5H,6H,7H-pyrazolo[1,5-a]pyrazine-3-amido]cyclopropyl}methoxy)benzoic acid

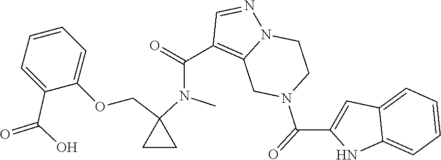 [SMILES: CN(C(=O)c1cnn2c1CN(C(=O)c1cc3ccccc3[nH]1)CC2)C1(COc2ccccc2C(=O)O)CC1]

Rt 2.56 mins (Method A2) [M+H]⁺ 514.2

1H NMR (400 MHz, DMSO-d6) δ 11.70 (s, 1H), 8.05-7.71 (m, 1H), 7.69-7.49 (m, 2H), 7.49-7.30 (m, 2H), 7.29-6.76 (m, 5H), 5.42-4.88 (m, 2H), 4.67-3.81 (m, 6H), 3.25-2.95 (m, 3H), 1.48-0.79 (m, 4H).

### Example 62

### 3-({1-[N-methyl-5-(1H-indole-2-carbonyl)-4H,5H,6H,7H-pyrazolo[1,5-a]pyrazine-3-amido]cyclopropyl}methoxy)benzoic acid

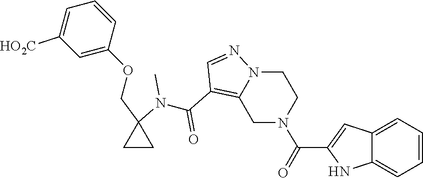 [SMILES: CN(C(=O)c1cnn2c1CN(C(=O)c1cc3ccccc3[nH]1)CC2)C1(COc2cccc(C(=O)O)c2)CC1]

Rt 2.70 mins (Method A2) [M+H]⁺ 514.2

1H NMR (400 MHz, DMSO-d6) δ 11.72 (s, 1H), 8.31-7.73 (m, 1H), 7.65 (d, J=8.0 Hz, 1H), 7.57-7.27 (m, 4H), 7.21 (t, J=7.5 Hz, 1H), 7.17-7.00 (m, 2H), 6.94 (s, 1H), 5.45-4.89 (m, 2H), 4.53-3.97 (m, 6H), 3.10 (s, 3H), 1.56-0.63 (m, 4H).

### Example 63

### 4′-(1H-indole-2-carbonyl)-13′-methyl-4′, 8′,9′,13′-tetraazaspiro[cyclopropane-1,12′-tricyclo[7.5.0.0²,]tetradecane]-1′,7′-dien-14′-one

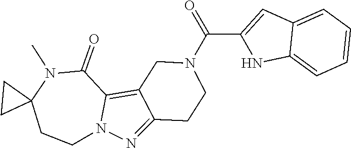 [SMILES: CN1C(=O)c2c3c(nn2CCC12CC2)CCN(C(=O)c1cc2ccccc2[nH]1)C3]

Rt 3.16 mins (Method A2) [M+H]⁺ 390.2

1H NMR (400 MHz, DMSO-d6) δ 11.65 (s, 1H), 7.63 (d, J=8.0 Hz, 1H), 7.42 (d, J=8.2 Hz, 1H), 7.20 (t, J=7.6 Hz, 1H), 7.06 (t, J=7.5 Hz, 1H), 6.87 (s, 1H), 5.25-4.50 (m, 2H), 4.33 (t, J=6.8 Hz, 2H), 4.15-3.83 (m, 2H), 3.09-2.72 (m, 5H), 2.12 (t, J=7.1 Hz, 2H), 0.88-0.64 (m, 2H), 0.60-0.40 (m, 2H).

### Example 64

### 4′-(6-chloro-5-fluoro-1H-indole-2-carbonyl)-13′-methyl-4′,8′,9′,13′-tetraazaspiro[cyclopropane-1,12′-tricyclo[7.5.0.0², Q]tetradecane]-1′,7′-dien-14′-one

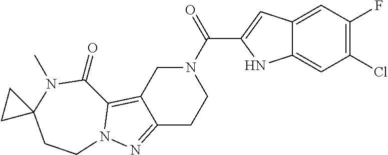 [SMILES: CN1C(=O)c2c3c(nn2CCC12CC2)CCN(C(=O)c1cc2cc(F)c(Cl)cc2[nH]1)C3]

Rt 3.48 mins (Method A2) [M+H]⁺ 442.1/444.1

1H NMR (400 MHz, DMSO-d6) δ 11.90 (s, 1H), 7.64 (d, J=10.1 Hz, 1H), 7.54 (d, J=6.4 Hz, 1H), 6.90 (s, 1H), 5.14-4.52 (m, 2H), 4.38-4.27 (m, 2H), 4.10-3.86 (m, 2H), 2.99-2.71 (m, 5H), 2.18-2.07 (m, 2H), 0.89-0.64 (m, 2H), 0.58-0.41 (m, 2H).

### Example 65

### 4-{1-[N-methyl-5-(4,5-difluoro-1H-indole-2-carbonyl)-2H,4H,5H,6H,7H-pyrazolo[4,3-c]pyridine-3-amido]cyclopropyl}benzoic acid

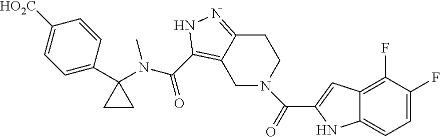 [SMILES: CN(C(=O)c1[nH]nc2c1CN(C(=O)c1cc3c(F)c(F)ccc3[nH]1)CC2)C1(c2ccc(C(=O)O)cc2)CC1]

Rt 3.46 mins (Method B2) [M+H]⁺ 520.1

1H NMR (400 MHz, DMSO-d6) δ 13.35-12.72 (m, 1H), 12.07 (s, 1H), 7.83 (d, J=7.8 Hz, 2H), 7.23 (d, J=5.8 Hz, 2H), 7.11 (s, 2H), 6.97 (s, 1H), 5.21-4.59 (m, 2H), 4.13-3.75 (m, 2H), 3.41 (s, 2H), 3.08-2.73 (m, 3H), 1.54-1.22 (m, 4H).”

### Example 66

### 4-{1-[N-methyl-5-(5-fluoro-4-methyl-1H-indole-2-carbonyl)-2H,4H,5H,6H,7H-pyrazolo[4,3-c]pyridine-3-amido]cyclopropyl}benzoic acid

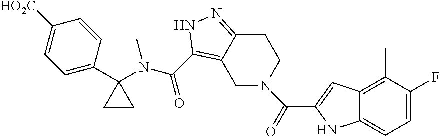 [SMILES: Cc1c(F)ccc2[nH]c(C(=O)N3CCc4n[nH]c(C(=O)N(C)C5(c6ccc(C(=O)O)cc6)CC5)c4C3)cc12]

Rt 3.48 mins (Method B2) [M+H]⁺ 516.1

1H NMR (400 MHz, DMSO-d6) δ 13.35-12.73 (m, 1H), 11.70 (s, 1H), 7.83 (d, J=8.0 Hz, 2H), 7.27-7.20 (m, 1H), 7.18-7.07 (m, 2H), 7.05-6.97 (m, 1H), 6.92 (s, 1H), 4.81 (s, 2H), 4.20-3.75 (m, 2H), 3.40 (s, 2H), 3.07-2.73 (m, 3H), 2.41 (s, 3H), 1.50-1.21 (m, 4H).

### Example 67

### 4-{1-[N-methyl-5-(6-fluoro-4-methyl-1H-indole-2-carbonyl)-2H,4H,5H,6H,7H-pyrazolo[4,3-c]pyridine-3-amido]cyclopropyl}benzoic acid

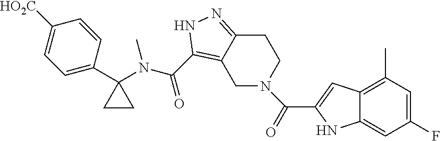 [SMILES: Cc1cc(F)cc2[nH]c(C(=O)N3CCc4n[nH]c(C(=O)N(C)C5(c6ccc(C(=O)O)cc6)CC5)c4C3)cc12]

Rt 3.50 mins (Method B2) [M+H]⁺ 516.1

1H NMR (400 MHz, DMSO-d6) δ 13.38-12.71 (m, 1H), 11.70 (s, 1H), 7.83 (d, J=8.1 Hz, 2H), 7.19-7.04 (m, 2H), 7.00-6.86 (m, 2H), 6.81-6.70 (m, 1H), 4.83 (s, 2H), 4.10-3.79 (m, 2H), 3.50-3.39 (m, 4H), 3.11-2.98 (m, 2H), 2.96-2.72 (m, 2H), 1.55-1.20 (m, 4H).

### Example 68

### 4′-(4-chloro-1H-indole-2-carbonyl)-13′-methyl-4′, 8′,9′,13′-tetraazaspiro[cyclopropane-1,12′-tricyclo[7.5.0.0²,7]tetradecane]-1′,7′-dien-14′-one

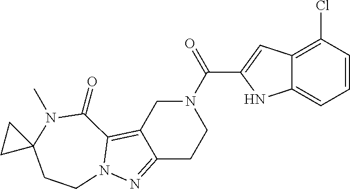 [SMILES: CN1C(=O)c2c3c(nn2CCC12CC2)CCN(C(=O)c1cc2c(Cl)cccc2[nH]1)C3]

Rt 3.41 mins (Method A2) [M+H]⁺ 424.1/426.1

1H NMR (400 MHz, DMSO-d6) δ 12.05 (s, 1H), 7.41 (d, J=8.0 Hz, 1H), 7.23-7.12 (m, 2H), 6.83 (s, 1H), 5.14-4.58 (m, 2H), 4.37-4.27 (m, 2H), 4.11-3.86 (m, 2H), 2.99-2.71 (m, 5H), 2.17-2.08 (m, 2H), 0.83-0.70 (m, 2H), 0.57-0.46 (m, 2H).

### Example 69

### 4-{1-[N-methyl-5-(4,6-dichloro-1H-indole-2-carbonyl)-4H,5H,6H,7H-pyrazolo[1,5-a]pyrazine-3-amido]cyclopropyl}benzoic acid

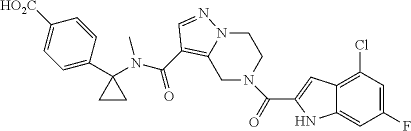 [SMILES: CN(C(=O)c1cnn2c1CN(C(=O)c1cc3c(Cl)cc(F)cc3[nH]1)CC2)C1(c2ccc(C(=O)O)cc2)CC1]

Rt 3.87 mins (Method B2) [M+H]⁺ 552.0/554.0

1H NMR (400 MHz, DMSO-d6) δ 12.24 (s, 1H), 8.00-7.75 (m, 2H), 7.46 (s, 1H), 7.28 (s, 1H), 7.15 (d, J=8.1 Hz, 2H), 7.02-6.89 (m, 2H), 5.51-4.88 (m, 2H), 4.45-3.93 (m, 4H), 3.04 (s, 3H), 1.74-1.29 (m, 4H)—signal of carboxylic acid not observed.

### Example 70

### 2-{1-[N-methyl-5-(4-chloro-1H-indole-2-carbonyl)-2H,4H,5H,6H,7H-pyrazolo[4,3-c]pyridine-3-amido]cyclopropyl}pyrimidine-5-carboxylic acid

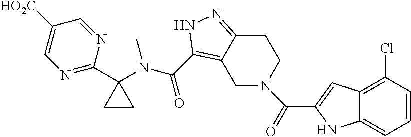 [SMILES: CN(C(=O)c1[nH]nc2c1CN(C(=O)c1cc3c(Cl)cccc3[nH]1)CC2)C1(c2ncc(C(=O)O)cn2)CC1]

Rt 3.40 mins (Method B2) [M+H]⁺ 520.0/522.0

1H NMR (400 MHz, DMSO-d6) δ 13.32-12.51 (m, 1H), 12.04 (s, 1H), 9.03 (s, 2H), 7.43-7.37 (m, 1H), 7.23-7.10 (m, 2H), 6.83 (s, 1H), 5.20-4.48 (m, 2H), 4.21-3.59 (m, 2H), 3.53-3.47 (m, 1H), 3.21-3.05 (m, 2H), 3.00-2.59 (m, 2H), 2.01-1.19 (m, 4H)—signal of carboxylic acid not observed.

### Example 71

### 2-{1-[N-methyl-5-(4,5-difluoro-1H-indole-2-carbonyl)-2H,4H,5H,6H,7H-pyrazolo[4,3-c]pyridine-3-amido]cyclopropyl}pyrimidine-5-carboxylic acid

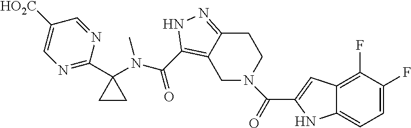 [SMILES: CN(C(=O)c1[nH]nc2c1CN(C(=O)c1cc3c(F)c(F)ccc3[nH]1)CC2)C1(c2ncc(C(=O)O)cn2)CC1]

Rt 3.35 mins (Method B2) [M+H]⁺ 522.1

### Example 72

### 2-{1-[N-methyl-5-(5-fluoro-4-methyl-1H-indole-2-carbonyl)-2H,4H,5H,6H,7H-pyrazolo[4,3-c]pyridine-3-amido]cyclopropyl}pyrimidine-5-carboxylic acid

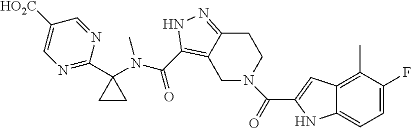 [SMILES: Cc1c(F)ccc2[nH]c(C(=O)N3CCc4n[nH]c(C(=O)N(C)C5(c6ncc(C(=O)O)cn6)CC5)c4C3)cc12]

Rt 3.38 mins (Method B2) [M+H]⁺ 518.1

### Example 73

### 2-{1-[N-methyl-5-(6-fluoro-4-methyl-1H-indole-2-carbonyl)-2H,4H,5H,6H,7H-pyrazolo[4,3-c]pyridine-3-amido]cyclopropyl}pyrimidine-5-carboxylic acid

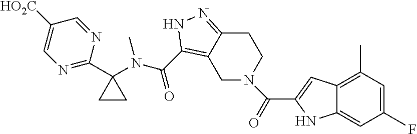 [SMILES: Cc1cc(F)cc2[nH]c(C(=O)N3CCc4n[nH]c(C(=O)N(C)C5(c6ncc(C(=O)O)cn6)CC5)c4C3)cc12]

Rt 3.39 mins (Method B2) [M+H]⁺ 518.1

### Example 74

### 4-{1-[N-methyl-5-(4-ethyl-6-fluoro-1H-indole-2-carbonyl)-4H,5H,6H,7H-pyrazolo[1,5-a]pyrazine-3-amido]cyclopropyl}benzoic acid

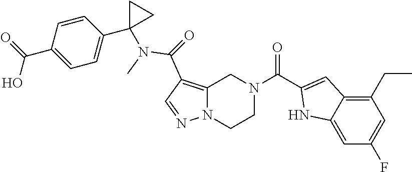 [SMILES: CCc1cc(F)cc2[nH]c(C(=O)N3CCn4ncc(C(=O)N(C)C5(c6ccc(C(=O)O)cc6)CC5)c4C3)cc12]

Rt 3.73 mins (Method B2) [M+H]⁺ 530.1

### Example 75

### 4-{1-[N-methyl-5-(4,6-difluoro-1H-indole-2-carbonyl)-4H,5H,6H,7H-pyrazolo[1,5-a]pyrazine-3-amido]cyclopropyl}benzoic acid

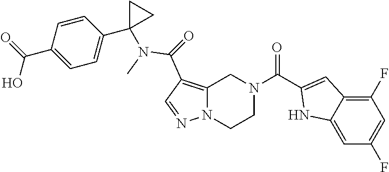 [SMILES: CN(C(=O)c1cnn2c1CN(C(=O)c1cc3c(F)cc(F)cc3[nH]1)CC2)C1(c2ccc(C(=O)O)cc2)CC1]

Rt 3.55 mins (Method B2) [M+H]⁺ 520.1

### Example 76

### 4-{1-[N-methyl-5-(4,7-difluoro-1H-indole-2-carbonyl)-4H,5H,6H,7H-pyrazolo[1,5-a]pyrazine-3-amido]cyclopropyl}benzoic acid

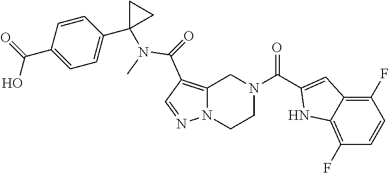 [SMILES: CN(C(=O)c1cnn2c1CN(C(=O)c1cc3c(F)ccc(F)c3[nH]1)CC2)C1(c2ccc(C(=O)O)cc2)CC1]

Rt 3.48 mins (Method B2) [M+H]⁺ 520.1

### Example 77

### 4-{1-[N-methyl-5-(5,6-difluoro-1H-indole-2-carbonyl)-4H,5H,6H,7H-pyrazolo[1,5-a]pyrazine-3-amido]cyclopropyl}benzoic acid

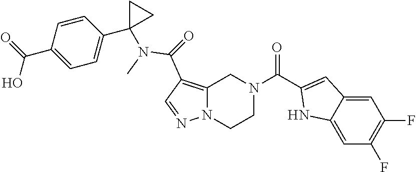 [SMILES: CN(C(=O)c1cnn2c1CN(C(=O)c1cc3cc(F)c(F)cc3[nH]1)CC2)C1(c2ccc(C(=O)O)cc2)CC1]

Rt 3.49 mins (Method B2) [M+H]⁺ 520.2

### Example 78

### 4-{1-[N-methyl-5-(4,5,6-trifluoro-1H-indole-2-carbonyl)-4H,5H,6H,7H-pyrazolo[1,5-a]pyrazine-3-amido]cyclopropyl}benzoic acid

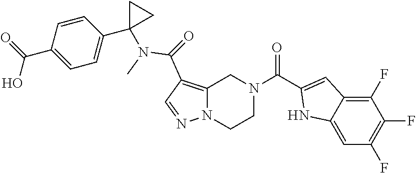 [SMILES: CN(C(=O)c1cnn2c1CN(C(=O)c1cc3c(F)c(F)c(F)cc3[nH]1)CC2)C1(c2ccc(C(=O)O)cc2)CC1]

Rt 3.62 mins (Method B2) [M+H]⁺ 538.1

### Example 79

### 12′-(5-fluoro-1H-indole-2-carbonyl)-4′-methyl-4′,7′, 8′,12′-tetraazaspiro[cyclopropane-1,5′-tricyclo[7.4.0.0²,7]tridecane]-1′,8′-dien-3′-one

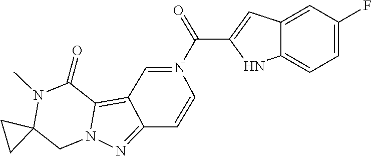

Rt 3.28 mins (Method A2) [M+H]⁺ 394.2

1H NMR (400 MHz, DMSO-d6) δ 11.77 (s, 1H), 7.41 (dt, J=8.4, 4.0 Hz, 2H), 7.06 (td, J=9.3, 2.6 Hz, 1H), 6.87 (s, 1H), 5.26-4.57 (m, 2H), 4.28-4.17 (m, 2H), 4.11-3.86 (m, 2H), 2.97-2.70 (m, 5H), 1.25-1.14 (m, 2H), 0.95-0.86 (m, 2H).

### Example 80

### 4-{1-[N-methyl-5-(4-chloro-5-fluoro-1H-indole-2-carbonyl)-4H,5H,6H,7H-pyrazolo[1,5-a]pyrazine-3-amido]cyclopropyl}benzoic acid

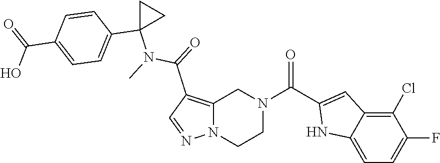 [SMILES: CN(C(=O)c1cnn2c1CN(C(=O)c1cc3c(Cl)c(F)ccc3[nH]1)CC2)C1(c2ccc(C(=O)O)cc2)CC1]

Rt 3.64 mins (Method B2) [M+H]⁺ 536.1/538.1

1H NMR (400 MHz, DMSO-d6) δ 12.49-11.91 (m, 1H), 8.02-7.74 (m, 2H), 7.44 (dd, J=8.9, 4.0 Hz, 1H), 7.27 (t, J=9.4 Hz, 1H), 7.15 (d, J=8.1 Hz, 2H), 7.00-6.93 (m, 2H), 5.71-4.73 (m, 2H), 4.48-3.94 (m, 4H), 3.04 (s, 3H), 1.73-1.29 (m, 4H)—signal of carboxylic acid not observed

### Example 81

### 4-{1-[N-methyl-5-(4-chloro-6-fluoro-1H-indole-2-carbonyl)-4H,5H,6H,7H-pyrazolo[1,5-a]pyrazine-3-amido]cyclopropyl}benzoic acid

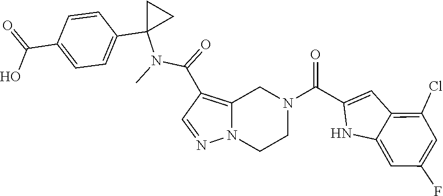 [SMILES: CN(C(=O)c1cnn2c1CN(C(=O)c1cc3c(Cl)cc(F)cc3[nH]1)CC2)C1(c2ccc(C(=O)O)cc2)CC1]

Rt 3.68 mins (Method B2) [M+H]⁺ 536.1/538.2

### Example 82

### 4-{1-[N-methyl-5-(4-methyl-1H-indole-2-carbonyl)-4H,5H,6H,7H-pyrazolo[1,5-a]pyrazine-3-amido]cyclopropyl}benzoic acid

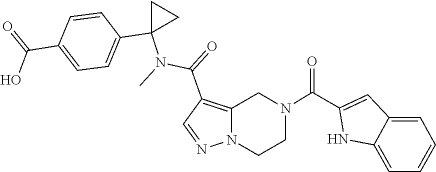 [SMILES: CN(C(=O)c1cnn2c1CN(C(=O)c1cc3ccccc3[nH]1)CC2)C1(c2ccc(C(=O)O)cc2)CC1]

Rt 3.49 mins (Method B2) [M+H]⁺ 498.1

### Example 83

### 4-{1-[N-methyl-5-(4-ethyl-1H-indole-2-carbonyl)-4H,5H,6H,7H-pyrazolo[1,5-a]pyrazine-3-amido]cyclopropyl}benzoic acid

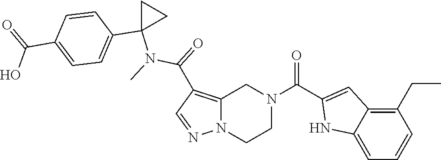 [SMILES: CCc1cccc2[nH]c(C(=O)N3CCn4ncc(C(=O)N(C)C5(c6ccc(C(=O)O)cc6)CC5)c4C3)cc12]

Rt 3.65 mins (Method B2) [M+H]⁺ 512.2

### Example 84

### 3-{1-[5-(1H-indole-2-carbonyl)-4H,5H,6H,7H-pyrazolo[1,5-a]pyrazine-3-amido]cyclopropyl}-1,2-oxazole-5-carboxylic acid

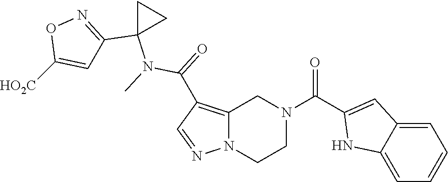 [SMILES: CN(C(=O)c1cnn2c1CN(C(=O)c1cc3ccccc3[nH]1)CC2)C1(c2cc(C(=O)O)on2)CC1]

Rt 2.53 mins (Method A2) [M+H]⁺ 475.1

1H NMR (400 MHz, DMSO-d6) δ 11.71 (s, 1H), 7.66 (d, J=8.0 Hz, 1H), 7.44 (d, J=8.1 Hz, 1H), 7.37-6.81 (m, 4H), 6.39 (s, 1H), 5.42-4.91 (m, 2H), 4.45-3.95 (m, 4H), 3.04 (s, 3H), 1.74-1.19 (m, 4H).

### Example 85

### 12′-(4,5-difluoro-1H-indole-2-carbonyl)-4′-methyl-4′,7′,8′,12′-tetraazaspiro[cyclopropane-1,5′-tricyclo[7.4.0.0²,7]tridecane]-1′,8′-dien-3′-one

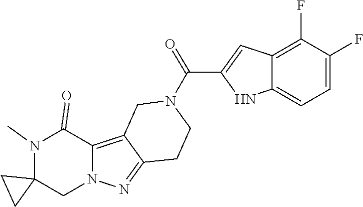 [SMILES: CN1C(=O)c2c3c(nn2CC12CC2)CCN(C(=O)c1cc2c(F)c(F)ccc2[nH]1)C3]

Rt 3.40 mins (Method A2) [M+H]⁺ 412.1

1H NMR (400 MHz, DMSO-d6) δ 12.09 (s, 1H), 7.29-7.21 (m, 2H), 6.98 (s, 1H), 5.21-4.67 (m, 2H), 4.23 (s, 2H), 4.11-3.88 (m, 2H), 3.01-2.70 (m, 5H), 1.25-1.10 (m, 2H), 0.96-0.82 (m, 2H).

### Example 86

### 13′-ethyl-4′-(1H-indole-2-carbonyl)-4′,8′,9′,13′-tetraazaspiro[cyclopropane-1,12′-tricyclo[7.5.0.0²,7]tetradecane]-1′,7′-dien-14′-one

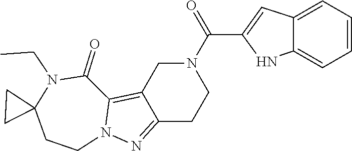 [SMILES: CCN1C(=O)c2c3c(nn2CCC12CC2)CCN(C(=O)c1cc2ccccc2[nH]1)C3]

Rt 3.32 mins (Method A2) [M+H]⁺ 404.2

1H NMR (400 MHz, DMSO-d6) δ 11.65 (s, 1H), 7.63 (d, J=7.9 Hz, 1H), 7.42 (d, J=8.2 Hz, 1H), 7.19 (ddd, J=8.2, 7.0, 1.2 Hz, 1H), 7.06 (ddd, J=8.1, 7.1, 1.0 Hz, 1H), 6.87 (s, 1H), 5.17-4.46 (m, 2H), 4.34 (t, J=6.8 Hz, 2H), 4.10-3.89 (m, 2H), 3.53-3.36 (m, 2H), 3.01-2.72 (m, 2H), 2.16-2.03 (m, 2H), 1.21 (t, J=7.3 Hz, 3H), 0.84-0.68 (m, 2H), 0.62-0.47 (m, 2H).

### Example 87

### 12′-(4-chloro-5-fluoro-1H-indole-2-carbonyl)-4′-methyl-4′,7′,8′,12′-tetraazaspiro[cyclopropane-1,5′-tricyclo[7.4.0.0²,7]tridecane]-1′,8′-dien-3′-one

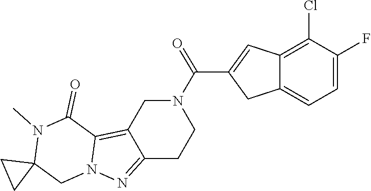 [SMILES: CN1C(=O)c2c3c(nn2CC12CC2)CCN(C(=O)C1=Cc2c(ccc(F)c2Cl)C1)C3]

Rt 3.52 mins (Method A2) [M+H]⁺ 428.1/430.1

1H NMR (400 MHz, DMSO-d6) δ 12.15 (s, 1H), 7.41 (dd, J=8.9, 3.9 Hz, 1H), 7.28-7.20 (m, 1H), 6.89 (s, 1H), 5.21-4.69 (m, 2H), 4.23 (s, 2H), 4.10-3.88 (m, 2H), 2.96-2.69 (m, 5H), 1.25-1.14 (m, 2H), 0.96-0.82 (m, 2H).

### Example 88

### 12′-(6-fluoro-4-methyl-1H-indole-2-carbonyl)-4′-methyl-4′,7′,8′,12′-tetraazaspiro[cyclopropane-1,5′-tricyclo[7.4.0.0²,7]tridecane]-1′,8′-dien-3′-one

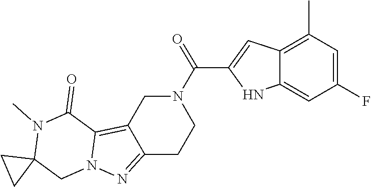 [SMILES: Cc1cc(F)cc2[nH]c(C(=O)N3CCc4nn5c(c4C3)C(=O)N(C)C3(CC3)C5)cc12]

Rt 3.44 mins (Method A2) [M+H]⁺ 408.2

1H NMR (400 MHz, DMSO-d6) δ 11.71 (s, 1H), 6.99-6.90 (m, 2H), 6.76 (dd, J=10.7, 2.3 Hz, 1H), 5.14-4.66 (m, 2H), 4.22 (s, 2H), 4.11-3.91 (m, 2H), 2.95-2.81 (m, 2H), 2.76 (s, 3H), 2.51 (s, 3H), 1.23-1.14 (m, 2H), 0.94-0.87 (m, 2H).

### Example 89

### 13′-(2-hydroxyethyl)-4′-(1H-indole-2-carbonyl)-4′, 8′,9′,13′-tetraazaspiro[cyclopropane-1,12′-tricyclo[7.5.0.0²,7]tetradecane]-1′,7′-dien-14′-one

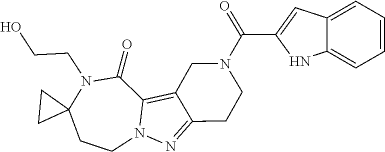 [SMILES: O=C(c1cc2ccccc2[nH]1)N1CCc2nn3c(c2C1)C(=O)N(CCO)C1(CC3)CC1]

Step 1: 5-(tert-butoxycarbonyl)-1-((2-(trimethylsilyl)ethoxy)methyl)-4,5,6,7-tetrahydro-1H-pyrazolo[4,3-c]pyridine-3-carboxylic acid (0.272 g, 0.684 mmol) and 2-(1-((2-(benzyloxy)ethyl)amino)cyclopropyl)ethyl benzoate hydrochloride (0.257 g, 0.684 mmol) were dissolved in pyridine (5 mL). The mixture was cooled to −12° C., phosphoryl chloride (0.127 mL, 1.367 mmol) was added and the reaction mixture was stirred for 3 h. The reaction mixture was concentrated in vacuo and the residue was stripped with heptane and dissolved in dichloromethane. The organic layer was washed with 1M KHSO₄, brine, dried over sodium sulfate and concentrated in vacuo. The resulting brown oil was dissolved in dichloromethane and purified by column chromatography (EtOAc in heptanes, 0% to 100%) to obtain tert-butyl 3-((1-(2-(benzoyloxy)ethyl)cyclopropyl)(2-(benzyloxy)ethyl)carbamoyl)-1-((2-(trimethylsilyl)ethoxy)methyl)-1,4,6,7-tetrahydro-5H-pyrazolo [4,3-c]pyridine-5-carboxylate as a colourles oil (0.388 g, 79% yield).

Step 2: Tert-butyl 3-((1-(2-(benzoyloxy)ethyl)cyclopropyl)(2-(benzyloxy)ethyl)carbamoyl)-1-((2-(trimethylsilyl)ethoxy)methyl)-1,4,6,7-tetrahydro-5H-pyrazolo[4,3-c]pyridine-5-carboxylate (0.388 g, 0.540 mmol) was dissolved in 4M HCl in dioxane (10 mL, 40.0 mmol) and stirred overnight. The reaction mixture was concentrated in vacuo. The residue was stripped with dichloromethane to obtain 2-(1-(N-(2-(benzyloxy)ethyl)-4,5,6,7-tetrahydro-1H-pyrazolo[4,3-c]pyridine-3-carboxamido)cyclopropyl)ethyl benzoate dihydrochloride (303 mg, quant. yield).

Step 3: 2-(1-(N-(2-(benzyloxy)ethyl)-4,5,6,7-tetrahydro-1H-pyrazolo[4,3-c]pyridine-3-carboxamido)cyclopropyl)ethyl benzoate dihydrochloride (0.303 g, 0.540 mmol) was suspended in dichloromethane (10 mL) and Et₃N (0.165 mL, 1.187 mmol) was added. Subsequently, boc-anhydride (0.138 mL, 0.594 mmol) was added and the mixture was stirred at r.t. for 1.5 h. The reaction mixture was quenched with saturated NH₄Cl and the water layer was extracted with dichloromethane. The combined organic layers were washed with brine, dried over sodium sulfate and concentrated in vacuo. The resulting oil was dissolve in dichloromethane and was purified by column chromatography (EtOAc in heptanes, 0% to 100%) to obtain tert-butyl 3-((1-(2-(benzoyloxy)ethyl)cyclopropyl)(2-(benzyloxy)ethyl)carbamoyl)-1,4,6,7-tetrahydro-5H-pyrazolo[4,3-c]pyridine-5-carboxylate as a white foam (0.165 g, 51% yield).

Step 4: Tert-butyl 3-((1-(2-(benzoyloxy)ethyl)cyclopropyl)(2-(benzyloxy)ethyl)carbamoyl)-1,4,6,7-tetrahydro-5H-pyrazolo[4,3-c]pyridine-5-carboxylate (0.165 g, 0.280 mmol) was dissolved in tetrahydrofuran (5 mL). To this water (5 mL) was added, followed by lithium hydroxide monohydrate (0.035 g, 0.841 mmol). The mixture was stirred at r.t. overnight.

Additonal lithium hydroxide monohydrate (0.035 g, 0.841 mmol) was added and the mixture was stirred for anohter 3 h. The reaction mixture was acidified with 1M HCl (1.682 mL, 1.682 mmol) and was concentrated in vacuo. The residue was stripped with toluene and purified by preparative HPLC to obtain tert-butyl 3-((2-(benzyloxy)ethyl)(1-(2-hydroxyethyl)cyclopropyl)carbamoyl)-1,4,6,7-tetrahydro-5H-pyrazolo [4,3-c]pyridine-5-carboxylate (0.100 g, 73% yield).

Step 5: Tert-butyl 3-((2-(benzyloxy)ethyl)(1-(2-hydroxyethyl)cyclopropyl)carbamoyl)-1,4,6,7-tetrahydro-5H-pyrazolo[4,3-c]pyridine-5-carboxylate (0.100 g, 0.206 mmol) was dissolved in tetrahydrofuran (15 mL). To this triphenylphosphine (0.070 g, 0.268 mmol) was added. A solution of diisopropyl azodicarboxylate (0.052 mL, 0.268 mmol) in tetrahydrofuran (5 mL) was added dropwise and the mixture was stirred at 80° C. After 2 h additonal diisopropyl azodicarboxylate (0.020 mL, 0.103 mmol) and triphenylphosphine (0.054 g, 0.206 mmol) were added. The mixture was stirred at 80° C. for 2 h. The reaction mixture was poured into water (100 mL) and extracted with EtOAc (2×200 mL). The combined organic layers were washed with brine (300 mL). The organic layer was dried over sodium sulfate and concentrated in vacuo. The residue taken up in dichloromethane and was purified by column chromatography (EtOAc in heptanes, 10% to 100%) to obtain tert-butyl 10′-(2-(benzyloxy)ethyl)-11′-oxo-3′,4′,7′,8′,10′,11′-hexahydrospiro[cyclopropane-1,9′-pyrido[4′,3′:3,4]pyrazolo [1,5-a][1,4]diazepine]-2′(1′H)-carboxylate (0.098 g, 62% yield).

Step 6: Tert-butyl 10′-(2-(benzyloxy)ethyl)-11′-oxo-3′,4′,7′,8′,10′,11′-hexahydrospiro[cyclopropane-1,9′-pyrido[4′,3′:3,4]pyrazolo[1,5-a]1[1,4]diazepine]-2′(1′H)-carboxylate (0.098 g, 0.210 mmol) was dissolved in EtOH (5 mL). To this palladium on carbon (0.050 g, 0.047 mmol) was added and the mixture was brought under hydrogen atmosphere and was stirred at r.t. overnight. The reaction mixture was filtered over Celite and flushed with MeOH and concentracted in vacuo. The residue was purified by preparative HPLC to obtain tert-butyl 10′-(2-hydroxyethyl)-11′-oxo-3′,4′,7′,8′,10′,11′-hexahydrospiro[cyclopropane-1,9′-pyrido[4′,3′:3,4]pyrazolo[1,5-a][1,4]diazepine]-2′(1′H)-carboxylate (0.030 g, 37% yield).

Step 7: Tert-butyl 10′-(2-hydroxyethyl)-11′-oxo-3′,4′,7′,8′,10′,11′-hexahydrospirol[cyclopropane-1,9′-pyrido[4′,3′:3,4]pyrazolo[1,5-a][1,4]diazepine]-2′(1′H)-carboxylate (0.030 g, 0.080 mmol) was dissolved in 4M HCl in dioxane (5 mL, 20.00 mmol).

After stirring the reaction reaction for 2 h, it was concentrated in vacuo and the residue was stripped with dichloromethane to obtain 10′-(2-hydroxyethyl)-1′,2′,3′,4′,7′,8′-hexahydrospiro[cyclopropane-1,9′-pyrido[4′,3′:3,4]pyrazolo[1,5-a][1,4]diazepin]-11′(10′H)-one hydrochloride (25 mg, quant. yield).

Step 8: 10′-(2-hydroxyethyl)-1′,2′,3′,4′,7′,8′-hexahydrospiro[cyclopropane-1,9′-pyrido[4′,3′:3,4]pyrazolo[1,5-a][1,4]diazepin]-11′(10′H)-one hydrochloride (0.025 g, 0.080 mmol) was dissolved in N,N-dimethylformamide (1 mL). To this Et₃N (0.056 mL, 0.400 mmol) was added. In a separate vial HATU (0.036 g, 0.096 mmol) and 1H-indole-2-carboxylic acid (0.013 g, 0.080 mmol) were stirred in N,N-dimethylformamide (1 mL) for 10 mins. This solution was added to the former solution and the mixture was stirred at r.t overnight. The mixture was quenched with water (0.250 mL) and the solution was filtered and flushed with DMSO (0.2 mL) and purified by preparative HPLC to obtain 10′-(2-hydroxyethyl)-2′-(1H-indole-2-carbonyl)-1′,2′,3′,4′,7′,8′-hexahydrospiro[cyclopropane-1,9′-pyrido[4′,3′:3,4]pyrazolo[1,5-a][1,4]diazepin]-11′(10′H)-one as a white solid (0.020 g, 59.7% yield).

Rt 2.98 mins (Method A2) [M+H]⁺ 420.2

1H NMR (400 MHz, DMSO-d6) δ 11.65 (s, 1H), 7.63 (d, J=8.0 Hz, 1H), 7.42 (d, J=8.0 Hz, 1H), 7.19 (ddd, J=8.1, 6.9, 1.2 Hz, 1H), 7.06 (ddd, J=7.9, 6.8, 1.0 Hz, 1H), 6.87 (s, 1H), 5.12-4.55 (m, 3H), 4.36 (t, J=6.6 Hz, 2H), 4.11-3.88 (m, 2H), 3.68-3.56 (m, 2H), 3.55-3.38 (m, 2H), 2.98-2.72 (m, 2H), 2.20-1.99 (m, 2H), 0.88-0.67 (m, 2H), 0.58-0.42 (m, 2H).

### Example 90

### 13′-[2-(benzyloxy)ethyl]-4′-(1H-indole-2-carbonyl)-4′,8′,9′,13′-tetraazaspiro[cyclopropane-1,12′-tricyclo[7.5.0.0²,7]tetradecane]-1′,7′-dien-14′-one

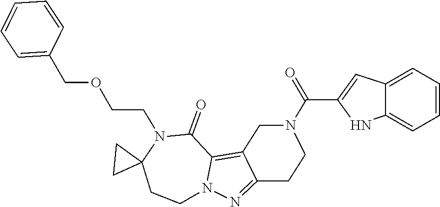 [SMILES: O=C(c1cc2ccccc2[nH]1)N1CCc2nn3c(c2C1)C(=O)N(CCOCc1ccccc1)C1(CC3)CC1]

Rt 1.72 mins (Method J) [M+H]⁺ 510.4

1H NMR (400 MHz, DMSO-d6) δ 11.65 (d, J=2.2 Hz, 1H), 7.63 (d, J=7.8 Hz, 1H), 7.42 (d, J=8.2 Hz, 1H), 7.38-7.25 (m, 5H), 7.19 (ddd, J=8.2, 6.9, 1.2 Hz, 1H), 7.06 (t, J=7.5 Hz, 1H), 6.87 (s, 1H), 5.14-4.58 (m, 2H), 4.51 (s, 2H), 4.33-4.17 (m, 2H), 4.14-3.89 (m, 2H), 3.80-3.54 (m, 4H), 2.98-2.71 (m, 2H), 2.14-1.94 (m, 2H), 0.88-0.72 (m, 2H), 0.59-0.47 (m, 2H).

### Example 91

### 4-[7-(1H-indole-2-carbonyl)-5H,6H,7H,8H-imidazo[1,5-a]pyrazine-1-carbonyl]-8-oxa-4-azaspiro[2.6]nonane

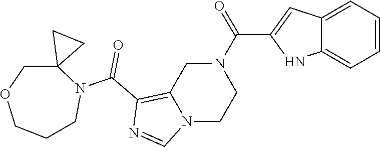 [SMILES: O=C(c1cc2ccccc2[nH]1)N1CCn2cnc(C(=O)N3CCCOCC34CC4)c2C1]

Rt 1.20 mins (Method H) [M+H]⁺ 420.2

1H NMR (400 MHz, DMSO-d6) δ 11.71 (s, 1H), 7.73 (s, 1H), 7.66 (d, J=7.9 Hz, 1H), 7.44 (d, J=8.2 Hz, 1H), 7.21 (t, J=7.6 Hz, 1H), 7.07 (t, J=7.4 Hz, 1H), 6.94 (s, 1H), 5.58-4.75 (m, 2H), 4.71-3.94 (m, 6H), 3.92-3.37 (m, 4H), 2.03-1.76 (m, 2H), 0.94-0.68 (m, 4H).

### Example 92

### 4-[7-(1H-indole-2-carbonyl)-5H,6H,7H,8H-imidazo[1,5-a]pyrazine-1-carbonyl]-4-azaspiro[2.5]octan-7-ol

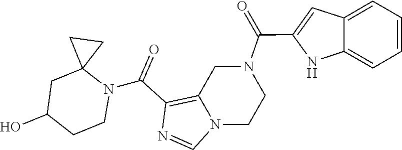 [SMILES: O=C(c1cc2ccccc2[nH]1)N1CCn2cnc(C(=O)N3CCC(O)CC34CC4)c2C1]

Rt 1.04 mins (Method H) [M+H]⁺ 420.2

1H NMR (400 MHz, DMSO-d6) δ 11.71 (s, 1H), 7.71 (s, 1H), 7.67 (d, J=8.0 Hz, 1H), 7.44 (d, J=8.2 Hz, 1H), 7.21 (t, J=7.6 Hz, 1H), 7.07 (t, J=7.5 Hz, 1H), 6.93 (s, 1H), 5.42-4.71 (m, 3H), 4.67-4.59 (m, 1H), 4.34-3.99 (m, 4H), 3.82-3.70 (m, 1H), 3.28-3.07 (m, 1H), 2.01-1.68 (m, 2H), 1.45-1.03 (m, 2H), 0.88-0.76 (m, 2H), 0.55-0.39 (m, 2H).

### Example 93

### 4-{1-[N-methyl-7-(1H-indole-2-carbonyl)-5H,6H,7H,8H-imidazo[1,5-a]pyrazine-1-amido]cyclopropyl}benzoic acid

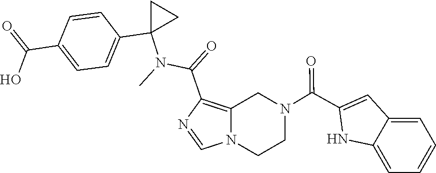 [SMILES: CN(C(=O)c1ncn2c1CN(C(=O)c1cc3ccccc3[nH]1)CC2)C1(c2ccc(C(=O)O)cc2)CC1]

Rt 1.30 mins (Method H) [M+H]⁺ 484.4

1H NMR (400 MHz, DMSO-d6) δ 11.71 (s, 1H), 7.90-7.74 (m, 3H), 7.70-7.60 (m, 1H), 7.52-7.38 (m, 1H), 7.25-7.17 (m, 1H), 7.16-7.00 (m, 3H), 6.97-6.89 (m, 1H), 5.51-4.82 (m, 2H), 4.43-3.86 (m, 4H), 3.55-3.47 (m, 2H), 3.03-2.90 (m, 1H), 1.54-1.17 (m, 4H)—proton of carboxylic acid not observed.

### Example 94

### 2-{1-[N-methyl-7-(1H-indole-2-carbonyl)-5H,6H,7H,8H-imidazo[1,5-a]pyrazine-1-amido]cyclopropyl}pyrimidine-5-carboxylic acid

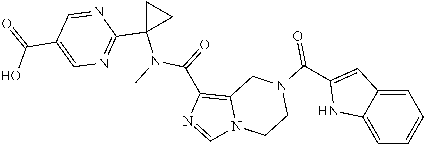 [SMILES: CN(C(=O)c1ncn2c1CN(C(=O)c1cc3ccccc3[nH]1)CC2)C1(c2ncc(C(=O)O)cn2)CC1]

Rt 1.22 mins (Method H) [M+H]⁺ 486.4

1H NMR (400 MHz, DMSO-d6) δ 11.70 (s, 1H), 9.05 (s, 2H), 7.82-7.34 (m, 3H), 7.25-7.16 (m, 1H), 7.11-7.01 (m, 1H), 6.95-6.87 (m, 1H), 5.44-4.84 (m, 2H), 4.37-3.86 (m, 4H), 3.64-3.59 (m, 1H), 3.15-3.02 (m, 2H), 2.01-1.87 (m, 1H), 1.63-1.30 (m, 3H)—carboxylic acid proton not observed.

### Example 95

### 4-[5-(1H-indole-2-carbonyl)-4H,5H,6H,7H-[1,2]oxazolo[4,5-c]pyridine-3-carbonyl]-4-azaspiro[2.5]octan-7-ol

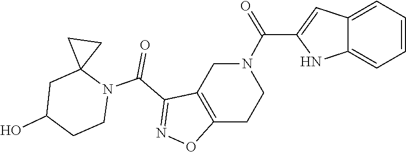 [SMILES: O=C(c1cc2ccccc2[nH]1)N1CCc2onc(C(=O)N3CCC(O)CC34CC4)c2C1]

Rt 3.15 mins (Method A2) [M+H]⁺ 421.1

1H NMR (400 MHz, DMSO-d6) δ 11.67 (s, 1H), 7.65 (d, J=8.0 Hz, 1H), 7.43 (d, J=8.3 Hz, 1H), 7.25-7.17 (m, 1H), 7.07 (t, J=7.5 Hz, 1H), 6.92 (s, 1H), 5.31-4.63 (m, 3H), 4.49-3.62 (m, 4H), 3.16-2.76 (m, 3H), 2.06-1.77 (m, 2H), 1.57-1.18 (m, 2H), 1.09-0.43 (m, 4H).

### Example 96

### 4-[5-(1H-indole-2-carbonyl)-4H,5H,6H,7H-[1,2]oxazolo[4,5-c]pyridine-3-carbonyl]-8-oxa-4-azaspiro[2.6]nonane

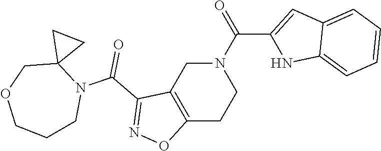 [SMILES: O=C(c1cc2ccccc2[nH]1)N1CCc2onc(C(=O)N3CCCOCC34CC4)c2C1]

Rt 3.39 mins (Method A2) [M+H]⁺ 421.2

1H NMR (400 MHz, DMSO-d6) δ 11.68 (s, 1H), 7.65 (d, J=8.0 Hz, 1H), 7.43 (d, J=8.2 Hz, 1H), 7.21 (t, J=7.6 Hz, 1H), 7.07 (t, J=7.4 Hz, 1H), 6.97-6.85 (m, 1H), 5.39-4.49 (m, 2H), 4.13-3.94 (m, 2H), 3.91-3.71 (m, 2H), 3.70-3.61 (m, 1H), 3.60-3.48 (m, 1H), 3.08-2.89 (m, 2H), 2.05-1.85 (m, 2H), 1.03-0.73 (m, 4H)—one signal (2H) coincides with water signal.

### Example 97

### 4-{1-[N-methyl-5-(1H-indole-2-carbonyl)-4H,5H,6H,7H-[1,2]oxazolo[4,5-c]pyridine-3-amido]cyclopropyl}benzoic acid

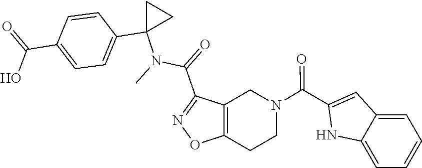 [SMILES: CN(C(=O)c1noc2c1CN(C(=O)c1cc3ccccc3[nH]1)CC2)C1(c2ccc(C(=O)O)cc2)CC1]

Rt 2.76 mins (Method A2) [M+H]⁺ 485.2

1H NMR (400 MHz, DMSO-d6) δ 11.68 (s, 1H), 7.94-7.80 (m, 2H), 7.69-7.61 (m, 1H), 7.43 (d, J=8.4 Hz, 1H), 7.32-7.11 (m, 3H), 7.10-7.02 (m, 1H), 6.96-6.86 (m, 1H), 5.44-4.56 (m, 2H), 4.12-3.78 (m, 2H), 3.11-2.76 (m, 5H), 1.65-1.26 (m, 4H)—proton of carboxylic acid not observed.

### Example 98

### 2-{1-[N-methyl-5-(1H-indole-2-carbonyl)-4H,5H,6H,7H-[1,2]oxazolo[4,5-c]pyridine-3-amido]cyclopropyl}pyrimidine-5-carboxylic acid

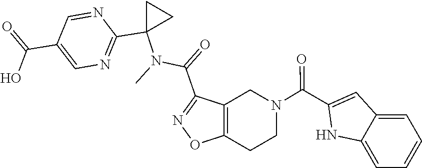 [SMILES: CN(C(=O)c1noc2c1CN(C(=O)c1cc3ccccc3[nH]1)CC2)C1(c2ncc(C(=O)O)cn2)CC1]

Step 1: Tert-butyl 3-((1-(5-(methoxycarbonyl)pyrimidin-2-yl)cyclopropyl)(methyl)carbamoyl)-6,7-dihydroisoxazolo[4,5-c]pyridine-5(4H)-carboxylate (0.030 g, 0.066 mmol) was dissolved in 4M HCl in dioxane (3 mL, 12.00 mmol) and the mixture was stirred overnight. The reaction mixture was concentrated and co-evaporated with dichloromethane (2×5 mL) to obtain methyl 2-(1-(N-methyl-4,5,6,7-tetrahydroisoxazolo[4,5-c]pyridine-3-carboxamido)cyclopropyl)pyrimidine-5-carboxylate hydrochloride as a beige solid (0.026 g, quant. yield).

Step 2: To a mixture of 1H-indole-2-carboxylic acid (10.64 mg, 0.066 mmol), methyl 2-(1-(N-methyl-4,5,6,7-tetrahydroisoxazolo[4,5-c]pyridine-3-carboxamido)cyclopropyl)pyrimidine-5-carboxylate hydrochloride (0.026 g, 0.066 mmol) and Et₃N (0.046 mL, 0.330 mmol) in N,N-dimethylformamide (0.5 mL) was added HATU (0.026 g, 0.069 mmol). After stirring the reaction mixture for 2 h, a solution of lithium hydroxide monohydrate (0.042 g, 1 mmol) in water (0.5 mL) was added and was stirred for 1 h. The reaction mixture was quenched with 6M aqueous HCl (0.2 mL) and was stirred at r.t. overnight. The reaction mixture was filtered and purified by preparative HPLC-MS to afford the product as a white fluffy solid (0.023 g, 68% yield).

Rt 2.67 mins (Method A2) [M+H]⁺ 487.1

1H NMR (400 MHz, DMSO-d6) δ 11.67 (s, 1H), 9.22-8.92 (m, 2H), 7.72-7.58 (m, 1H), 7.48-7.39 (m, 1H), 7.26-7.15 (m, 1H), 7.11-7.01 (m, 1H), 6.95-6.85 (m, 1H), 5.24-4.66 (m, 2H), 4.14-3.82 (m, 2H), 3.14-2.89 (m, 5H), 1.97-1.81 (m, 1H), 1.72-1.48 (m, 3H)-proton of carboxylic acid not observed.

### Example 99

### 5-(4,6-dichloro-1H-indole-2-carbonyl)-N-{1-[(difluoromethoxy)methyl]cyclopropyl}-4H,5H,6H,7H-[1,2]oxazolo[4,5-c]pyridine-3-carboxamide

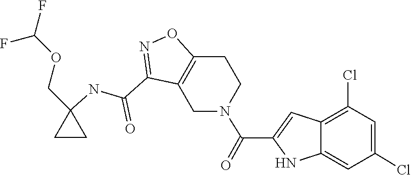

Prepared as described for Example 100.

Rt 4.10 mins (Method A2) [M+H]⁺ 498.9/501.1

1H NMR (400 MHz, DMSO) δ 12.21 (s, 1H), 9.11 (s, 1H), 7.44 (s, 1H), 7.26 (s, 1H), 6.93 (s, 1H), 6.67 (t, J=76.1 Hz, 1H), 5.18-4.51 (m, 2H), 4.22-3.84 (m, 4H), 3.20-2.85 (m, 2H), 0.98-0.71 (m, 4H).

### Example 100

### 5-(4-chloro-5-fluoro-1H-indole-2-carbonyl)-N-{1-[(difluoromethoxy)methyl]cyclopropyl}-4H,5H,6H,7H-[1,2]oxazolo[4,5-c]pyridine-3-carboxamide

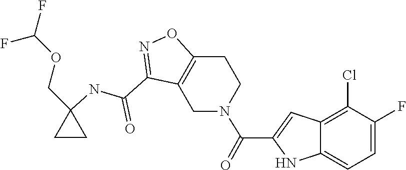

Step 1: To a solution of 5-(tert-butoxycarbonyl)-4,5,6,7-tetrahydroisoxazolo[4,5-c]pyridine-3-carboxylic acid (120 mg, 0.447 mmol) in N,N-dimethylformamide (0.6 mL) was added HATU (170 mg, 0.447 mmol). The resulting yellow solution was stirred at r.t. for 20 mins, then a solution of 1-((difluoromethoxy)methyl)cyclopropan-1-amine hydrochloride (78 mg, 0.447 mmol) in N,N-dimethylformamide (0.7 mL) was added, followed by Et₃N (0.312 mL, 2.237 mmol). After stirring for 30 mins the reaction mixture was filtered and purified by preparative HPLC to afford tert-butyl 3-((1-((difluoromethoxy)methyl)cyclopropyl)carbamoyl)-6,7-dihydroisoxazolo[4,5-c]pyridine-5(4H)-carboxylate as an off-white solid (173 mg, 69% yield).

Step 2: Tert-butyl 3-((1-((difluoromethoxy)methyl)cyclopropyl)carbamoyl)-6,7-dihydroisoxazolo[4,5-c]pyridine-5(4H)-carboxylate (78 mg, 0.201 mmol) was dissolved in 4M HCl in dioxane (1107 μL, 4.43 mmol) and the resulting solution was stirred at r.t. After 2 h the reaction mixture was diluted with dioxane (5 mL) and concentrated in vacuo. The residue was co-evaporated with toluene (2×5 mL) to give N-(1-((difluoromethoxy)methyl)cyclopropyl)-4,5,6,7-tetrahydroisoxazolo[4,5-c]pyridine-3-carboxamide hydrochloride as an off-white solid (65 mg, quant. yield).

Step 3: To 4-chloro-5-fluoro-1H-indole-2-carboxylic acid (14.2 mg, 0.067 mmol) in N,N-dimethylformamide (0.5 mL) was added HATU (0.025 g, 0.067 mmol) and the reaction mixture was stirred at r.t. for 15 mins. Then, a solution of N-(1-((difluoromethoxy)methyl)cyclopropyl)-4,5,6,7-tetrahydroisoxazolo[4,5-c]pyridine-3-carboxamide hydrochloride (0.022 g, 0.067 mmol) in N,N-dimethylformamide (0.8 mL) was added, followed by the addition of Et₃N (0.047 mL, 0.335 mmol). After 45 mins, the reaction mixture was filtered through a micro filter and purified by preparative HPLC to afford the product as a fluffy white solid (19 mg, 59% yield).

Rt 3.94 mins (Method A2) [M+H]⁺ 483.1/485.1

1H NMR (400 MHz, DMSO) δ 12.16 (s, 1H), 9.12 (s, 1H), 7.42 (dd, J=9.0, 3.9 Hz, 1H), 7.33-7.19 (m, 1H), 6.93 (s, 1H), 6.67 (t, J=76.1 Hz, 1H), 4.82 (s, 2H), 3.99 (d, J=39.9 Hz, 4H), 3.06 (s, 2H), 0.86 (d, J=13.5 Hz, 4H).

### Example 101

### 3-chloro-4-({1-[N-methyl-5-(1H-indole-2-carbonyl)-4H,5H,6H,7H-pyrazolo[1,5-a]pyrazine-3-amido]cyclopropyl}methoxy)benzoic acid

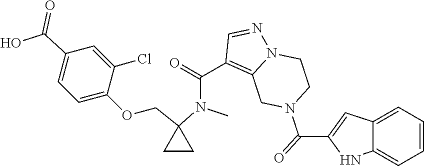 [SMILES: CN(C(=O)c1cnn2c1CN(C(=O)c1cc3ccccc3[nH]1)CC2)C1(COc2ccc(C(=O)O)cc2Cl)CC1]

Rt 2.76 mins (Method A2) [M+H]⁺ 548.2/550.1

1H NMR (400 MHz, DMSO) δ 11.70 (s, 1H), 8.21-7.72 (m, 3H), 7.64 (d, J=8.1 Hz, 1H), 7.43 (d, J=8.3 Hz, 1H), 7.21 (t, J=7.5 Hz, 2H), 7.07 (t, J=7.4 Hz, 1H), 6.94 (s, 1H), 5.37-4.91 (m, 2H), 4.64-4.03 (m, 6H), 1.45-0.78 (m, 4H).

### Example 102

### 4-[5-(1H-indole-2-carbonyl)-4H,5H,6H,7H-[1,2]oxazolo[4,3-c]pyridine-3-carbonyl]-4-azaspiro[2.5]octan-7-ol

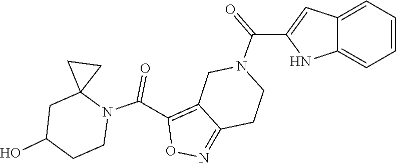 [SMILES: O=C(c1cc2ccccc2[nH]1)N1CCc2noc(C(=O)N3CCC(O)CC34CC4)c2C1]

Rt 1.33 mins (Method J) [M+H]⁺ 421.4

1H NMR (400 MHz, DMSO) δ 11.67 (s, 1H), 7.65 (d, J=7.9 Hz, 1H), 7.43 (d, J=8.2 Hz, 1H), 7.21 (t, J=7.6 Hz, 1H), 7.07 (t, J=7.5 Hz, 1H), 6.92 (s, 1H), 5.13-4.71 (m, 3H), 4.48-3.74 (m, 4H), 3.57-3.42 (m, 1H), 3.12-2.80 (m, 2H), 2.12-1.60 (m, 2H), 1.54-1.21 (m, 2H), 0.99-0.66 (m, 2H), 0.63-0.50 (m, 2H).

### Example 103

### 5-(6-chloro-5-fluoro-1H-indole-2-carbonyl)-N-[(2R)-1,1,1-trifluoropropan-2-yl]-4H,5H,6H,7H-[1,2]oxazolo[4,5-c]pyridine-3-carboxamide

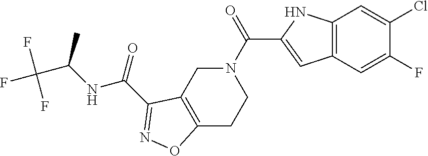 [SMILES: C[C@@H](NC(=O)c1noc2c1CN(C(=O)c1cc3cc(F)c(Cl)cc3[nH]1)CC2)C(F)(F)F]

Rt 1.71 mins (Method H) [M+H]⁺ 459.2/461.2

1H NMR (400 MHz, DMSO) δ 11.92 (s, 1H), 9.61 (s, 1H), 7.66 (d, J=9.9 Hz, 1H), 7.56 (d, J=6.5 Hz, 1H), 6.96 (s, 1H), 5.25-4.85 (m, 2H), 4.84-4.68 (m, 1H), 4.12-3.84 (m, 2H), 3.13-2.92 (m, 2H), 1.36 (d, J=7.1 Hz, 3H).”

### Example 104

### 5-(4-chloro-1H-indole-2-carbonyl)-N-[(2R)-1,1,1-trifluoropropan-2-yl]-4H,5H,6H,7H-[1,2]oxazolo[4,5-c]pyridine-3-carboxamide

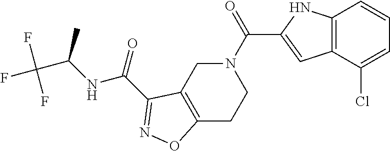 [SMILES: C[C@@H](NC(=O)c1noc2c1CN(C(=O)c1cc3c(Cl)cccc3[nH]1)CC2)C(F)(F)F]

Rt 1.70 mins (Method H) [M+H]⁺ 441.2/443.2

### Example 105

### 5-(4,5-difluoro-1H-indole-2-carbonyl)-N-[(2R)-1,1,1-trifluoropropan-2-yl]-4H,5H,6H,7H-[1,2]oxazolo[4,5-c]pyridine-3-carboxamide

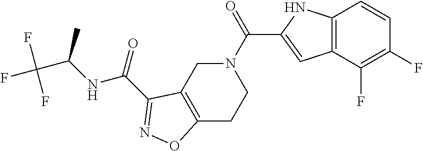 [SMILES: C[C@@H](NC(=O)c1noc2c1CN(C(=O)c1cc3c(F)c(F)ccc3[nH]1)CC2)C(F)(F)F]

Rt 1.65 mins (Method H) [M+H]⁺ 443.2

### Example 106

### 5-(5-fluoro-4-methyl-1H-indole-2-carbonyl)-N-[(2R)-1,1,1-trifluoropropan-2-yl]-4H,5H,6H,7H-[1,2]oxazolo[4,5-c]pyridine-3-carboxamide

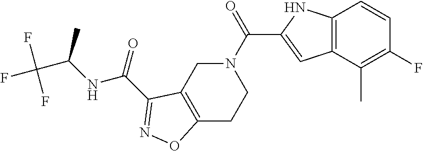 [SMILES: Cc1c(F)ccc2[nH]c(C(=O)N3CCc4onc(C(=O)N[C@H](C)C(F)(F)F)c4C3)cc12]

Rt 1.67 mins (Method H) [M+H]⁺ 439.2

### Example 107

### 5-(6-fluoro-4-methyl-1H-indole-2-carbonyl)-N-[(2R)-1,1,1-trifluoropropan-2-yl]-4H,5H,6H,7H-[1,2]oxazolo[4,5-c]pyridine-3-carboxamide

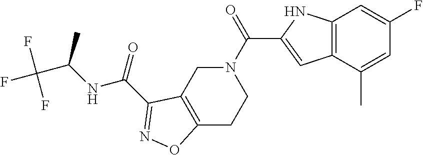 [SMILES: Cc1cc(F)cc2[nH]c(C(=O)N3CCc4onc(C(=O)N[C@H](C)C(F)(F)F)c4C3)cc12]

Rt 1.68 mins (Method H) [M+H]⁺ 439.2

### Example 108

### 5-(1H-indole-2-carbonyl)-N-methyl-N-{1-[(2r,5r)-5-amino-1,3-dioxan-2-yl]cyclopropyl}-4H,5H,6H,7H-[1,2]oxazolo[4,5-c]pyridine-3-carboxamide

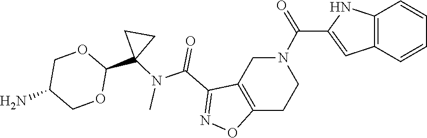 [SMILES: CN(C(=O)c1noc2c1CN(C(=O)c1cc3ccccc3[nH]1)CC2)C1([C@H]2OC[C@H](N)CO2)CC1]

Rt 1.36 mins (Method J) [M+H]⁺ 466.4.

1H NMR (400 MHz, DMSO) δ 11.68 (s, 1H), 7.69-7.60 (m, 1H), 7.46-7.39 (m, 1H), 7.24-7.17 (m, 1H), 7.11-7.03 (m, 1H), 6.93 (s, 1H), 5.02-4.51 (m, 3H), 4.25-3.74 (m, 5H), 3.29-2.94 (m, 8H), 2.92-2.72 (m, 1H), 0.95-0.70 (m, 4H).

### Example 109

### 4-{1-[N-methyl-5-(1H-indole-2-carbonyl)-4H,5H,6H,7H-[1,2]oxazolo[4,3-c]pyridine-3-amido]cyclopropyl}benzoic acid

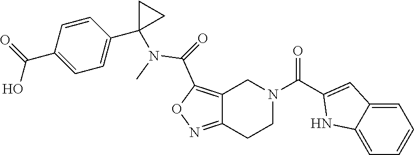 [SMILES: CN(C(=O)c1onc2c1CN(C(=O)c1cc3ccccc3[nH]1)CC2)C1(c2ccc(C(=O)O)cc2)CC1]

Rt 3.58 mins (Method B2) [M+H]⁺ 485.2

1H NMR (400 MHz, DMSO) δ 11.68 (s, 1H), 7.94-7.82 (m, 2H), 7.69-7.61 (m, 1H), 7.43 (d, J=8.2 Hz, 1H), 7.25-7.12 (m, 3H), 7.11-7.03 (m, 1H), 6.96-6.88 (m, 1H), 4.99 (s, 2H), 4.17-3.81 (m, 2H), 3.09 (s, 3H), 3.00-2.84 (m, 2H), 1.58-1.22 (m, 4H)—proton of carboxylic acid not observed.

### Example 110

### 4-{1-[N-methyl-5-(4,6-dichloro-5-fluoro-1H-indole-2-carbonyl)-4H,5H,6H,7H-pyrazolo [1,5-a]pyrazine-3-amido]cyclopropyl}benzoic acid

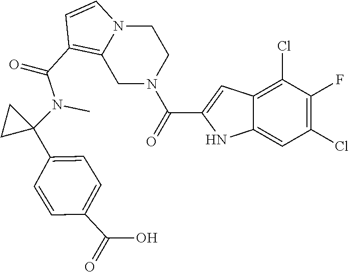 [SMILES: CN(C(=O)c1ccn2c1CN(C(=O)c1cc3c(Cl)c(F)c(Cl)cc3[nH]1)CC2)C1(c2ccc(C(=O)O)cc2)CC1]

Rt 3.85 mins (Method B2) [M+H]⁺ 570.1/572.1

1H NMR (400 MHz, DMSO) δ 12.27 (br s, 1H), 8.02-7.80 (m, 2H), 7.67-7.50 (m, 1H), 7.15 (d, J=8.0 Hz, 2H), 7.01 (s, 1H), 6.96 (s, 1H), 5.79-4.70 (m, 2H), 4.54-3.83 (m, 4H), 3.04 (s, 3H), 1.78-1.21 (m, 4H).—proton of carboxylic acid not observed.

### Example 111

### 5-(5,6-difluoro-1H-indole-2-carbonyl)-N-[(2R)-1,1,1-trifluoropropan-2-yl]-4H,5H,6H,7H-[1,2]oxazolo[4,5-c]pyridine-3-carboxamide

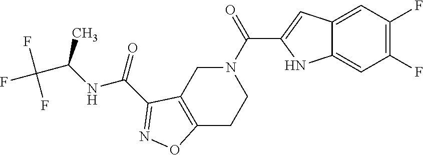 [SMILES: C[C@@H](NC(=O)c1noc2c1CN(C(=O)c1cc3cc(F)c(F)cc3[nH]1)CC2)C(F)(F)F]

Step 1: Tert-butyl (R)-3-((1,1,1-trifluoropropan-2-yl)carbamoyl)-6,7-dihydroisoxazolo[4,5-c]pyridine-5(4H)-carboxylate (0.120 g, 0.330 mmol) was dissolved in 4M HCl in dioxane (4 mL, 16.00 mmol) and the mixture was stirred overnight. The reaction mixture was concentrated in vacuo and co-evaporated with dichloromethane (2×5 mL) to obtain (R)-N-(1,1,1-trifluoropropan-2-yl)-4,5,6,7-tetrahydroisoxazolo[4,5-c]pyridine-3-carboxamide hydrochloride as a light beige solid (0.099 g, 94% yield).

Step 2: To 5,6-difluoro-1H-indole-2-carboxylic acid (0.018, 0.083 mmol) in N,N-dimethylformamide (0.4 mL), was added HATU (0.033 g, 0.087 mmol) and the mixture was stirred in a closed vial for 30 mins. To this a solution of (R)-N-(1,1,1-trifluoropropan-2-yl)-4,5,6,7-tetrahydroisoxazolo[4,5-c]pyridine-3-carboxamide hydrochloride (0.025 g, 0.083 mmol) in N,N-dimethylformamide (0.4 mL) and Et₃N (0.1 mL) was added and the mixture was stirred for three days. The reaction mixture was filtered and purified by preparative HPLC-MS to afford the product as a fluffy white solid (0.016 g, 44% yield).

Rt 1.65 mins (Method J) [M+H]⁺ 443.2

1H NMR (400 MHz, DMSO) δ 12.17-11.59 (m, 1H), 9.83-9.38 (m, 1H), 7.67 (dd, J=11.0, 8.0 Hz, 1H), 7.36 (dd, J=11.0, 7.0 Hz, 1H), 6.95 (s, 1H), 5.35-4.81 (m, 2H), 4.81-4.63 (m, 1H), 4.17-4.03 (m, 1H), 4.03-3.73 (m, 1H), 3.17-2.90 (m, 2H), 1.36 (d, J=7.1 Hz, 3H).

### Example 112

### 5-(4,6-difluoro-1H-indole-2-carbonyl)-N-[(2R)-1,1,1-trifluoropropan-2-yl]-4H,5H,6H,7H-[1,2]oxazolo[4,5-c]pyridine-3-carboxamide

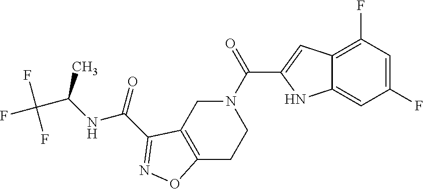 [SMILES: C[C@@H](NC(=O)c1noc2c1CN(C(=O)c1cc3c(F)cc(F)cc3[nH]1)CC2)C(F)(F)F]

Prepared as described for Example 111.

Rt 1.68 mins (Method J) [M+H]⁺ 443.2

1H NMR (400 MHz, DMSO) δ 12.38-11.81 (m, 1H), 9.92-9.30 (m, 1H), 7.05 (dd, J=9.4, 2.1 Hz, 1H), 7.00 (s, 1H), 6.93 (td, J=10.4, 2.1 Hz, 1H), 5.45-4.83 (m, 2H), 4.83-4.66 (m, 1H), 4.18-4.04 (m, 1H), 4.04-3.81 (m, 1H), 3.18-2.80 (m, 2H), 1.36 (d, J=7.1 Hz, 3H).

### Example 113

### 5-(4,6-dichloro-1H-indole-2-carbonyl)-N-[(2R)-1,1,1-trifluoropropan-2-yl]-4H,5H,6H,7H-[1,2]oxazolo[4,5-c]pyridine-3-carboxamide

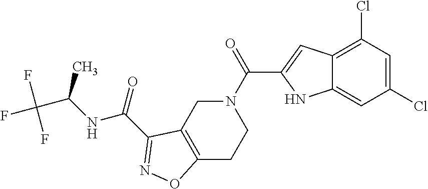 [SMILES: C[C@@H](NC(=O)c1noc2c1CN(C(=O)c1cc3c(Cl)cc(Cl)cc3[nH]1)CC2)C(F)(F)F]

Prepared as described for Example 111.

Rt 1.87 mins (Method J) [M+H]⁺ 475.2/477.2

1H NMR (400 MHz, DMSO) δ 12.22 (s, 1H), 9.61 (s, 1H), 7.44 (s, 1H), 7.29-7.22 (m, 1H), 6.93 (s, 1H), 5.46-4.83 (m, 2H), 4.83-4.67 (m, 1H), 4.20-4.03 (m, 1H), 4.03-3.72 (m, 1H), 3.19-2.79 (m, 2H), 1.36 (d, J=7.0 Hz, 3H).

### Example 114

### 5-(6-chloro-5-fluoro-1H-indole-2-carbonyl)-N-[(2R)-1,1,1-trifluoropropan-2-yl]-4H,5H,6H,7H-[1,2]oxazolo[4,3-c]pyridine-3-carboxamide

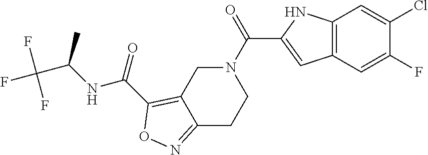 [SMILES: C[C@@H](NC(=O)c1onc2c1CN(C(=O)c1cc3cc(F)c(Cl)cc3[nH]1)CC2)C(F)(F)F]

Rt 4.14 mins (Method B2) [M+H]⁺ 459.1/461.1

1H NMR (400 MHz, DMSO) δ 11.93 (s, 1H), 9.61 (s, 1H), 7.66 (d, J=9.9 Hz, 1H), 7.56 (d, J=6.4 Hz, 1H), 6.96 (s, 1H), 5.20-4.85 (m, 2H), 4.84-4.67 (m, 1H), 4.13-3.85 (m, 2H), 3.13-2.92 (m, 2H), 1.36 (d, J=7.1 Hz, 3H).

### Example 115

### 5-(5,6-difluoro-1H-indole-2-carbonyl)-N-[(2R)-1,1,1-trifluoropropan-2-yl]-4H,5H,6H,7H-[1,2]oxazolo[4,3-c]pyridine-3-carboxamide

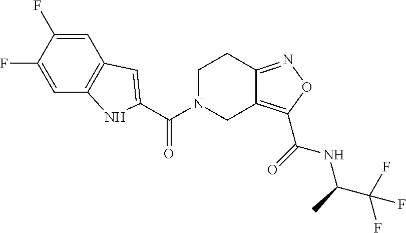 [SMILES: C[C@@H](NC(=O)c1onc2c1CN(C(=O)c1cc3cc(F)c(F)cc3[nH]1)CC2)C(F)(F)F]

Rt 3.98 mins (Method B2) [M+H]⁺ 443.1

### Example 116

### 5-(4-chloro-5-fluoro-1H-indole-2-carbonyl)-N-[(2R)-1,1,1-trifluoropropan-2-yl]-4H,5H,6H,7H-[1,2]oxazolo[4,3-c]pyridine-3-carboxamide

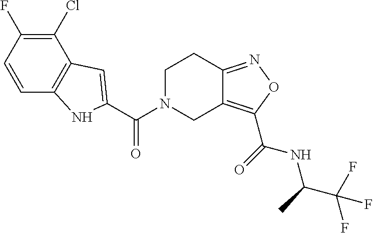 [SMILES: C[C@@H](NC(=O)c1onc2c1CN(C(=O)c1cc3c(Cl)c(F)ccc3[nH]1)CC2)C(F)(F)F]

Rt 4.13 mins (Method B2) [M+H]⁺ 459.1/461.1

### Example 117

### 5-(4,5-difluoro-1H-indole-2-carbonyl)-N-[(2R)-1,1,1-trifluoropropan-2-yl]-4H,5H,6H,7H-[1,2]oxazolo[4,3-c]pyridine-3-carboxamide

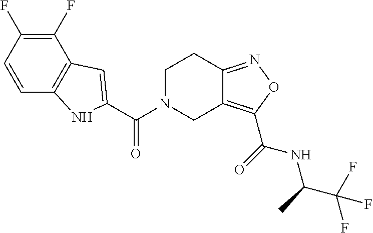 [SMILES: C[C@@H](NC(=O)c1onc2c1CN(C(=O)c1cc3c(F)c(F)ccc3[nH]1)CC2)C(F)(F)F]

Rt 4.01 mins (Method B2) [M+H]⁺ 443.1

### Example 118

### 5-(4-chloro-1H-indole-2-carbonyl)-N-[(2R)-1,1,1-trifluoropropan-2-yl]-4H,5H,6H,7H-[1,2]oxazolo[4,3-c]pyridine-3-carboxamide

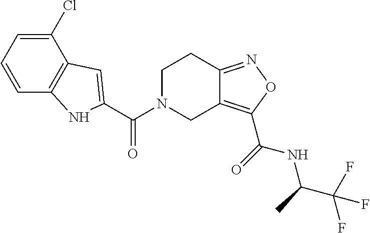 [SMILES: C[C@@H](NC(=O)c1onc2c1CN(C(=O)c1cc3c(Cl)cccc3[nH]1)CC2)C(F)(F)F]

Rt 4.10 mins (Method B2) [M+H]⁺ 441.1/443.1

### Example 119

### 5-(4-chloro-5-fluoro-1H-indole-2-carbonyl)-N-[(2R)-1,1,1-trifluoropropan-2-yl]-4H,5H,6H,7H-[1,2]oxazolo[4,5-c]pyridine-3-carboxamide

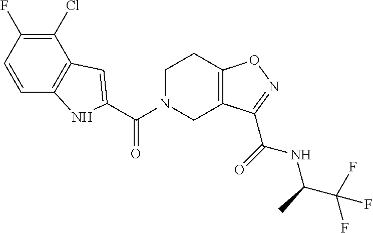 [SMILES: C[C@@H](NC(=O)c1noc2c1CN(C(=O)c1cc3c(Cl)c(F)ccc3[nH]1)CC2)C(F)(F)F]

Rt 1.74 mins (Method J) [M+H]⁺ 459.2/461.2

1H NMR (400 MHz, DMSO) δ 12.56-11.63 (m, 1H), 10.33-9.10 (m, 1H), 7.43 (dd, J=8.9, 4.0 Hz, 1H), 7.30-7.21 (m, 1H), 6.94 (s, 1H), 5.36-4.83 (m, 2H), 4.83-4.69 (m, 1H), 4.16-4.04 (m, 1H), 4.04-3.86 (m, 1H), 3.17-2.89 (m, 2H), 1.36 (d, J=7.0 Hz, 3H).

### Example 120

### 4′-(4,5-difluoro-1H-indole-2-carbonyl)-13′-(2-hydroxyethyl)-4′, 8′,9′,13′-tetraazaspiro[cyclopropane-1,12′-tricyclo[7.5.0.0²,7]tetradecane]-1′,7′-dien-14′-one

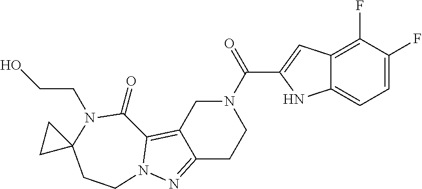 [SMILES: O=C(c1cc2c(F)c(F)ccc2[nH]1)N1CCc2nn3c(c2C1)C(=O)N(CCO)C1(CC3)CC1]

Rt 1.33 mins (Method J) [M+H]⁺ 456.2

1H NMR (400 MHz, DMSO) δ 12.09 (s, 1H), 7.29-7.21 (m, 2H), 7.03-6.92 (m, 1H), 5.15-4.56 (m, 3H), 4.45-4.28 (m, 2H), 4.10-3.86 (m, 2H), 3.70-3.56 (m, 2H), 3.53-3.40 (m, 2H), 3.02-2.70 (m, 2H), 2.16-2.05 (m, 2H), 0.90-0.66 (m, 2H), 0.57-0.45 (m, 2H).

### Example 121

### 4′-(5,6-difluoro-1H-indole-2-carbonyl)-13′-(2-hydroxyethyl)-4′, 8′,9′,13′-tetraazaspiro[cyclopropane-1,12′-tricyclo[7.5.0.0²,7]tetradecane]-1′,7′-dien-14′-one

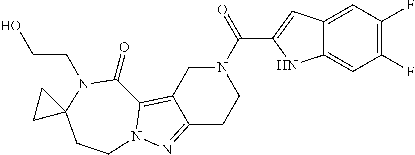 [SMILES: O=C(c1cc2cc(F)c(F)cc2[nH]1)N1CCc2nn3c(c2C1)C(=O)N(CCO)C1(CC3)CC1]

Rt 1.32 mins (Method J) [M+H]⁺ 456.4

1H NMR (400 MHz, DMSO) δ 11.86 (s, 1H), 7.65 (dd, J=11.0, 8.1 Hz, 1H), 7.35 (dd, J=11.0, 7.0 Hz, 1H), 6.97-6.81 (m, 1H), 5.16-4.51 (m, 3H), 4.36 (t, J=6.9 Hz, 2H), 4.05-3.94 (m, 2H), 3.67-3.57 (m, 2H), 3.53-3.41 (m, 2H), 2.99-2.72 (m, 2H), 2.16-2.05 (m, 2H), 0.89-0.66 (m, 2H), 0.58-0.44 (m, 2H).

### Example 122

### 4′-(6-chloro-5-fluoro-1H-indole-2-carbonyl)-13′-(2-hydroxyethyl)-4′,8′,9′,13′-tetraazaspiro[cyclopropane-1,12′-tricyclo[7.5.0.0²,7]tetradecane]-1′,7′-dien-14′-one

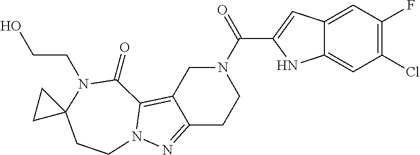 [SMILES: O=C(c1cc2cc(F)c(Cl)cc2[nH]1)N1CCc2nn3c(c2C1)C(=O)N(CCO)C1(CC3)CC1]

Rt 1.39 mins (Method J) [M+H]⁺ 472.2/474.4

1H NMR (400 MHz, DMSO) δ 11.91 (s, 1H), 7.65 (d, J=10.0 Hz, 1H), 7.54 (d, J=6.5 Hz, 1H), 7.03-6.81 (m, 1H), 5.08-4.57 (m, 3H), 4.36 (t, J=6.9 Hz, 2H), 4.11-3.90 (m, 2H), 3.67-3.56 (m, 2H), 3.54-3.39 (m, 2H), 3.02-2.73 (m, 2H), 2.21-1.96 (m, 2H), 0.89-0.65 (m, 2H), 0.58-0.41 (m, 2H).

### Example 123

### 4′-(1H-indole-2-carbonyl)-13′-(2-methoxyethyl)-4′, 8′,9′,13′-tetraazaspiro[cyclopropane-1,12′-tricyclo[7.5.0.0²,7]tetradecane]-1′,7′-dien-14′-one

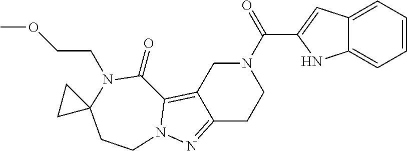 [SMILES: COCCN1C(=O)c2c3c(nn2CCC12CC2)CCN(C(=O)c1cc2ccccc2[nH]1)C3]

Rt 1.40 mins (Method H) [M+H]⁺ 434.4

1H NMR (400 MHz, DMSO) δ 11.65 (s, 1H), 7.63 (d, J=8.0 Hz, 1H), 7.42 (d, J=8.3 Hz, 1H), 7.24-7.16 (m, 1H), 7.09-7.02 (m, 1H), 6.93-6.82 (m, 1H), 5.15-4.50 (m, 2H), 4.32 (t, J=6.6 Hz, 2H), 4.12-3.89 (m, 2H), 3.66-3.45 (m, 4H), 3.28 (s, 3H), 2.99-2.70 (m, 2H), 2.17-1.96 (m, 2H), 0.88-0.68 (m, 2H), 0.62-0.44 (m, 2H).

### Example 124

### 13′-[2-(dimethylamino)ethyl]-4′-(1H-indole-2-carbonyl)-4′,8′,9′,13′-tetraazaspiro[cyclopropane-1,12′-tricyclo[7.5.0.0²,7]tetradecane]-1′,7′-dien-14′-one

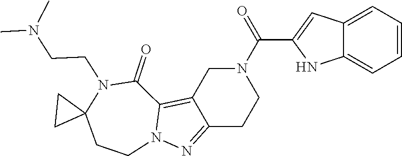 [SMILES: CN(C)CCN1C(=O)c2c3c(nn2CCC12CC2)CCN(C(=O)c1cc2ccccc2[nH]1)C3]

Rt 2.50 mins (Method B2) [M+H]⁺ 447.2

1H NMR (400 MHz, DMSO) δ 11.65 (s, 1H), 7.63 (d, J=7.9 Hz, 1H), 7.42 (d, J=8.2 Hz, 1H), 7.23-7.15 (m, 1H), 7.10-7.02 (m, 1H), 6.93-6.80 (m, 1H), 5.26-4.52 (m, 2H), 4.42-4.33 (m, 2H), 4.07-3.94 (m, 2H), 3.65-3.40 (m, 2H), 3.02-2.72 (m, 2H), 2.48-2.41 (m, 2H), 2.30-1.95 (m, 8H), 0.91-0.66 (m, 2H), 0.63-0.41 (m, 2H).

### Example 125

### 4′-(1H-indole-2-carbonyl)-13′-[2-(4-methylpiperazin-1-yl)ethyl]-4′,8′,9′,13′-tetraazaspiro[cyclopropane-1,12′-tricyclo[7.5.0.0²,7]tetradecane]-1′,7′-dien-14′-one

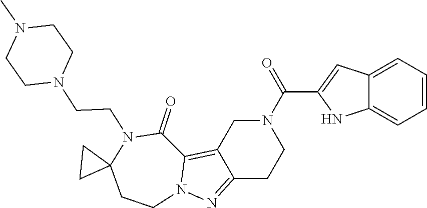 [SMILES: CN1CCN(CCN2C(=O)c3c4c(nn3CCC23CC3)CCN(C(=O)c2cc3ccccc3[nH]2)C4)CC1]

Step 1: Tert-butyl 10′-(2-hydroxyethyl)-11′-oxo-3′,4′,7′,8′,10′,11′-hexahydrospirol[cyclopropane-1,9′-pyrido[4′,3′:3,4]pyrazolo[1,5-a][1,4]diazepine]-2′(1′H)-carboxylate (60 mg, 0.159 mmol) (see Example 89) was dissolved in dichloromethane (3 mL) and Dess-Martin periodinane (101 mg, 0.239 mmol) was added. After stirring for 1 h the reaction mixture was diluted with EtOAc (10 mL). The resulting white suspension was washed with a saturated aqueous solution of Na₂S₂O₃ (10 mL). The layers were separated and the water layer was extracted with EtOAc (10 mL). The combined organic layers were washed with a saturated aqueous solution of NaHCO₃, dried over sodium sulfate, concentrated in vacuo, and stripped with dichloromethane. The resulting solidified oil was dissolved in dichloromethane (1 mL) and was purified by column chromatography (MeOH in dichloromethane, 0% to 10%) to yield tert-butyl 11′-oxo-10′-(2-oxoethyl)-3′,4′,7′,8′,10′,11′-hexahydrospiro[cyclopropane-1,9′-pyrido[4′,3′:3,4]pyrazolo[1,5-a][1,4]diazepine]-2′(1′H)-carboxylate as a solidifying oil (43 mg, 72% yield).

Step 2: Tert-butyl 11′-oxo-10′-(2-oxoethyl)-3′,4′,7′,8′,10′,11′-hexahydrospiro[cyclopropane-1,9′-pyrido[4′,3′:3,4]pyrazolo[1,5-a][1,4]diazepine]-2′(1′H)-carboxylate (21 mg, 0.056 mmol) was dissolved in dichloromethane (0.5 mL) and 1-methylpiperazine (9.33 μL, 0.084 mmol) was added, followed by sodium triacetoxyborohydride (17.83 mg, 0.084 mmol) and the mixture was stirred overnight. The reaction mixture was partitioned between EtOAc (10 mL) and saturated aqueous solution of NaHCO₃ (10 mL). The layers were separated and the aqueous layer was extracted with EtOAc (10 mL). The combined organic layers were washed with brine (10 mL), dried over sodium sulfate, concentrated in vacuo, and stripped with dichloromethane. The residue was dissolved in dichloromethane (2 mL) and was purified by column chromatography (7M NH₃ in MeOH in dichloromethane, 0% to 10%) affording tert-butyl 10′-(2-(4-methylpiperazin-1-yl)ethyl)-11′-oxo-3′,4′,7′,8′,10′,11′-hexahydrospiro[cyclopropane-1,9′-pyrido[4′,3′:3,4]pyrazolo[1,5-a][1,4]diazepine]-2′(1′H)-carboxylate as a colorless oil (15 mg, 58% yield).

Step 3: Tert-butyl 10′-(2-(4-methylpiperazin-1-yl)ethyl)-11′-oxo-3′,4′,7′,8′,10′,11′-hexahydrospirol[cyclopropane-1,9′-pyrido[4′,3′:3,4]pyrazolo[1,5-a]1[1,4]diazepine]-2′(1′H)-carboxylate (15 mg, 0.033 mmol) was dissolved in dichloromethane (0.1 mL) and 4M HCl in dioxane (1 mL, 4.00 mmol) was added. After 3 h, the reaction mixture was concentrated and stripped with dichloromethane to yield 10′-(2-(4-methylpiperazin-1-yl)ethyl)-1′,2′,3′,4′,7′,8′-hexahydrospirol[cyclopropane-1,9′-pyrido[4′,3′:3,4]pyrazolo[1,5-a][1,4]diazepin]-11′(10′H)-one dihydrochloride as a white solid (14 mg, quant. yield).

Step 4: Indole-2-carboxylic acid (6.32 mg, 0.039 mmol) was dissolved in N,N-dimethylformamide (400 μL) followed by Et₃N (10 μL, 0.072 mmol) and HATU (13.67 mg, 0.036 mmol) and the mixture was stirred for 10 mins. In a separate vial, 10′-(2-(4-methylpiperazin-1-yl)ethyl)-1′,2′,3′,4′,7′,8′-hexahydrospiro[cyclopropane-1,9′-pyrido[4′,3′:3,4]pyrazolo[1,5-a][1,4]diazepin]-11′(10′H)-one dihydrochloride (14.1 mg, 0.033 mmol) was suspended in N,N-dimethylformamide (400 μL) and Et₃N (20 μL, 0.143 mmol) was added followed by a drop of water. The mixtures were combined and stirred overnight. The reaction mixture was filtered and purified by preparative HPLC to afford the product as a white solid (12.5 mg, 76% yield).

Rt 0.92 mins (Method H) [M+H]⁺ 502.4

1H NMR (400 MHz, DMSO) δ 11.65 (s, 1H), 7.63 (d, J=8.0 Hz, 1H), 7.42 (d, J=8.2 Hz, 1H), 7.19 (t, J=7.6 Hz, 1H), 7.06 (t, J=7.5 Hz, 1H), 6.94-6.79 (m, 1H), 5.20-4.55 (m, 2H), 4.53-4.33 (m, 2H), 4.16-3.89 (m, 2H), 3.77-3.38 (m, 2H), 2.97-2.71 (m, 2H), 2.61-2.52 (m, 2H), 2.49-1.85 (m, 13H), 0.91-0.63 (m, 2H), 0.61-0.41 (m, 2H).

### Example 126

### 5-(4-chloro-1H-indole-2-carbonyl)-N-{1-[(difluoromethoxy)methyl]cyclopropyl}-4H,5H,6H,7H-[1,2]oxazolo[4,5-c]pyridine-3-carboxamide

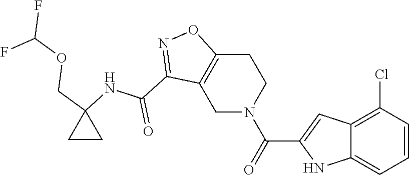 [SMILES: O=C(NC1(COC(F)F)CC1)c1noc2c1CN(C(=O)c1cc3c(Cl)cccc3[nH]1)CC2]

Rt 3.88 mins (Method A2) [M+H]⁺ 465.0/467.0

1H NMR (400 MHz, DMSO) δ 12.08 (s, 1H), 9.12 (s, 1H), 7.41 (d, J=8.0 Hz, 1H), 7.32-7.10 (m, 2H), 6.98-6.40 (m, 2H), 5.18-4.49 (m, 2H), 4.24-3.82 (m, 4H), 3.22-2.84 (m, 2H), 1.00-0.70 (m, 4H).

### Example 127

### 5-(4,5-difluoro-1H-indole-2-carbonyl)-N-{1-[(difluoromethoxy)methyl]cyclopropyl}-4H,5H,6H,7H-[1,2]oxazolo[4,5-c]pyridine-3-carboxamide

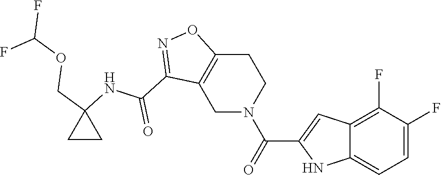 [SMILES: O=C(NC1(COC(F)F)CC1)c1noc2c1CN(C(=O)c1cc3c(F)c(F)ccc3[nH]1)CC2]

Rt 3.79 mins (Method A2) [M+H]⁺ 467.0

1H NMR (400 MHz, DMSO) δ 12.11 (s, 1H), 9.12 (s, 1H), 7.33-7.17 (m, 2H), 7.04 (s, 1H), 6.67 (t, J=76.0 Hz, 1H), 5.13-4.53 (m, 2H), 4.15-3.82 (m, 4H), 3.21-2.86 (m, 2H), 1.01-0.73 (m, 4H).

### Example 128

### N-{1-[(difluoromethoxy)methyl]cyclopropyl}-5-(6-fluoro-4-methyl-1H-indole-2-carbonyl)-4H,5H,6H,7H-[1,2]oxazolo[4,5-c]pyridine-3-carboxamide

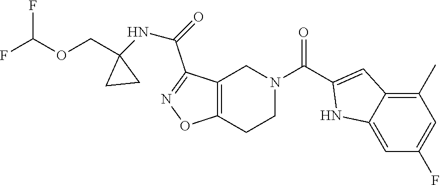 [SMILES: Cc1cc(F)cc2[nH]c(C(=O)N3CCc4onc(C(=O)NC5(COC(F)F)CC5)c4C3)cc12]

Rt 3.89 mins (Method A2) [M+H]⁺ 463.1

1H NMR (400 MHz, DMSO) δ 11.73 (s, 1H), 9.11 (s, 1H), 7.00-6.93 (m, 2H), 6.90-6.43 (m, 2H), 4.98-4.63 (m, 2H), 4.15-3.86 (m, 4H), 3.14-2.96 (m, 2H), 2.52 (s, 3H), 0.94-0.80 (m, 4H).

### Example 129

### 5-(6-chloro-5-fluoro-1H-indole-2-carbonyl)-N-{1-[(difluoromethoxy)methyl]cyclopropyl}-4H,5H,6H,7H-[1,2]oxazolo[4,5-c]pyridine-3-carboxamide

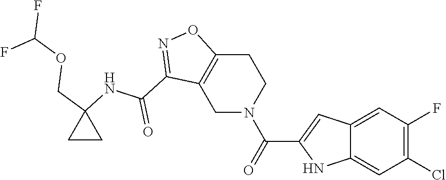 [SMILES: O=C(NC1(COC(F)F)CC1)c1noc2c1CN(C(=O)c1cc3cc(F)c(Cl)cc3[nH]1)CC2]

Rt 3.91 mins (Method A2) [M+H]⁺ 483.0/485.0

1H NMR (400 MHz, DMSO) δ 11.92 (s, 1H), 9.11 (s, 1H), 7.66 (d, J=10.0 Hz, 1H), 7.55 (d, J=6.4 Hz, 1H), 6.95 (s, 1H), 6.67 (t, J=76.1 Hz, 1H), 5.15-4.49 (m, 2H), 4.18-3.80 (m, 4H), 3.20-2.78 (m, 2H), 1.01-0.70 (m, 4H).

### Example 130

### N-{1-[(difluoromethoxy)methyl]cyclopropyl}-5-(1H-indole-2-carbonyl)-4H,5H,6H,7H-[1,2]oxazolo[4,5-c]pyridine-3-carboxamide

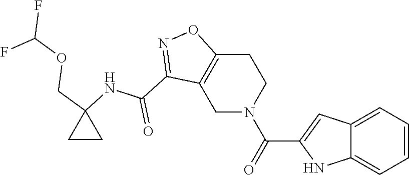 [SMILES: O=C(NC1(COC(F)F)CC1)c1noc2c1CN(C(=O)c1cc3ccccc3[nH]1)CC2]

Rt 3.68 mins (Method A2) [M+H]⁺ 431.1

1H NMR (400 MHz, DMSO) δ 11.67 (s, 1H), 9.11 (s, 1H), 7.65 (d, J=8.1 Hz, 1H), 7.43 (d, J=8.2 Hz, 1H), 7.21 (t, J=7.5 Hz, 1H), 7.07 (t, J=7.5 Hz, 1H), 6.93 (s, 1H), 6.67 (t, J=76.2 Hz, 1H), 5.04-4.64 (m, 2H), 4.14-3.86 (m, 4H), 3.15-2.94 (m, 2H), 0.92-0.80 (m, 4H).

### Example 131

### 5-(5,6-difluoro-1H-indole-2-carbonyl)-N-{1-[(difluoromethoxy)methyl]cyclopropyl}-4H,5H,6H,7H-[1,2]oxazolo[4,3-c]pyridine-3-carboxamide

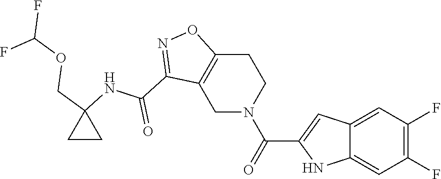 [SMILES: O=C(NC1(COC(F)F)CC1)c1noc2c1CN(C(=O)c1cc3cc(F)c(F)cc3[nH]1)CC2]

Rt 3.81 mins (Method A2) [M+H]⁺ 467.1

1H NMR (400 MHz, DMSO) δ 11.88 (s, 1H), 9.12 (s, 1H), 7.75-7.56 (m, 1H), 7.36 (dd, J=11.0, 7.0 Hz, 1H), 6.95 (s, 1H), 6.67 (t, J=76.1 Hz, 1H), 5.18-4.55 (m, 2H), 4.16-3.84 (m, 4H), 3.17-2.89 (m, 2H), 0.93-0.79 (m, 4H).

### Example 132

### N-{1-[(difluoromethoxy)methyl]cyclopropyl}-5-(1H-indole-2-carbonyl)-4H,5H,6H,7H-[1,2]oxazolo[4,3-c]pyridine-3-carboxamide

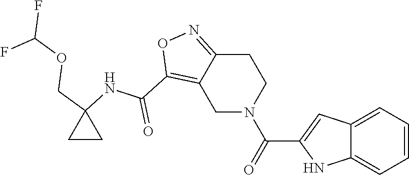 [SMILES: O=C(NC1(COC(F)F)CC1)c1onc2c1CN(C(=O)c1cc3ccccc3[nH]1)CC2]

Rt 1.53 mins (Method H) [M−H] 429.2

1H NMR (400 MHz, DMSO) δ 11.68 (s, 1H), 9.32 (s, 1H), 7.66 (d, J=8.0 Hz, 1H), 7.44 (d, J=8.2 Hz, 1H), 7.24-7.18 (m, 1H), 7.10-7.04 (m, 1H), 6.93 (s, 1H), 6.67 (t, J=76.2 Hz, 1H), 5.25-4.77 (m, 2H), 4.11-3.87 (m, 4H), 3.12-2.92 (m, 2H), 0.93-0.74 (m, 4H).

### Example 133

### 5-(4-chloro-1H-indole-2-carbonyl)-N-{1-[(difluoromethoxy)methyl]cyclopropyl}-4H,5H,6H,7H-[1,2]oxazolo[4,3-c]pyridine-3-carboxamide

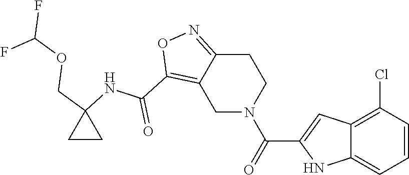 [SMILES: O=C(NC1(COC(F)F)CC1)c1onc2c1CN(C(=O)c1cc3c(Cl)cccc3[nH]1)CC2]

Rt 1.65 mins (Method H) [M−H] 463.2/465.2 1H NMR (400 MHz, DMSO) δ 12.08 (s, 1H), 9.32 (s, 1H), 7.42 (d, J=8.0 Hz, 1H), 7.24-7.12 (m, 2H), 6.90 (s, 1H), 6.67 (t, J=76.1 Hz, 1H), 5.20-4.75 (m, 2H), 4.08-3.87 (m, 4H), 3.08-2.91 (m, 2H), 0.93-0.77 (m, 4H).

### Example 134

### 4′-(1H-indole-2-carbonyl)-13′-[2-(morpholin-4-yl)ethyl]-4′,8′,9′,13′-tetraazaspiro[cyclopropane-1,12′-tricyclo[7.5.0.0²,7]tetradecane]-1′,7′-dien-14′-one

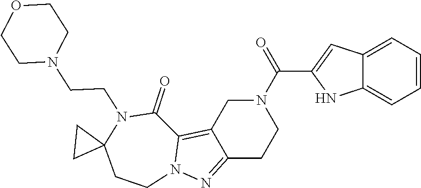 [SMILES: O=C(c1cc2ccccc2[nH]1)N1CCc2nn3c(c2C1)C(=O)N(CCN1CCOCC1)C1(CC3)CC1]

Rt 0.91 mins (Method H) [M+H]⁺ 489.4

1H NMR (400 MHz, DMSO) δ 11.63 (s, 1H), 7.63 (d, J=8.0 Hz, 1H), 7.42 (d, J=8.2 Hz, 1H), 7.19 (t, J=7.6 Hz, 1H), 7.06 (t, J=7.5 Hz, 1H), 6.86 (s, 1H), 5.25-4.56 (m, 2H), 4.44 (t, J=6.8 Hz, 2H), 4.16-3.86 (m, 2H), 3.64-3.45 (m, 6H), 2.95-2.72 (m, 2H), 2.60-2.52 (m, 2H), 2.47-2.34 (m, 4H), 2.18-1.94 (m, 2H), 0.95-0.63 (m, 2H), 0.63-0.38 (m, 2H).

### Example 135

### 5-(4,6-difluoro-1H-indole-2-carbonyl)-N-{1-n[(difluoromethoxy)methyl]cyclopropyl}-4H,5H,6H,7H-[1,2]oxazolo[4,5-c]pyridine-3-carboxamide

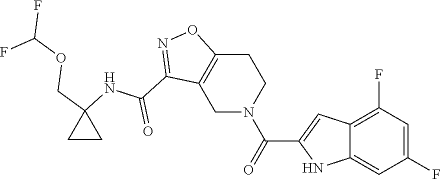 [SMILES: O=C(NC1(COC(F)F)CC1)c1noc2c1CN(C(=O)c1cc3c(F)cc(F)cc3[nH]1)CC2]

Prepared as described for Example 100.

Rt 3.87 mins (Method A2) [M+H]⁺ 467.1

1H NMR (400 MHz, DMSO) δ 12.12 (s, 1H), 9.12 (s, 1H), 7.12-6.43 (m, 4H), 5.15-4.49 (m, 2H), 4.20-3.81 (m, 4H), 3.23-2.85 (m, 2H), 0.98-0.72 (m, 4H).

### Example 136

### 2-{1-[N-methyl-5-(6-chloro-5-fluoro-1H-indole-2-carbonyl)-2H,4H,5H,6H,7H-pyrazolo[4,3-c]pyridine-3-amido]cyclopropyl}pyrimidine-5-carboxylic acid

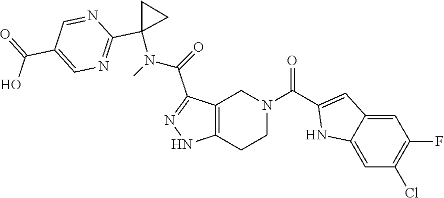 [SMILES: CN(C(=O)c1n[nH]c2c1CN(C(=O)c1cc3cc(F)c(Cl)cc3[nH]1)CC2)C1(c2ncc(C(=O)O)cn2)CC1]

Rt 3.49 mins (Method B2) [M+H]⁺ 538.0/540.0

### Example 137

### 4-{1-[5-(1H-indole-2-carbonyl)-4H,5H,6H,7H-pyrazolo[1,5-a]pyrazine-3-amido]cyclopropyl}benzoic acid

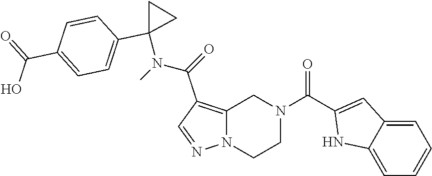 [SMILES: CN(C(=O)c1cnn2c1CN(C(=O)c1cc3ccccc3[nH]1)CC2)C1(c2ccc(C(=O)O)cc2)CC1]

Rt 3.30 mins (Method B2) [M+H]⁺ 470.1

1H NMR (400 MHz, DMSO-d6) δ 11.70 (s, 1H), 8.90 (s, 1H), 8.12 (s, 1H), 7.82 (d, J=8.3 Hz, 2H), 7.63 (d, J=8.0 Hz, 1H), 7.43 (d, J=8.3 Hz, 1H), 7.25-7.16 (m, 3H), 7.05 (t, J=7.5 Hz, 1H), 6.93 (s, 1H), 5.38-4.98 (m, 2H), 4.39-4.08 (m, 4H), 1.30 (d, J=8.2 Hz, 4H). One signal (1H) coincides with water signal.

### Example 138

### 7-(1H-indole-2-carbonyl)-N-[(2R)-1,1,1-trifluoropropan-2-yl]-5H,6H,7H,8H-imidazo[1,5-a]pyrazine-1-carboxamide

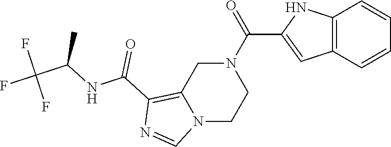 [SMILES: C[C@@H](NC(=O)c1ncn2c1CN(C(=O)c1cc3ccccc3[nH]1)CC2)C(F)(F)F]

Rt 1.45 mins (Method H) [M+H]⁺ 406.2

1H NMR (400 MHz, DMSO-d6) δ 11.71 (s, 1H), 8.33 (d, J=9.3 Hz, 1H), 7.81 (s, 1H), 7.66 (d, J=8.0 Hz, 1H), 7.45 (d, J=8.2 Hz, 1H), 7.22 (t, J=7.6 Hz, 1H), 7.07 (t, J=7.5 Hz, 1H), 6.95 (s, 1H), 5.54-4.86 (m, 2H), 4.82-4.69 (m, 1H), 4.38-3.97 (m, 4H), 1.33 (d, J=7.1 Hz, 3H).

### Example 139

### 4-(1H-indole-2-carbonyl)-13-methyl-4,8,9,13-tetraazatricyclo[7.5.0.0²,7]tetradeca-1,7-dien-14-one

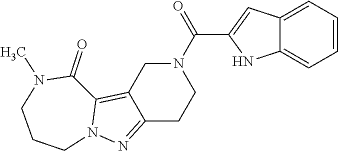 [SMILES: CN1CCCn2nc3c(c2C1=O)CN(C(=O)c1cc2ccccc2[nH]1)CC3]

Rt 2.97 mins (Method A2) m/z 364 [M+H]⁺

1H NMR (400 MHz, DMSO) δ 11.63 (s, 1H), 7.64 (d, J=7.9 Hz, 1H), 7.42 (d, J=8.2 Hz, 1H), 7.19 (t, J=7.5 Hz, 1H), 7.06 (t, J=7.4 Hz, 1H), 6.87 (s, 1H), 5.16-4.60 (m, 2H), 4.39-4.17 (m, 2H), 4.13-3.83 (m, 2H), 3.43-3.34 (m, 2H), 3.14-2.70 (m, 5H), 2.24-2.09 (m, 2H).

### Example 140

### 4′-(2-hydroxyethyl)-12′-(1H-indole-2-carbonyl)-4′,7′,8′,12′-tetraazaspiro[cyclopropane-1,5′-tricyclo[7.4.0.0²,7]tridecane]-1′,8′-dien-3′-one

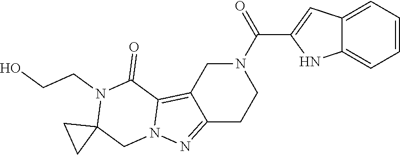 [SMILES: O=C(c1cc2ccccc2[nH]1)N1CCc2nn3c(c2C1)C(=O)N(CCO)C1(CC1)C3]

Rt 1.22 mins (Method H) m/z 406 [M+H]⁺

1H NMR (400 MHz, DMSO) δ 11.65 (s, 1H), 7.65 (d, J=7.9 Hz, 1H), 7.43 (d, J=8.2 Hz, 1H), 7.20 (t, J=7.5 Hz, 1H), 7.06 (t, J=7.4 Hz, 1H), 6.89 (s, 1H), 5.21-4.79 (m, 2H), 4.79-4.66 (m, 1H), 4.17 (s, 2H), 4.09-3.91 (m, 2H), 3.50-3.35 (m, 4H), 2.97-2.74 (m, 2H), 1.18-0.89 (m, 4H).

### Example 141

### 5-(5,6-difluoro-1H-indole-2-carbonyl)-N-{1-[(difluoromethoxy)methyl]cyclopropyl}-4H,5H,6H,7H-[1,2]oxazolo[4,3-c]pyridine-3-carboxamide

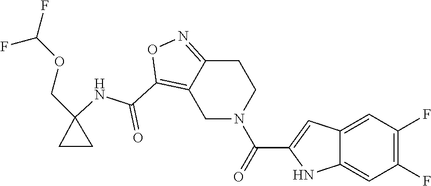 [SMILES: O=C(NC1(COC(F)F)CC1)c1onc2c1CN(C(=O)c1cc3cc(F)c(F)cc3[nH]1)CC2]

Rt 1.6 mins (Method H) m/z 465 [M+H]⁺

1H NMR (400 MHz, DMSO) δ 11.88 (s, 1H), 9.32 (s, 1H), 7.76-7.61 (m, 1H), 7.40-7.32 (m, 1H), 6.95 (s, 1H), 6.67 (t, J=76.1 Hz, 1H), 5.20-4.77 (m, 2H), 4.12-3.83 (m, 4H), 3.12-2.89 (m, 2H), 0.96-0.78 (m, 4H).

### Example 142

### 5-(4-chloro-5-fluoro-1H-indole-2-carbonyl)-N-{1-[(difluoromethoxy)methyl]cyclopropyl}-4H,5H,6H,7H-[1,2]oxazolo[4,3-c]pyridine-3-carboxamide

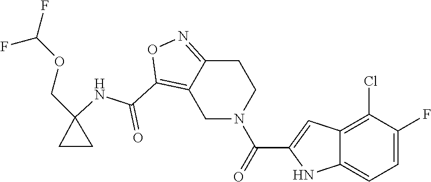 [SMILES: O=C(NC1(COC(F)F)CC1)c1onc2c1CN(C(=O)c1cc3c(Cl)c(F)ccc3[nH]1)CC2]

Rt 1.67 mins (Method H) m/z 481/483 [M+H]⁺

1H NMR (400 MHz, DMSO) δ 12.16 (s, 1H), 9.32 (s, 1H), 7.50-7.36 (m, 1H), 7.31-7.23 (m, 1H), 6.93 (s, 1H), 6.67 (t, J=76.1 Hz, 1H), 5.33-4.73 (m, 2H), 3.97 (d, J=23.4 Hz, 4H), 3.16-2.91 (m, 2H), 1.07-0.72 (m, 4H).

### Example 143

### 5-(4,5-difluoro-1H-indole-2-carbonyl)-N-{1-[(difluoromethoxy)methyl]cyclopropyl}-4H,5H,6H,7H-[1,2]oxazolo[4,3-c]pyridine-3-carboxamide

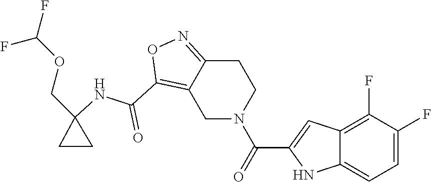 [SMILES: O=C(NC1(COC(F)F)CC1)c1onc2c1CN(C(=O)c1cc3c(F)c(F)ccc3[nH]1)CC2]

Rt 1.61 mins (Method H) m/z 465 [M+H]⁺

1H NMR (400 MHz, DMSO) δ 12.10 (s, 1H), 9.32 (s, 1H), 7.31-7.15 (m, 2H), 7.03 (s, 1H), 6.67 (t, J=76.2 Hz, 1H), 5.18-4.73 (m, 2H), 4.12-3.79 (m, 4H), 3.10-2.81 (m, 2H), 0.95-0.74 (m, 4H).

### Example 144

### 5-(6-chloro-5-fluoro-1H-indole-2-carbonyl)-N-{1-[(difluoromethoxy)methyl]cyclopropyl}-4H,5H,6H,7H-[1,2]oxazolo[4,3-c]pyridine-3-carboxamide

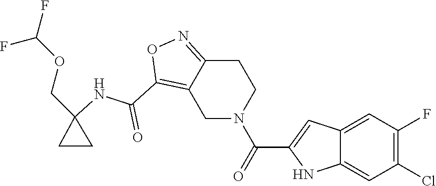 [SMILES: O=C(NC1(COC(F)F)CC1)c1onc2c1CN(C(=O)c1cc3cc(F)c(Cl)cc3[nH]1)CC2]

Rt 1.67 mins (Method H) m/z 481/483 [M+H]⁺

1H NMR (400 MHz, DMSO) δ 11.92 (s, 1H), 9.32 (s, 1H), 7.69-7.62 (m, 1H), 7.59-7.50 (m, 1H), 6.95 (s, 1H), 6.58 (d, J=76.1 Hz, 1H), 5.25-4.62 (m, 2H), 4.08-3.81 (m, 4H), 3.12-2.82 (m, 2H), 0.97-0.70 (m, 4H).

### Example 145

### 4-(1H-indole-2-carbonyl)-12,13-dimethyl-4,8,9,13-tetraazatricyclo[7.5.0.0²,7]tetradeca-1,7-dien-14-one

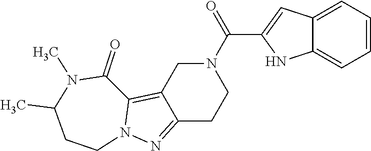 [SMILES: CC1CCn2nc3c(c2C(=O)N1C)CN(C(=O)c1cc2ccccc2[nH]1)CC3]

Rt 1.31 mins (Method H) m/z 378 [M+H]⁺

1H NMR (400 MHz, DMSO) δ 11.64 (s, 1H), 7.64 (d, J=8.0 Hz, 1H), 7.42 (d, J=8.2 Hz, 1H), 7.23-7.15 (m, 1H), 7.10-7.02 (m, 1H), 6.86 (s, 1H), 5.26-4.52 (m, 2H), 4.40-4.31 (m, 1H), 4.31-4.18 (m, 1H), 4.13-3.82 (m, 2H), 3.78-3.66 (m, 1H), 3.04-2.91 (m, 3H), 2.89-2.72 (m, 2H), 2.31-2.18 (m, 1H), 2.18-2.04 (m, 1H), 1.18 (d, J=6.8 Hz, 3H).

### Example 146

### 4′-(1H-indole-2-carbonyl)-13′-[2-(trifluoromethoxy)ethyl]-4′,8′,9′,13′-tetraazaspiro[cyclopropane-1,12′-tricyclo[7.5.0.0²,7]tetradecane]-1′,7′-dien-14′-one

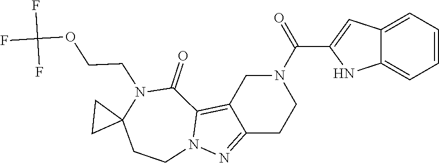 [SMILES: O=C(c1cc2ccccc2[nH]1)N1CCc2nn3c(c2C1)C(=O)N(CCOC(F)(F)F)C1(CC3)CC1]

Rt 1.62 mins (Method H) m/z 488 [M+H]⁺

1H NMR (400 MHz, DMSO) δ 11.65 (s, 1H), 7.63 (d, J=7.9 Hz, 1H), 7.42 (d, J=8.2 Hz, 1H), 7.23-7.15 (m, 1H), 7.09-7.02 (m, 1H), 6.96-6.77 (m, 1H), 5.15-4.48 (m, 2H), 4.42-4.21 (m, 4H), 4.16-3.88 (m, 2H), 3.85-3.61 (m, 2H), 3.06-2.69 (m, 2H), 2.17-2.02 (m, 2H), 0.93-0.70 (m, 2H), 0.69-0.46 (m, 2H).

### Example 147

### 13′-(2,2-difluoroethyl)-4′-(1H-indole-2-carbonyl)-4′,8′,9′,13′-tetraazaspiro[cyclopropane-1,12′-tricyclo[7.5.0.0²,7]tetradecane]-1′,7′-dien-14′-one

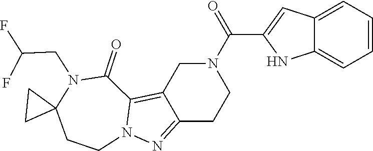 [SMILES: O=C(c1cc2ccccc2[nH]1)N1CCc2nn3c(c2C1)C(=O)N(CC(F)F)C1(CC3)CC1]

Rt 1.52 mins (Method J) m/z 440 [M+H]⁺

1H NMR (400 MHz, DMSO) δ 11.65 (s, 1H), 7.63 (d, J=8.0 Hz, 1H), 7.42 (d, J=8.1 Hz, 1H), 7.24-7.15 (m, 1H), 7.06 (t, J=7.4 Hz, 1H), 6.98-6.78 (m, 1H), 6.32 (t, J=55.6 Hz, 1H), 5.16-4.50 (m, 2H), 4.43-4.27 (m, 2H), 4.12-3.93 (m, 2H), 3.93-3.72 (m, 2H), 3.04-2.74 (m, 2H), 2.20-2.01 (m, 2H), 0.96-0.74 (m, 2H), 0.66-0.46 (m, 2H).

### Example 148

### 4′-(1H-indole-2-carbonyl)-13′-(2,2,2-trifluoroethyl)-4′,8′,9′,13′-tetraazaspiro[cyclopropane-1,12′-tricyclo[7.5.0.0²,7]tetradecane]-1′,7′-dien-14′-one

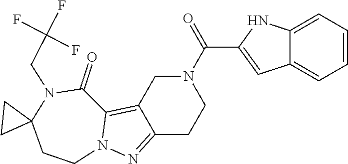 [SMILES: O=C(c1cc2ccccc2[nH]1)N1CCc2nn3c(c2C1)C(=O)N(CC(F)(F)F)C1(CC3)CC1]

Rt 3.61 mins (Method A2) m/z 458.1 [M+H]⁺

1H NMR (400 MHz, DMSO) δ 11.65 (s, 1H), 7.63 (d, J=7.9 Hz, 1H), 7.42 (d, J=8.1 Hz, 1H), 7.20 (t, J=7.5 Hz, 1H), 7.06 (t, J=7.4 Hz, 1H), 6.87 (s, 1H), 5.24-4.54 (m, 2H), 4.48-4.14 (m, 4H), 4.02 (s, 2H), 3.03-2.73 (m, 2H), 2.23-1.94 (m, 2H), 0.92 (s, 2H), 0.61 (s, 2H).

### Example 149

### methyl 2-[4′-(1H-indole-2-carbonyl)-14′-oxo-4′, 8′,9′,13′-tetraazaspiro[cyclopropane-1,12′-tricyclo[7.5.0.0²,7]tetradecane]-1′,7′-dien-13′-yl]acetate

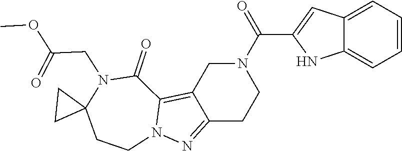 [SMILES: COC(=O)CN1C(=O)c2c3c(nn2CCC12CC2)CCN(C(=O)c1cc2ccccc2[nH]1)C3]

Rt 3.25 mins (Method A2) m/z 448.2 [M+H]⁺

1H NMR (400 MHz, DMSO) δ 11.65 (s, 1H), 7.63 (d, J=8.0 Hz, 1H), 7.42 (d, J=8.3 Hz, 1H), 7.19 (t, J=7.5 Hz, 1H), 7.06 (t, J=7.4 Hz, 1H), 6.87 (s, 1H), 5.11-4.55 (m, 2H), 4.55-4.33 (m, 2H), 4.33-3.81 (m, 4H), 3.67 (s, 3H), 3.03-2.73 (m, 2H), 2.24-2.01 (m, 2H), 0.94-0.66 (m, 2H), 0.66-0.41 (m, 2H).

### Example 150

### 5-(4-chloro-1H-indole-2-carbonyl)-N-[(2R)-1,1-difluoropropan-2-yl]-4H,5H,6H,7H-[1,2]oxazolo[4,3-c]pyridine-3-carboxamide

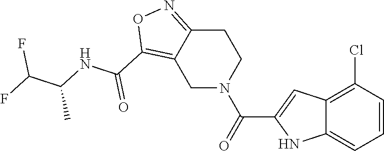 [SMILES: C[C@@H](NC(=O)c1onc2c1CN(C(=O)c1cc3c(Cl)cccc3[nH]1)CC2)C(F)F]

Rt 1.63 mins (Method H) m/z 421/423 [M+H]⁺

1H NMR (400 MHz, DMSO) δ 12.23-11.86 (m, 1H), 9.48-8.93 (m, 1H), 7.41 (d, J=8.0 Hz, 1H), 7.20 (t, J=7.8 Hz, 1H), 7.15 (d, J=7.5 Hz, 1H), 6.89 (s, 1H), 6.20-5.80 (m, 1H), 5.28-4.70 (m, 2H), 4.46-4.22 (m, 1H), 4.17-3.87 (m, 2H), 3.17-2.88 (m, 2H), 1.22 (d, J=7.0 Hz, 3H).

### Example 151

### 5-(4,5-difluoro-1H-indole-2-carbonyl)-N-[(2R)-1,1-difluoropropan-2-yl]-4H,5H,6H,7H-[1,2]oxazolo[4,3-c]pyridine-3-carboxamide

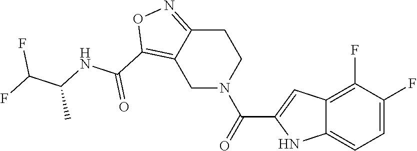 [SMILES: C[C@@H](NC(=O)c1onc2c1CN(C(=O)c1cc3c(F)c(F)ccc3[nH]1)CC2)C(F)F]

Rt 1.58 mins (Method H) m/z 423 [M+H]⁺

1H NMR (400 MHz, DMSO) δ 12.31-11.91 (m, 1H), 9.50-9.03 (m, 1H), 7.26-7.17 (m, 2H), 7.02 (s, 1H), 6.20-5.77 (m, 1H), 5.38-4.63 (m, 2H), 4.45-4.21 (m, 1H), 4.21-3.90 (m, 2H), 3.21-2.84 (m, 2H), 1.22 (d, J=7.0 Hz, 3H).

### Example 152

### 5-(4-chloro-5-fluoro-1H-indole-2-carbonyl)-N-[(2R)-1,1-difluoropropan-2-yl]-4H,5H,6H,7H-[1,2]oxazolo[4,3-c]pyridine-3-carboxamide

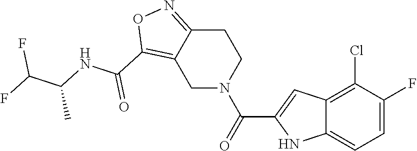 [SMILES: C[C@@H](NC(=O)c1onc2c1CN(C(=O)c1cc3c(Cl)c(F)ccc3[nH]1)CC2)C(F)F]

Rt 1.64 mins (Method H) m/z 439/441 [M+H]⁺

1H NMR (400 MHz, DMSO) δ 12.23-12.09 (m, 1H), 9.33-9.14 (m, 1H), 7.42 (dd, J=8.9, 4.0 Hz, 1H), 7.25 (t, J=9.4 Hz, 1H), 6.93 (s, 1H), 6.17-5.81 (m, 1H), 5.40-4.65 (m, 2H), 4.44-4.24 (m, 1H), 4.13-3.86 (m, 2H), 3.20-2.86 (m, 2H), 1.22 (d, J=7.0 Hz, 3H).

### Example 153

### N-[(2R)-1,1-difluoropropan-2-yl]-5-(1H-indole-2-carbonyl)-6-methyl-4H,5H,6H,7H-[1,2]oxazolo[4,3-c]pyridine-3-carboxamide

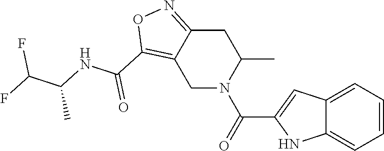 [SMILES: CC1Cc2noc(C(=O)N[C@H](C)C(F)F)c2CN1C(=O)c1cc2ccccc2[nH]1]

Rt 1.57 mins (Method H) m/z 401 [M+H]⁺

1H NMR (400 MHz, DMSO) δ 12.09-11.12 (m, 1H), 9.64-8.92 (m, 1H), 7.65 (d, J=8.0 Hz, 1H), 7.44 (d, J=8.2 Hz, 1H), 7.21 (t, J=7.6 Hz, 1H), 7.07 (t, J=7.4 Hz, 1H), 6.91 (s, 1H), 6.19-5.83 (m, 1H), 5.45 (dd, J=18.0, 4.5 Hz, 1H), 5.30-5.19 (m, 1H), 4.76-4.16 (m, 2H), 3.25-3.01 (m, 1H), 2.92 (d, J=16.4 Hz, 1H), 1.27-1.16 (m, 6H).

### Example 154

### 5-(1H-indole-2-carbonyl)-6-methyl-N-[(2R)-1,1,1-trifluoropropan-2-yl]-4H,5H,6H,7H-[1,2]oxazolo[4,3-c]pyridine-3-carboxamide

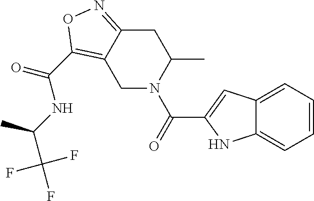 [SMILES: CC1Cc2noc(C(=O)N[C@H](C)C(F)(F)F)c2CN1C(=O)c1cc2ccccc2[nH]1]

Rt 1.65 mins (Method H) m/z 419 [M+H]⁺

1H NMR (400 MHz, DMSO) δ 11.66 (s, 1H), 9.59 (s, 1H), 7.65 (d, J=8.0 Hz, 1H), 7.44 (d, J=8.2 Hz, 1H), 7.24-7.17 (m, 1H), 7.11-7.03 (m, 1H), 6.91 (s, 1H), 5.45 (dd, J=18.1, 5.0 Hz, 1H), 5.31-5.21 (m, 1H), 4.89-4.71 (m, 1H), 4.71-4.23 (m, 1H), 3.24-3.00 (m, 1H), 2.93 (d, J=16.4 Hz, 1H), 1.41-1.31 (m, 3H), 1.24-1.16 (m, 3H).

### Example 155

### 5-(5,6-difluoro-1H-indole-2-carbonyl)-N-[(2R)-1,1-difluoropropan-2-yl]-4H,5H,6H,7H-[1,2]oxazolo[4,3-c]pyridine-3-carboxamide

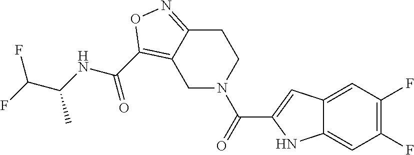 [SMILES: C[C@@H](NC(=O)c1onc2c1CN(C(=O)c1cc3cc(F)c(F)cc3[nH]1)CC2)C(F)F]

Rt 1.57 mins (Method H) m/z 423 [M+H]⁺

1H NMR (400 MHz, DMSO) δ 12.45-11.25 (m, 1H), 9.86-8.72 (m, 1H), 7.72-7.59 (m, 1H), 7.35 (dd, J=11.0, 7.0 Hz, 1H), 6.94 (s, 1H), 6.18-5.82 (m, 1H), 5.26-4.71 (m, 2H), 4.44-4.24 (m, 1H), 4.14-3.89 (m, 2H), 3.15-2.85 (m, 2H), 1.22 (d, J=7.0 Hz, 3H).

### Example 156

### 5-(6-chloro-5-fluoro-1H-indole-2-carbonyl)-N-[(2R)-1,1-difluoropropan-2-yl]-4H,5H,6H,7H-[1,2]oxazolo[4,3-c]pyridine-3-carboxamide

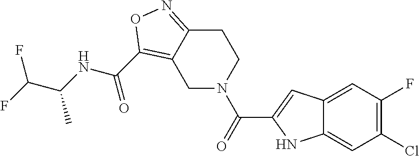 [SMILES: C[C@@H](NC(=O)c1onc2c1CN(C(=O)c1cc3cc(F)c(Cl)cc3[nH]1)CC2)C(F)F]

Rt 1.65 mins (Method H) m/z 439/441 [M+H]⁺

1H NMR (400 MHz, DMSO) δ 11.93 (s, 1H), 9.25 (s, 1H), 7.66 (d, J=9.9 Hz, 1H), 7.56 (d, J=6.4 Hz, 1H), 6.95 (s, 1H), 6.20-5.80 (m, 1H), 5.34-4.56 (m, 2H), 4.45-4.19 (m, 1H), 4.11-3.86 (m, 2H), 3.23-2.80 (m, 2H), 1.22 (d, J=7.0 Hz, 3H).

Selected compounds of the invention were assayed in capsid assembly and HBV replication assays, as described below and a representative group of these active compounds is shown in Table 1 (capsid assembly assay) and Table 2 (HBV replication assay).

### Biochemical Capsid Assembly Assay

The screening for assembly effector activity was done based on a fluorescence quenching assay published by Zlotnick et al. (2007). The C-terminal truncated core protein containing 149 amino acids of the N-terminal assembly domain fused to a unique cysteine residue at position 150 and was expressed in E. coli using the pET expression system (Merck Chemicals, Darmstadt). Purification of core dimer protein was performed using a sequence of size exclusion chromatography steps. In brief, the cell pellet from 1 L BL21 (DE3) Rosetta2 culture expressing the coding sequence of core protein cloned NdeI/XhoI into expression plasmid pET21b was treated for 1 h on ice with a native lysis buffer (Qproteome Bacterial Protein Prep Kit; Qiagen, Hilden). After a centrifugation step the supernatant was precipitated during 2 h stirring on ice with 0.23 g/ml of solid ammonium sulfate. Following further centrifugation the resulting pellet was resolved in buffer A (100 mM Tris, pH 7.5; 100 mM NaCl; 2 mM DTT) and was subsequently loaded onto a buffer A equilibrated CaptoCore 700 column (GE HealthCare, Frankfurt). The column flow through containing the assembled HBV capsid was dialyzed against buffer N (50 mM NaHCO₃ pH 9.6; 5 mM DTT) before urea was added to a final concentration of 3M to dissociate the capsid into core dimers for 1.5 h on ice. The protein solution was then loaded onto a 1L Sephacryl S300 column. After elution with buffer N core dimer containing fractions were identified by SDS-PAGE and subsequently pooled and dialyzed against 50 mM HEPES pH 7.5; 5 mM DTT. To improve the assembly capacity of the purified core dimers a second round of assembly and disassembly starting with the addition of 5 M NaCl and including the size exclusion chromatography steps described above was performed. From the last chromatography step core dimer containing fractions were pooled and stored in aliquots at concentrations between 1.5 to 2.0 mg/ml at −80° C.

Immediately before labelling the core protein was reduced by adding freshly prepared DTT in a final concentration of 20 mM. After 40 min incubation on ice storage buffer and DTT was removed using a Sephadex G-25 column (GE HealthCare, Frankfurt) and 50 mM HEPES, pH 7.5. For labelling 1.6 mg/ml core protein was incubated at 4° C. and darkness overnight with BODIPY-FL maleimide (Invitrogen, Karlsruhe) in a final concentration of 1 mM. After labelling the free dye was removed by an additional desalting step using a Sephadex G-25 column. Labelled core dimers were stored in aliquots at 4° C. In the dimeric state the fluorescence signal of the labelled core protein is high and is quenched during the assembly of the core dimers to high molecular capsid structures. The screening assay was performed in black 384 well microtiter plates in a total assay volume of 10 μl using 50 mM HEPES pH 7.5 and 1.0 to 2.0 μM labelled core protein. Each screening compound was added in 8 different concentrations using a 0.5 log-unit serial dilution starting at a final concentration of 100 μM, 31.6 μM or 10 μM, In any case the DMSO concentration over the entire microtiter plate was 0.5%. The assembly reaction was started by the injection of NaCl to a final concentration of 300 μM which induces the assembly process to approximately 25% of the maximal quenched signal. 6 min after starting the reaction the fluorescence signal was measured using a Clariostar plate reader (BMG Labtech, Ortenberg) with an excitation of 477 nm and an emission of 525 nm. As 100% and 0% assembly control HEPES buffer containing 2.5 M and 0 M NaCl was used. Experiments were performed thrice in triplicates. EC₅₀ values were calculated by non-linear regression analysis using the Graph Pad Prism 6 software (GraphPad Software, La Jolla, USA).

Determination of HBV DNA from the Supernatants of HepAD38 Cells

The anti-HBV activity was analysed in the stable transfected cell line HepAD38, which has been described to secrete high levels of HBV virion particles (Ladner et al., 1997). In brief, HepAD38 cells were cultured at 37° C. at 5% CO₂ and 95% humidity in 200 μl maintenance medium, which was Dulbecco's modified Eagle's medium/Nutrient Mixture F-12 (Gibco, Karlsruhe), 10% fetal bovine serum (PAN Biotech Aidenbach) supplemented with 50 μg/ml penicillin/streptomycin (Gibco, Karlsruhe), 2 mM L-glutamine (PAN Biotech, Aidenbach), 400 μg/ml G418 (AppliChem, Darmstadt) and 0.3 μg/ml tetracycline. Cells were subcultured once a week in a 1:5 ratio, but were usually not passaged more than ten times. For the assay 60,000 cells were seeded in maintenance medium without any tetracycline into each well of a 96-well plate and treated with serial half-log dilutions of test compound. To minimize edge effects the outer 36 wells of the plate were not used but were filled with assay medium. On each assay plate six wells for the virus control (untreated HepAD38 cells) and six wells for the cell control (HepAD38 cells treated with 0.3 μg/ml tetracycline) were allocated, respectively. In addition, one plate set with reference inhibitors like BAY 41-4109, entecavir, and lamivudine instead of screening compounds were prepared in each experiment. In general, experiments were performed thrice in triplicates. At day 6 HBV DNA from 100 μl filtrated cell culture supernatant (AcroPrep Advance 96 Filter Plate, 0.45 μM Supor membran, PALL GmbH, Dreieich) was automatically purified on the MagNa Pure LC instrument using the MagNA Pure 96 DNA and Viral NA Small Volume Kit (Roche Diagnostics, Mannheim) according to the instructions of the manufacturer. EC₅₀ values were calculated from relative copy numbers of HBV DNA In brief, 5 μl of the 100 μl eluate containing HBV DNA were subjected to PCR LC480 Probes Master Kit (Roche) together with 1 μM antisense primer tgcagaggtgaagcgaagtgcaca, 0.5 μM sense primer gacgtcctttgtttacgtcccgtc, 0.3 μM hybprobes acggggcgcacctctctttacgcgg-FL and LC640-ctccccgtctgtgccttctcatctgc-PH (TIBMolBiol, Berlin) to a final volume of 12.5 μl. The PCR was performed on the Light Cycler 480 real time system (Roche Diagnostics, Mannheim) using the following protocol: Pre-incubation for 1 min at 95° C., amplification: 40 cycles×(10 sec at 95° C., 50 sec at 60° C., 1 sec at 70° C.), cooling for 10 sec at 40° C. Viral load was quantitated against known standards using HBV plasmid DNA of pCH-9/3091 (Nassal et al., 1990, Cell 63: 1357-1363) and the LightCycler 480 SW 1.5 software (Roche Diagnostics, Mannheim) and EC₅₀ values were calculated using non-linear regression with GraphPad Prism 6 (GraphPad Software Inc., La Jolla, USA).

### Cell Viability Assay

Using the AlamarBlue viability assay cytotoxicity was evaluated in HepAD38 cells in the presence of 0.3 μg/ml tetracycline, which blocks the expression of the HBV genome. Assay condition and plate layout were in analogy to the anti-HBV assay, however other controls were used. On each assay plate six wells containing untreated HepAD38 cells were used as the 100% viability control, and six wells filled with assay medium only were used as 0% viability control.

In addition, a geometric concentration series of cycloheximide starting at 60 μM final assay concentration was used as positive control in each experiment. After six days incubation period Alamar Blue Presto cell viability reagent (ThermoFisher, Dreieich) was added in 1/11 dilution to each well of the assay plate. After an incubation for 30 to 45 min at 37° C. the fluorescence signal, which is proportional to the number of living cells, was read using a Tecan Spectrafluor Plus plate reader with an excitation filter 550 nm and emission filter 595 nm, respectively. Data were normalized into percentages of the untreated control (100% viability) and assay medium (0% viability) before CC₅₀ values were calculated using non-linear regression and the GraphPad Prism 6.0 (GraphPad Software, La Jolla, USA). Mean EC₅₀ and CC₅₀ values were used to calculate the selectivity index (SI=CC₅₀/EC₅₀) for each test compound.

In vivo efficacy models HBV research and preclinical testing of antiviral agents are limited by the narrow species- and tissue-tropism of the virus, the paucity of infection models available and the restrictions imposed by the use of chimpanzees, the only animals fully susceptible to HBV infection.

Alternative animal models are based on the use of HBV-related hepadnaviruses and various antiviral compounds have been tested in woodchuck hepatitis virus (WHV) infected woodchucks or in duck hepatitis B virus (DHBV) infected ducks or in woolly monkey HBV (WM-HBV) infected tupaia (overview in Dandri et al., 2017, Best Pract Res Clin Gastroenterol 31, 273-279). However, the use of surrogate viruses has several limitations. For example is the sequence homology between the most distantly related DHBV and HBV is only about 40% and that is why core protein assembly modifiers of the HAP family appeared inactive on DHBV and WHV but efficiently suppressed HBV (Campagna et al., 2013, J. Virol. 87, 6931-6942). Mice are not HBV permissive but major efforts have focused on the development of mouse models of HBV replication and infection, such as the generation of mice transgenic for the human HBV (HBV tg mice), the hydrodynamic injection (HDI) of HBV genomes in mice or the generation of mice having humanized livers and/or humanized immune systems and the intravenous injection of viral vectors based on adenoviruses containing HBV genomes (Ad-HBV) or the adenoassociated virus (AAV-HBV) into immune competent mice (overview in Dandri et al., 2017, Best Pract Res Clin Gastroenterol 31, 273-279). Using mice transgenic for the full HBV genome the ability of murine hepatocytes to produce infectious HBV virions could be demonstrated (Guidotti et al., 1995, J. Virol., 69: 6158-6169). Since transgenic mice are immunological tolerant to viral proteins and no liver injury was observed in HBV-producing mice, these studies demonstrated that HBV itself is not cytopathic. HBV transgenic mice have been employed to test the efficacy of several anti-HBV agents like the polymerase inhibitors and core protein assembly modifiers (Weber et al., 2002, Antiviral Research 54 69-78; Julander et al., 2003, Antivir. Res., 59: 155-161), thus proving that HBV transgenic mice are well suitable for many type of preclinical antiviral testing in vivo.

As described in Paulsen et al., 2015, PLOSone, 10: e0144383 HBV-transgenic mice (Tg [HBV1.3 fsX-3′5′]) carrying a frameshift mutation (GC) at position 2916/2917 could be used to demonstrate antiviral activity of core protein assembly modifiers in vivo. In brief, The HBV-transgenic mice were checked for HBV-specific DNA in the serum by qPCR prior to the experiments (see section “Determination of HBV DNA from the supernatants of HepAD38 cells”). Each treatment group consisted of five male and five female animals approximately 10 weeks age with a titer of 107-10¹ virions per mL serum. Compounds were formulated as a suspension in a suitable vehicle such as 2% DMSO/98% tylose (0.5% Methylcellulose/99.5% PBS) or 50% PEG400 and administered per os to the animals one to three times/day for a 10 day period. The vehicle served as negative control, whereas 1 μg/kg entecavir in a suitable vehicle was the positive control. Blood was obtained by retro bulbar blood sampling using an Isoflurane Vaporizer. For collection of terminal heart puncture six hours after the last treatment blood or organs, mice were anaesthetized with isoflurane and subsequently sacrificed by CO₂ exposure. Retro bulbar (100-150 μl) and heart puncture (400-500 μl) blood samples were collected into a Microvette 300 LH or Microvette 500 LH, respectively, followed by separation of plasma via centrifugation (10 min, 2000 g, 4° C.). Liver tissue was taken and snap frozen in liquid N₂. All samples were stored at −80° C. until further use. Viral DNA was extracted from 50 μl plasma or 25 mg liver tissue and eluted in 50 μl AE buffer (plasma) using the DNeasy 96 Blood & Tissue Kit (Qiagen, Hilden) or 320 μl AE buffer (liver tissue) using the DNeasy Tissue Kit (Qiagen, Hilden) according to the manufacturer's instructions. Eluted viral DNA was subjected to qPCR using the LightCycler 480 Probes Master PCR kit (Roche, Mannheim) according to the manufacturer's instructions to determine the HBV copy number. HBV specific primers used included the forward primer 5′-CTG TAC CAA ACC TTC GGA CGG-3′, the reverse primer 5′-AGG AGA AAC GGG CTG AGG C-3′ and the FAM labelled probe FAM-CCA TCA TCC TGG GCT TTC GGA AAA TT-BBQ. One PCR reaction sample with a total volume of 20 μl contained 5 μl DNA eluate and 15 μl master mix (comprising 0.3p M of the forward primer, 0.3 μM of the reverse primer, 0.15 μM of the FAM labelled probe). qPCR was carried out on the Roche LightCycler1480 using the following protocol: Pre-incubation for 1 min at 95° C., amplification: (10 sec at 95° C., 50 sec at 60° C., 1 sec at 70° C.)×45 cycles, cooling for 10 sec at 40° C. Standard curves were generated as described above. All samples were tested in duplicate. The detection limit of the assay is ˜50 HBV DNA copies (using standards ranging from 250-2.5×107 copy numbers). Results are expressed as HBV DNA copies/10p plasma or HBV DNA copies/100 ng total liver DNA (normalized to negative control).

It has been shown in multiple studies that not only transgenic mice are a suitable model to proof the antiviral activity of new chemical entities in vivo the use of hydrodynamic injection of HBV genomes in mice as well as the use of immune deficient human liver chimeric mice infected with HBV positive patient serum have also frequently used to profile drugs targeting HBV (Li et al., 2016, Hepat. Mon. 16: e34420; Qiu et al., 2016, J. Med. Chem. 59: 7651-7666; Lutgehetmann et al., 2011, Gastroenterology, 140: 2074-2083). In addition chronic HBV infection has also been successfully established in immunecompetent mice by inoculating low doses of adenovirus- (Huang et al., 2012, Gastroenterology 142: 1447-1450) or adeno-associated virus (AAV) vectors containing the HBV genome (Dion et al., 2013, J Virol. 87: 5554-5563). This models could also be used to demonstrate the in vivo antiviral activity of novel anti-HBV agents.

TABLE 1
Capsid assembly assay
In Table 1, “A” represents an IC₅₀ < 5 μM;
“B” represents 5 μM < IC₅₀ < 10 μM;
“C” represents IC₅₀ < 100 μM

| | Assembly |
|---|---|
| Example | activity |
| Example 2 | A |
| Example 4 | A |
| Example 5 | A |
| Example 6 | A |
| Example 7 | A |
| Example 8 | A |
| Example 10 | A |
| Example 11 | A |
| Example 12 | A |
| Example 13 | A |
| Example 14 | A |
| Example 15 | A |
| Example 16 | A |
| Example 17 | A |
| Example 18 | A |
| Example 19 | C |
| Example 20 | A |
| Example 21 | A |
| Example 22 | A |
| Example 23 | A |
| Example 24 | A |
| Example 26 | A |
| Example 29 | C |
| Example 30 | A |
| Example 31 | A |
| Example 32 | A |
| Example 33 | A |
| Example 34 | A |
| Example 35 | A |
| Example 36 | A |
| Example 37 | A |
| Example 38 | A |
| Example 39 | A |
| Example 40 | A |
| Example 41 | A |
| Example 42 | A |
| Example 43 | A |
| Example 44 | A |
| Example 45 | A |
| Example 46 | A |
| Example 47 | A |
| Example 48 | A |
| Example 49 | A |
| Example 50 | A |
| Example 51 | A |
| Example 52 | A |
| Example 53 | A |
| Example 54 | A |
| Example 55 | A |
| Example 56 | B |
| Example 57 | A |
| Example 58 | A |
| Example 59 | A |
| Example 60 | A |
| Example 61 | A |
| Example 62 | A |
| Example 63 | A |
| Example 64 | A |
| Example 65 | A |
| Example 66 | A |
| Example 67 | A |
| Example 68 | A |
| Example 69 | A |
| Example 70 | A |
| Example 71 | A |
| Example 72 | A |
| Example 73 | A |
| Example 74 | A |
| Example 75 | A |
| Example 76 | A |
| Example 77 | A |
| Example 78 | A |
| Example 79 | A |
| Example 80 | A |
| Example 81 | A |
| Example 82 | A |
| Example 83 | A |
| Example 84 | A |
| Example 85 | A |
| Example 86 | A |
| Example 87 | A |
| Example 88 | A |
| Example 89 | A |
| Example 90 | A |
| Example 95 | A |
| Example 96 | A |
| Example 97 | A |
| Example 98 | A |
| Example 101 | A |
| Example 102 | A |
| Example 103 | A |
| Example 104 | A |
| Example 105 | A |
| Example 106 | A |
| Example 107 | A |
| Example 108 | A |
| Example 109 | A |
| Example 110 | A |
| Example 111 | A |
| Example 112 | A |
| Example 115 | A |
| Example 118 | A |
| Example 120 | A |
| Example 121 | A |
| Example 122 | A |
| Example 123 | A |
| Example 124 | B |
| Example 125 | A |
| Example 132 | A |
| Example 133 | A |
| Example 134 | A |
| Example 136 | A |
| Example 137 | A |
| Example 138 | A |
| Example 139 | A |
| Example 140 | A |
| Example 141 | A |
| Example 143 | A |
| Example 144 | A |
| Example 145 | A |
| Example 146 | A |
| Example 147 | A |
| Example 148 | A |
| Example 149 | A |

TABLE 2
HBV Replication assay
In Table 1,
“+++” represents an EC₅₀ < 1 μM;
“++” represents 1 μM < EC₅₀ < 10 μM;
“+” represents EC₅₀ < 100 μM

| | Cell |
|---|---|
| Example | activity |
| Example 1 | ++ |
| Example 2 | +++ |
| Example 4 | +++ |
| Example 5 | +++ |
| Example 6 | +++ |
| Example 7 | +++ |
| Example 8 | +++ |
| Example 9 | +++ |
| Example 10 | + |
| Example 11 | + |
| Example 12 | +++ |
| Example 13 | ++ |
| Example 14 | +++ |
| Example 15 | +++ |
| Example 16 | +++ |
| Example 17 | +++ |
| Example 18 | +++ |
| Example 21 | +++ |
| Example 22 | +++ |
| Example 23 | +++ |
| Example 24 | ++ |
| Example 25 | ++ |
| Example 26 | ++ |
| Example 30 | ++ |
| Example 31 | + |
| Example 33 | +++ |
| Example 34 | ++ |
| Example 35 | +++ |
| Example 37 | +++ |
| Example 38 | +++ |
| Example 39 | +++ |
| Example 40 | +++ |
| Example 41 | ++ |
| Example 42 | ++ |
| Example 43 | ++ |
| Example 45 | ++ |
| Example 46 | +++ |
| Example 47 | +++ |
| Example 48 | +++ |
| Example 49 | ++ |
| Example 50 | +++ |
| Example 51 | +++ |
| Example 52 | +++ |
| Example 53 | +++ |
| Example 54 | +++ |
| Example 55 | ++ |
| Example 57 | ++ |
| Example 60 | ++ |
| Example 61 | ++ |
| Example 62 | + |
| Example 63 | +++ |
| Example 64 | +++ |
| Example 68 | +++ |
| Example 69 | +++ |
| Example 74 | +++ |
| Example 75 | +++ |
| Example 76 | +++ |
| Example 77 | +++ |
| Example 78 | +++ |
| Example 79 | +++ |
| Example 80 | +++ |
| Example 81 | +++ |
| Example 82 | +++ |
| Example 83 | +++ |
| Example 84 | +++ |
| Example 85 | +++ |
| Example 86 | +++ |
| Example 87 | +++ |
| Example 88 | +++ |
| Example 89 | +++ |
| Example 90 | +++ |
| Example 91 | NT |
| Example 95 | +++ |
| Example 96 | +++ |
| Example 97 | +++ |
| Example 98 | ++ |
| Example 103 | +++ |
| Example 104 | +++ |
| Example 105 | +++ |
| Example 106 | +++ |
| Example 107 | +++ |
| Example 108 | +++ |
| Example 109 | +++ |
| Example 110 | +++ |

1. Compound of Formula I 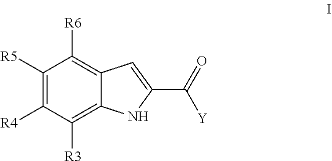
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising 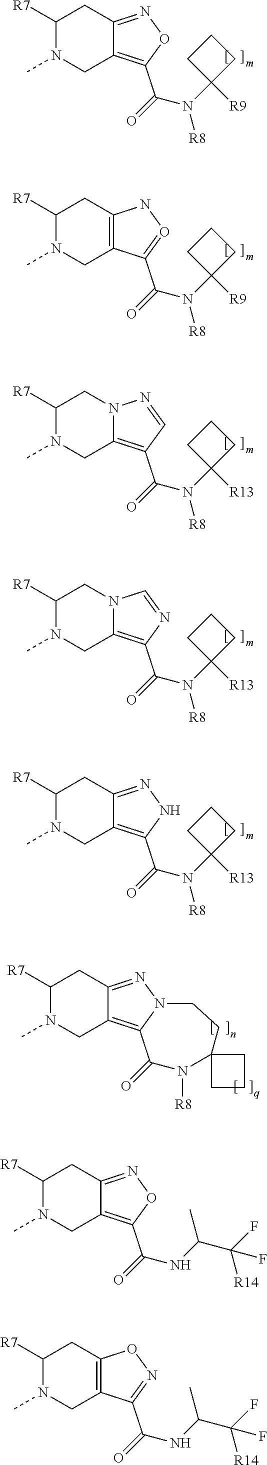
R7 is selected from the group comprising H, D, and C1-C6-alkyl
R8 is selected from the group comprising H, methyl, CD₃ ethyl, 2,2-difluoroethyl, 2,2,2-trifluoroethyl, 2-hydroxyethyl, CH₂CH₂—O—CH₂—C6-aryl, CH₂CH₂—O—C1-C3-alkyl, CH₂CH₂-N-(C1-C3-alkyl)₂, CH₂CH₂OCF₃, CH₂—C(O)—O—C1-C3-alkyl, 2-(4-methylpiperazin-1-yl)ethyl, 2-(morpholin-4-yl)ethyl and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, 1,3-dioxanyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂—O—C1-C3-alkyl optionally substituted with 1, 2 or 3 groups each independently selected from C1-C4-alkyl, OH, OCHF₂, OCF₃, carboxy, amino and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, CH₂-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
R14 is H or F
m is 0 or 1
n is 0, 1 or 2
q is 0 or 1,
wherein the dashed line is a covalent bond between C(O) and Y,
or a pharmaceutically acceptable salt thereof or a solvate of a compound of Formula I or the pharmaceutically acceptable salt thereof or a prodrug of a compound of Formula I or a pharmaceutically acceptable salt or a solvate thereof.

2. A compound of Formula I according to claim 1 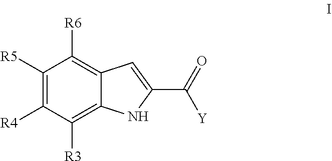
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising 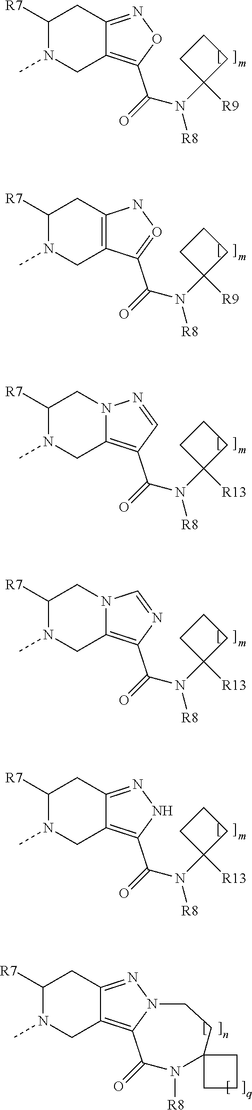
R7 is selected from the group comprising H, D, and C1-C6-alkyl
R8 is selected from the group comprising H, methyl, CD₃ ethyl, 2,2-difluoroethyl, 2,2,2-trifluoroethyl, 2-hydroxyethyl, and cyclopropyl
R9 is selected from the group comprising H, C1-C6-alkyl, phenyl, pyridyl, pyrimidinyl, pyrazinyl, pyridazinyl, triazinyl, oxazolyl, isoxazolyl, imidazolyl, pyrazolyl, CH₂OH, CH₂—O—C6-aryl, CH₂CH₂OH, CH₂—O—CH₂CH₂CH₂OH, CH₂—O—CH₂CH₂OH, CH₂OCHF₂, CH₂—O—C3-C5-cycloalkyl, CH₂—O—C(O)—C6-aryl, and CH₂—O—C1-C3-alkyl optionally substituted with 1, 2 or 3 groups each independently selected from C1-C4-alkyl, OH, OCHF₂, OCF₃, carboxy and halo
R8 and R9 are optionally connected to form a spirocyclic ring system consisting of 2 or 3 C3-C7 rings, optionally substituted with 1, 2, or 3 groups selected from OH, OCHF₂, OCF₃ carboxy and halo
R13 is selected from the group comprising CH₂—O—CH₂CH₂CH₂OH, CH₂—O—CH₂CH₂OH, CH₂—O—C6-aryl, CH₂—O-carboxyphenyl, carboxyphenyl, carboxypyridyl, carboxypyrimidinyl, carboxypyrazinyl, carboxypyridazinyl, carboxytriazinyl, carboxyoxazolyl, carboxyimidazolyl, carboxypyrazolyl, or carboxyisoxazolyl optionally substituted with 1, 2 or 3 groups each independently selected from the group C1-C4-alkyl and halo
m is 0 or 1
n is 0, 1 or 2
q is 0 or 1,
wherein the dashed line is a covalent bond between C(O) and Y,
or a pharmaceutically acceptable salt thereof or a solvate of a compound of Formula I or the pharmaceutically acceptable salt thereof or a prodrug of a compound of Formula I or a pharmaceutically acceptable salt or a solvate thereof.

3. A compound of Formula I according to claim 1 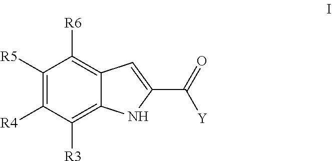
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising 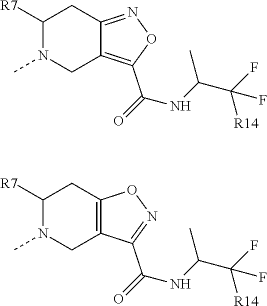
R7 is selected from the group comprising H, D, and C1-C6-alkyl
R14 is H or F
wherein the dashed line is a covalent bond between C(O) and Y,
or a pharmaceutically acceptable salt thereof or a solvate of a compound of Formula I or the pharmaceutically acceptable salt thereof or a prodrug of a compound of Formula I or a pharmaceutically acceptable salt or a solvate thereof.

4. A compound of Formula I according to claim 1 that is a compound of Formula IIb 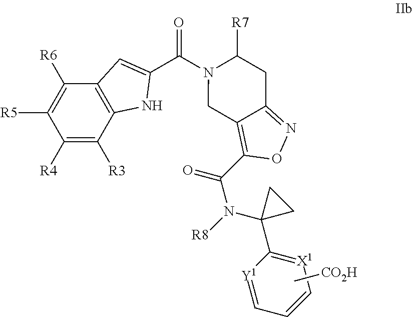
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X¹ and Y¹ are for each position independently selected from CH and N,
or a pharmaceutically acceptable salt thereof or a solvate of a compound of Formula IIb or the pharmaceutically acceptable salt thereof or a prodrug of a compound of Formula IIb or a pharmaceutically acceptable salt or a solvate thereof.

5. A compound of Formula I according to claim 1 that is a compound of Formula IIc 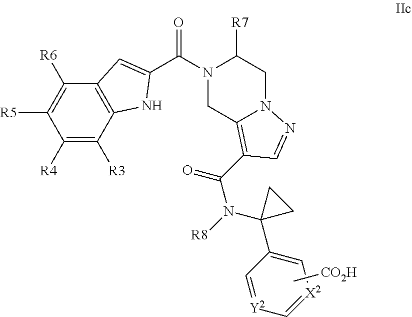
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X² and Y² are for each position independently selected from CH and N, or a pharmaceutically acceptable salt thereof or a solvate of a compound of Formula IIc or the pharmaceutically acceptable salt thereof or a prodrug of a compound of Formula JIc or a pharmaceutically acceptable salt or a solvate thereof.

6. A compound of Formula I according to claim 1 that is a compound of Formula IIa 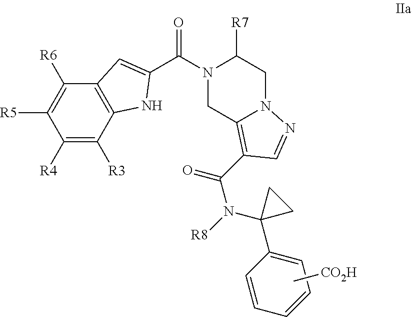
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl,
or a pharmaceutically acceptable salt thereof or a solvate of a compound of Formula IIa or the pharmaceutically acceptable salt thereof or a prodrug of a compound of Formula IIa or a pharmaceutically acceptable salt or a solvate thereof.

7. A compound of Formula I according to claim 1 that is a compound of Formula IIIb 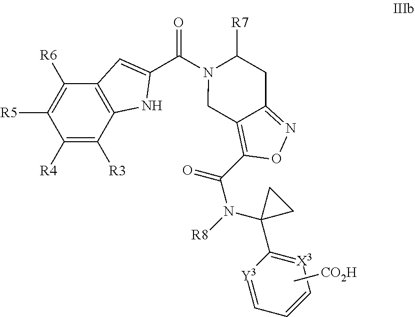
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X³ and Y³ are for each position independently selected from CH and N,
or a pharmaceutically acceptable salt thereof or a solvate of a compound of Formula IIIb or the pharmaceutically acceptable salt thereof or a prodrug of a compound of Formula 11b or a pharmaceutically acceptable salt or a solvate thereof.

8. A compound of Formula I according to claim 1 that is a compound of Formula IIIc 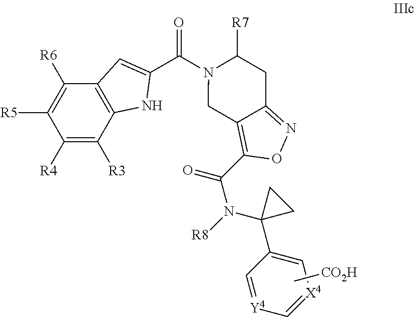
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁴ and Y⁴ are for each position independently selected from CH and N, or a pharmaceutically acceptable salt thereof or a solvate of a compound of Formula IIIc or the pharmaceutically acceptable salt thereof or a prodrug of a compound of Formula IIIc or a pharmaceutically acceptable salt or a solvate thereof.

9. A compound of Formula I according to claim 1 that is a compound of Formula IIIa 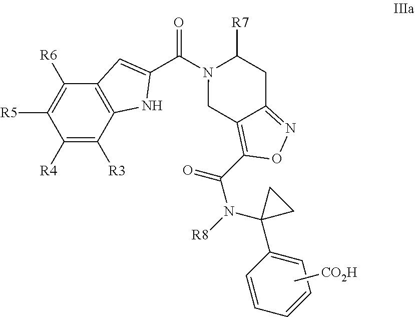
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl,
or a pharmaceutically acceptable salt thereof or a solvate of a compound of Formula IIIa or the pharmaceutically acceptable salt thereof or a prodrug of a compound of Formula IIIa or a pharmaceutically acceptable salt or a solvate thereof.

10. A compound of Formula I according to claim 1 that is a compound of Formula IVb 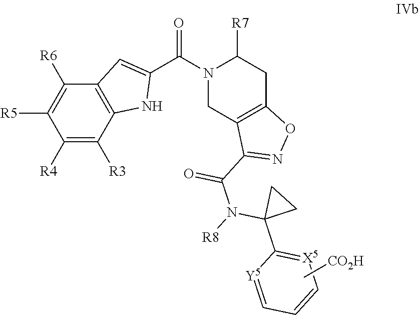
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁵ and Y⁵ are for each position independently selected from CH and N, or a pharmaceutically acceptable salt thereof or a solvate of a compound of Formula IVb or the pharmaceutically acceptable salt thereof or a prodrug of a compound of Formula IVb or a pharmaceutically acceptable salt or a solvate thereof.

11. A compound of Formula I according to claim 1 that is a compound of Formula IVc 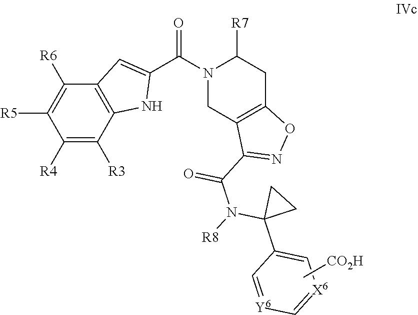
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁶ and Y⁶ are for each position independently selected from CH and N,
or a pharmaceutically acceptable salt thereof or a solvate of a compound of Formula IVc or the pharmaceutically acceptable salt thereof or a prodrug of a compound of Formula IVc or a pharmaceutically acceptable salt or a solvate thereof.

12. A compound of Formula I according to claim 1 that is a compound of Formula IVa 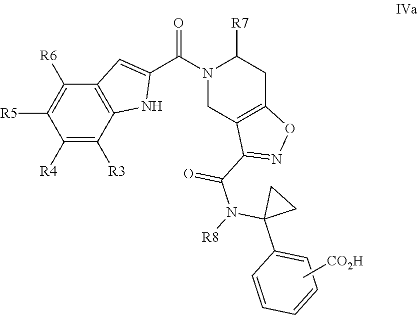
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl,
or a pharmaceutically acceptable salt thereof or a solvate of a compound of Formula IVa or the pharmaceutically acceptable salt thereof or a prodrug of a compound of Formula IVa or a pharmaceutically acceptable salt or a solvate thereof.

13. A compound of Formula I according to claim 1 that is a compound of Formula Vb 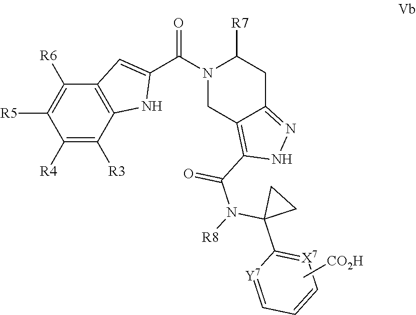
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁷ and Y⁷ are for each position independently selected from CH and N,
or a pharmaceutically acceptable salt thereof or a solvate of a compound of Formula Vb or the pharmaceutically acceptable salt thereof or a prodrug of a compound of Formula Vb or a pharmaceutically acceptable salt or a solvate thereof.

14. A compound of Formula I according to claim 1 that is a compound of Formula Vc 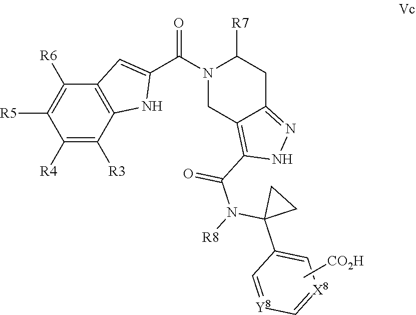
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁸ and Y⁸ are for each position independently selected from CH and N,
or a pharmaceutically acceptable salt thereof or a solvate of a compound of Formula Vc or the pharmaceutically acceptable salt thereof or a prodrug of a compound of Formula Vc or a pharmaceutically acceptable salt or a solvate thereof.

15. A compound of Formula I according to claim 1 that is a compound of Formula Va 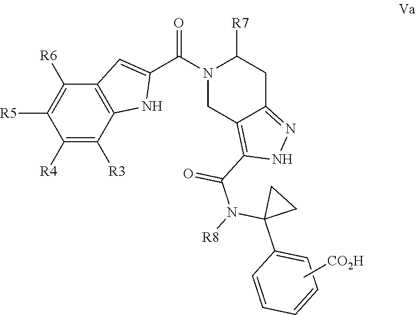
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl,
or a pharmaceutically acceptable salt thereof or a solvate of a compound of Formula Va or the pharmaceutically acceptable salt thereof or a prodrug of a compound of Formula Va or a pharmaceutically acceptable salt or a solvate thereof.

16. A compound of Formula I according to claim 1 that is a compound of Formula VIb 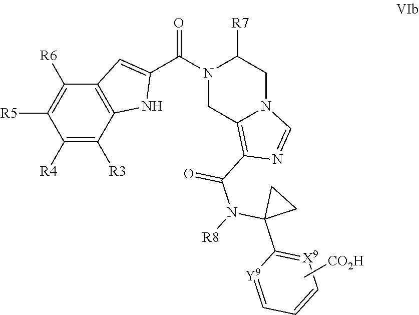
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X⁹ and Y⁹ are for each position independently selected from CH and N,
or a pharmaceutically acceptable salt thereof or a solvate of a compound of Formula VIb or the pharmaceutically acceptable salt thereof or a prodrug of a compound of Formula VIb or a pharmaceutically acceptable salt or a solvate thereof.

17. A compound of Formula I according to claim 1 that is a compound of Formula VIc 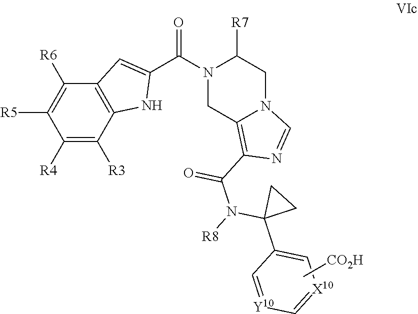
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
X¹⁰ and Y¹⁰ are for each position independently selected from CH and N,
or a pharmaceutically acceptable salt thereof or a solvate of a compound of Formula VIc or the pharmaceutically acceptable salt thereof or a prodrug of a compound of Formula VIc or a pharmaceutically acceptable salt or a solvate thereof.

18. A compound of Formula I according to claim 1 that is a compound of Formula VIa 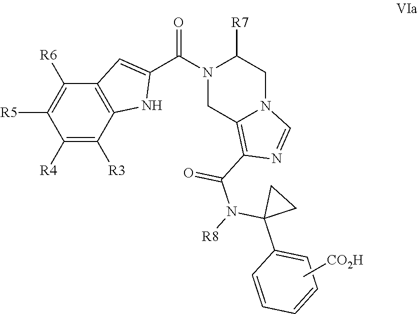
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl,
or a pharmaceutically acceptable salt thereof or a solvate of a compound of Formula VIa or the pharmaceutically acceptable salt thereof or a prodrug of a compound of Formula VIa or a pharmaceutically acceptable salt or a solvate thereof.

19. A compound of Formula I according to claim 1 that is a compound of Formula VII 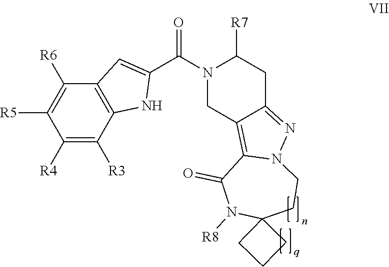
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C6-alkyl
R8 is selected from the group comprising H, methyl, CD₃, ethyl, 2,2-difluoroethyl, 2-hydroxyethyl, cyclopropyl, and 2,2,2-trifluoroethyl
q is 0, or 1
n is 0, 1 or 2,
or a pharmaceutically acceptable salt thereof or a solvate of a compound of Formula VII or the pharmaceutically acceptable salt thereof or a prodrug of a compound of Formula VII or a pharmaceutically acceptable salt or a solvate thereof.

20. A compound of Formula I according to claim 1, that is a compound of Formula IX 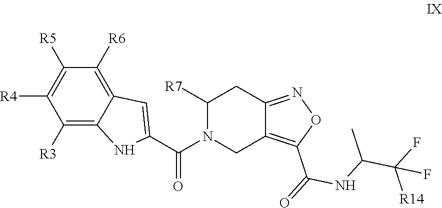
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D, and C1-C6-alkyl
R14 is H or F
or a pharmaceutically acceptable salt thereof or a solvate of a compound of Formula IX or the pharmaceutically acceptable salt thereof or a prodrug of a compound of Formula IX or a pharmaceutically acceptable salt or a solvate thereof.

21. A compound of Formula I according to claim 1 that is a compound of Formula X 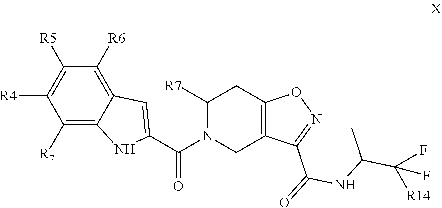
in which
R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D, and C1-C6-alkyl
R14 is H or F
or a pharmaceutically acceptable salt thereof or a solvate of a compound of Formula X or the pharmaceutically acceptable salt thereof or a prodrug of a compound of Formula X or a pharmaceutically acceptable salt or a solvate thereof.

22. A compound of Formula I according to claim 1,
or a pharmaceutically acceptable salt thereof or a solvate of a compound of Formula I or the pharmaceutically acceptable salt thereof or a prodrug of a compound of Formula I or a pharmaceutically acceptable salt or a solvate thereof,
wherein the prodrug is selected from the group consisting of esters and amides, preferably alkyl esters of fatty acids.

23. (canceled)

24. A pharmaceutical composition comprising a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof, together with a pharmaceutically acceptable carrier.

25. A method of treating an HBV infection in an individual in need thereof, comprising administering to the individual a therapeutically effective amount of a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof.

26. A method for the preparation of a compound of Formula I as defined in claim 1 by reacting a compound of Formula VIII 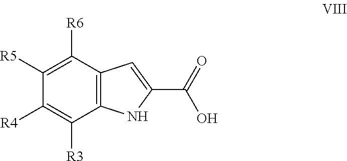
in which R3, R4, R5 and R6 are as defined in claim 1, with a compound selected from 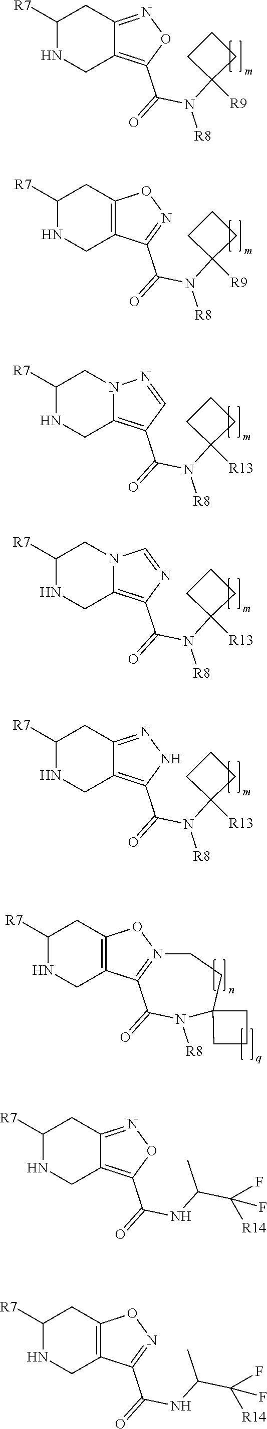
in which R7, R8, R9, R13, R14, m, n and q are as defined in claim 1.

27. A method for the preparation of a compound of Formula I according to claim 26, wherein a compound of Formula VIII 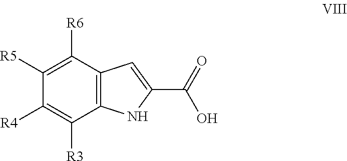
in which R3, R4, R5, and R6, are for each position independently selected from the group comprising H, F, Cl, Br, I, CF₃, CF₂H, C1-C4-alkyl, CF₂CH₃,
reacts with a compound selected from 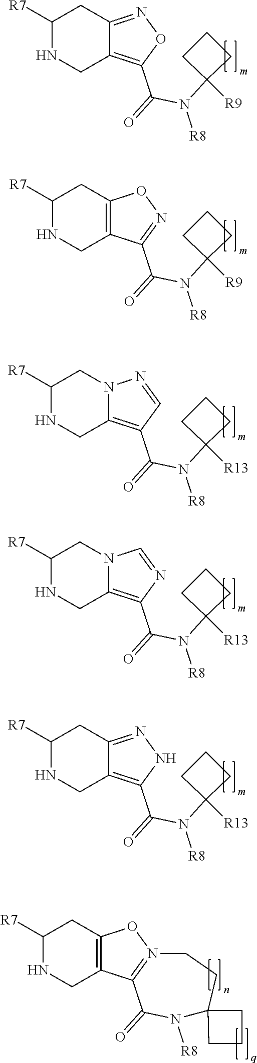
in which R7, R8, R9, R13, m, n and q are as defined in claim 2.
